(19) United States
(12) Patent Application Publication
(10) Pub. No.: US 2023/0355762 A1
(43) Pub. Date: Nov. 9, 2023

(54) COMPOSITIONS AND METHODS FOR THE TREATMENT OF CANCER USING NEXT GENERATION ENGINEERED T CELL THERAPY

(71) Applicant: PACT PHARMA, INC., South San Francisco, CA (US)

(72) Inventors: Michal Mass, Redwood City, CA (US); William Lu, Oakland, CA (US); Kyle Jacoby, Burlingame, CA (US); Michael Mayne Dubreuil, Palo Alto, CA (US); Barbara Sennino, San Francisco, CA (US); Stefanie Mandl-Cashman, San Francisco, CA (US); James S. Byers, III, San Francisco, CA (US)

(73) Assignee: PACT PHARMA, INC., South San Francisco, CA (US)

(21) Appl. No.: 18/306,927

(22) Filed: Apr. 25, 2023

Related U.S. Application Data
(63) Continuation of application No. PCT/US2021/056737, filed on Oct. 27, 2021.
(60) Provisional application No. 63/106,122, filed on Oct. 27, 2020.

(51) Int. Cl. A61K 39/00; C07K 14/725; C12N 15/90; C12N 15/85; C12N 5/0783

(52) U.S. Cl. CPC A61K 39/4632; C07K 14/7051; A61K 39/4611; C12N 15/907; C12N 15/85; C12N 5/0636; C12N 2800/107

(57) ABSTRACT

Compositions comprising and methods for the treatment of cancer using a NeoTCR based cell therapy with a secondary Payload in an expression construct.

### CROSS-REFERENCE TO RELATED APPLICATIONS

This application is a continuation application of International Patent Application No. PCT/US21/56737, filed on Oct. 27, 2021, which claims priority to U.S. Provisional Application No. 63/106,122, filed on Oct. 27, 2020, the contents of each of which are incorporated in their entirety, and to each of which priority is claimed.

### SEQUENCE LISTINGS

A Sequence Listing conforming to the rules of WIPO Standard ST.26 is hereby incorporated by reference. Said Sequence Listing has been filed as an electronic document via PatentCenter in ASCII format encoded as XML. The electronic document, created on Apr. 25, 2023, is entitled “0875200290_ST26.xml”, and is 349,973 bytes in size.

### BACKGROUND OF THE INVENTION

Human cancers are comprised of normal cells that have undergone a genetic or conversion to become abnormal cancer cells. In doing so, cancer cells begin to express proteins and other antigens that are distinct from those expressed by normal cells. These aberrant tumor antigens can be used by the body's innate immune system to specifically target and kill cancer cells. However, cancer cells employ various mechanisms to prevent immune cells from efficiently mounting an attack to effectively kill cancer cells and eradicate cancer. Nonetheless, immune responses do occur and it is possible to isolate and detect individual T cells, for example, that recognize antigens on the cancer cells and have the ability to kill such cancer cells. Accordingly, the antigens on the cancer cells can be detected and T cells can be engineered to recognize and kill cells that express such cancer cells. However, simply engineering an immune cell to detect a cancer antigen (e.g., by engineering the immune cell to express a NeoTCR that is specific to the cancer antigen) to elicit a cascade of events leading to the killing of the cancer antigen-expressing cell may not be sufficient for the treatment of cancers. In certain circumstances, in addition to the expression of the NeoTCR, it is also advantageous to knock in one or more additional genes into the immune cell. Furthermore, expression of NeoTCRs results in personalized therapies for each patient in need thereof. Specifically, each patient will have a different cancer phenotype and thus the NeoTCRs need to be identified in each patient for a cell therapy that is designed specifically for such patient. In order to do this, viral gene-editing methods are not practical or feasible. Instead, non-viral gene-editing methods are needed to efficiently and cost-effectively engineer immune cells for personalized therapies. Accordingly, there is a need to develop methods to express at least one NeoTCR and at least a Payload using non-viral methods.

### SUMMARY OF THE INVENTION

The present disclosure provides cells comprising an exogenous TCR and at least one Payload.

In certain embodiments, the present disclosure provides a cell comprising an exogenous polynucleotide comprising an exogenous enhancer, an insulator, a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising an exogenous enhancer, a pause element, a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising a Woodchuck Hepatitis Virus Posttranscriptional Regulatory Element (WPRE), a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising an insulator, a Woodchuck Hepatitis Virus Posttranscriptional Regulatory Element (WPRE), a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising an insulator, a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising a sequence encoding an exogenous TCR and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising a sequence encoding an exogenous TCR, a first sequence encoding a Payload, and a second sequence encoding a Payload, wherein the exogenous polynucleotide is integrated at an endogenous locus within the genome of the cell, wherein the sequence encoding an exogenous TCR and the first sequence encoding a Payload are under control of an endogenous promoter and the second sequence encoding a Payload is under control of an exogenous promoter.

In certain embodiments, the present disclosure provides a cell comprising an exogenous polynucleotide comprising a sequence encoding an exogenous TCR and a sequence encoding at least one Payload, wherein the exogenous polynucleotide is integrated at an endogenous locus within the genome of the cell and is under control of an endogenous promoter.

In certain embodiments, the insulator is an HS4 insulator or an IS2 insulator. In certain embodiments, the insulator comprises a nucleotide sequence that is at least about 80% identical to the nucleotide sequence set forth in SEQ ID NO: 18 or SEQ ID NO: 19. In certain embodiments, the insulator comprises the nucleotide sequence set forth in SEQ ID NO: 18 or SEQ ID NO: 19.

In certain embodiments, the WPRE comprises a nucleotide sequence that is at least about 80% identical to the nucleotide sequence set forth in SEQ ID NO: 20. In certain embodiments, the WPRE comprises the nucleotide sequence set forth in SEQ ID NO: 20.

In certain embodiments, the exogenous enhancer is a CMV enhancer, a TCRα enhancer, or a TCRβ enhancer. In certain embodiments, the exogenous enhancer is a TCRα enhancer. In certain embodiments, the TCRα enhancer comprises a nucleotide sequence that is at least about 80% identical to the nucleotide sequence set forth in SEQ ID NO: 32. In certain embodiments, the TCRα enhancer comprises the nucleotide sequence set forth in SEQ ID NO: 32. In certain embodiments, the exogenous enhancer is a CMV enhancer. In certain embodiments, the CMV enhancer comprises a nucleotide sequence that is at least about 80% identical to the nucleotide sequence set forth in SEQ ID NO. 33. In certain embodiments, the CMV enhancer comprises the nucleotide sequence set forth in SEQ ID NO: 33.

In certain embodiments, the exogenous promoter is a constitutive promoter. In certain embodiments, the constitutive promoter is an MDN promoter, an EF1α promoter, an ACTB promoter, a PGK promoter, or a U6 promoter. In certain embodiments, the constitutive promoter is an MDN promoter. In certain embodiments, the constitutive promoter is an EF1α promoter. In certain embodiments, the exogenous promoter is an inducible promoter. In certain embodiments, the inducible promoter is an AP1 promoter, an NFAT promoter, an NF-κB promoter, or an NR4A-responsive promoter.

In certain embodiments, the sequence encoding an exogenous TCR comprises a TCRα gene sequence or a TCRβ gene sequence. In certain embodiments, the sequence encoding an exogenous TCR comprises a TCRα gene sequence and a TCRβ gene sequence. In certain embodiments, the sequence encoding an exogenous TCR further comprises a sequence encoding a P2A peptide, a sequence encoding a signal sequence, a sequence encoding a protease cleavage peptide, or a combination thereof. In certain embodiments, the sequence encoding an exogenous TCR comprises a TCRα gene sequence, a TCRβ gene sequence, a sequence encoding a P2A peptide, a sequence encoding a signal sequence, a sequence encoding a protease cleavage peptide, or a combination thereof.

In certain embodiments, the sequence encoding an exogenous TCR comprises, from 5′ end to 3′ end, a first sequence encoding a P2A peptide, a first sequence encoding a signal sequence, a TCRβ gene sequence, a sequence encoding a protease cleavage peptide, a second sequence encoding a P2A peptide, a second sequence encoding a signal sequence, and a TCRα gene sequence. In certain embodiments, the sequence encoding an exogenous TCR comprises, from 5′ end to 3′ end, a first sequence encoding a P2A peptide, a first sequence encoding a signal sequence, a TCRβ gene sequence, a sequence encoding a protease cleavage peptide, a second sequence encoding a P2A peptide, a second sequence encoding a signal sequence, a TCRα gene sequence, and a poly-adenylation sequence. In certain embodiments, the sequence encoding an exogenous TCR comprises, from 5′ end to 3′ end, a first sequence encoding a P2A peptide, a first sequence encoding a signal sequence, a TCRα gene sequence, a sequence encoding a protease cleavage peptide, a second sequence encoding a P2A peptide, a second sequence encoding a signal sequence, a TCRβ gene sequence, and a poly-adenylation sequence.

In certain embodiments, the at least one Payload is selected from the group consisting of a cytokine receptors trap, a ligand trap, an angiogenesis factor, an apoptotic factor, an inhibitory protein, an extracellular matrix modulator, a soluble TCR, a homing signal, an enzyme, a modulator of reactive oxygen species, a competitive ligand inhibitor, a protein that binds to receptors and sterically hinders receptor function, and an inhibitory RNA molecule.

In certain embodiments, the 3′ of the at least one Payload comprises a STOP codon. In certain embodiments, the 3′ of the at least one Payload comprises a sequence encoding a 2A peptide and a sequence encoding a protease cleavage peptide. In certain embodiments, the 3′ of the at least one Payload comprises a poly-adenylation sequence.

In certain embodiments, the at least one Payload is an inhibitory RNA molecule. In certain embodiments, the inhibitory RNA molecule is a shRNA, a miRNA, or a miRNA cluster. In certain embodiments, the inhibitory RNA molecule is a miRNA. In certain embodiments, the miRNA comprises a first flanking sequence and a second flanking sequence. In certain embodiments, the first flanking sequence and the second flanking sequence are derived from miR-155, miR-30, miR-17/92, miR-122, or miR-21. In certain embodiments, the inhibitory RNA molecule is flanked by a splice donor site or a splice acceptor site. In certain embodiments, the inhibitory RNA molecule is flanked by a splice donor site and a splice acceptor site.

In certain embodiments, the sequences encoding a P2A peptide are codon diverged. In certain embodiments, the sequences encoding a protease cleavage peptide are codon diverged. In certain embodiments, the sequences encoding a signal sequence are codon diverged.

In certain embodiments, the exogenous TCR recognizes a cancer neoantigen. In certain embodiments, the neoantigen is a private neoantigen. In certain embodiments, the sequence encoding an exogenous TCR is obtained from a subject.

In certain embodiments, the cell is a primary cell. In certain embodiments, the cell is a patient-derived cell. In certain embodiments, the cell is a lymphocyte. In certain embodiments, the cell is a T cell. In certain embodiments, the cell is a young T cell. In certain embodiments, the cell is CD45RA+, CD62L+, CD28+, CD95−, CCR7+, and CD27+. In certain embodiments, the cell is CD45RA+, CD62L+, CD28+, CD95+, CD27+, CCR7+. In certain embodiments, the cell is CD45RO+, CD62L+, CD28+, CD95+, CCR7+, CD27+, CD127+.

In certain embodiments, the endogenous locus within the genome of the cell is a TCR locus. In certain embodiments, the TCR locus is a TRAC locus or a TRBC locus. In certain embodiments, the TCR locus is a TRAC locus and a TRBC locus. In certain embodiments, the endogenous promoter is a TRAC promoter. In certain embodiments, the endogenous promoter is a TRBC promoter.

In certain embodiments, the cell, the sequence encoding an exogenous TCR, and the sequence of the neoantigen are obtained from the same subject.

In certain embodiments, the present disclosure provides a polynucleotide comprising an exogenous enhancer, an insulator, a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising an exogenous enhancer, a pause element, a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising a Woodchuck Hepatitis Virus Posttranscriptional Regulatory Element (WPRE), a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising an insulator, a Woodchuck Hepatitis Virus Posttranscriptional Regulatory Element (WPRE), a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising an insulator, a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising a first homology arm, a second homology arm, a sequence encoding an exogenous TCR, a first sequence encoding a Payload, and a second sequence encoding a Payload, wherein the first and second homology arms are homologous to a TRAC or TRBC locus and wherein the second sequence encoding a Payload is under control of an exogenous promoter.

In certain embodiments, the present disclosure provides a polynucleotide comprising a sequence encoding a first homology arm, a second homology arm, an exogenous TCR, and a sequence encoding at least one Payload, wherein the first and second homology arms are homologous to a TRAC or TRBC locus.

In certain embodiments, the insulator is an HS4 insulator or an IS2 insulator. In certain embodiments, the insulator comprises a nucleotide sequence that is at least about 80% identical to the nucleotide sequence set forth in SEQ ID NO: 18 or SEQ ID NO: 19. In certain embodiments, the insulator comprises the nucleotide sequence set forth in SEQ ID NO: 18 or SEQ ID NO: 18. In certain embodiments, the WPRE comprises a nucleotide sequence that is at least about 80% identical to the nucleotide sequence set forth in SEQ ID NO: 20. In certain embodiments, the WPRE comprises the nucleotide sequence set forth in SEQ ID NO: 20.

In certain embodiments, the exogenous enhancer is a CMV enhancer, a TCRα enhancer, or a TCRβ enhancer. In certain embodiments, the exogenous enhancer is a TCRα enhancer. In certain embodiments, the TCRα enhancer comprises a nucleotide sequence that is at least about 80% identical to the nucleotide sequence set forth in SEQ ID NO: 32. In certain embodiments, the TCRα enhancer comprises the nucleotide sequence set forth in SEQ ID NO: 32. In certain embodiments, the exogenous enhancer is a CMV enhancer. In certain embodiments, the CMV enhancer comprises a nucleotide sequence that is at least about 80% identical to the nucleotide sequence set forth in SEQ ID NO: 33. In certain embodiments, the CMV enhancer comprises the nucleotide sequence set forth in SEQ ID NO: 33.

In certain embodiments, the exogenous promoter is a constitutive promoter. In certain embodiments, the constitutive promoter is an MDN promoter, an EF1α promoter, an ACTB promoter, a PGK promoter, or a U6 promoter. In certain embodiments, the constitutive promoter is an MDN promoter. In certain embodiments, the constitutive promoter is an EF1α promoter. In certain embodiments, the exogenous promoter is an inducible promoter. In certain embodiments, the inducible promoter is an AP1 promoter, an NFAT promoter, an NF-κB promoter, or an NR4A-responsive promoter.

In certain embodiments, the sequence encoding an exogenous TCR comprises a TCRα gene sequence or a TCRβ gene sequence. In certain embodiments, the sequence encoding an exogenous TCR comprises a TCRα gene sequence and a TCRβ gene sequence. In certain embodiments, the sequence encoding an exogenous TCR further comprises a sequence encoding a P2A peptide, a sequence encoding a signal sequence, a sequence encoding a protease cleavage peptide, or a combination thereof. In certain embodiments, the sequence encoding an exogenous TCR comprises a TCRα gene sequence, a TCRβ gene sequence, a sequence encoding a P2A peptide, a sequence encoding a signal sequence, a sequence encoding a protease cleavage peptide, or a combination thereof.

In certain embodiments, the sequence encoding an exogenous TCR comprises, from 5′ end to 3′ end, a first sequence encoding a P2A peptide, a first sequence encoding a signal sequence, a TCRβ gene sequence, a sequence encoding a protease cleavage peptide, a second sequence encoding a P2A peptide, a second sequence encoding a signal sequence, and a TCRα gene sequence. In certain embodiments, the sequence encoding an exogenous TCR comprises, from 5′ end to 3′ end, a first sequence encoding a P2A peptide, a first sequence encoding a signal sequence, a TCRβ gene sequence, a sequence encoding a protease cleavage peptide, a second sequence encoding a P2A peptide, a second sequence encoding a signal sequence, a TCRα gene sequence, and a poly-adenylation sequence. In certain embodiments, the sequence encoding an exogenous TCR comprises, from 5′ end to 3′ end, a first sequence encoding a P2A peptide, a first sequence encoding a signal sequence, a TCRα gene sequence, a sequence encoding a protease cleavage peptide, a second sequence encoding a P2A peptide, a second sequence encoding a signal sequence, a TCRβ gene sequence, and a poly-adenylation sequence. In certain embodiments, the at least one Payload is selected from the group consisting of a cytokine receptors trap, a ligand trap, an angiogenesis factor, an apoptotic factor, an inhibitory protein, an extracellular matrix modulator, a soluble TCR, a homing signal, an enzyme, a modulator of reactive oxygen species, a competitive ligand inhibitor, a protein that binds to receptors and sterically hinders receptor function, and an inhibitory RNA molecule.

In certain embodiments, the 3′ of the at least one Payload comprises a STOP codon. In certain embodiments, the 3′ of the at least one Payload comprises a sequence encoding a 2A peptide and a sequence encoding a protease cleavage peptide. In certain embodiments, the 3′ of the at least one Payload comprises a poly-adenylation sequence.

In certain embodiments, the at least one Payload is an inhibitory RNA molecule. In certain embodiments, the inhibitory RNA molecule is a shRNA, a miRNA, or a miRNA cluster. In certain embodiments, the inhibitory RNA molecule is a miRNA. In certain embodiments, the miRNA comprises a first flanking sequence and a second flanking sequence. In certain embodiments, the first flanking sequence and the second flanking sequence are derived from miR-155, miR-30, miR-17/92, miR-122, or miR-21. In certain embodiments, the inhibitory RNA molecule is flanked by a splice donor site or a splice acceptor site. In certain embodiments, the inhibitory RNA molecule is flanked by a splice donor site and a splice acceptor site.

In certain embodiments, the sequences encoding a P2A peptide are codon diverged. In certain embodiments, the sequences encoding a protease cleavage peptide are codon diverged. In certain embodiments, the sequences encoding a signal sequence are codon diverged. In certain embodiments, the exogenous TCR recognizes a cancer neoantigen. In certain embodiments, the neoantigen is a private neoantigen. In certain embodiments, the sequence encoding an exogenous TCR is obtained from a single subject.

In certain embodiments, the polynucleotide is a circular polynucleotide. In certain embodiments, the circular polynucleotide is a plasmid or a nanoplasmid. In certain embodiments, the polynucleotide is a linear polynucleotide.

In certain embodiments, the present disclosure further provides a vector comprising any one of the polynucleotide disclosed herein. In certain embodiments, the vector is a non-viral vector. In certain embodiments, the present disclosure provides a cell comprising any one of the polynucleotides or vectors disclosed herein.

In certain embodiments, the present disclosure provides a method of modifying a cell. In certain embodiments, the method comprises introducing into the cell any one of the polynucleotides or vectors disclosed herein. In certain embodiments, the method further comprises recombining the polynucleotide or vector into an endogenous locus of the cell.

In certain embodiments, the introducing occurs via electroporation. In certain embodiments, the recombining comprises cleavage of the endogenous locus by a nuclease. In certain embodiments, the recombining further comprises recombination of the polynucleotide into the endogenous locus by homology-directed repair.

In certain embodiments, the nuclease is a Clustered Regularly Interspaced Short Palindromic Repeats (CRISPR) family nuclease, or a functional fragment thereof. In certain embodiments, the nuclease further comprises a gRNA.

In certain embodiments, the method further comprises culturing the cell in the presence of at least one cytokine. In certain embodiments, the at least one cytokine comprises IL2, IL7, IL15, or a combination thereof. In certain embodiments, the at least one cytokine comprises IL7 and IL15.

In certain embodiments, the cell is a primary cell. In certain embodiments, the cell is a patient-derived cell. In certain embodiments, the cell is a lymphocyte. In certain embodiments, the cell is a T cell. In certain embodiments, the cell is a young T cell. In certain embodiments, the cell is CD45RA+, CD62L+, CD28+, CD95−, CCR7+, and CD27+. In certain embodiments, the cell is CD45RA+, CD62L+, CD28+, CD95+, CD27+, CCR7+. In certain embodiments, the cell is CD45RO+, CD62L+, CD28+, CD95+, CCR7+, CD27+, CD127+.

In certain embodiments, the present disclosure also provides a cell modified by any one of the methods disclosed herein.

In certain embodiments, the present disclosure provides a composition comprising an effective amount of the any one of the cells disclosed herein. In certain embodiments, the composition is a pharmaceutical composition that further comprises a pharmaceutically acceptable excipient. In certain embodiments, the composition is administered to a patient in need thereof for the treatment of cancer.

In certain embodiments, the composition comprises a cryopreservation agent. In certain embodiments, the composition comprises serum albumin. In certain embodiments, the composition comprises Plasma-Lyte A, HSA, and CryoStor CS10.

In certain embodiments, the present disclosure provides methods of treating cancer in a subject in need thereof. In certain embodiments, the method comprises administering a therapeutically effective amount of any one of the cells disclosed herein. In certain embodiments, the method comprises administering a therapeutically effective amount of any one of the compositions disclosed herein. In certain embodiments, prior to administering, a non-myeloablative lymphodepletion regimen is administered to the subject.

In certain embodiments, the cancer is a solid tumor or a liquid tumor. In certain embodiments, the solid tumor is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinomas, renal cell cancers, testicular cancer, sarcomas, and colorectal cancer. In certain embodiments, the liquid tumor is selected from the group consisting of follicular lymphoma, leukemia, and multiple myeloma.

In certain embodiments, the present disclosure provides a kit comprising any one of the cells, the polynucleotides, the vectors, or the compositions disclosed herein. In certain embodiments, the kit further comprises written instructions for treating cancer.

### BRIEF DESCRIPTION OF THE DRAWINGS

FIGS. 1A-1C show an example of a NeoE TCR cassette and gene editing methods that can be used to make NeoTCR Products. FIG. 1A shows a schematic representing the general targeting strategy used for integrating neoantigen-specific TCR constructs (NeoTCRs) into the TCRα locus. FIGS. 1B and 1C show a neoantigen-specific TCR construct design used for integrating a NeoTCR into the TCRα locus wherein the cassette is shown with signal sequences (“SS”), protease cleavage sites (“P”), and 2A peptides (“2A”). FIG. 1B shows a target TCRα locus (endogenous TRAC, top panel) and its CRISPR Cas9 target site (horizontal stripes, cleavage site designated by the arrow), and the circular plasmid HR template (bottom panel) with the polynucleotide encoding the NeoTCR, which is located between left and right homology arms (“LHA” and “RHA” respectively) prior to integration. FIG. 1C shows the integrated NeoTCR in the TCRα locus (top panel), the transcribed and spliced NeoTCR mRNA (middle panel), and translation and processing of the expressed NeoTCR (bottom panel).

FIGS. 2A and 2B illustrate representative Secondary Promoter Constructs. FIG. 2A shows a diagram of Format 1 of the Secondary Promoter Constructs used to express a broad selection of knock-in (KI) targets. The aim of Format 1 constructs is to decouple the NeoTCR expression from a secondary KI gene (i.e., a Payload). The Format 1 constructs are modular such that the Payload, the TCR, the promoter, and insulators can be changed to suit the needs of the genetic engineering goals. As shown, the architecture of Format 1 constructs comprises a left homology arm, a linker sequence, a 2A sequence, a signal sequence, a full length TCR beta gene, a protease cleavage sequence, a linker, a 2A sequence, a signal sequence, a full length TCR alpha gene, a poly-A signal sequence, an insulator sequence, a promoter region, an optional Kozak sequence, a Payload of interest, and a right homology arm; all of which are retained within a backbone. FIG. 2B shows an alternate modified version of Format 1 wherein a poly-A signal sequence is incorporated after the Payload. The abbreviations for the elements of Format 1 used in FIGS. 2A and 2B are 2A (2A sequence), SS (signal sequence), P (protease cleavage site), I (insulator).

FIGS. 3A and 3B illustrate representative Secondary Promoter Constructs. FIG. 3A shows a diagram of an example of Format 1 of the Secondary Promoter Constructs used to express a broad selection of KI targets. As shown, this example of a Format 1 construct comprises a left homology arm, a GSG linker, a P2A sequence, an HGH signal sequence, a full length TCR beta gene, a furin cleavage sequence, a GSG linker, a P2A sequence, an HGH signal sequence, a full length TCR alpha gene, a poly-A signal sequence, an insulator sequence, a promoter region, a Kozak sequence, a Payload of interest, and a right homology arm; all of which are retained within a backbone. FIG. 3B shows an alternate example of Format 1 wherein a poly-A signal sequence is incorporated after the Payload. The abbreviations for the Insulator used in Format 1 used in FIGS. 3A and 3B is “I”.

FIGS. 4A-4C illustrate representative Secondary Promoter Constructs. FIG. 4A shows a diagram of Format 2 of the Secondary Promoter Constructs used to express a broad selection of KI targets. The aim of Format 2 constructs is to decouple the NeoTCR expression from a secondary KI gene (i.e., a Payload). The Format 2 constructs are modular such that the Payload, the TCR, the promoter, and insulators can be changed to suit the needs of the genetic engineering goals. As shown, the architecture of Format 2 constructs comprises a left homology arm, a linker sequence, a 2A sequence, a signal sequence, a full length TCR beta gene, a protease cleavage sequence, a linker sequence, a 2A sequence, a signal sequence, a full length TCR alpha gene, a poly-A signal sequence, an insulator sequence, a promoter region, a Kozak sequence (optional), a Payload of interest, a WPRE, a poly-A signal sequence, and a right homology arm; all of which are retained within a backbone. FIG. 4B shows an alternate version of Format 2 wherein the post-transcriptional regulatory element is not included and instead an insulator is incorporated following the second poly-A signal sequence. FIG. 4C shows an alternate version of Format 2 wherein the post-transcriptional regulatory element is not included and no insulator is incorporated following the second poly-A signal sequence. The abbreviations for the elements of Format 2 used in FIGS. 4A-4C are 2A (2A sequence), SS (signal sequence), P (protease cleavage site), I (insulator).

FIGS. 5A-5C illustrate representative Secondary Promoter Constructs. FIG. 5A shows a diagram of an example of Format 2 of the Secondary Promoter Constructs used to express a broad selection of knock-in (KI) targets. As shown, the architecture of this example of Format 2 constructs comprise a left homology arm; GSG linker; P2A sequence; HGH signal sequence; full length TCR beta gene; Furin cleavage sequence; GSG linker; P2A sequence; HGH signal sequence; full length TCR alpha gene; poly-A signal sequence; insulator sequence; promoter region; Kozak sequence; Payload of interest; WPRE; poly-A signal sequence; right homology arm; all of which are retained within a backbone. FIG. 5B shows an example of one of the alternate versions of Format 2 wherein the post-transcriptional regulatory element is not included and instead an insulator is incorporated following the second poly-A signal sequence. FIG. 5C shows an example of one of the alternate versions of Format 2 wherein the post-transcriptional regulatory element is not included and no insulator is incorporated following the second poly-A signal sequence. The abbreviation for the Insulator used in Format 1 used in FIGS. 5A-5C is “I”.

FIG. 6 shows a diagram of Format 3 of the Secondary Promoter Constructs used to express a broad selection of KI targets. The aim of Format 3 constructs is to decouple the NeoTCR expression from a secondary KI gene (i.e., a Payload). The Format 3 constructs are modular such that the Payload, the TCR, the promoter, and insulators can be changed to suit the needs of the genetic engineering goals. As shown, the architecture of Format 3 constructs comprises a left homology arm, a linker sequence, a 2A sequence, a signal sequence, a full length TCR beta gene, a protease cleavage sequence, a linker sequence, a 2A sequence, a signal sequence, a full length TCR alpha gene, a poly-A signal sequence, a WPRE, a Payload of interest, a Kozak sequence (optional), a promoter region, and a right homology arm; all of which are retained within a backbone. The abbreviations for the elements of Format 1 used in FIG. 6 are 2A (2A sequence), SS (signal sequence), P (protease cleavage site), I (insulator).

FIG. 7 shows a diagram of an example of Format 3 of the Secondary Promoter Constructs used to express a broad selection of KI targets. The aim of Format 3 constructs is to decouple the NeoTCR expression from a secondary KI gene (i.e., a Payload). The Format 3 constructs are modular such that the Payload, the TCR, the promoter, and insulators can be changed to suit the needs of the genetic engineering goals. As shown, the architecture of this example of Format 3 constructs comprise a left homology arm, a GSG linker, a P2A sequence, an HGH signal sequence, a full length TCR beta gene, a Furin cleavage sequence, a GSG linker, a P2A sequence, an HGH signal sequence, a full length TCR alpha gene, a poly-A signal sequence, a WPRE, a Payload of interest, a Kozak sequence, a promoter region, and a right homology arm; all of which are retained within a backbone. The abbreviation for the Insulator used in Format 1 used in FIG. 7 is “I”.

FIG. 8 shows a diagram of Format 4 of the Secondary Promoter Constructs used to express a broad selection of KI targets. The aim of Format 4 is to control the expression of the Payload and the TCR off of the endogenous TRAC or TRBC promoter. Unlike Formats 1-3 that only have the TCR controlled by the TRAC or TRBC promoter and have the Payloads controlled by a secondary promoter, Format 4 controls both the TCR and the Payload off of the single, endogenous TRAC or TRBC promoter.

FIGS. 9A and 9B illustrate the reduction of NeoTCR expression in cells transduced with Secondary Promoter Constructs including the indicated promoters. FIG. 9A shows the reduced expression of NeoTCR in constructs comprising a secondary promoter as measured by dextramer labeling. FIG. 9B shows tumor-killing induced by cells transduced with a construct with or without a secondary promoter.

FIGS. 10A-10C illustrate the effects of different genomic elements on NeoTCR expression in constructs comprising a secondary promoter. FIG. 10A shows the effect of enhancer addition in constructs comprising a secondary promoter. FIG. 10B shows the effect of replacement of STOP codon with a sequence encoding Furin and P2A after the Payload. FIG. 10C shows the effects due to the addition of termination elements in constructs comprising a secondary promoter.

FIG. 11 shows Secondary Promoter Constructs disclosed herein.

FIGS. 12A-12C illustrate the effect of Secondary Promoter Constructs on gene editing and exogenous TCR expression. FIG. 12A shows the percentage of edited cells with the indicated different constructs. FIG. 12B shows expression levels of NeoTCR induced by different Secondary Promoter Constructs. FIG. 12C shows expression levels of mCherry induced by indicated Secondary Promoter Constructs. Details of the constructs can be found in the Example section.

FIGS. 13A-13D illustrate the effects of different Secondary Promoter Constructs on NeoTCR expression and Payload expression. FIGS. 13A and 13B show representative results of NeoTCR expression induced by different Secondary Promoter Constructs. FIGS. 13C and 13D show representative results of Payload (e.g., mCherry) expression induced by the indicated Secondary Promoter Constructs. Details of the constructs can be found in the Example section.

FIG. 14 shows additional Secondary Promoter Constructs.

FIGS. 15A-15D illustrate gene expression and gene editing obtained by Secondary Promoter Constructs depicted in FIG. 14. FIGS. 15A and 15B show NeoTCR gene expression levels in cells expressing the indicated Secondary Promoter Constructs. FIGS. 15C and 15D show Payload expression (e.g., mCherry) induced by different Secondary Promoter Constructs. Details of the constructs can be found in the Example section.

FIGS. 16A-16H show additional Secondary Promoter Constructs disclosed herein. Details of these Secondary Promoter Constructs can be found in Example 8. shRNA: inhibitory RNA molecule.

FIGS. 17A-17C illustrate the effects of secondary promoters upstream of inhibitory RNA molecules. FIG. 17A shows representative Secondary Promoter Constructs disclosed herein. FIG. 17B shows gene expression levels of CBLB in cells including different Secondary Promoter Constructs. FIG. 17C shows the expression levels of NeoTCR. MND>mirE(CBLB): MND promoter upstream of mir-E targeting CBLB (“mir-E(CBLB)”); MND>eSIBR(CBLB): MND promoter upstream of eSIBR targeting CBLB (“eSIBR(CBLB)”); MND>eSIBR(CBLB).mirE(A2AR): MND promoter upstream of eSIBR(CBLB) and mir-E targeting A2AR (“mir-E(A2AR)”); MND>mirE(A2AR).eSIBR(CBLB): MND promoter upstream of mir-E(A2AR) and eSIBR(CBLB); hPGK>mirE(CBLB): hPGk promoter upstream of mir-E(CBLB); U6>mir22(CBLB): U6 promoter upstream of mir-22 targeting CBLB.

FIGS. 18A-18C illustrate Secondary Promoter Constructs including a Payload and an inhibitory RNA. FIG. 18A shows representative Secondary Promoter Constructs disclosed herein. FIG. 18B shows gene expression levels of CBLB in cells including different Secondary Promoter Constructs. FIG. 18C shows gene expression levels of a Payload (mCherry) in cells including different Secondary Promoter Constructs. MND>mirE(CBLB): MND promoter upstream of mir-E targeting CBLB (“mir-E(CBLB)”); MND>mirE(CBLB).mCherry: MND promoter upstream of mir-E(CBLB) and mCherry as Payload; MND>mCherry.mirE(CBLB): MND promoter upstream of mCherry and mir-E(CBLB).

FIGS. 19A-19E illustrate Secondary Promoter Constructs including a promoter and an inhibitory RNA. FIG. 19A shows representative Secondary Promoter Constructs disclosed herein.

FIG. 19B shows gene expression levels of CBLB in cells including different Secondary Promoter Constructs. FIG. 19C shows the expression levels of NeoTCR. FIG. 19D shows gene editing levels. FIG. 19E shows the percentage of viability. MND>mirE(CBLB): MND promoter upstream of mir-E targeting CBLB (“mir-E(CBLB)”); hPGK>mirE(CBLB): hPGk promoter upstream of mir-E(CBLB); U6>mir22(CBLB): U6 promoter upstream of mir-22 targeting CBLB; MND>eSIBR(CBLB): MND promoter upstream of eSIBR targeting CBLB (“eSIBR(CBLB)”); 1: control; 2: MND promoter upstream of mir-E targeting CBLB (“mir-E(CBLB)”) and Payload (mCherry); 3: MND promoter upstream of mir-E(CBLB); 4: MND promoter upstream of Payload and mir-E(CBLB); 5: MND promoter upstream of splice donor site, mir-E(CBLB), and splice acceptor site; 6: MND promoter upstream of eSIBR targeting CBLB (“eSIBR(CBLB)”) and mir-E(CBLB); 7: MND promoter upstream of eSIBR(CBLB); 8: hPGK promoter upstream of mir-E(CBLB); 9: MND promoter upstream of mir-E targeting A2AR and eSIBR(CBLB); 10: U6 promoter upstream of mir-22 targeting CBLB.

FIGS. 20A-20D illustrate Secondary Promoter Constructs including multiple Payloads or inhibitory RNA molecules. FIG. 20A shows representative Secondary Promoter Constructs disclosed herein. FIG. 20B shows gene expression levels of CBLB in cells including different Secondary Promoter Constructs. FIG. 20C shows gene expression levels of A2AR. FIG. 20D shows the expression levels of NeoTCR. MND>mirE(CBLB): MND promoter upstream of mir-E targeting CBLB (“mir-E(CBLB)”); MND>eSIBR(CBLB): MND promoter upstream of eSIBR targeting CBLB (“eSIBR(CBLB)”); MND>eSIBR(CBLB).mirE(A2AR): MND promoter upstream of eSIBR(CBLB) and mir-E targeting A2AR (“mir-E(A2AR)”); MND>mirE(A2AR).eSIBR(CBLB): MND promoter upstream of mir-E(A2AR) and eSIBR(CBLB).

FIGS. 21A-21C illustrate Secondary Promoter Constructs including miRNA clusters. FIG. 21A shows representative Secondary Promoter Constructs disclosed herein. FIG. 21B shows gene expression levels of TGFBR2 in cells including different Secondary Promoter Constructs. FIG. 21C shows the expression levels of NeoTCR. TCR089(0.3 kb): control; TCR089+TGFBR2KO: NeoTCR and TGFBR2 knockout; U6>mir22(TGFBR2).6T: U6 promoter upstream of mir-22 targeting TGFBR2 and termination sequence; MND>mir106(CBLB-A2AR-TGFBR2): MND promoter upstream of mir-106 targeting CBLB, A2AR, and TGFBR2; MND>mirE(A2AR): MND promoter upstream of mir-E targeting A2AR; U6>mirE(A2AR).6T: U6 promoter upstream of mir-E targeting A2AR and termination sequence.

FIGS. 22A-22C illustrate Secondary Promoter Constructs including splice acceptor site and splice donor site. FIG. 22A shows representative Secondary Promoter Constructs disclosed herein. FIG. 22B shows gene expression levels of CBLB in cells including different Secondary Promoter Constructs. FIG. 21C shows the expression levels of NeoTCR. MND>mirE(CBLB): MND promoter upstream of mir-E targeting CBLB (“mir-E(CBLB)”); MND>SD.mirE(CBLB).SA: MND promoter upstream of splice donor site, mir-E(CBLB), and splice acceptor site.

FIG. 23 shows representative Secondary Promoter Constructs disclosed herein.

### DETAILED DESCRIPTION

The present disclosure provides adoptive cell therapies comprising a first and a second exogenous gene and having enhanced activity and efficacy against tumor cells. The present disclosure is based, in part, on the ability of the inventors to decouple the gene expression of the first and second exogenous genes resulting in elevated and maintained expression levels of a sequence encoding an exogenous TCR (e.g., a NeoTCR) and a Payload that potentiates and improves the activity of the cells (e.g., cytotoxicity, cell proliferation, and/or cell persistence). The present disclosure also provides methods for producing the cells, and compositions disclosed herein. Finally, the present disclosure provides methods of using such cells and composition for treating and/or preventing cancer.

Non-limiting embodiments of the present disclosure are described by the present description and examples. For purposes of clarity of disclosure and not by way of limitation, the detailed description is divided into the following subsections:
1. Definitions;
2. NeoTCR Products;
3. Secondary Promoter Products;
4. Methods of Treatment;
5. Articles of Manufacture;
6. Therapeutic Compositions and Methods of Manufacturing;
7. Kits; and
8. Exemplary Embodiments.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

It is understood that aspects and embodiments of the invention described herein include “comprising,” “consisting,” and “consisting essentially of” aspects and embodiments. The terms “comprises” and “comprising” are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

The term “antibody” as used herein is used in the broadest sense and encompasses various antibody structures, including but not limited to monoclonal antibodies, polyclonal antibodies, multispecific antibodies (e.g., bispecific and tri-specific antibodies), and antibody fragments (e.g., bis-Fabs) so long as they exhibit the desired antigen-binding activity. “Antibody Fragment” as used herein refers to a molecule other than an intact antibody that comprises a portion of an intact antibody that binds the antigen to which the intact antibody binds. Examples of antibody fragments include but are not limited to bis-Fabs; Fv; Fab; Fab, Fab′-SH; F(ab′)₂; diabodies; linear antibodies; single-chain antibody molecules (e.g., scFv); and multispecific antibodies formed from antibody fragments.

The terms “Cancer” and “Tumor” are used interchangeably herein. As used herein, the terms “Cancer” or “Tumor” refer to all neoplastic cell growth and proliferation, whether malignant or benign, and all pre-cancerous and cancerous cells and tissues. The terms are further used to refer to or describe the physiological condition in mammals that is typically characterized by unregulated cell growth/proliferation. Cancer can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Cancer includes cancers, such as sarcomas, carcinomas, or plasmacytomas (a malignant tumor of the plasma cells). Examples of cancer include, but are not limited to, those described herein. The terms “Cancer” or “Tumor” and “Proliferative Disorder” are not mutually exclusive as used herein.

As used herein, “sequence identity” or “identity” (in the context of two nucleic acid or polypeptide sequences) refers to the residues in the two sequences that are the same when aligned for maximum correspondence over a specified comparison window. When the percentage of sequence identity is used about proteins it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art.

As used herein, “percentage of sequence identity” refers to the value determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for the optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Methods of alignment of sequences for comparison include, without any limitation, the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877. Computer implementations of these mathematical algorithms include, without any limitation, CLUSTAL, CLUSTALW, CLUSTALOMEGA, ALIGN, ALIGN PLUS, GAP, BESTFIT, BLAST, FASTA, TFASTA, BLASTN, BLASTX, BLASTP, TBLASTN, and TBLASTX.

A “conservative substitution” or a “conservative amino acid,” refers to the substitution of amino acid with a chemically or functionally similar amino acid. Conservative substitution tables providing similar amino acids are well known in the art. In certain embodiments, acidic amino acids D and E are conservative substitutions for one another; basic amino acids K, R, and H are conservative substitutions for one another; hydrophilic uncharged amino acids S, T, N. and Q are conservative substitutions for one another; aliphatic uncharged amino acids G, A, V, L, and I are conservative substitutions for one another; non-polar uncharged amino acids C, M, and P are conservative substitutions for one another; aromatic amino acids F, Y, and W are conservative substitutions for one another; A, S, and T are conservative substitutions for one another; D and E are conservative substitutions for one another; N and Q are conservative substitutions for one another; R and K are conservative substitutions for one another; I, L, and M are conservative substitutions for one another; F, Y, and W are conservative substitutions for one another; A and G are conservative substitutions for one another; D and E are conservative substitutions for one another; N and Q are conservative substitutions for one another; R, K and H are conservative substitutions for one another; I, L, M, and V are conservative substitutions for one another; F, Y and W are conservative substitutions for one another; S and T are conservative substitutions for one another; and C and M are conservative substitutions for one another. Additional conservative substitutions may be found, for example, in Creighton, Proteins: Structures and Molecular Properties 2nd ed. (1993) W. H. Freeman & Co., New York, NY.

“Treat,” “Treatment,” and “treating” are used interchangeably and as used herein mean obtaining beneficial or desired results including clinical results. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, the NeoTCR Product of the invention is used to delay the development of a proliferative disorder (e.g., cancer) or to slow the progression of such disease.

“Dextramer” as used herein means a multimerized neoepitope-HLA complex that specifically binds to its cognate NeoTCR.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen-recognizing receptor or capable of suppressing an immune response via receptor-ligand binding.

As used herein, the terms “neoantigen”, “neoepitope” or “neoE” refer to a newly formed antigenic determinant that arises, e.g., from a somatic mutation(s) and is recognized as “non-self” A mutation giving rise to a “neoantigen”, “neoepitope” or “neoE” can include a frameshift or non-frameshift indel, missense or nonsense substitution, splice site alteration (e.g., alternatively spliced transcripts), genomic rearrangement or gene fusion, any genomic or expression alterations, or any post-translational modifications. In certain embodiments, the neoantigen can be a private neoantigen. As used herein, the term “private neoantigen” refers to neoantigens that are exclusively expressed and present in a subject having certain cancer. For clarity, a private neoantigen is a neoantigen that cannot be used for another patient. In certain embodiments, the neoantigen can be a “public neoantigen”. The term “public neoantigen,” as used herein, refers to neoantigens that are shared by more than one subject.

“NeoTCR” and “NeoE TCR,” as used herein, mean a neoepitope-specific T cell receptor that is introduced into a T cell, e.g., by gene-editing methods. As used herein, the term “exogenous TCR” may be used in place of “NeoTCR”.

“NeoTCR Cells” as used herein means one or more cells precision-engineered to express one or more NeoTCRs. In certain embodiments, the cells are T cells. In certain embodiments, the T cells are CD8+ and/or CD4+ T cells. In certain embodiments, the CD8+ and/or CD4+ T cells are autologous cells from the patient for whom a NeoTCR Product will be administered. The terms “NeoTCR Cells” and “NeoTCR-P1 T cells” and “NeoTCR-P1 cells” are used interchangeably herein.

“NeoTCR Product” as used herein means a pharmaceutical formulation comprising one or more NeoTCR Cells. NeoTCR Product consists of autologous precision genome-engineered CD8+ and CD4+ T cells. Using a targeted DNA-mediated non-viral precision genome engineering approach, expression of the endogenous TCR is eliminated and replaced by a patient-specific NeoTCR isolated from peripheral CD8+ T cells targeting the tumor-exclusive neoepitope. In certain embodiments, the resulting engineered CD8+ or CD4+ T cells express NeoTCRs on their surface of native sequence, native expression levels, and native TCR function. The sequences of the NeoTCR external binding domain and cytoplasmic signaling domains are unmodified from the TCR isolated from native CD8+ T cells. Regulation of the NeoTCR gene expression is driven by the native endogenous TCR promoter positioned upstream of where the NeoTCR gene cassette is integrated into the genome. Through this approach, native levels of NeoTCR expression are observed in unstimulated and antigen-activated T cell states. The NeoTCR Product manufactured for each patient represents a defined dose of autologous CD8+ and/or CD4+ T cells that are precision genome engineered to express a single neoE-specific TCR cloned from neoE-specific CD8+ T cells individually isolated from the peripheral blood of that same patient.

“NeoTCR Viral Product” as used herein has the same definition of NeoTCR Product except that the genome engineering is performed using viral-mediated methods.

“Secondary Promoter Construct” as used herein refers to a construct comprising elements to express a NeoTCR and elements to express a Payload. Formats 1-4, along with the variations thereof described herein, are representative examples of Secondary Promoter Constructs. Additional information can be found in Section 3 below.

“Secondary Promoter Cell” as used herein refers to a NeoTCR Cell that is engineered to express a Payload under the control of a different promoter than the promoter used to express the NeoTCR.

“Secondary Promoter Product” as used herein refers to a NeoTCR Product that is engineered to express a Payload under the control of a different promoter than the promoter used to express the NeoTCR.

“Payload” as used herein refers to a second or more gene, in addition to the NeoTCR, to be knocked into an immune cell using gene editing methods described herein. Examples of Payloads include but are not limited to the one disclosed in Section 3.1.1. below.

“Pharmaceutical Formulation” refers to a preparation that is in such form as to permit the biological activity of an active ingredient contained therein to be effective, and which contains no additional components which are unacceptably toxic to a subject to which the formulation would be administered. For clarity, DMSO at quantities used in a NeoTCR Product is not considered unacceptably toxic.

“Promoter region” or “promoter” as used herein means the region of the construct that encodes a promoter that controls the expression of the Payload in the Secondary Promoter Constructs.

As used herein, the term “enhancer” refers to a DNA control element that enhances the levels of expression of the gene when a specific transcription factor is bound. Unlike a promoter, an enhancer does not stimulate the expression of the gene on its own. Enhancers are frequently found in the upstream (5′) region of the gene.

A “subject,” “patient,” or an “individual” for purposes of treatment refers to any animal classified as a mammal, including humans, domestic and farm animals, and zoo, sports, or pet animals, such as dogs, horses, cats, cows, etc. Preferably, the mammal is human.

“TCR” as used herein means T cell receptor.

“2A” and “2A peptide” are used interchangeably herein and mean a class of 18-22 amino acid long, viral, self-cleaving peptides that are able to mediate cleavage of peptides during translation in eukaryotic cells. Four well-known members of the 2A peptide class are T2A, P2A, E2A, and F2A.

The T2A peptide was first identified in the Thosea asigna virus 2A. The P2A peptide was first identified in the porcine teschovirus-1 2A. The E2A peptide was first identified in the equine rhinitis A virus. The F2A peptide was first identified in the foot-and-mouth disease virus. The self-cleaving mechanism of the 2A peptides is a result of ribosome skipping the formation of a glycyl-prolyl peptide bond at the C-terminus of the 2A. Specifically, the 2A peptides have a C-terminal conserved sequence that is necessary for the creation of steric hindrance and ribosome skipping. The ribosome skipping can result in one of three options: 1) successful skipping and recommencement of translation resulting in two cleaved proteins (the upstream of the 2A protein which is attached to the complete 2A peptide except for the C-terminal proline and the downstream of the 2A protein which is attached to one proline at the N-terminal; 2) successful skipping but ribosome fall-off that results in discontinued translation and only the protein upstream of the 2A; or 3) unsuccessful skipping and continued translation (i.e., a fusion protein).

The term “endogenous” as used herein refers to a nucleic acid molecule or polypeptide that is normally expressed in a cell or tissue.

The term “exogenous” as used herein refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

“Young” or “Younger” or “Young T cell” as it relates to T cells means memory stem cells (TMSC) and central memory cells (TCM). These cells have T cell proliferation upon specific activation and are competent for multiple cell divisions. They also have the ability to engraft after re-infusion, to rapidly differentiate into effector T cells upon exposure to their cognate antigen and target and kill tumor cells, as well as to persist for ongoing cancer surveillance and control.

“WPRE,” as used herein, refers to a woodchuck hepatitis virus post-transcriptional regulatory element that increases transgene expression.

As used herein, a “STOP codon” or “termination codon” is a nucleotide triplet within a messenger RNA that signals the termination of the translation process of a protein. Most codons in messenger RNA correspond to the addition of an amino acid to a growing polypeptide chain, which can ultimately become a protein; stop codons signal the termination of this process by binding release factors, which cause the ribosomal subunits to disassociate, releasing the amino acid chain.

“Kozak” or “Kozak sequence,” as used herein, refers to a nucleic acid motif that functions as the protein translation initiation site in certain eukaryotic mRNA transcripts.

“Insulator” or “transcriptional insulator,” as used herein, refer to a class of DNA sequence elements that possess a common ability to protect genes from inappropriate signals emanating from their surrounding environment. Insulators can be used to restrict the interaction of enhancers or silencers on promoters in a gene expression system. Insulators can set boundaries on the actions of enhancer and silencer elements and so partition the eukaryotic genome into regulatory domains. Physiologically, the transcriptional repressor CTCF (CCCTC-binding factor) binds through multiple zinc fingers (of which it has eleven) to a range of unrelated DNA sequences and functions as a transcriptional insulator, repressor, or activator, depending on the context of the binding site.

“Signal sequence” as used herein is a peptide that can be included at the N-terminus of a newly synthesized protein for the purpose of trafficking the newly synthesized protein to its intended and/or engineered location inside or outside of the cell.

### 2. NeoTCR Products

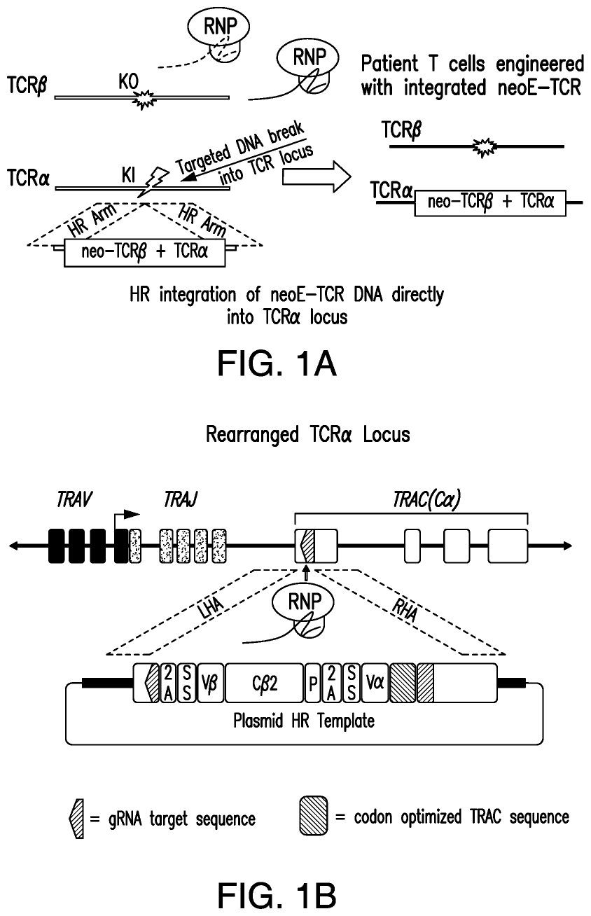

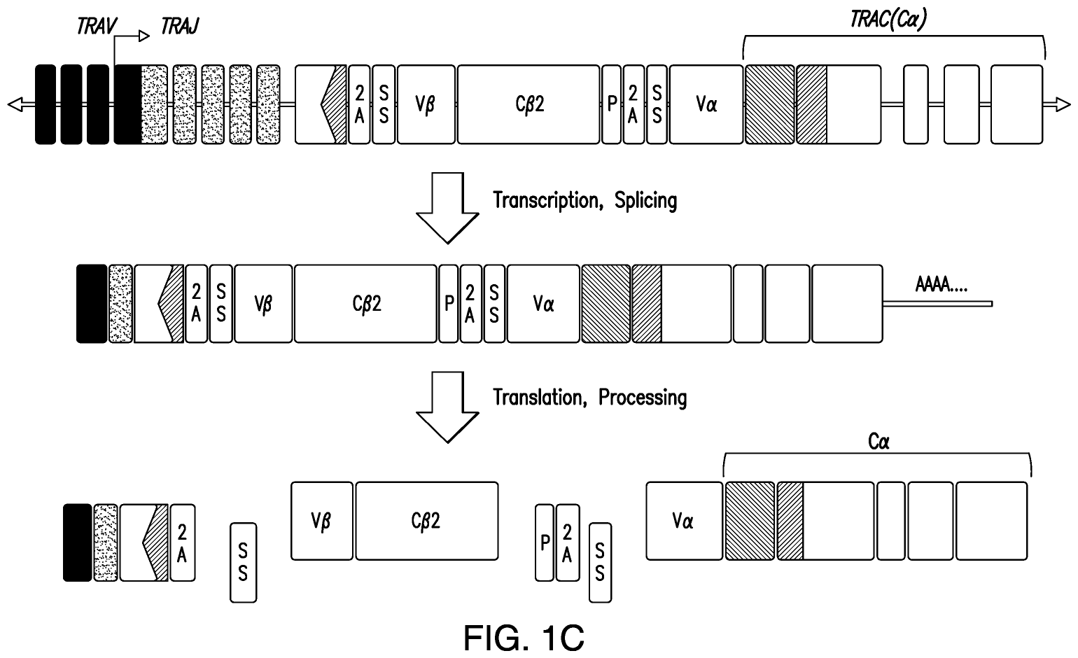

In certain embodiments, using the gene-editing technology and NeoTCR isolation technology described in PCT/US2020/17887 and PCT/US2019/025415, which are incorporated herein in their entireties, NeoTCRs are cloned in autologous CD8+ and CD4+ T cells from the same patient with cancer by precision genome engineered (using a DNA-mediated (non-viral) method as described in FIGS. 1A-1C) to express the NeoTCR. In other words, the NeoTCRs that are tumor-specific are identified in cancer patients, such NeoTCRs are then cloned, and then the cloned NeoTCRs are inserted into the cancer patient's own T cells. Importantly, once expressed in the cells (e.g., CD8 T cells), the NeoTCR can recognize a private neoantigen. NeoTCR expressing T cells are then expanded in a manner that preserves a “young” T cell phenotypes, resulting in a NeoTCR-P1 product (i.e., a NeoTCR Product) in which the majority of the T cells exhibit T memory stem cell and T central memory phenotypes. These ‘young’ or ‘younger’ or less-differentiated T cell phenotypes are described to confer improved engraftment potential and prolonged persistence post-infusion. Thus, the administration of NeoTCR Product, consisting significantly of ‘young’ T cell phenotypes, has the potential to benefit patients with cancer, through improved engraftment potential, prolonged persistence post-infusion, and rapid differentiation into effector T cells to eradicate tumor cells throughout the body.

Ex vivo mechanism-of-action studies were also performed with NeoTCR Products manufactured with T cells from patients with cancer. Comparable gene editing efficiencies and functional activities, as measured by antigen-specificity of T cell killing activity, proliferation, and cytokine production, were observed demonstrating that the manufacturing process described herein is successful in generating products with T cells from patients with cancer as starting material.

In certain embodiments, the NeoTCR Product manufacturing process involves electroporation of dual ribonucleoprotein species of CRISPR-Cas9 nucleases bound to guide RNA sequences, with each species targeting the genomic TCRα and the genomic TCRβ loci. The specificity of targeting Cas9 nucleases to each genomic locus has been previously described in the literature as being highly specific. Comprehensive testing of the NeoTCR Product was performed in vitro and in silico analyses to survey possible off-target genomic cleavage sites, using COSMID and GUIDE-seq, respectively. Multiple NeoTCR Products or comparable cell products from healthy donors were assessed for cleavage of the candidate off-target sites by deep sequencing, supporting the published evidence that the selected nucleases are highly specific. Further aspects of the precision genome engineering process have been assessed for safety. No evidence of genomic instability following precision genome engineering was found in assessing multiple NeoTCR Products by targeted locus amplification (TLA) or standard FISH cytogenetics. No off-target integration anywhere into the genome of the NeoTCR sequence was detected. No evidence of residual Cas9 was found in the cell product.

The comprehensive assessment of the NeoTCR Product and precision genome engineering process indicates that the NeoTCR Product will be well tolerated following infusion back to the patient.

The genome engineering approach described herein enables the highly efficient generation of bespoke NeoTCR Cells (i.e., NeoTCR Products) for personalized adoptive cell therapy for patients with solid and liquid tumors. Furthermore, the engineering method is not restricted to the use in T cells and has also been applied successfully to other primary cell types, including natural killer and hematopoietic stem cells.

### 3. Secondary Promoter Products

In certain embodiments, the present disclosure provides Secondary Promoter Products. In certain embodiments, the NeoTCR Cells described can express a Payload under the control of a different promoter than the promoter used to express the NeoTCR to produce a Secondary Promoter Cell. In certain embodiments, the NeoTCR Cells described herein are further engineered to express a Payload under the control of a different promoter than the promoter used to express the NeoTCR to produce a Secondary Promoter Cell. Such Secondary Promoter Cells can be formulated into Secondary Promoter Products for the treatment of an immune disease or disorder.

In certain embodiments, the immune disease or disorder is a primary immune disease or disorder (i.e., a disease or disorder that a subject is born with). In certain embodiments, the immune disease or disorder is a secondary disorder (i.e., acquired disease or disorder). In certain embodiments, the immune disease selected from the group comprising cancers, autoimmune diseases and disorders, viral infections, bacterial infections, retroviral infections, and neurological diseases and disorders. In certain embodiments, the immune disease is cancer. In certain embodiments, the immune disease is an autoimmune disease.

In certain embodiments, a single-step precision genome engineering method is performed in order for the NeoTCR insertion and the Payload insertion into a primary cell to occur in the same reaction. In certain embodiments, the NeoTCR and the Payload are encoded by the same polynucleotide. In certain embodiments, the primary cell is a T cell. In certain embodiments, the primary cell is an NK cell. In certain embodiments, this single-step process reduces potential T cell heterogeneity that would otherwise be present if multiple reactions and multiple polynucleotides (e.g., DNA templates) were used. In certain embodiments, this single-step process reduces potential NK cell heterogeneity that would otherwise be present if multiple reactions and multiple polynucleotides (e.g., DNA templates) were used.

In certain embodiments, the single-step precision genome engineering method described herein allows for one or more Payloads to be engineered in and/or one or more removal (i.e., silencing) of one or more genes endogenous to the primary cell.

In certain embodiments, the Secondary Promoter Cells of the Secondary Promoter Products are designed to target molecules on the tumor. Non-limiting examples of molecules expressed on tumors that the Secondary Promoter Cells and Secondary Promoter Products can target include but are not limited to antigens, neoantigens, private neoantigens, public neoantigens, receptors, cell surface molecules, cytokine receptors, and oncogenic pathway inhibitors or enhancers.

In certain embodiments, the Secondary Promoter Cells of the Secondary Promoter Products are designed to express a protein made by the Payload only upon activation of the Secondary Promoter Cells. In certain embodiments, the activated Secondary Promoter Cells are activated T cells.

In certain embodiments, the Secondary Promoter Cells of the Secondary Promoter Products are designed to secrete a protein made by the Payload only upon activation of the Secondary Promoter Cells. In certain embodiments, the activated Secondary Promoter Cells are activated T cells.

In certain embodiments, the Secondary Promoter Cells of the Secondary Promoter Products are designed to constitutively express a protein made by the Payload.

In certain embodiments, the Secondary Promoter Cells of the Secondary Promoter Products are designed to constitutively secrete a protein made by the Payload.

### 3.1. Secondary Promoter Constructs

Secondary Promoter Constructs disclosed herein (including but not limited to Formats 1-4 described herein and diagramed in the figures) are designed to allow for the dual expression of an exogenous TCR (e.g., a NeoTCR) and a Payload when transfected or transduced into a cell. In certain embodiments, the method of gene insertion is non-viral transfection. In certain embodiments, the non-viral transfection methods used are those described herein. In certain embodiments, the cell is a primary human cell. In certain embodiments, the primary human cell is a T cell.

In certain embodiments, one or more of the following key elements are included in the Secondary Promoter Constructs: an element to promote translation of transcripts (e.g., enhancer), a poly-adenylation (poly-A) sequence, a promoter, a pause element, a woodchuck hepatitis virus post-transcriptional regulatory element (WPRE), a scaffold/matrix attachment region, and an insulator.

In certain embodiments, the Secondary Promoter Constructs are modular, as each element should be thought of as a broad and general category. For instance, any poly-A signal sequence known to one of skill in the art could be used in the modular construct. For instance, any element to promote translation of transcripts applicable to the intended cell and Payload known to one of skill in the art could be used in the modular construct. For instance, any applicable promoter for the intended cell and Payload known to one of skill in the art could be used in the modular construct. For instance, any applicable insulator for the intended cell and Payload known to one of skill in the art could be used in the modular construct.

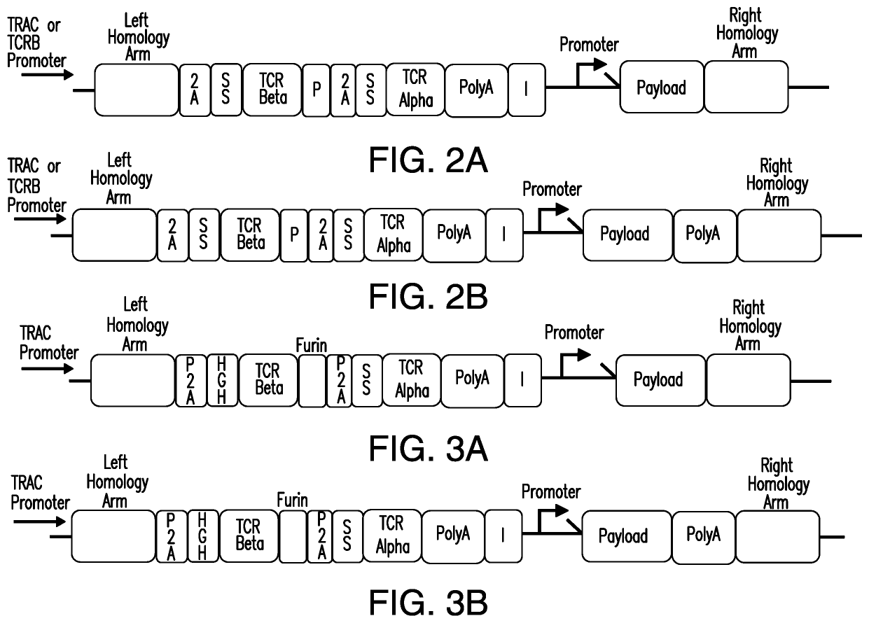

In certain embodiments, the Secondary Promoter Construct designs comprise a Payload, a regulatory element to increase the translation of the Payload transcript, a transcription stop signal, a promoter, and optionally one or more insulators. In certain embodiments, the Secondary Promoter Construct design is described in FIGS. 2A-8, 11, and 14. In certain embodiments, the Secondary Promoter Construct design is designated as “Format 1,” “Format 2,” “Format 3,” or “Format 4.”

3.1.1. Payloads

In certain embodiments, the Secondary Promoter Constructs disclosed herein include a Payload. In certain embodiments, the Payload of the Secondary Promoter Constructs is selected from the group comprising a cytokine receptors trap or ligand trap, an angiogenesis factors, an apoptotic factor, an inhibitory protein for various cell processes (e.g., inhibition of macrophage activation), an extracellular matrix modulator, a soluble TCRs, a soluble chimeric antigen receptor (CAR), a homing signal, an enzyme, a modulator of reactive oxygen species, a competitive ligand inhibitor, and a protein that binds to receptors and sterically hinders receptor function.

In certain embodiments, the Payload is a cytokine receptor trap or a ligand trap. In certain embodiments, the cytokine receptor trap or ligand trap selected as a Payload acts as a cytokine sink. Cytokine receptor traps recognize certain inflammatory cytokines with high affinity and specificity but are structurally incapable of signaling or presenting the agonist to signaling receptor complexes. They act as a molecular trap for the agonist and signaling receptor components. Non-limiting examples of cytokine receptor trap include TNFR1, TNFR2, TGFBR1, TGFBR2, TGFBR3, IL4, CSF1R, CXCR1, CXCR2, CCR2, and CCR5. In certain embodiments, the cytokine receptor trap is a TGFBR2. In certain embodiments, the TGFBR2 comprises an amino acid sequence that is at least about 80%, about 85%, about 90%, about 95%, about 96%, about 97%, about 98%, or about 99% identical to the amino sequence set forth in SEQ ID NO: 1. In certain embodiments, the TGFBR2 comprises the amino sequence set forth in SEQ ID NO: 1. In certain embodiments, the TGFBR2 consists of the amino sequence set forth in SEQ ID NO: 1. SEQ ID NO: 1 is provided below.

[SEQ ID NO: 1]
MGRGLLRGLWPLHIVLWTRIASTIPPHVQKSVNNDMIVTD
NNGAVKFPQLCKFCDVRFSTCDNQKSCMSNCSITSICEKP
QEVCVAVWRKNDENITLETVCHDPKLPYHDFILEDAASPK
CIMKEKKKPGETFFMCSCSSDECNDNIIFSEEYNTSNPDL
LLVIFQVTGISLLPPLGVAISVIIIFYCYRVNRQQKLSS

In certain embodiments, the TGFBR2 is encoded by the nucleotide sequence set forth in SEQ ID NO: 2 provided below.

[SEQ ID NO: 2]
ATGGGTCGGGGGCTGCTCAGGGGCCTGTGGCCGCTGCACA
TCGTCCTGTGGACGCGTATCGCCAGCACGATCCCACCGCA
CGTTCAGAAGTCGGTTAATAACGACATGATAGTCACTGAC
AACAACGGTGCAGTCAAGTTTCCACAACTGTGTAAATTTT
GTGATGTGAGATTTTCCACCTGTGACAACCAGAAATCCTG
CATGAGCAACTGCAGCATCACCTCCATCTGTGAGAAGCCA
CAGGAAGTCTGTGTGGCTGTATGGAGAAAGAATGACGAGA
ACATAACACTAGAGACAGTTTGCCATGACCCCAAGCTCCC
CTACCATGACTTTATTCTGGAAGATGCTGCTTCTCCAAAG
TGCATTATGAAGGAAAAAAAAAAGCCTGGTGAGACTTTCT
TCATGTGTTCCTGTAGCTCTGATGAGTGCAATGACAACAT
CATCTTCTCAGAAGAATATAACACCAGCAATCCTGACTTG
TTGCTAGTCATATTTCAAGTGACAGGCATCAGCCTCCTGC
CACCACTGGGAGTTGCCATATCTGTCATCATCATCTTCTA
CTGCTACCGCGTTAACCGGCAGCAGAAGCTGAGTTCA

Additional information and examples of cytokine traps can be found in Economides et al., Nature medicine 9.1 (2003): 47-52.

In certain embodiments, the Payload is an angiogenesis factor. In certain embodiments, the angiogenesis factor selected as a Payload reshapes tumor vasculature. Non-limiting examples of angiogenesis factors include FGF1, FGF2, VEGF, PDGF, ANGPT1, ANGPT2, DLL4, MMP1, MMP2, MMP3, MMP47, MMP8, MMP9, MMP10, MMP11, MMP12, MMP13, MMP14, MMP15, MMP16, MMP17, MMP24, MMP25, MMP28, SEMA3A, SEMA3B, SEMA3C, SEMA3D, SEMA3E, SEMA3F, SEMA3G, urokinase, and tissue plasminogen activator.

In certain embodiments, the Payload is an apoptotic factor. In certain embodiments, the apoptotic factor selected as a Payload protects a Secondary Promoter Cell from apoptosis. In certain embodiments, the apoptotic factor selected as a Payload induces apoptosis in a target cell (e.g., a tumor cell). Non-limiting examples of apoptotic factors include FAS, FASL, TNFα, XIAP, cIAPI, c-IAP2, NAIP, API4, Bcl-2, cFLIP, BNIP3, FADD, AKT, and NK-κB.

In certain embodiments, the Payload is an inhibitory protein. In certain embodiments, the inhibitory protein selected as a Payload inhibits macrophage activity. Non-limiting examples of inhibitory proteins include anti-TIM3 antibodies, anti-PD1 antibodies, anti-CD32b antibodies, and anti-CD200R antibodies.

In certain embodiments, the Payload is an anti-PD1 antibody. In certain embodiment, the Payload is an anti-PD1 svFv. In certain embodiments, the anti-PD1 svFv comprises an amino acid sequence that is at least about 80%, about 85%, about 90%, about 95%, about 96%, about 97%, about 98%, or about 99% identical to the amino sequence set forth in SEQ ID NO: 3. In certain embodiments, the anti-PD1 svFv comprises the amino sequence set forth in SEQ ID NO: 3. In certain embodiments, the anti-PD1 svFv consists of the amino sequence set forth in SEQ ID NO: 3. SEQ ID NO: 3 is provided below.

[SEQ ID NO: 3]
MYRMQLLSCIALSLALVTNSEIVLTQSPATLSLSPGERAT
LSCRASQSVSSYLAWYQQKPGQAPRLLIYDASNRATGIPA
RFSGSGSGTDFTLTISSLEPEDFAVYYCQQSSNWPRTFGQ
GTKVEIKGGGGSGGGGSGGGGSQVQLVESGGGVVQPGRSL
RLDCKASGITFSNSGMHWVRQAPGKGLEWVAVIWYDGSKR
YYADSVKGRFTISRDNSKNTLFLQMNSLRAEDTAVYYCAT
NDDYWGQGTLVTVSS

In certain embodiments, the anti-PD1 svFv is encoded by the nucleotide sequence set forth in SEQ ID NO: 4 provided below.

[SEQ ID NO: 4]
ATGTACAGGATGCAACTCCTGTCTTGCATTGCACTAAGTC
TTGCACTTGTCACAAACAGTGAGATCGTGCTGACTCAGTC
CCCTGCTACCCTGTCCCTGTCCCCCGGTGAGCGTGCTACC
CTGTCCTGCCGCGCTTCCCAGAGCGTGTCCTCCTACCTGG
CCTGGTATCAACAAAAGCCCGGCCAAGCTCCCCGCCTGCT
GATCTACGACGCCTCCAACCGCGCTACTGGCATCCCTGCT
CGCTTCTCCGGTTCCGGTTCCGGCACTGACTTCACTCTGA
CCATCTCCTCCCTGGAGCCCGAGGATTTCGCTGTGTACTA
CTGCCAGCAGTCCTCCAACTGGCCCCGTACCTTCGGTCAG
GGCACCAAGGTGGAGATCAAGGGTGGAGGCGGTTCAGGCG
GAGGTGGCTCTGGCGGTGGCGGAAGCCAGGTCCAGCTGGT
GGAGAGCGGTGGTGGTGTGGTGCAGCCCGGTCGTTCCCTG
CGTCTGGATTGCAAAGCCTCCGGCATCACCTTCTCCAACA
GCGGCATGCACTGGGTGCGTCAGGCTCCTGGTAAGGGCCT
GGAGTGGGTGGCTGTGATCTGGTACGATGGCTCCAAGCGC
TACTACGCCGACTCCGTGAAGGGTCGTTTCACCATCTCCC
GCGACAACAGCAAGAACACCCTGTTCCTGCAGATGAACTC
CCTGCGTGCCGAGGACACCGCCGTCTACTACTGCGCCACC
AACGACGACTACTGGGGTCAGGGCACTCTGGTGACCGTCT
CCTCC

In certain embodiments, the Payload is an extracellular matrix modulator. In certain embodiments, the extracellular matrix modulator selected as a Payload makes the tumor more physically accessible to immune cells. Non-limiting examples of extracellular matrix modulator include MATN1, MATN2, MATN3, MATN4, CTSB, CTSG, CEMIP, HPSE, HYAL1, HYAL2, HYAL3, HYAL4, HYAL5, PLOD1, PLOD2, PLOD3, ELANE, PLG, SULF1, SULF2, and TMPRSS6.

In certain embodiments, the Payload is a homing signal. This homing signal can guide the Secondary Construct Cell towards the tumor core through chemotaxis mechanisms. In certain embodiments, the homing signal is a cytokine or a chemokine. In certain embodiments, the homing signal is a cytokine receptor or a chemokine receptor. Non-limiting examples of homing signal include IFNGR2, IL6R+IL6ST, IL12RB1, IL12RB2, CXCR3, CXCR6, CCR2, CCR5, CCR7, CCR1, CCR3, CCR4, CCR6, CCR8, CX3CR1, CXCR1, CXCR2, CXCR4, CXCR6, XCR1, and CCR10.

In certain embodiments, the Payload is a modulator of reactive oxygen species (ROS). In certain embodiments, the modulators of reactive oxygen species are selected as a Payload because cancer cells typically have elevated ROS burden and neutrophils do create oxidative bursts as a way of destroying cancer cells. Non-limiting examples of modulators of ROS include nicotinamide adenine dinucleotide phosphate oxidase, nitric oxide synthase, and xanthine oxidase.

In certain embodiments, the competitive ligand inhibitor selected as a Payload binds to receptors on tumor cells and prevents activation.

In certain embodiments, the Payload of the Secondary Promoter Constructs is a transcription factor, florescent proteins, cytokine/chemokine and their receptor, cytokine fusion, cytokine receptor fusion, switch receptor (an extracellular domain which binds an inhibitory signal and an intracellular stimulatory domain), costimulatory receptor and their ligand, dominant-negative and null mutant immune checkpoint receptor, immune checkpoint antagonist, antigen-binding receptor or fragment and their coreceptor, coreceptor fusion, metabolic regulator, antibody, transpeptidase, protease, sheddase, growth factor and growth regulator, scaffold protein, adaptor protein, kinase, phosphatase, ITAM containing receptor, methyltransferase loss of function or null mutant, methylcytosine loss of function or null mutant, toll-like receptor, glycosylase, glycosidase, glycosyltransferase, DNA repair protein, inducible caspase, Truncated LNGFR or EGFR, cytosolic DNA sensor, GPCR and their ligand, leucine zipper fusion, Cas protein, guide RNA, short hairpin RNA, innate immune agonist, or drug resistance marker.

3.1.2. Inhibitory RNA Molecules

In certain embodiments, the Secondary Promoter Constructs disclosed herein include an inhibitory RNA molecule. In certain embodiments, the Payload is an inhibitory RNA molecule. As used here, the terms “inhibitory RNA” or “inhibitory RNA molecule” refer to an RNA molecule capable of interfering with, or suppressing, the expression of a gene (e.g., RNA interference or RNAi). In certain embodiments, the inhibitory RNA molecule is capable of interfering with, or suppressing, the expression of a single gene. In certain embodiments, the inhibitory RNA molecules are capable of interfering with, or suppressing, the expression of at least two or more, at least three or more, at least four or more, or at least five or more genes. In certain embodiments, the inhibitory RNA molecule comprises a small interference RNA (siRNA), a small hairpin RNA (shRNA), a microRNA (miRNA), or a combination thereof.

In certain embodiments, the inhibitory RNA molecules are positioned in the Secondary Promoter Construct in a series or multiplex arrangement such that multiple inhibitory RNA molecules are simultaneously expressed from a single polycistronic transcript. In certain embodiments, the inhibitory RNA molecules are positioned in the Payload in a series or multiplex arrangement such that multiple inhibitory RNA molecules are simultaneously expressed from a single polycistronic transcript. In certain embodiments, the inhibitory RNA molecules can be linked to each other by non-functional linker sequence(s).

In certain embodiments, the inhibitory RNA molecule comprises a small hairpin RNA (shRNA). In certain embodiments, the inhibitory RNA molecule comprises a microRNA (miRNA). A miRNA is an RNA molecule (artificial or naturally occurring) of about 22 nucleotides in length that can be used to silence target gene expression via RNA interference (RNAi).

In certain embodiments, the inhibitory RNA molecule is a naturally occurring miRNA. In certain embodiments, the inhibitory RNA molecule is an artificial miRNA. Artificial miRNAs can be produced by placing an RNA sequence directed against a target gene in a miRNA framework (e.g., microRNA sequences for processing and loop). In certain embodiments, the inhibitory RNA molecule comprises, from 5′ to 3′, a first miRNA flanking sequence, a first stem, a loop, a second stem that is partially or fully complementary to the first stem, and a second miRNA flanking sequence. In certain embodiments, the first and second miRNA flanking sequences are derived from a naturally occurring miRNA. For example, without any limitation, the first and second miRNA flanking sequences are derived from miR-155, miR-30, miR-17/92, miR-122, and miR-21. In certain embodiments, the first and second miRNA flanking sequences are derived from a miR-155. In certain embodiments, the first and second miRNA flanking sequences are derived from a miR-30. In certain embodiments, the first and second miRNA flanking sequences are derived from a miR-30a.

In certain embodiments, the inhibitory RNA molecule comprises a SIBR scaffold. In certain embodiments, the inhibitory RNA molecules comprise an eSIBR scaffold. Additional details on the eSIBR scaffold can be found in Fowler et al., Nucleic acids research 44.5 (2016): e48-e48, the content of which is incorporated by reference in its entirety.

In certain embodiments, the inhibitory RNA molecule comprises a miR-E scaffold. Additional details on the miR-E scaffold can be found in Fellmann et al., Cell reports 5.6 (2013): 1704-1713, the content of which is incorporated by reference in its entirety.

In certain embodiments, the inhibitory RNA molecules comprise a microRNA cluster. A miRNA cluster is a set of two or more miRNAs, which are transcribed from physically adjacent miRNA genes. miRNA genes can be found either in protein-coding or non-coding regions of transcription units (TUs). Typically, miRNAs in a cluster are transcribed in the same orientation and are not separated by a transcription unit or a miRNA in the opposite orientation. In certain embodiments, the miRNA cluster includes two miRNAs. In certain embodiments, the miRNA cluster includes three miRNAs. In certain embodiments, the miRNA cluster includes four miRNAs. In certain embodiments, the miRNA cluster includes five miRNAs. In certain embodiments, the miRNA cluster includes six miRNAs. In certain embodiments, the miRNA cluster derives from naturally occurring miRNA clusters. Non-limiting examples of naturally occurring miRNA clusters include miR-17/92, miR-23b/27b/24, and miR-106.

In certain embodiments, the inhibitory RNA molecules target a gene selected from the group consisting of A2AR, CBLB, TGFBR2, or a combination thereof.

In certain embodiments, the inhibitory RNA molecule targets a CBLB transcript. In certain embodiments, the inhibitory RNA molecule comprises a nucleotide sequence that is at least about 80%, about 85%, about 90%, about 95%, about 96%, about 97%, about 98%, or about 99% identical to the nucleotide sequence set forth in SEQ ID NO: 5. In certain embodiments, the inhibitory RNA molecule comprises the nucleotide sequence set forth in SEQ ID NO: 5. In certain embodiments, the inhibitory RNA molecule consists of the nucleotide sequence set forth in SEQ ID NO: 5.

In certain embodiments, the inhibitory RNA molecule comprises a nucleotide sequence that is at least about 80%, about 85%, about 90%, about 95%, about 96%, about 97%, about 98%, or about 99% identical to the nucleotide sequence set forth in SEQ ID NO: 6. In certain embodiments, the inhibitory RNA molecule comprises the nucleotide sequence set forth in SEQ ID NO: 6. In certain embodiments, the inhibitory RNA molecule consists of the nucleotide sequence set forth in SEQ ID NO: 6.

In certain embodiments, the inhibitory RNA molecule comprises a nucleotide sequence that is at least about 80%, about 85%, about 90%, about 95%, about 96%, about 97%, about 98%, or about 99% identical to the nucleotide sequence set forth in SEQ ID NO: 7. In certain embodiments, the inhibitory RNA molecule comprises the nucleotide sequence set forth in SEQ ID NO: 7. In certain embodiments, the inhibitory RNA molecule consists of the nucleotide sequence set forth in SEQ ID NO: 7. SEQ ID NOs: 5-7 are provided below.

[SEQ ID NO: 5]
TCGACTTCTTAACCCAACAGAAGGCTCGAGAAGGTATATT
GCTGTTGACAGTGAGCGAAAGATGTCAAGATTGAGCCTTT
AGTGAAGCCACAGATGTAAAGGCTCAATCTTGACATCTTG
TGCCTACTGCCTCGGACTTCAAGGGGCTACTTTAGGA
[SEQ ID NO: 6]
GTCGACTGGAGGCTTGCTTTGGGCTGTATGCTGAAGGCTC
AATCTTGACATCTTGTTTTGGCCTCTGACTGACAAGATTC
AGATTGAGCCTTGGACACAAGGCCCTTTATCAGCACTCAC
ATGGAACAAATGGCCACCGTGGGAGGATGACAACTCGAG
[SEQ ID NO: 7]
GATGTCAAGATTGAGCCTTGCCCTGACCCAGCAAGGCTCA
ATCTTGACATC

In certain embodiments, the inhibitory RNA molecule targets a CBLB transcript and an A2AR transcript. In certain embodiments, the inhibitory RNA molecule comprises a nucleotide sequence that is at least about 80%, about 85%, about 90%, about 95%, about 96%, about 97%, about 98%, or about 99% identical to the nucleotide sequence set forth in SEQ ID NO: 8. In certain embodiments, the inhibitory RNA molecule comprises the nucleotide sequence set forth in SEQ ID NO: 8. In certain embodiments, the inhibitory RNA molecule consists of the nucleotide sequence set forth in SEQ ID NO: 8.

In certain embodiments, the inhibitory RNA molecule comprises a nucleotide sequence that is at least about 80%, about 85%, about 90%, about 95%, about 96%, about 97%, about 98%, or about 99% identical to the nucleotide sequence set forth in SEQ ID NO: 9. In certain embodiments, the inhibitory RNA molecule comprises the nucleotide sequence set forth in SEQ ID NO: 9. In certain embodiments, the inhibitory RNA molecule consists of the nucleotide sequence set forth in SEQ ID NO: 9. SEQ ID NO: 8 and SEQ ID NO: 9 are provided below.

[SEQ ID NO: 8]
TGGAGGCTTGCTTTGGGCTGTATGCTGAAGGCTCAATCTT
GACATCTTGTTTTGGCCTCTGACTGACAAGATTCAGATTG
AGCCTTGGACACAAGGCCCTTTATCAGCACTCACATGGAA
CAAATGGCCACCGTGGGAGGATGACAAGTCGACTCGACTT
CTTAACCCAACAGAAGGCTCGAGAAGGTATATTGCTGTTG
ACAGTGAGCGCCAGACCTTCCGCAAGATCATTTAGTGAAG
CCACAGATGTAAATGATCTTGCGGAAGGTCTGGTGCCTAC
TGCCTCGGACTTCAAGGGGCTACTTTAGGA
[SEQ ID NO: 9]
TCGACTTCTTAACCCAACAGAAGGCTCGAGAAGGTATATT
GCTGTTGACAGTGAGCGCCAGACCTTCCGCAAGATCATTT
AGTGAAGCCACAGATGTAAATGATCTTGCGGAAGGTCTGG
TGCCTACTGCCTCGGACTTCAAGGGGCTACTTTAGGAGTC
GACTGGAGGCTTGCTTTGGGCTGTATGCTGAAGGCTCAAT
CTTGACATCTTGTTTTGGCCTCTGACTGACAAGATTCAGA
TTGAGCCTTGGACACAAGGCCCTTTATCAGCACTCACATG
GAACAAATGGCCACCGTGGGAGGATGACAA

In certain embodiments, the inhibitory RNA molecule targets a CBLB transcript, an A2AR transcript, and a TGFBR2. In certain embodiments, the inhibitory RNA molecule comprises a nucleotide sequence that is at least about 80%, about 85%, about 90%, about 95%, about 96%, about 97%, about 98%, or about 99% identical to the nucleotide sequence set forth in SEQ ID NO: 10. In certain embodiments, the inhibitory RNA molecule comprises the nucleotide sequence set forth in SEQ ID NO: 10. In certain embodiments, the inhibitory RNA molecule consists of the nucleotide sequence set forth in SEQ ID NO: 10. SEQ ID NO: 10 is provided below.

[SEQ ID NO: 10]
CTGACTACATCACAGCAGCATACGTGGAGATGAGGCGAGA
GGCTTGGGCTAGTAAGGATGCCACCTATACTTCTGCCCGG
ACCCTGCTGGCTATCCTGCGCCTTTCCACTGCTCTGGTAA
GTGCCCAAATTGCTGGAGGGCCATCTGTTTTGACCCTTAA
AGGGGTAGCTCCTTACCGTGCTCTCATTGCCGCCTCCCCA
CCTCCCGCTCGAGCCCTGCCGGGGCGATGTCAAGATTGAG
CCTTGCTTTGTGTAGGCAAGGCTCAATCTTGACATCGCTC
CAGCAGGGCACGCAATGCTTCGTGGAGGGAAAGGCCTTTT
CCCCACTTCTTAACCTTCACTGAGAGGGTGGTTGGGGTCT
GTTTCACTCCATGTGTCCTAGATCCTGTGCTACAGACCTT
CCTTTCTGTCCTCCCGTCTTGGAATTCAGTCCTGGGGGCT
GGAATGATCTTGCGGAAGGTCTGTGTGATTACCCAACCAG
ACCTCCGCAAGTATCATATATAGCCCCCGGGTTCCGTTCT
CTCTGCCAATTGTCTTCTTGGCTGAGCTCCCCAAGCTCCA
TCTGTCATGCTGGGGAGCCCAGTGGCGTTCAAAAGGGTCT
GGTCTCCCTCACAGGACAGCTGAACTCTAGAAGTCCTGGG
GGCTCGATGAAGAAAGTCTCACCAGGCTGTGATTACCCAA
CGCCTGGTAGACTTTACTTCAGCGCGAGCCCCCGGGACAC
ACCGCGGATGCTGGGGGGAGGGGGGATTCCACTCCTGTTT
TGTGAGTAGGCGACCCATGGGCTGCCCAGCCTTAAAGCCA
GAACAAGGGTGTCCCCTGACCTCGTTCCACTGCCCTCCTC
CCGTTCCCATCTTTCCCCCCTACCTTCCCCTTAGGCACGT
CTGAGAATGGTGGATGTGGTGGAGAAAGAAGATGTGAATG
AAGCCATCAGGCTAATGGAGATGTCAAAGGACTCTCTTCT
AGGAGACAAGGGGCAGACA

In certain embodiments, the inhibitory RNA molecule is flanked by a splice acceptor site. A “splice acceptor site” is a polynucleotide site at the 3′ of an intron and regulates the splicing of RNA. In certain embodiments, the inhibitory RNA molecule is flanked by a splice donor site. A “splice donor site” is a polynucleotide site at the 5′ of an intron and regulates the splicing of RNA. In certain embodiments, the inhibitory RNA molecule is flanked by a splice acceptor site and a splice donor site. In certain embodiments, the inhibitory RNA molecule is flanked at its 5′ by a splice acceptor site. In certain embodiments, the inhibitory RNA molecule is flanked at its 3′ by a splice acceptor site. In certain embodiments, the inhibitory RNA molecule is flanked at its 5′ by a splice donor site. In certain embodiments, the inhibitory RNA molecule is flanked at its 3′ by a splice donor site.

In certain embodiments, the splice acceptor site comprises a nucleotide sequence that is at least about 80%, about 85%, about 90%, about 95%, about 96%, about 97%, about 98%, or about 99% identical to the nucleotide sequence set forth in SEQ ID NO: 11. In certain embodiments, the splice acceptor site comprises the nucleotide sequence set forth in SEQ ID NO. 11. In certain embodiments, the splice acceptor site consists of the nucleotide sequence set forth in SEQ ID NO: 11. SEQ ID NO: 11 is provided below.

[SEQ ID NO: 11]
TTCCCCTTAGGCACGTCTGAGAATGGTGGATGTGGTGGAG
AAAGAAGATGTGAATGAAGCCATCAGGCTAATGGAGATGT
CAAAGGACTCTCTTCTAGGAGACAAGGGGCAGACA

In certain embodiments, the splice acceptor site comprises a nucleotide sequence that is at least about 80%, about 85%, about 90%, about 95%, about 96%, about 97%, about 98%, or about 99% identical to the nucleotide sequence set forth in SEQ ID NO: 12. In certain embodiments, the splice acceptor site comprises the nucleotide sequence set forth in SEQ ID NO: 12. In certain embodiments, the splice acceptor site consists of the nucleotide sequence set forth in SEQ ID NO: 12. SEQ ID NO: 12 is provided below.

[SEQ ID NO: 12]
TGTCCCACAGATATCCAGAACCCCGACCCCGCCGTGTACC
AGCTGCGGGACAGCAAGAGCAGCGACAAGAGCGTGTGCCT
GTT

In certain embodiments, the splice donor site comprises a nucleotide sequence that is at least about 80%, about 85%, about 90%, about 95%, about 96%, about 97%, about 98%, or about 99% identical to the nucleotide sequence set forth in SEQ ID NO: 13. In certain embodiments, the splice donor site comprises the nucleotide sequence set forth in SEQ ID NO: 13. In certain embodiments, the splice donor site consists of the nucleotide sequence set forth in SEQ ID NO: 13. SEQ ID NO: 13 is provided below.

[SEQ ID NO: 13]
CTGACTACATCACAGCAGCATACGTGGAGATGAGGCGAGA
GGCTTGGGCTAGTAAGGATGCCACCTATACTTCTGCCCGG
ACCCTGCTGGCTATCCTGCGCCTTTCCACTGCTCTGGTAA
GTGCCC

3.1.3. Poly-Adenylation Sequence (Poly-A)

In certain embodiments, the Secondary Promoter Constructs disclosed herein include a poly-adenylation signal sequence (poly-A). The poly-A signal sequence is a long chain of adenine nucleotides that is added to a messenger RNA (mRNA) molecule during RNA processing to increase the stability of the molecule. Typically, a poly-A signal sequence is between about 100 and about 250 residues long. The poly-A signal sequence makes the RNA molecule more stable and prevents its degradation. Additionally, the poly-A signal sequence allows the mature messenger RNA molecule to be exported from the nucleus and translated into a protein by ribosomes in the cytoplasm.

In certain embodiments, the poly-adenylation signal sequence is a type of DNA sequence that mediates the addition of a poly-A signal sequence to the 3′ end of the mRNA transcript. In certain embodiments, the poly-A signal sequence acts as a transcription stop signal. In certain embodiments, the addition of a poly-A signal sequence is required for the production of mature mRNA. In certain embodiments, the poly-A signal sequence is important for nuclear export, translation, and mRNA stability.

In certain embodiments, a poly-A signal sequence is only included after the TCR gene to decouple transcription of the TCR genes from the transcription of a Payload. In essence, adding the poly-A signal sequence at this position leads to the production of two separate transcripts. Accordingly, when combined with a secondary promoter, the poly-A signal sequence allows the decoupling of the expression of the Payload from the expression of the TCR. In certain embodiments and as shown in Format 2, a second poly-A signal was added even though only the first poly-A signal sequence is required for the production of two distinct transcripts.

In certain embodiments, the Secondary Promoter Construct includes one poly-A signal sequence. In certain non-limiting embodiments, the poly-A signal sequence can be a simian virus 40 (SV40) poly-A signal sequence, an SV40 poly-A signal sequence, a human growth hormone (hGH) poly-A signal sequence, a bovine growth hormone (BGH) poly-A signal sequence, or a rabbit beta-globin (rbGlob) poly-A signal sequence. In certain embodiments, the Secondary Promoter Construct includes two poly-A signal sequences. In certain embodiments, the two poly-A signal sequences are the same. In certain embodiments, the two poly-A signal sequences are different.

In certain embodiments, the poly-A signal sequence used in the Secondary Promoter Constructs is the BGH poly-A signal sequence. In certain embodiments, the BGH poly-A signal sequence comprises a nucleotide sequence that is at least about 80%, about 85%, about 90%, about 95%, about 96%, about 97%, about 98%, or about 99% identical to the nucleotide sequence set forth in SEQ ID NO: 14. In certain embodiments, the BGH poly-A signal sequence comprises the nucleotide sequence set forth in SEQ ID NO: 14. In certain embodiments, the BGH poly-A signal sequence consists of the nucleotide sequence set forth in SEQ ID NO: 14. SEQ ID NO: 14 is provided below.

[SEQ ID NO: 14]
TGTGCCTTCTAGTTGCCAGCCATCTGTTGTTTGCCCCTCC
CCCGTGCCTTCCTTGACCCTGGAAGGTGCCACTCCCACTG
TCCTTTCCTAATAAAATGAGGAAATTGCATCGCATTGTCT
GAGTAGGTGTCATTCTATTCTGGGGGGTGGGGTGGGGCAG
GACAGCAAGGGGGAGGATTGGGAAGACAATAGCAGGCATG
CTGGGGATGCGGTGGGCTCTATGGC

In certain embodiments, the poly-A signal sequence used in the Secondary Promoter Constructs is the SV40 poly-A signal sequence. In certain embodiments, the SV40 poly-A signal sequence comprises a nucleotide sequence that is at least about 80%, about 85%, about 90%, about 95%, about 96%, about 97%, about 98%, or about 99% identical to the nucleotide sequence set forth in SEQ ID NO: 15. In certain embodiments, the SV40 poly-A signal sequence comprises the nucleotide sequence set forth in SEQ ID NO: 15. In certain embodiments, the SV40 poly-A signal sequence consists of the nucleotide sequence set forth in SEQ ID NO: 15. SEQ ID NO: 15 is provided below.

[SEQ ID NO: 15]
GCTTTATTTGTGAAATTTGTGATGCTATTGCTTTATTTGT
AACCATTATAAGCTGCAATAAACAAGTTAACAACAACAAT
TGCATTCATTTTATGTTTCAGGTTCAGGGGGAGATGTGGG
AGGTTTTTTAAAGC

In certain embodiments, the SV40 poly-A signal sequence further comprises an SV40 upstream element. In certain embodiments, the SV40 upstream element comprises a nucleotide sequence that is at least about 80%, about 85%, about 90%, about 95%, about 96%, about 97%, about 98%, or about 99% identical to the nucleotide sequence set forth in SEQ ID NO: 3. In certain embodiments, the SV40 upstream element comprises the nucleotide sequence set forth in SEQ ID NO: 16. In certain embodiments, the SV40 upstream element consists of the nucleotide sequence set forth in SEQ ID NO: 16. SEQ ID NO: 16 is provided below.

[SEQ ID NO: 16]
TTTATTTGTGAAATTTGTGATGCTATTGCTTTATTTGTAACCAT

In certain embodiments, the poly-A signal sequence used in the Secondary Promoter Constructs is a 6T sequence. In certain embodiments, the 6T sequence comprises a nucleotide sequence that is at least about 80%, about 85%, about 90%, about 95%, about 96%, about 97%, about 98%, or about 99% identical to the nucleotide sequence set forth in SEQ ID NO: 17. In certain embodiments, the 6T poly-A signal sequence comprises the nucleotide sequence set forth in SEQ ID NO: 17. In certain embodiments, the 6T poly-A signal sequence consists of the nucleotide sequence set forth in SEQ ID NO: 17. SEQ ID NO: 17 is provided below.

[SEQ ID NO: 17]
TTTTTT

In certain embodiments, the poly-A signal sequence used in the Secondary Promoter Constructs shown in FIGS. 2A and 3A is the BGH poly-A signal sequence. In certain embodiments, the poly-A signal sequence used in the Secondary Promoter Constructs shown in FIGS. 2A and 3A is the SV40 poly-A signal sequence.

In certain embodiments, the first poly-A signal sequence used in the Secondary Promoter Constructs shown in FIGS. 2B and 3B is the BGH poly-A signal sequence and the first poly-A signal sequence used in the Secondary Promoter Constructs shown in FIGS. 2B and 3B is the SV40 poly-A signal sequence. In certain embodiments, the first poly-A signal sequence used in the Secondary Promoter Constructs shown in FIGS. 2B and 3B is the SV40 poly-A signal sequence and the first poly-A signal sequence used in the Secondary Promoter Constructs shown in FIGS. 2B and 3B is the BGH poly-A signal sequence.

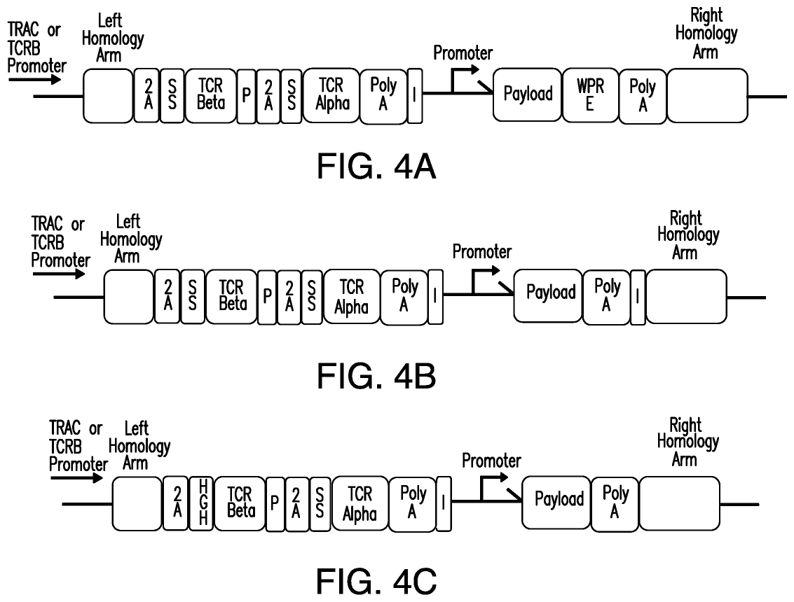

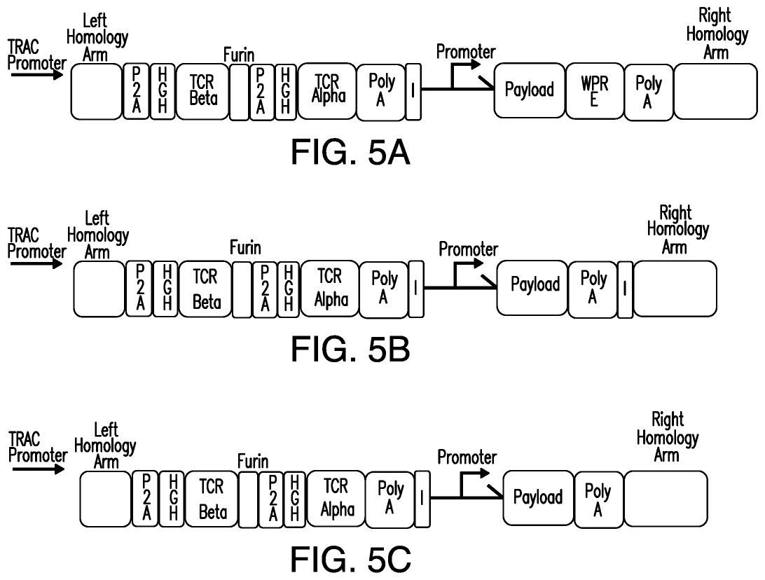

In certain embodiments, the first poly-A signal sequence used in the Secondary Promoter Constructs shown in FIGS. 4A-4C and 5A-5C is the BGH poly-A signal sequence, and the first poly-A signal sequence used in the Secondary Promoter Constructs shown in FIGS. 4A-4C and 5A-5C is the SV40 poly-A signal sequence. In certain embodiments, the first poly-A signal sequence used in the Secondary Promoter Constructs shown in FIGS. 4A-4C and 5A-5C is the SV40 poly-A signal sequence, and the first poly-A signal sequence used in the Secondary Promoter Constructs shown in FIGS. 4A-4C and 5A-5C is the BGH poly-A signal sequence.

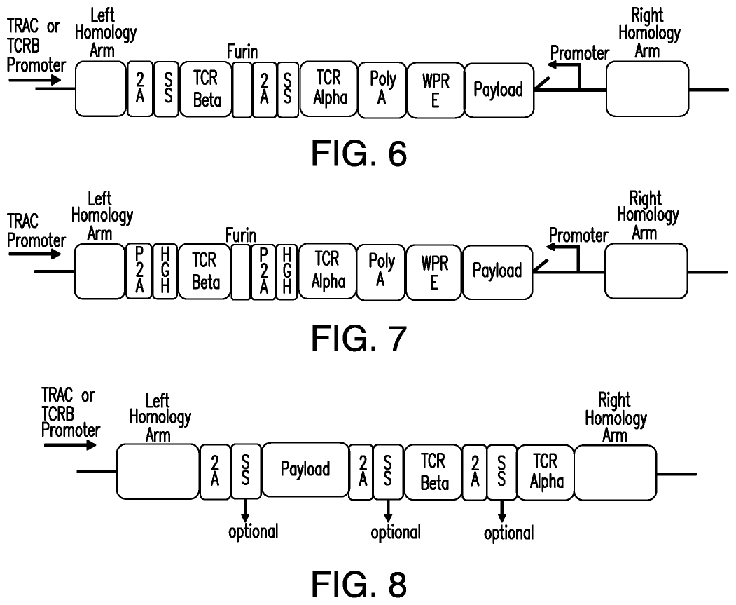

In certain embodiments, the poly-A signal sequence used in the Secondary Promoter Constructs shown in FIGS. 6 and 7 is the BGH poly-A signal sequence. In certain embodiments, the poly-A signal sequence used in the Secondary Promoter Constructs shown in FIGS. 6 and 7 is the SV40 poly-A signal sequence.

3.1.4. Insulators

In certain embodiments, the Secondary Promoter Constructs disclosed herein include a transcriptional insulator or insulator. Insulators are a type of DNA sequence that helps divide the genome into distinct “genetic neighborhoods.” Insulators can help prevent regulatory elements designed to affect the expression of one gene from also affecting the expression of another nearby gene.

In certain embodiments, insulators can reduce promoter interference. Promoter interference is a molecular event characterized by the perturbation of one transcription unit by another. For example, without any limitation, promoter interference can occur between the transcription units of the genes of the Secondary Promoter Constructs (e.g., the sequence encoding an exogenous TCR and the Payload). Additional information on promoter interference can be found in Eszterhas et al., Molecular and Cellular Biology 22.2 (2002): 469-479.

In certain embodiments, promoter interference occurs when the expression of the Payload reduces the TCR expression. Accordingly, in certain embodiments, an insulator can be inserted into the Secondary Promoter Construct to prevent reduction in TCR expression.

In certain embodiments, insulators reduce promoter leakiness. Accordingly, in certain embodiments, insulators can be used to reduce transgene silencing.

In certain embodiments, an insulator is added to the construct between the TCR gene and the Payload. In certain embodiments, an insulator is added to the construct between the TCR genes and the promoter region.

In certain embodiments, the insulators used in the Secondary Promoter Constructs are HS4 or IS2. Additional examples of insulators encompassed by the present disclosure include, without any limitation, CTCF insulator, Cohesin insulator, TFIIIC insulator, Condensin insulator, p68 insulator, PARP1 insulator, Bptf insulator, TGF-β insulator, and Kaiso insulator. Further information and examples of insulators can be found in Liu et al., Nature biotechnology 33.2 (2015): 198-203.

In certain embodiments, the insulator is an HS4 insulator. In certain embodiments, the HS4 insulator comprises a nucleotide sequence that is at least about 80%, about 85%, about 90%, about 95%, about 96%, about 97%, about 98%, or about 99% identical to the nucleotide sequence set forth in SEQ ID NO: 18. In certain embodiments, the HS4 insulator comprises the nucleotide sequence set forth in SEQ ID NO: 18. In certain embodiments, the HS4 insulator consists of the nucleotide sequence set forth in SEQ ID NO: 18. SEQ ID NO: 18 is provided below:

[SEQ ID NO: 18]
GAGCTCACGGGGACAGCCCCCCCCCAAAGCCCCCAGGGAT
GTAATTACGTCCCTCCCCCGCTAGGGGGCAGCAGCGAGCC
GCCCGGGGCTCCGCTCCGGTCCGGCGCTCCCCCCGCATCC
CCGAGCCGGCAGCGTGCGGGGACAGCCCGGGCACGGGGAA
GGTGGCACGGGATCGCTTTCCTCTGAACGCTTCTCGCTGC
TCTTTGAGCCTGCAGACACCTGGGGGGATACGGGGAAAAA
GCTT

In certain embodiments, the insulator is an IS2 insulator. In certain embodiments, the IS2 insulator comprises a nucleotide sequence that is at least about 80%, about 85%, about 90%, about 95%, about 96%, about 97%, about 98%, or about 99% identical to the nucleotide sequence set forth in SEQ ID NO: 19. In certain embodiments, the IS2 insulator comprises the nucleotide sequence set forth in SEQ ID NO: 19. In certain embodiments, the IS2 insulator consists of the nucleotide sequence set forth in SEQ ID NO: 19. SEQ ID NO: 19 is provided below:

[SEQ ID NO: 19]
CGGGGACAGCCCCCCCCCAAAGCCCCCAGGGATGTAATTA
CGTCCCTCCCCCGCTAGGGGGCAGCAGCGAGCCGCCCGGG
GCTCCGCTCCGGTCCGGCGCTCCCCCCGCATCCCCGAGCC
GGCAGCGTGCGGGGACAGCCCGGGCACGGGGAAGGTGGCA
CGGGATCGCTTTCCTCTGAACGCTTCTCGCTGCTCTTTGA
GCCTGCAGACACCTGGGGGGATACGGGGAAAATGTGTCTG
AGCCTGCATGTTTGATGGTGTCTGGATGCAAGCAGAAGGG
GTGGAAGAGCTTGCCTGGAGAGATACAGCTGGGTCAGTAG
GACTGGGACAGGCAGCTGGAGAATTGCCATGTAGATGTTC
ATACAATCGTCAAATCATGAAGGCTGGAAAAGCCCTCCAA
GATCCCCAAGACCAACCCCAACCCACCCACCGTGCCCACT
GGCCATGTCCCTCAGTGCCACATCCCCACAGTTCTTCATC
ACCTCCAGGGACGGTGACCCCCCCACCTCCGTGGGCAGCT
GTGCCACTGCAGCACCGCTCTTTGGAGAAGGTAAATCTTG
CTAAATCCAGCCCGACCCTCCCCTGGCACAACGTAAGGCC
ATTATCTCTCATCCAACTCCAGGACGGAGTCAGTGAGAAT
ATTTAAATAAACTTATAAATTGTGAGAGAAATTAATGAAT
GTCTAAGTTAATGCAGAAACGGAGGCTCCTCATTTATTTT
TGAACTTAAAGACTTAATATTGTGAAGGTATACTTTCTTT
AATAATAAGCCTGCGCCCAATATGTTCACCCCAAAAAAGC
TGTTTGTTAACTTGTCAACCTCATTTAAAATATATAAGAA
ACAGCCCAAAGACAATAACAAAAGAATAATAAAAAAGAAT
GAAATATGTAATTCTTTCAGAGTAAAAATCACACCCATGA
CCTGGCCACTGAGGGCTTGATCAATTCACTTTGAATTTGG
CATTAAATACCATTAAGGTATATTAACTGATTTTAAAATA
AGATATATTC

In certain embodiments, the Secondary Promoter Construct includes one insulator. In certain embodiments, the Secondary Promoter Construct includes two insulators. In certain embodiments, the two insulators are the same. In certain embodiments, the two insulators are different.

In certain embodiments, the insulator used in Format 1 constructs is HS4 (FIGS. 2A, 2B, 3A, and 3B). In certain embodiments, the insulator used in Format 1 is IS2 (FIGS. 2A, 2B, 3A, and 3B).

In certain embodiments, the insulator used in Format 2 constructs that only use 1 insulator is HS4 (FIGS. 4A, 4C, 5A, and 5C). In certain embodiments, the insulator used in Format 2 constructs that only use 1 insulator is IS2 (FIGS. 4A, 4C, 5A, and 5C). In certain embodiments where Format 2 constructs use two insulators (FIGS. 4B and 5B), either both insulators are HS4, both insulators are IS2, the first insulator is HS4 and the second insulator is IS2, or the first insulator is IS2 and the second insulator is HS4.

In certain embodiments, while not shown in the figures, insulators can be inserted in any construct disclosed herein, and where constructs contain an insulator a second insulator can be added.

3.1.5. Additional Elements to Increase Translation of Transcripts

In mammalian cells, mRNA lacking in intronic regions do not get properly processed. This results in the production of abysmally low levels of proteins. Introns are ubiquitous in most eukaryotes, yet intron-free genes are commonly used for gene delivery as a method of significantly decreasing the Payload size and nucleic acid toxicity.

In certain embodiments, sequence elements that promote translation of transcripts are introduced into the Secondary Promoter Constructs. In certain embodiments, sequence elements that promote normal processing of intron-free transcripts are introduced into the Secondary Promoter Constructs.

In certain embodiments, the Secondary Promoter Construct includes an element to increase the translation of transcripts. In certain embodiments, the element to increase translation of transcripts is a woodchuck hepatitis virus post-transcriptional regulatory element (WPRE). The WPRE can increase gene expression from a variety of vectors and is most effective when placed downstream of the gene, proximal to the polyadenylation signal.

In certain embodiments, the sequence element that promotes normal processing of intron-free transcripts and that is introduced into the Secondary Promoter Construct is a woodchuck hepatitis virus post-transcriptional regulatory element (WPRE). In certain embodiments, WPREs are often used to promote normal processing of intron-free transcripts, resulting in normal levels of protein expression.

In certain embodiments, the WPRE is a WPRE3 element. In certain embodiments, the WPRE3 element comprises a nucleotide sequence that is at least about 80%, about 85%, about 90%, about 95%, about 96%, about 97%, about 98%, or about 99% identical to the nucleotide sequence set forth in SEQ ID NO: 20. In certain embodiments, the WPRE3 element comprises the nucleotide sequence set forth in SEQ ID NO: 20. In certain embodiments, the WPRE3 element consists of the nucleotide sequence set forth in SEQ ID NO: 20. SEQ ID NO: 20 is provided below:

[SEQ ID NO: 20]
GATAATCAACCTCTGGATTACAAAATTTGTGAAAGATTGA
CTGGTATTCTTAACTATGTTGCTCCTTTTACGCTATGTGG
ATACGCTGCTTTAATGCCTTTGTATCATGCTATTGCTTCC
CGTATGGCTTTCATTTTCTCCTCCTTGTATAAATCCTGGT
TAGTTCTTGCCACGGCGGAACTCATCGCCGCCTGCCTTGC
CCGCTGCTGGACAGGGGCTCGGCTGTTGGGCACTGACAAT
TCCGTGGTG

In certain embodiments, the element to increase translation of transcripts is a transcriptional pause element. This “pause element” can regulate gene expression at the level of RNA synthesis in both prokaryotes and eukaryotes, serving to coordinate the appearance of RNA with its utilization in cellular functions, and to modulate the interaction of regulatory proteins with RNA polymerase (RNAP).

In certain embodiments, the pause element is a MAZ4 pause element. In certain embodiments, the MAZ4 pause element comprises a nucleotide sequence that is at least about 80%, about 85%, about 90%, about 95%, about 96%, about 97%, about 98%, or about 99% identical to the nucleotide sequence set forth in SEQ ID NO: 21. In certain embodiments, the MAZ4 pause element comprises the nucleotide sequence set forth in SEQ ID NO: 21. In certain embodiments, the MAZ4 pause element consists of the nucleotide sequence set forth in SEQ ID NO: 21. SEQ ID NO: 21 is provided below:

[SEQ ID NO: 21]
CCTGGCCTTGGGGGAGGGGGAGGCCAGAATGAGAGCTCCT
GGCCTTGGGGGAGGGGGAGGCCAGAATGACTCGACCTGGC
CTTGGGGGAGGGGGAGGCCAGAATGAGAGCTCCTGGCCTT
GGGGGAGGGGGAGGCCAGAATGA

In certain embodiments, the Secondary Promoter Constructs described herein are designed to integrate into the genome in a manner such that the transcripts of the TCR and the Payload include intronic regions (see, e.g., Format 1; FIGS. 2A, 2B, 3A, and 3C). In certain embodiments, the Secondary Promoter Constructs described herein are designed such that the transcript of the Payload lacks intronic regions (see, e.g., Formats 2 and 3; FIGS. 4A-4C, 5A-5C, 6, and 7). In certain embodiments, in order to address the lack of intronic regions of the Payload transcript (see, e.g., Formats 2 and 3; FIGS. 4A-4C, 5A-5C, 6, and 7), a sequence element that promotes translation of transcripts is introduced prior to the poly-A signal sequence in order to mediate proper processing prior to translation of the promoter transcript. In certain embodiments, the sequence element that promotes translation of transcripts is WPRE (see, e.g., Formats 2 and 3; FIGS. 4A, 5A, 6, and 7).

In certain embodiments, the WPRE element shown in FIGS. 4A, 5A, 6, and 7 as diagrams of Formats 2 and 3 can be substituted with wPRE3, HPRE, or wPRE-O.

In certain embodiments, the WPRE element shown in FIGS. 4A, 5A, 6, and 7 as diagrams of Formats 2 and 3 is WPRE3 (i.e., WPRE element shown in the figures is replaced with WPRE3).

In certain embodiments, the WPRE element shown in FIGS. 4A, 5A, 6, and 7 as diagrams of Formats 2 and 3 is WPRE3 (i.e., WPRE element shown in the figures is replaced with WPRE3 as a preferred sequence element to promote translation of the transcript).

3.1.6. Secondary Promoters

In certain embodiments, the Secondary Promoter Construct includes a promoter. In certain embodiments, the promoter for the expression of the Payload of the Secondary Promoter Construct can be a eukaryotic promoter, a mammalian promoter, a viral promoter, a synthetic promoter, a minimal promoter, a hybrid promoter, a tissue-specific promoter, an inducible promoter, or a constitutive promoter.

In certain embodiments, the promoter for the expression of the Payload of the Secondary Promoter Construct is a constitutive promoter (i.e., a promoter that displays stable gene expression patterns over time). In certain embodiments, a constitutive promoter is selected in cases where the Payload of interest has a low toxicity profile and in cases where there is no clear benefit in linking protein expression to a specific time or place. In certain non-limiting examples, the constitutive promoter is the EF-1α promoter, the hACTB promoter, the hPGK promoter, the MND promoter, or the U6 promoter.

In certain embodiments, the constitutive promoter is an EF-1α promoter. In certain embodiments, the EF-1α promoter comprises a nucleotide sequence that is at least about 80%, about 85%, about 90%, about 95%, about 96%, about 97%, about 98%, or about 99% identical to the nucleotide sequence set forth in SEQ ID NO: 22. In certain embodiments, the EF-1α promoter comprises the nucleotide sequence set forth in SEQ ID NO: 22. In certain embodiments, the EF-1α promoter consists of the nucleotide sequence set forth in SEQ ID NO: 22. SEQ ID NO: 22 is provided below:

[SEQ ID NO: 22]
CGTGAGGCTCCGGTGCCCGTCAGTGGGCAGAGCGCACATC
GCCCACAGTCCCCGAGAAGTTGGGGGGAGGGGTCGGCAAT
TGAACCGGTGCCTAGAGAAGGTGGCGCGGGGTAAACTGGG
AAAGTGATGTCGTGTACTGGCTCCGCCTTTTTCCCGAGGG
TGGGGGAGAACCGTATATAAGTGCAGTAGTCGCCGTGAAC
GTTCTTTTTCGCAACGGGTTTGCCGCCAGAACACAGGTAA
GTGCCGTGTGTGGTTCCCGCGGGCCTGGCCTCTTTACGGG
TTATGGCCCTTGCGTGCCTTGAATTACTTCCACCTGGCTG
CAGTACGTGATTCTTGATCCCGAGCTTCGGGTTGGAAGTG
GGTGGGAGAGTTCGAGGCCTTGCGCTTAAGGAGCCCCTTC
GCCTCGTGCTTGAGTTGAGGCCTGGCCTGGGCGCTGGGGC
CGCCGCGTGCGAATCTGGTGGCACCTTCGCGCCTGTCTCG
CTGCTTTCGATAAGTCTCTAGCCATTTAAAATTTTTGATG
ACCTGCTGCGACGCTTTTTTTCTGGCAAGATAGTCTTGTA
AATGCGGGCCAAGATCTGCACACTGGTATTTCGGTTTTTG
GGGCCGCGGGCGGCGACGGGGCCCGTGCGTCCCAGCGCAC
ATGTTCGGCGAGGCGGGGCCTGCGAGCGCGGCCACCGAGA
ATCGGACGGGGGTAGTCTCAAGCTGGCCGGCCTGCTCTGG
TGCCTGGCCTCGCGCCGCCGTGTATCGCCCCGCCCTGGGC
GGCAAGGCTGGCCCGGTCGGCACCAGTTGCGTGAGCGGAA
AGATGGCCGCTTCCCGGCCCTGCTGCAGGGAGCTCAAAAT
GGAGGACGCGGCGCTCGGGAGAGCGGGCGGGTGAGTCACC
CACACAAAGGAAAAGGGCCTTTCCGTCCTCAGCCGTCGCT
TCATGTGACTCCACGGAGTACCGGGCGCCGTCCAGGCACC
TCGATTAGTTCTCGAGCTTTTGGAGTACGTCGTCTTTAGG
TTGGGGGGAGGGGTTTTATGCGATGGAGTTTCCCCACACT
GAGTGGGTGGAGACTGAAGTTAGGCCAGCTTGGCACTTGA
TGTAATTCTCCTTGGAATTTGCCCTTTTTGAGTTTGGATC
TTGGTTCATTCTCAAGCCTCAGACAGTGGTTCAAAGTTTT
TTTCTTCCATTTCAGGTGTCGTGA

In certain embodiments, the EF-1α promoter comprises a nucleotide sequence that is at least about 80%, about 85%, about 90%, about 95%, about 96%, about 97%, about 98%, or about 99% identical to the nucleotide sequence set forth in SEQ ID NO: 23. In certain embodiments, the EF-1α promoter comprises the nucleotide sequence set forth in SEQ ID NO: 23. In certain embodiments, the EF-1α promoter consists of the nucleotide sequence set forth in SEQ ID NO: 23. SEQ ID NO: 23 is provided below:

[SEQ ID NO: 23]
GAGGCTCCGGTGCCCGTCAGTGGGCAGAGCGCACATCGCC
CACAGTCCCCGAGAAGTTGGGGGGAGGGGTCGGCAATTGA
ACCGGTGCCTAGAGAAGGTGGCGCGGGGTAAACTGGGAAA
GTGATGTCGTGTACTGGCTCCGCCTTTTTCCCGAGGGTGG
GGGAGAACCGTATATAAGTGCAGTAGTCGCCGTGAACGTT
CTTTTTCGCAACGGGTTTGCCGCCAGAACACAG

In certain embodiments, the constitutive promoter is a hACTB promoter. In certain embodiments, the hACTB promoter comprises a nucleotide sequence that is at least about 80%, about 85%, about 90%, about 95%, about 96%, about 97%, about 98%, or about 99% identical to the nucleotide sequence set forth in SEQ ID NO: 24. In certain embodiments, the hACTB promoter comprises the nucleotide sequence set forth in SEQ ID NO: 24. In certain embodiments, the hACTB promoter consists of the nucleotide sequence set forth in SEQ ID NO: 24. SEQ ID NO: 24 is provided below.

[SEQ ID NO: 24]
ACTGCCTGGCCACTCCATGCCCTCCAAGAGCTCCTTCTGC
AGGAGCGTACAGAACCCAGGGCCCTGGCACCCGTGCAGAC
CCTGGCCCACCCCACCTGGGCGCTCAGTGCCCAAGAGATG
TCCACACCTAGGATGTCCCGCGGTGGGTGGGGGGCCCGAG
AGACGGGCAGGCCGGGGGCAGGCCTGGCCATGCGGGGCCG
AACCGGGCACTGCCCAGCGTGGGGCGCGGGGGCCACGGCG
CGCGCCCCCAGCCCCCGGGCCCAGCACCCCAAGGCGGCCA
ACGCCAAAACTCTCCCTCCTCCTCTTCCTCAATCTCGCTC
TCGCTCTTTTTTTTTTTCGCAAAAGGAGGGGAGAGGGGGT
AAAAAAATGCTGCACTGTGCGGCGAAGCCGGTGAGTGAGC
GGCGCGGGGCCAATCAGCGTGCGCCGTTCCGAAAGTTGCC
TTTTATGGCTCGAGCGGCCGCGGCGGCGCCCTATAAAACC
CAGCGGCGCGACGCGCCACCACCGCCGAGACCGCGTCCGC
CCCGCGAGCACAGAGCCTCGCCTTTGCCGA

In certain embodiments, the constitutive promoter is an hPGK promoter. In certain embodiments, the hPGK promoter comprises a nucleotide sequence that is at least about 80%, about 85%, about 90%, about 95%, about 96%, about 97%, about 98%, or about 99% identical to the nucleotide sequence set forth in SEQ ID NO: 25. In certain embodiments, the hPGK promoter comprises the nucleotide sequence set forth in SEQ ID NO: 25. In certain embodiments, the hPGK promoter consists of the nucleotide sequence set forth in SEQ ID NO: 25. SEQ ID NO: 25 is provided below:

[SEQ ID NO: 25]
TCGAATTCCACGGGGTTGGGGTTGCGCCTTTTCCAAGGCA
GCCCTGGGTTTGCGCAGGGACGCGGCTGCTCTGGGCGTGG
TTCCGGGAAACGCAGCGGCGCCGACCCTGGGTCTCGCACA
TTCTTCACGTCCGTTCGCAGCGTCACCCGGATCTTCGCCG
CTACCCTTGTGGGCCCCCCGGCGACGCTTCCTGCTCCGCC
CCTAAGTCGGGAAGGTTCCTTGCGGTTCGCGGCGTGCCGG
ACGTGACAAACGGAAGCCGCACGTCTCACTAGTACCCTCG
CAGACGGACAGCGCCAGGGAGCAATGGCAGCGCGCCGACC
GCGATGGGCTGTGGCCAATAGCGGCTGCTCAGCAGGGCGC
GCCGAGAGCAGCGGCCGGGAAGGGGCGGTGCGGGAGGCGG
GGTGTGGGGCGGTAGTGTGGGCCCTGTTCCTGCCCGCGCG
GTGTTCCGCATTCTGCAAGCCTCCGGAGCGCACGTCGGCA
GTCGGCTCCCTCGTTGACCGAATCACCGACCTCTCTCCCC
AG

In certain embodiments, the constitutive promoter is an MND promoter. In certain embodiments, the MND promoter comprises a nucleotide sequence that is at least about 80%, about 85%, about 90%, about 95%, about 96%, about 97%, about 98%, or about 99% identical to the nucleotide sequence set forth in SEQ ID NO: 26. In certain embodiments, the MND promoter comprises the nucleotide sequence set forth in SEQ ID NO: 26. In certain embodiments, the MND promoter consists of the nucleotide sequence set forth in SEQ ID NO: 26. SEQ ID NO: 26 is provided below:

[SEQ ID NO: 26]
GAACAGAGAAACAGGAGAATATGGGCCAAACAGGATATCT
GTGGTAAGCAGTTCCTGCCCCGGCTCAGGGCCAAGAACAG
TTGGAACAGCAGAATATGGGCCAAACAGGATATCTGTGGT
AAGCAGTTCCTGCCCCGGCTCAGGGCCAAGAACAGATGGT
CCCCAGATGCGGTCCCGCCCTCAGCAGTTTCTAGAGAACC
ATCAGATGTTTCCAGGGTGCCCCAAGGACCTGAAATGACC
CTGTGCCTTATTTGAACTAACCAATCAGTTCGCTTCTCGC
TTCTGTTCGCGCGCTTCTGCTCCCCGAGCTCTATATAAGC
AGAGCTCGTTTAGTGAACCGTCAGATCGCCTGGAGACGCC
ATCCACGCTGTTTTGACTTCCATAGAAG

In certain embodiments, the constitutive promoter is a U6 promoter. In certain embodiment the U6 promoter comprises a nucleotide sequence that is at least about 80%, about 85%, about 90%, about 95%, about 96%, about 97%, about 98%, or about 99% identical to the nucleotide sequence set forth in SEQ ID NO: 27. In certain embodiments, the U6 promoter comprises the nucleotide sequence set forth in SEQ ID NO: 27. In certain embodiments, the U6 promoter consists of the nucleotide sequence set forth in SEQ ID NO: 27. SEQ ID NO: 27 is provided below:

[SEQ ID NO: 27]
GAGGGCCTATTTCCCATGATTCCTTCATATTTGCATATAC
GATACAAGGCTGTTAGAGAGATAATTGGAATTAATTTGAC
TGTAAACACAAAGATATTAGTACAAAATACGTGACGTAGA
AAGTAATAATTTCTTGGGTAGTTTGCAGTTTTAAAATTAT
GTTTTAAAATGGACTATCATATGCTTACCGTAACTTGAAA
GTATTTCGATTTCTTGGCTTTATATATCTTGTGGAAAGGA
CGAAACACC

In certain embodiments, the promoter for the expression of the Payload of the Secondary Promoter Construct is an inducible promoter (i.e., a type of conditional promoter where the promoter is in its “on” state only under certain conditions). In certain embodiments, the inducible promoter is generally in its “off” state, unless and until it receives a signal that induces switching into the “on” state. In certain embodiments, the inducible promoter allows for a basal level of gene expression even if in the “off” state (i.e., a phenomenon known as promoter leakiness).

In certain embodiments, the inducible promoter is a TCR activation triggered inducible promoter. In certain embodiments, the TCR activation triggered inducible promoter works as follows: upon TCR engagement a transcription factor is activated (i.e. AP-1, NFAT, NF-κB); these activated transcription factors will bind the inducible promoter and initiate a switch from the “off” state to the “on” state. In certain embodiments, the TCR activation triggered inducible promoter may contain either additional or a reduced number of response elements when compared to the wild-type promoter. In certain embodiments, the TCR activation triggered inducible promoter is used to localize a Payload of interest to the tumor site. In certain embodiments, the benefit of localizing a Payload of interest to the tumor site is to allow for high levels of the Payload to be predominantly expressed in response to TCR signaling at the tumor site. In certain embodiments, this is the site maximal TCR engagement. In certain embodiments, localizing a Payload of interest to the tumor site allows for the use of Payloads with high toxicity profiles because the use of an inducible promoter can limit the systemic availability of toxic Payloads. In certain embodiments, the TCR activation trigged inducible promoter comprises a sequence of at least one regulatory element of transcription factors induced by TCR activation. In certain embodiments, the TCR activation triggered inducible promoter is the AP-1 responsive, NFAT responsive, NF-κB responsive promoter, or an NR4A-responsive promoter.

In certain embodiments, the TCR activation triggered inducible promoter is a promoter that can be activated by pathways associated with the TCR activation or T cell pathways or by induction with an activation agent (for example but not limited to a small molecule or polypeptide).

In certain embodiments, the inducible promoter is an AP-1 promoter. In certain embodiments, the AP-1 promoter comprises a nucleotide sequence that is at least about 80%, about 85%, about 90%, about 95%, about 96%, about 97%, about 98%, or about 99% identical to the nucleotide sequence set forth in SEQ ID NO: 28. In certain embodiments, the AP-1 promoter comprises the nucleotide sequence set forth in SEQ ID NO: 28. In certain embodiments, the AP-1 promoter consists of the nucleotide sequence set forth in SEQ ID NO: 28. SEQ ID NO: 28 is provided below:

[SEQ ID NO: 28]
TGAGTCAGTGACTCAGTGAGTCAGTGACTCAGTGAGTCAG
TGACTCAGTGAGTCAGTGACTCAGGAATTCAGCTTGAATA
AAATGAATATTAGAAGCTGTTAGAATAAGAGAAAATGACA
GAGGAAAACTGAAAGGGAGAACTGAAAGTGGGAAATTCCT
CTGAGGCAGAAAGGACCATCCCTTATAAATAGCACAGGCC
ATGAAGGAAGATCATTCTCACTGCAGCCTTTGACAGCCTT
TGCCTCATCTTG

In certain embodiments, the inducible promoter is an NFAT promoter. In certain embodiments, the NFAT promoter comprises a nucleotide sequence that is at least about 80%, about 85%, about 90%, about 95%, about 96%, about 97%, about 98%, or about 99% identical to the nucleotide sequence set forth in SEQ ID NO: 29. In certain embodiments, the NFAT promoter comprises the nucleotide sequence set forth in SEQ ID NO: 29. In certain embodiments, the NFAT promoter consists of the nucleotide sequence set forth in SEQ ID NO: 29. SEQ ID NO: 29 is provided below:

[SEQ ID NO: 29]
AGGAGGAAAAACTGTTTCATACAGAAGGCGTCAATTAGGA
GGAAAAACTGTTTCATACAGAAGGCGTCAATTAGGAGGAA
AAACTGTTTCATACAGAAGGCGTCAATTGGTCCCATCGAA
TTAGGAGGAAAAACTGTTTCATACAGAAGGCGTCAATTAG
GAGGAAAAACTGTTTCATACAGAAGGCGTCAATTAGGAGG
AAAAACTGTTTCATACAGAAGGCGTCAATTGGTCCCGGGA
CATTTTGACACCCCCATAATATTTTTCCAGAATTAACAGT
ATAAATTGCATCTCTTGTTCAAGAGTTCCCTATCACTCTC
TTTAATCACTACTCACAGTAACCTCAACTCCTG

In certain embodiments, the inducible promoter is an NF-κB promoter. In certain embodiments, the NF-κB promoter comprises a nucleotide sequence that is at least about 80%, about 85%, about 90%, about 95%, about 96%, about 97%, about 98%, or about 99% identical to the nucleotide sequence set forth in SEQ ID NO: 30. In certain embodiments, the NF-κB promoter comprises the nucleotide sequence set forth in SEQ ID NO: 30. In certain embodiments, the NF-κB promoter consists of the nucleotide sequence set forth in SEQ ID NO: 30. SEQ ID NO: 30 is provided below:

[SEQ ID NO: 30]
GGGACTTTCCGCTTGGGGACTTTCCGCTGGGGACTTTCCG
CTGGGGACTTTCCGCTGGGGACTTTCCGAATTCAGCTTGA
ATAAAATGAATATTAGAAGCTGTTAGAATAAGAGAAAATG
ACAGAGGAAAACTGAAAGGGAGAACTGAAAGTGGGAAATT
CCTCTGAGGCAGAAAGGACCATCCCTTATAAATAGCACAG
GCCATGAAGGAAGATCATTCTCACTGCAGCCTTTGACAGC
CTTTGCCTCATCTTG

In certain embodiments, the inducible promoter is an NR4A-responsive promoter. In certain embodiments, the NR4A-responsive promoter comprises a nucleotide sequence that is at least about 80%, about 85%, about 90%, about 95%, about 96%, about 97%, about 98%, or about 99% identical to the nucleotide sequence set forth in SEQ ID NO: 31. In certain embodiments, the NR4A-responsive promoter comprises the nucleotide sequence set forth in SEQ ID NO: 31. In certain embodiments, the NR4A-responsive promoter consists of the nucleotide sequence set forth in SEQ ID NO: 31. SEQ ID NO: 31 is provided below:

[SEQ ID NO: 31]
CTCGAGAAAGGTCACCAATTAAAGGTCACCAATTAAAGGT
CACCAATTAAAGGTCACCAATTAAAGGTCACCAATTAAAG
GTCACCAATTAAAGGTCACCAATTAAAGGTCACCAATT

In certain embodiments, the promoter for the expression of the Payload of the Secondary Promoter Construct is an EF1a core (cEF1a) promoter, an hACTB promoter, an hPGK promoter, an MND promoter, an AP-1 responsive promoter, an NFAT responsive promoter, or an NF-κB responsive promoter.

In certain embodiments, the promoter for the expression of the Payload of the Secondary Promoter Construct is a TRAC promoter. In certain embodiments, the promoter for the expression of the Payload of the Secondary Promoter Construct is a TCRβ promoter. In certain embodiments, the promoter for the expression of the Payload of the Secondary Promoter Construct of Format 3 (FIG. 8) is the TRAC promoter. In certain embodiments, the promoter for the expression of the Payload of the Secondary Promoter Construct of Format 3 (FIG. 8) is the TCRβ promoter.

In certain embodiments, the promoter for the expression of the Payload of the Secondary Promoter Construct of Format 1 (FIGS. 2A, 2B, 3A, and 3C) is the EF1a core promoter. In certain embodiments, the promoter for the expression of the Payload of the Secondary Promoter Construct of Format 1 (FIGS. 2A, 2B, 3A, and 3C) is the hACTB promoter. In certain embodiments, the promoter for the expression of the Payload of the Secondary Promoter Construct of Format 1 (FIGS. 2A, 2B, 3A, and 3C) is the hPGK promoter. In certain embodiments, the promoter for the expression of the Payload of the Secondary Promoter Construct of Format 1 (FIGS. 2A, 2B, 3A, and 3C) is the MND promoter. In certain embodiments, the promoter for the expression of the Payload of the Secondary Promoter Construct of Format 1 (FIGS. 2A, 2B, 3A, and 3C) is the AP-1 responsive promoter. In certain embodiments, the promoter for the expression of the Payload of the Secondary Promoter Construct of Format 1 (FIGS. 2A, 2B, 3A, and 3C) is the NFAT responsive promoter. In certain embodiments, the promoter for the expression of the Payload of the Secondary Promoter Construct of Format 1 (FIGS. 2A, 2B, 3A, and 3C) is the NF-κB promoter.

In certain embodiments, the promoter for the expression of the Payload of the Secondary Promoter Construct of Format 2 (FIGS. 4A-4C and 5A-5C) is the EF1a core promoter. In certain embodiments, the promoter for the expression of the Payload of the Secondary Promoter Construct of Format 2 (FIGS. 4A-4C and 5A-5C) is the hACTB promoter. In certain embodiments, the promoter for the expression of the Payload of the Secondary Promoter Construct of Format 2 (FIGS. 4A-4C and 5A-5C) is the hPGK promoter. In certain embodiments, the promoter for the expression of the Payload of the Secondary Promoter Construct of Format 2 (FIGS. 4A-4C and 5A-5C) is the MND promoter. In certain embodiments, the promoter for the expression of the Payload of the Secondary Promoter Construct of Format 2 (FIGS. 4A-4C and 5A-5C) is the AP-1 responsive promoter. In certain embodiments, the promoter for the expression of the Payload of the Secondary Promoter Construct of Format 2 (FIGS. 4A-4C and 5A-5C) is the NFAT responsive promoter. In certain embodiments, the promoter for the expression of the Payload of the Secondary Promoter Construct of Format 2 (FIGS. 4A-4C and 5A-5C) is the NF-κB promoter.

In certain embodiments, the promoter for the expression of the Payload of the Secondary Promoter Construct of Format 3 (FIGS. 6 and 7) is the EF1a core promoter. In certain embodiments, the promoter for the expression of the Payload of the Secondary Promoter Construct of Format 3 (FIGS. 6 and 7) is the hACTB promoter. In certain embodiments, the promoter for the expression of the Payload of the Secondary Promoter Construct of Format 3 (FIGS. 6 and 7) is the hPGK promoter. In certain embodiments, the promoter for the expression of the Payload of the Secondary Promoter Construct of Format 3 (FIGS. 6 and 7) is the MND promoter. In certain embodiments, the promoter for the expression of the Payload of the Secondary Promoter Construct of Format 3 (FIGS. 6 and 7) is the AP-1 responsive promoter. In certain embodiments, the promoter for the expression of the Payload of the Secondary Promoter Construct of Format 3 (FIGS. 6 and 7) is the NFAT responsive promoter. In certain embodiments, the promoter for the expression of the Payload of the Secondary Promoter Construct of Format 3 (FIGS. 6 and 7) is the NF-κB promoter.

3.1.7. Enhancers

In certain embodiments, the Secondary Promoter Construct includes an enhancer. Enhancers are cis-acting elements of DNA, usually about 10 to about 300 bp in length, that act on a promoter to increase its transcription. Many enhancer sequences are now known from mammalian genes. For example, without any limitation, enhancer sequences are derived from the globin gene, the elastase gene, the albumin gene, the α-fetoprotein gene, and the insulin gene.

In certain embodiments, the enhancer is a T cell receptor (TCR) enhancer. In certain embodiments, the TCR enhancer can be a TCRα enhancer, a TCRβ enhancer, a TCRγ enhancer, or a TCRδ enhancer. In certain embodiments, the TCR enhancer comprises a TCRα enhancer. In certain embodiments, the TCRα enhancer comprises a nucleotide sequence that is at least about 80%, about 85%, about 90%, about 95%, about 96%, about 97%, about 98%, or about 99% identical to the nucleotide sequence set forth in SEQ ID NO: 32. In certain embodiments, the TCRα enhancer comprises the nucleotide sequence set forth in SEQ ID NO: 32. In certain embodiments, the TCRα enhancer consists of the nucleotide sequence set forth in SEQ ID NO: 32. SEQ ID NO: 32 is provided below:

[SEQ ID NO: 32]
GGGCTGGGGCGGTCCCCTCCCATTTCCATGACGTCATGGT
TACCAAGAGGGGCAAGTAGGGCACCCTTTGAAGCTCTCCC
GCAGAAGCCACATCCTCTGGAAAGAAGAGTTTATAATACT
GAGTTAGAGATAGCATCGCCCCAGGCCACGTGCCGAGGGG
AGCAGGCTGGGCCGTTACACCACCCCCCAACCGCAGGTGC
AGCAAGGCCAACATGCCAGGCTGGGAGGGGCTGCCGGCCC
CTCGTTGAG

In certain embodiments, the enhancer derives from a eukaryotic cell virus. Non-limiting examples of enhancers derived from eukaryotic cell virus include the SV40 enhancer on the late side of the replication origin (bp 100-270), the cytomegalovirus early promoter enhancer, the polyoma enhancer on the late side of the replication origin, and adenovirus enhancers.

In certain embodiments, the enhancer is a cytomegalovirus (CMV) enhancer. In certain embodiments, the CMV comprises a nucleotide sequence that is at least about 80%, about 85%, about 90%, about 95%, about 96%, about 97%, about 98%, or about 99% identical to the nucleotide sequence set forth in SEQ ID NO: 33. In certain embodiments, the CMV enhancer comprises the nucleotide sequence set forth in SEQ ID NO: 33. In certain embodiments, the CMV enhancer consists of the nucleotide sequence set forth in SEQ ID NO: 33. SEQ ID NO: 33 is provided below:

[SEQ ID NO: 33]
CGTTACATAACTTACGTTAAATGGCCCGCCTGGCTGACCG
CCCAACGACCCCCGCCCATTGACGTCAATAATGATGTATG
TTCCCATAGTAACGCCAATAGGGACTTTCCATTGACGTCA
ATGGGTGGAGTATTTACGGTAAACTGCCCACTTGGCAGTA
CATCAAGTGTATCATATCCAAGTACGCCCCCTATTGACGT
CAATGACGGTAAATGGCCCGCCTGGCATTATGCCCAGTAC
ATGACCTTATGGGACTTTCCTACTTGGCAGTACATCTACG
TATTAGTCATCGCTATTACCATG

In certain embodiments, the enhancer can be operably linked to a Kozak sequence. In certain embodiments, the Kozak sequence comprises the nucleotide sequence set forth in SEQ ID NO: 34. In certain embodiments, the Kozak sequence consists of the nucleotide sequence set forth in SEQ ID NO: 34. SEQ ID NO: 34 is provided below.

[SEQ ID NO: 34]
GCCACC

3.1.8. Homology Recombination Templates

In certain embodiments, the Secondary Promoter Construct is a homologous recombination (HR) template nucleic acid sequence for insertion into an endogenous locus of a cell.

In certain embodiments, the present disclosure provides genome editing of a cell by introducing and recombining a homologous recombination (HR) template nucleic acid sequence into an endogenous locus of a cell. In certain embodiments, the HR template nucleic acid sequence is linear. In certain embodiments, the HR template nucleic acid sequence is circular. In certain embodiments, the circular HR template can be a plasmid, minicircle, or nanoplasmid. In certain embodiments, the HR template nucleic acid sequence comprises a first homology arm and second homology arm. In certain embodiments, the homology arms can be of about 300 bases to about 2,000 bases. For example, each homology arm can be 1,000 bases. In certain embodiments, the homology arms can be homologous to first and second endogenous sequences of the cell. In certain embodiments, the endogenous locus is a TCR locus. For example, the first and second endogenous sequences are within a TCR alpha locus or a TCR beta locus.

In certain embodiments, the first homology arm comprises a nucleotide sequence that is at least about 80%, about 85%, about 90%, about 95%, about 96%, about 97%, about 98%, or about 99% identical to the nucleotide sequence set forth in SEQ ID NO: 35. In certain embodiments, the first homology arm comprises the nucleotide sequence set forth in SEQ ID NO: 35. In certain embodiments, the first homology arm consists of the nucleotide sequence set forth in SEQ ID NO: 35. In certain embodiments, the first homology arm comprises about 300 consecutive bases, about 400 consecutive bases, about 500 consecutive bases, about 600 consecutive bases, about 700 consecutive bases, about 800 consecutive bases, or about 900 consecutive bases of SEQ ID NO: 35. In certain embodiments, the first homology arm comprises about 300 consecutive bases of SEQ ID NO: 35. In certain embodiments, the first homology arm comprises about 400 consecutive bases of SEQ ID NO: 35. In certain embodiments, the first homology arm comprises about 600 consecutive bases of SEQ ID NO: 35.

In certain embodiments, the second homology arm comprises a nucleotide sequence that is at least about 80%, about 85%, about 90%, about 95%, about 96%, about 97%, about 98%, or about 99% identical to the nucleotide sequence set forth in SEQ ID NO: 36. In certain embodiments, the second homology arm comprises the nucleotide sequence set forth in SEQ ID NO: 36. In certain embodiments, the second homology arm consists of the nucleotide sequence set forth in SEQ ID NO: 36. In certain embodiments, the second homology arm comprises about 300 consecutive bases, about 400 consecutive bases, about 500 consecutive bases, about 600 consecutive bases, about 700 consecutive bases, about 800 consecutive bases, or about 900 consecutive bases of SEQ ID NO: 36. In certain embodiments, the second homology arm comprises about 300 consecutive bases of SEQ ID NO: 36. In certain embodiments, the second homology arm comprises about 400 consecutive bases of SEQ ID NO: 36. In certain embodiments, the second homology arm comprises about 600 consecutive bases of SEQ ID NO: 36.

SEQ ID NO: 35 and SEQ ID NO: 36 are provided below.

[SEQ ID NO: 35]
ACATTAAAAACACAAAATCCTACGGAAATACTGAAGAATGAGTCTCAGC
ACTAAGGAAAAGCCTCCAGCAGCTCCTGCTTTCTGAGGGTGAAGGATAG
ACGCTGTGGCTCTGCATGACTCACTAGCACTCTATCACGGCCATATTCT
GGCAGGGTCAGTGGCTCCAACTAACATTTGTTTGGTACTTTACAGTTTA
TTAAATAGATGTTTATATGGAGAAGCTCTCATTTCTTTCTCAGAAGAGC
CTGGCTAGGAAGGTGGATGAGGCACCATATTCATTTTGCAGGTGAAATT
CCTGAGATGTAAGGAGCTGCTGTGACTTGCTCAAGGCCTTATATCGAGT
AAACGGTAGTGCTGGGGCTTAGACGCAGGTGTTCTGATTTATAGTTCAA
AACCTCTATCAATGAGAGAGCAATCTCCTGGTAATGTGATAGATTTCCC
AACTTAATGCCAACATACCATAAACCTCCCATTCTGCTAATGCCCAGCC
TAAGTTGGGGAGACCACTCCAGATTCCAAGATGTACAGTTTGCTTTGCT
GGGCCTTTTTCCCATGCCTGCCTTTACTCTGCCAGAGTTATATTGCTGG
GGTTTTGAAGAAGATCCTATTAAATAAAAGAATAAGCAGTATTATTAAG
TAGCCCTGCATTTCAGGTTTCCTTGAGTGGCAGGCCAGGCCTGGCCGTG
AACGTTCACTGAAATCATGGCCTCTTGGCCAAGATTGATAGCTTGTGCC
TGTCCCTGAGTCCCAGTCCATCACGAGCAGCTGGTTTCTAAGATGCTAT
TTCCCGTATAAAGCATGAGACCGTGACTTGCCAGCCCCACAGAGCCCCG
CCCTTGTCCATCACTGGCATCTGGACTCCAGCCTGGGTTGGGGCAAAGA
GGGAAATGAGATCATGTCCTAACCCTGATCCTCTTGTCCCACAGATATC
CAGAACCCTGACCCTGCCGTGTACCAGCTGAGAGACTCTAAATCCAGTG
ACAAGTCTGTCTGCCTATTC
[SEQ ID NO: 36]
ACCGATTTTGATTCTCAAACAAATGTGTCACAAAGTAAGGATTCTGATG
TGTATATCACAGACAAAACTGTGCTAGACATGAGGTCTATGGACTTCAA
GAGCAACAGTGCTGTGGCCTGGAGCAACAAATCTGACTTTGCATGTGCA
AACGCCTTCAACAACAGCATTATTCCAGAAGACACCTTCTTCCCCAGCC
CAGGTAAGGGCAGCTTTGGTGCCTTCGCAGGCTGTTTCCTTGCTTCAGG
AATGGCCAGGTTCTGCCCAGAGCTCTGGTCAATGATGTCTAAAACTCCT
CTGATTGGTGGTCTCGGCCTTATCCATTGCCACCAAAACCCTCTTTTTA
CTAAGAAACAGTGAGCCTTGTTCTGGCAGTCCAGAGAATGACACGGGAA
AAAAGCAGATGAAGAGAAGGTGGCAGGAGAGGGCACGTGGCCCAGCCTC
AGTCTCTCCAACTGAGTTCCTGCCTGCCTGCCTTTGCTCAGACTGTTTG
CCCCTTACTGCTCTTCTAGGCCTCATTCTAAGCCCCTTCTCCAAGTTGC
CTCTCCTTATTTCTCCCTGTCTGCCAAAAAATCTTTCCCAGCTCACTAA
GTCAGTCTCACGCAGTCACTCATTAACCCACCAATCACTGATTGTGCCG
GCACATGAATGCACCAGGTGTTGAAGTGGAGGAATTAAAAAGTCAGATG
AGGGGTGTGCCCAGAGGAAGCACCATTCTAGTTGGGGGAGCCCATCTGT
CAGCTGGGAAAAGTCCAAATAACTTCAGATTGGAATGTGTTTTAACTCA
GGGTTGAGAAAACAGCTACCTTCAGGACAAAAGTCAGGGAAGGGCTCTC
TGAAGAAATGCTACTTGAAGATACCAGCCCTACCAAGGGCAGGGAGAGG
ACCCTATAGAGGCCTGGGACAGGAGCTCAATGAGAAAGGAGAAGAGCAG
CAGGCATGAGTTGAATGAAGGAGGCAGGGCCGGGTCACAGGGCCTTCTA
GGCCATGAGAGGGTAGACAG

In certain embodiments, the HR template comprises a TCR gene sequence. In non-limiting embodiments, the TCR gene sequence is a patient-specific TCR gene sequence. In non-limiting embodiments, the TCR gene sequence is tumor-specific. In non-limiting embodiments, the TCR gene sequence encodes a TCR recognizing a neoantigen. In certain embodiments, the neoantigen is a private neoantigen. In non-limiting embodiments, the TCR gene sequence can be identified and obtained using the methods described in PCT/US2020/017887, the content of which is herein incorporated by reference. In certain embodiments, the HR template comprises a TCR alpha gene sequence and a TCR beta gene sequence.

In certain embodiments, the HR template is a polycistronic polynucleotide. In certain embodiments, the HR template comprises sequences encoding for flexible polypeptide sequences (e.g., Gly-Ser-Gly sequence). In certain embodiments, the HR template comprises sequences encoding an internal ribosome entry site (IRES). In certain embodiments, the HR template comprises a 2A peptide (e.g., P2A, T2A, E2A, and F2A). In certain embodiments, the HR template comprises a protease cleavage site. In certain embodiments, the HR template comprises a signal sequence.

In certain embodiments, the flexible polypeptide encodes a glycine-serine-glycine sequence. In certain embodiments, the flexible polypeptide is encoded by a nucleotide sequence comprising the nucleotide sequence set forth in SEQ ID NO: 37 or SEQ ID NO: 38. In certain embodiments, the flexible polypeptide is encoded by a nucleotide sequence consisting of the nucleotide sequence set forth in SEQ ID NO: 37 or SEQ ID NO: 38. SEQ ID NO: 37 and SEQ ID NO: 38 are provided below.

[SEQ ID NO: 37]
GGCAGCGGC
[SEQ ID NO: 38]
GGCTCCGGA

In certain embodiments, the 2A peptide is a P2A peptide. In certain embodiments, the P2A peptide comprises the amino acid sequence set forth in SEQ ID NO: 39. In certain embodiments, the P2A peptide consists of the amino acid sequence set forth in SEQ ID NO: 39. In certain embodiments, the P2A peptide is encoded by a nucleotide sequence comprising the nucleotide sequence set forth in SEQ ID NO: 40 or SEQ ID NO: 41. In certain embodiments, the P2A peptide is encoded by a nucleotide sequence consisting of the nucleotide sequence set forth in SEQ ID NO: 40 or SEQ ID NO: 41. SEQ ID NOs: 39-41 are provided below.

[SEQ ID NO: 39]
ATNFSLLKQAGDVEENPGP
[SEQ ID NO: 40]
GCCACCAACTTCAGCCTGCTGAAGCAGGCCGGCGACGTGGAGGAGAACC
CCGGCCCT
[SEQ ID NO: 41]
GCCACTAACTTCTCCCTGTTGAAACAGGCTGGCGATGTTGAAGAAAACC
CCGGTCCT

In certain embodiments, the P2A peptide comprises, at its N-end, a flexible polypeptide comprising a glycine-serine-glycine sequence. In certain embodiments, the P2A peptide comprises, at its N-end, a flexible polypeptide consisting of a glycine-serine-glycine sequence.

In certain embodiments, the protease cleavage site is a Furin cleavage site. In certain embodiments, the Furin cleavage site is encoded by a nucleotide sequence comprising the nucleotide sequence set forth in SEQ ID NO: 42. In certain embodiments, the Furin cleavage site is encoded by a nucleotide sequence consisting of the nucleotide sequence set forth in SEQ ID NO: 42. SEQ ID NO: 42 is provided below.

[SEQ ID NO: 42]
CGGGCCAAGCGG

In certain embodiments, the signal sequence is a human growth hormone (HGH) signal sequence. In certain embodiments, the HGH signal sequence comprises the amino acid sequence set forth in SEQ ID NO: 43. In certain embodiments, the HGH signal sequence consists of the amino acid sequence set forth in SEQ ID NO: 43. In certain embodiments, the HGH signal sequence is encoded by a nucleotide sequence comprising the nucleotide sequence set forth in SEQ ID NO: 44 or SEQ ID NO: 45. In certain embodiments, the HGH signal sequence is encoded by a nucleotide sequence consisting of the nucleotide sequence set forth in SEQ ID NO: 44 or SEQ ID NO: 45. SEQ ID NOs: 43-45 are provided below.

[SEQ ID NO: 43]
MATGSRTSLLLAFGLLCLPWLQEGSA
[SEQ ID NO: 44]
ATGGCCACAGGCAGCAGAACATCTCTGCTGCTGGCCTTCGGACTGCTGT
GTCTGCCTTGGCTGCAAGAGGGTTCCGCC
[SEQ ID NO: 45]
ATGGCCACCGGCTCTAGAACAAGCCTGCTGCTCGCTTTTGGCCTGCTCT
GCCTCCCATGGCTCCAAGAAGGATCTGCT

In certain embodiments, the Secondary Promoter Constructs include multiple 2A peptides, flexible polypeptides, protease cleavage peptides, signal peptides, or combinations thereof. In certain embodiments, the multiple 2A peptides can have the same amino acid sequence. In certain embodiments, the multiple flexible polypeptides can have the same amino sequence. In certain embodiments, the multiple protease cleavage peptides can have the same amino acid sequence. In certain embodiments, the multiple signal peptides can have the same amino acid sequence. In certain embodiments, when the Secondary Promoter Constructs include multiple 2A peptides, flexible polypeptides, protease cleavage peptides, signal peptides, or combinations thereof, these amino acid sequences are encoded by codon diverged nucleotide sequences. Additional information on the HR template nucleic acids and methods of modifying a cell thereof can be found in International Patent Application no. PCT/US2018/058230, the content of which is herein incorporated by reference.

3.1.9. Exemplified Secondary Promoter Constructs

In certain embodiments, the Secondary Promoter Construct comprises a sequence encoding an exogenous TCR and at least one Payload. In certain embodiments, the sequence encoding an exogenous TCR encodes a TRCα and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the Secondary Promoter Construct further comprises a promoter and an insulator. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, an insulator, a promoter, a Payload, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a first poly-A signal sequence and a second poly-A signal sequence. In certain embodiments, the Secondary Promoter Construct further comprises a promoter, an insulator, and a WPRE element. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a first poly-A signal sequence, an insulator, a promoter, a Payload, a WPRE element, a second poly-A signal sequence, and a second homology arm.

In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a first poly-A signal sequence, a first insulator, a promoter, a Payload, a second poly-A signal sequence, a second insulator, and a second homology arm.

In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a first poly-A signal sequence, an insulator, a promoter, a Payload, a second poly-A signal sequence, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the Secondary Promoter Construct further comprises a promoter and a WPRE element. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a WPRE element, a Payload, a promoter, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct comprises, from 5′ to 3′, a first homology arm, a first 2A peptide, a first signal sequence peptide, a Payload, a protease cleavage site, a second 2A peptide, a second signal sequence, a TCRβ gene sequence, a second protease cleavage site, a third 2A peptide, a third signal sequence peptide, and a TRCα gene sequence.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence and an insulator. In certain embodiments, the 3′ of the at least one Payload comprises a STOP codon. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, an insulator, a promoter, a Payload having at its 3′ a termination codon, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the 3′ of the at least one Payload comprises a protease cleavage site and a 2A peptide. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a Payload, a second protease cleavage site, a third 2A peptide, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct comprises an enhancer. In certain embodiments, the Secondary Promoter Construct further comprises a poly-A signal sequence and a pause element. In certain embodiments, the 3′ of the at least one Payload comprises protease cleavage site and a 2A peptide. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, an enhancer, a first 2A peptide, a first signal sequence peptide, a TCRβ gene sequence, a protease cleavage site, a second 2A peptide, a second signal sequence peptide, a TRCα gene sequence, a poly-A signal sequence, a pause element, a promoter, a Payload, a second protease cleavage site, a third 2A peptide, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct comprises an enhancer. In certain embodiments, the Secondary Promoter Construct further comprises a poly-A signal sequence and an insulator. In certain embodiments, the 3′ of the at least one Payload comprises a protease cleavage site and a 2A peptide. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, an enhancer, a first 2A peptide, a first signal sequence peptide, a TCRβ gene sequence, a protease cleavage site, a second 2A peptide, a second signal sequence peptide, a TRCα gene sequence, a poly-A signal sequence, an insulator, a promoter, a Payload, a second protease cleavage site, a third 2A peptide, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence, an enhancer, and an insulator. In certain embodiments, the 3′ of the at least Payload comprises a STOP codon. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, an enhancer, a first 2A peptide, a first signal sequence peptide, a TCRβ gene sequence, a protease cleavage site, a second 2A peptide, a second signal sequence peptide, a TRCα gene sequence, a poly-A signal sequence, an insulator, a promoter, a Payload having at its 3′ a termination codon, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the at least one Payload comprises an inhibitory RNA molecule. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, an inhibitory RNA molecule, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence and a termination sequence. In certain embodiments, the at least one Payload comprises an inhibitory RNA molecule. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, an inhibitory RNA molecule, a termination sequence, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the at least one Payload comprises an inhibitory RNA molecule. In certain embodiments, the 3′ of the at least one Payload comprises a protease cleavage site and a 2A peptide. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a first Payload, a second Payload, a second protease cleavage site, a third 2A peptide, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the at least one Payload comprises an inhibitory RNA molecule. In certain embodiments, the at least one Payload comprises with a protease cleavage site and a 2A peptide. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a first Payload, a second protease cleavage site, a third 2A peptide, a second Payload, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the at least one Payload comprises an inhibitory RNA molecule. In certain embodiments, the inhibitory RNA molecule is flanked by a splice acceptor site, a splice donor site, or a combination thereof. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a splice donor site, an inhibitory RNA molecule, a splice acceptor site, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the at least one Payload comprises an inhibitory RNA molecule. In certain embodiments, the Secondary Promoter Construct further comprises a splice acceptor site. In certain embodiments, the Secondary Promoter Construct further comprises a first homology arm and a second homology arm. In certain embodiments, the Secondary Promoter Construct comprises, from 5′ to 3′, a first homology arm, an inhibitory RNA molecule, a splice acceptor site, a first 2A peptide, a first signal sequence peptide, a TCRβ gene sequence, a protease cleavage site, a second 2A peptide, a second signal sequence peptide, a TRCα gene sequence, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the at least one Payload comprises an inhibitory RNA molecule. In certain embodiments, the Secondary Promoter Construct further comprises a first homology arm and a second homology arm. In certain embodiments, the Secondary Promoter Construct comprises, from 5′ to 3′, a first homology arm, an inhibitory RNA molecule, a first 2A peptide, a first signal sequence peptide, a TCRβ gene sequence, a protease cleavage site, a second 2A peptide, a second signal sequence peptide, a TRCα gene sequence, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the at least one Payload comprises an inhibitory RNA molecule. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a first Payload, a second Payload, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the at least one Payload comprises a first inhibitory RNA molecule. In certain embodiments, the at least one Payload comprises a second inhibitory RNA molecule. In certain embodiments, the first and second inhibitory RNA molecules knock down the gene expression of a same gene. In certain embodiments, the first and second inhibitory RNA molecules knock down the gene expression of different genes. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a first inhibitory RNA molecule, a second inhibitory RNA molecule, and a second homology arm.

In certain embodiments, the Secondary Promoter Construct comprises a sequence encoding an exogenous TCR and at least one Payload. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the Payload is a miRNA cluster. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a miRNA cluster, and a second homology arm.

In certain embodiments, the Secondary Promoter Construct comprises a sequence encoding an exogenous TCR and at least two Payloads. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the Secondary Promoter Construct further comprises a poly-A signal sequence. In certain embodiments, the first Payload is an inhibitory RNA molecule. In certain embodiments, the inhibitory RNA molecule is flanked by a splice acceptor site, a splice donor site, or a combination thereof. In certain embodiments, the Secondary Promoter Construct further comprises a promoter. In certain embodiments, the Secondary Promoter Construct further comprises a first homology arm and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a first Payload, a splice donor site, an inhibitory RNA molecule, a splice acceptor site, and a second homology arm. In certain embodiments, the Secondary Promoter Construct comprises, from 5′ to 3′, a first homology arm, a first Payload, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a splice donor site, an inhibitory RNA molecule, a splice acceptor site, and a second homology arm. In certain embodiments, the Secondary Promoter Construct comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, an inhibitory RNA molecule, and a second homology arm.

In certain embodiments, when a Secondary Promoter Construct includes two or more sequences encoding a 2A peptide, the sequences encode for the same 2A peptide and can be codon diverged. For example, without any limitation, two sequences can encode for a P2A and be codon diverged. In certain embodiments, when a Secondary Promoter Construct includes a sequence encoding a 2A peptide, the Secondary Promoter Construct can include a sequence encoding for a GSG amino acid at the 5′ of the sequence encoding a 2A peptide. In certain embodiments, when a Secondary Promoter Construct includes two or more sequences encoding a signal peptide, the sequences encode for the same signal peptide and can be codon diverged. For example, without any limitation, two sequences can encode for a HGH signal peptide and be codon diverged.

In certain embodiments, the Secondary Promoter Construct comprises a TCRβ gene sequence comprising a nucleotide sequence that is at least about 80%, about 85%, about 90%, about 95%, about 96%, about 97%, about 98%, about 99% identical to the nucleotide sequence set forth in SEQ ID NO: 46. In certain embodiments, the TCRβ gene sequence comprises the nucleotide sequence set forth in SEQ ID NO: 46. In certain embodiments, TCRβ gene sequence consists of the nucleotide sequence set forth in SEQ ID NO: 46.

In certain embodiments, the TCRβ gene sequence encodes an amino acid sequence that is at least about 80%, about 85%, about 90%, about 95%, about 96%, about 97%, about 98%, about 99% identical to the amino acid sequence set forth in SEQ ID NO: 47. In certain embodiments, the TCRβ gene sequence encodes the amino acid sequence set forth in SEQ ID NO: 47. SEQ ID NOs: 46 and 47 are provided below.

[SEQ ID NO: 46]
GATGCTGGAATCACCCAGAGCCCAAGATACAAGATCACAGAGACAGGAA
GGCAGGTGACCTTGATGTGTCACCAGACTTGGAGCCACAGCTATATGTT
CTGGTATCGACAAGACCTGGGACATGGGCTGAGGCTGATCTATTACTCA
GCAGCTGCTGATATTACAGATAAAGGAGAAGTCTCCGATGGCTATGTTG
TCTCCAGATCCAAGACAGAGAATTTCCCCCTCACTCTGGAGTCAGCTAC
CCGCTCCCAGACATCTGTGTATTTCTGCGCCAGCAGTGAGGACAGTTAC
GAGCAGTACTTCGGGCCGGGCACCAGGCTCACGGTCACAGAGGACCTGA
AAAACGTGTTCCCTCCAAAAGTGGCCGTGTTCGAGCCTTCTGAGGCCGA
GATCAGCCACACACAGAAAGCCACACTCGTGTGTCTGGCTACCGGCTTC
TACCCCGATCACGTGGAACTGTCTTGGTGGGTCAACGGCAAAGAGGTGC
ACAGCGGCGTCAGCACAGATCCCCAGCCTCTGAAAGAACAGCCCGCTCT
GAACGACAGCCGCTACTGCCTGTCTAGCAGACTGAGAGTGTCCGCCACC
TTCTGGCAGAACCCCAGAAACCACTTCAGATGCCAGGTCCAGTTCTACG
GCCTGAGCGAGAACGATGAGTGGACCCAGGACAGAGCCAAGCCTGTGAC
ACAGATCGTGTCTGCCGAAGCCTGGGGCAGAGCCGATTGTGGCTTTACC
AGCGAGTCATACCAGCAGGGCGTGCTGTCTGCCACCATCCTGTATGAGA
TCCTGCTCGGCAAGGCCACACTGTACGCTGTGCTGGTGTCTGCTCTGGT
GCTGATGGCTATGGTCTCCCGGGAGCGCATCCCCGAGGCC
[SEQ ID NO: 47]
DAGITQSPRYKITETGRQVTLMCHQTWSHSYMFWYRQDLGHGLRLIYYS
AAADITDKGEVSDGYVVSRSKTENFPLTLESATRSQTSVYFCASSEDSY
EQYFGPGTRLTVTEDLKNVFPPKVAVFEPSEAEISHTQKATLVCLATGF
YPDHVELSWWVNGKEVHSGVSTDPQPLKEQPALNDSRYCLSSRLRVSAT
FWQNPRNHFRCQVQFYGLSENDEWTQDRAKPVTQIVSAEAWGRADCGFT
SESYQQGVLSATILYEILLGKATLYAVLVSALVLMAMVSRERIPEA

In certain non-limiting embodiments, the Secondary Promoter Construct comprises a Payload encoding an mCherry protein. In certain embodiments, the Secondary Promoter Construct comprises a nucleotide sequence that is at least about 80%, about 85%, about 90%, about 95%, about 96%, about 97%, about 98%, about 99% identical to the nucleotide sequence set forth in SEQ ID NOs: 48-64. In certain embodiments, the Secondary Promoter Construct comprises the nucleotide sequence set forth in SEQ ID NOs: 48-64. In certain embodiments, the Secondary Promoter Construct consists of the nucleotide sequence set forth in SEQ ID NOs: 48-64.

In certain embodiments, the at least one Payload (e.g., mCherry protein) of any one of SEQ ID NOs: 48-64 can be replaced by any Payload disclosed herein. For example, but without any limitation, the at least one Payload can be replaced by a sequence encoding a cytokine receptors trap or ligand trap, an angiogenesis factors, an apoptotic factor, an inhibitory protein for various cell processes (e.g., inhibition of macrophage activation), an extracellular matrix modulator, a soluble TCRs, a soluble chimeric antigen receptor (CAR), a homing signal, an enzyme, a modulator of reactive oxygen species, a competitive ligand inhibitor, a protein that binds to receptors and sterically hinders receptor function, or an inhibitory RNA molecule.

In certain embodiments, the Secondary Promoter Construct comprises nucleotides 1 to 3794 of SEQ ID NO: 48. In certain embodiments, the Secondary Promoter Construct comprises nucleotides 4503 to 7046 of SEQ ID NO: 48. In certain embodiments, the Secondary Promoter Construct comprises nucleotides 1 to 3821 and nucleotides 4503 to 7046 of SEQ ID NO: 48. SEQ ID NO: 48 is provided below.

[SEQ ID NO: 48]
GGTACCACATTAAAAACACAAAATCCTACGGAAATACTGAAGAATGAGTCTCAGCACTAAGGAAAAGCCTCCAGCAGCTC
CTGCTTTCTGAGGGTGAAGGATAGACGCTGTGGCTCTGCATGACTCACTAGCACTCTATCACGGCCATATTCTGGCAGGG
TCAGTGGCTCCAACTAACATTTGTTTGGTACTTTACAGTTTATTAAATAGATGTTTATATGGAGAAGCTCTCATTTCTTT
CTCAGAAGAGCCTGGCTAGGAAGGTGGATGAGGCACCATATTCATTTTGCAGGTGAAATTCCTGAGATGTAAGGAGCTGC
TGTGACTTGCTCAAGGCCTTATATCGAGTAAACGGTAGTGCTGGGGCTTAGACGCAGGTGTTCTGATTTATAGTTCAAAA
CCTCTATCAATGAGAGAGCAATCTCCTGGTAATGTGATAGATTTCCCAACTTAATGCCAACATACCATAAACCTCCCATT
CTGCTAATGCCCAGCCTAAGTTGGGGAGACCACTCCAGATTCCAAGATGTACAGTTTGCTTTGCTGGGCCTTTTTCCCAT
GCCTGCCTTTACTCTGCCAGAGTTATATTGCTGGGGTTTTGAAGAAGATCCTATTAAATAAAAGAATAAGCAGTATTATT
AAGTAGCCCTGCATTTCAGGTTTCCTTGAGTGGCAGGCCAGGCCTGGCCGTGAACGTTCACTGAAATCATGGCCTCTTGG
CCAAGATTGATAGCTTGTGCCTGTCCCTGAGTCCCAGTCCATCACGAGCAGCTGGTTTCTAAGATGCTATTTCCCGTATA
AAGCATGAGACCGTGACTTGCCAGCCCCACAGAGCCCCGCCCTTGTCCATCACTGGCATCTGGACTCCAGCCTGGGTTGG
GGCAAAGAGGGAAATGAGATCATGTCCTAACCCTGATCCTCTTGTCCCACAGATATCCAGAACCCTGACCCTGCCGTGTA
CCAGCTGAGAGACTCTAAATCCAGTGACAAGTCTGTCTGCCTATTCGAATTCGGCTCCGGAGCCACTAACTTCTCCCTGT
TGAAACAGGCTGGCGATGTTGAAGAAAACCCCGGTCCTATGGCCACCGGCTCTAGAACAAGCCTGCTGCTCGCTTTTGGC
CTGCTCTGCCTCCCATGGCTCCAAGAAGGATCTGCTGATGCTGGAATCACCCAGAGCCCAAGATACAAGATCACAGAGAC
AGGAAGGCAGGTGACCTTGATGTGTCACCAGACTTGGAGCCACAGCTATATGTTCTGGTATCGACAAGACCTGGGACATG
GGCTGAGGCTGATCTATTACTCAGCAGCTGCTGATATTACAGATAAAGGAGAAGTCTCCGATGGCTATGTTGTCTCCAGA
TCCAAGACAGAGAATTTCCCCCTCACTCTGGAGTCAGCTACCCGCTCCCAGACATCTGTGTATTTCTGCGCCAGCAGTGA
GGACAGTTACGAGCAGTACTTCGGGCCGGGCACCAGGCTCACGGTCACAGAGGACCTGAAAAACGTGTTCCCTCCAAAAG
TGGCCGTGTTCGAGCCTTCTGAGGCCGAGATCAGCCACACACAGAAAGCCACACTCGTGTGTCTGGCTACCGGCTTCTAC
CCCGATCACGTGGAACTGTCTTGGTGGGTCAACGGCAAAGAGGTGCACAGCGGCGTCAGCACAGATCCCCAGCCTCTGAA
AGAACAGCCCGCTCTGAACGACAGCCGCTACTGCCTGTCTAGCAGACTGAGAGTGTCCGCCACCTTCTGGCAGAACCCCA
GAAACCACTTCAGATGCCAGGTCCAGTTCTACGGCCTGAGCGAGAACGATGAGTGGACCCAGGACAGAGCCAAGCCTGTG
ACACAGATCGTGTCTGCCGAAGCCTGGGGCAGAGCCGATTGTGGCTTTACCAGCGAGTCATACCAGCAGGGCGTGCTGTC
TGCCACCATCCTGTATGAGATCCTGCTCGGCAAGGCCACACTGTACGCTGTGCTGGTGTCTGCTCTGGTGCTGATGGCTA
TGGTCTCCCGGGAGCGCATCCCCGAGGCCCGGGCCAAGCGGGGCAGCGGCGCCACCAACTTCAGCCTGCTGAAGCAGGCC
GGCGACGTGGAGGAGAACCCCGGCCCTATGGCCACAGGCAGCAGAACATCTCTGCTGCTGGCCTTCGGACTGCTGTGTCT
GCCTTGGCTGCAAGAGGGTTCCGCCGCCCAGTCAGTGACCCAGCCTGACATCCACATCACTGTCTCTGAAGGAGCCTCAC
TGGAGTTGAGATGTAACTATTCCTATGGGGCAACACCTTATCTCTTCTGGTATGTCCAGTCCCCCGGCCAAGGCCTCCAG
CTGCTCCTGAAGTACTTTTCAGGAGACACTCTGGTTCAAGGCATTAAAGGCTTTGAGGCTGAATTTAAGAGGAGTCAATC
TTCCTTCAATCTGAGGAAACCCTCTGTGCATTGGAGTGATGCTGCTGAGTACTTCTGTGCTGTGGGTGAATTGGACACAG
GCTTTCAGAAACTTGTATTTGGAACTGGCACCCGACTTCTGGTCAGTCCAAATATTCAGAACCCCGATCCTGCCGTGTAC
CAGCTGAGAGACAGCAAGAGCAGCGACAAGAGCGTGTGCCTGTTCACCGACTTCGACAGCCAGACCAACGTGTCCCAGAG
CAAGGACAGCGACGTGTACATCACCGACAAGACCGTGCTGGACATGCGGAGCATGGACTTCAAGAGCAACAGCGCCGTGG
CCTGGTCCAACAAGAGCGATTTCGCCTGCGCCAACGCCTTCAACAACAGCATTATCCCCGAGGACACATTCTTCCCAAGT
CCTGAGAGCAGCTGCGACGTGAAGCTGGTGGAAAAGAGCTTCGAGACAGACACCAACCTGAACTTCCAGAACCTGTCCGT
GATCGGCTTCCGCATCCTGCTGCTGAAAGTGGCCGGCTTCAACCTGCTGATGACCCTGAGACTGTGGTCCAGCTGATTTA
TTTGTGAAATTTGTGATGCTATTGCTTTATTTGTAACCATTCTAGCTTTATTTGTGAAATTTGTGATGCTATTGCTTTAT
TTGTAACCATTATAAGCTGCAATAAACAAGTTAACAACAACAATTGCATTCATTTTATGTTTCAGGTTCAGGGGGAGATG
TGGGAGGTTTTTTAAAGCGTCGACGAGCTCACGGGGACAGCCCCCCCCCAAAGCCCCCAGGGATGTAATTACGTCCCTCC
CCCGCTAGGGGGCAGCAGCGAGCCGCCCGGGGCTCCGCTCCGGTCCGGCGCTCCCCCCGCATCCCCGAGCCGGCAGCGTG
CGGGGACAGCCCGGGCACGGGGAAGGTGGCACGGGATCGCTTTCCTCTGAACGCTTCTCGCTGCTCTTTGAGCCTGCAGA
CACCTGGGGGGATACGGGGAAAAAGCTTCTCGAGGAACAGAGAAACAGGAGAATATGGGCCAAACAGGATATCTGTGGTA
AGCAGTTCCTGCCCCGGCTCAGGGCCAAGAACAGTTGGAACAGCAGAATATGGGCCAAACAGGATATCTGTGGTAAGCAG
TTCCTGCCCCGGCTCAGGGCCAAGAACAGATGGTCCCCAGATGCGGTCCCGCCCTCAGCAGTTTCTAGAGAACCATCAGA
TGTTTCCAGGGTGCCCCAAGGACCTGAAATGACCCTGTGCCTTATTTGAACTAACCAATCAGTTCGCTTCTCGCTTCTGT
TCGCGCGCTTCTGCTCCCCGAGCTCTATATAAGCAGAGCTCGTTTAGTGAACCGTCAGATCGCCTGGAGACGCCATCCAC
GCTGTTTTGACTTCCATAGAAGGGATCCGCCACCATGGTGTCCAAGGGCGAAGAGGACAACATGGCCATCATCAAAGAGT
TCATGCGGTTCAAGGTGCACATGGAAGGCAGCGTGAACGGCCACGAGTTCGAGATTGAAGGCGAAGGCGAGGGCAGACCT
TACGAGGGAACACAGACCGCCAAGCTGAAAGTGACCAAAGGCGGCCCTCTGCCTTTTGCCTGGGACATTCTGAGCCCTCA
GTTTATGTACGGCAGCAAGGCCTACGTGAAGCACCCCGCCGATATTCCCGACTACCTGAAGCTGAGCTTCCCCGAGGGCT
TCAAGTGGGAGAGAGTGATGAACTTCGAGGACGGCGGCGTGGTCACCGTGACTCAAGATAGCTCTCTGCAGGACGGCGAG
TTCATCTACAAAGTGAAGCTGCGGGGCACCAACTTTCCCTCTGATGGCCCCGTGATGCAGAAAAAGACAATGGGCTGGGA
AGCCAGCAGCGAGAGAATGTACCCTGAAGATGGCGCCCTGAAGGGCGAGATCAAGCAGCGGCTGAAACTGAAGGATGGCG
GCCACTACGACGCCGAAGTGAAAACCACCTACAAGGCCAAGAAACCCGTGCAGCTGCCTGGCGCCTACAACGTGAACATC
AAGCTGGACATCACCAGCCACAACGAGGACTACACCATCGTGGAACAGTACGAGAGAGCCGAAGGCAGACACAGCACAGG
CGGAATGGACGAGCTGTACAAGTGACATATGACCGATTTTGATTCTCAAACAAATGTGTCACAAAGTAAGGATTCTGATG
TGTATATCACAGACAAAACTGTGCTAGACATGAGGTCTATGGACTTCAAGAGCAACAGTGCTGTGGCCTGGAGCAACAAA
TCTGACTTTGCATGTGCAAACGCCTTCAACAACAGCATTATTCCAGAAGACACCTTCTTCCCCAGCCCAGGTAAGGGCAG
CTTTGGTGCCTTCGCAGGCTGTTTCCTTGCTTCAGGAATGGCCAGGTTCTGCCCAGAGCTCTGGTCAATGATGTCTAAAA
CTCCTCTGATTGGTGGTCTCGGCCTTATCCATTGCCACCAAAACCCTCTTTTTACTAAGAAACAGTGAGCCTTGTTCTGG
CAGTCCAGAGAATGACACGGGAAAAAAGCAGATGAAGAGAAGGTGGCAGGAGAGGGCACGTGGCCCAGCCTCAGTCTCTC
CAACTGAGTTCCTGCCTGCCTGCCTTTGCTCAGACTGTTTGCCCCTTACTGCTCTTCTAGGCCTCATTCTAAGCCCCTTC
TCCAAGTTGCCTCTCCTTATTTCTCCCTGTCTGCCAAAAAATCTTTCCCAGCTCACTAAGTCAGTCTCACGCAGTCACTC
ATTAACCCACCAATCACTGATTGTGCCGGCACATGAATGCACCAGGTGTTGAAGTGGAGGAATTAAAAAGTCAGATGAGG
GGTGTGCCCAGAGGAAGCACCATTCTAGTTGGGGGAGCCCATCTGTCAGCTGGGAAAAGTCCAAATAACTTCAGATTGGA
ATGTGTTTTAACTCAGGGTTGAGAAAACAGCTACCTTCAGGACAAAAGTCAGGGAAGGGCTCTCTGAAGAAATGCTACTT
GAAGATACCAGCCCTACCAAGGGCAGGGAGAGGACCCTATAGAGGCCTGGGACAGGAGCTCAATGAGAAAGGAGAAGAGC
AGCAGGCATGAGTTGAATGAAGGAGGCAGGGCCGGGTCACAGGGCCTTCTAGGCCATGAGAGGGTAGACAGGCTAGCCGC
GTTGCTGGCGTTTTTCCATAGGCTCCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTCCTGTTCCGACCCTGCCGCTTACC
GGATACCTGTCCGCCTTTCTCCCTTCGGGAAGCGTGGCGCTTTCTCATAGCTCACGCTGTAGGTATCTCAGTTCGGTGTA
GGTCGTTCGCTCCAAGCTGGGCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGATTAGCAGAGCGAGGTATGTA
GGCGGTGCTACAGAGTTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGTAGCGGTGGTTTTTTTG
TTTGCAAGCAGCAGATTACGCGCAGAAAAAAAGGATCTCAAGAAGATCCTTTGATCTTTAGAAAAACTCATCGAGCATCA
AATGAAACTGCAATTTATTCATATCAGGATTATCAATACCATATTTTTGAAAAAGCCGTTTCTGTAATGAAGGAGAAAAC
TCACCGAGGCAGTTCCATAGGATGGCAAGATCCTGGTATCGGTCTGCGATTCCGACTCGTCCAACATCAATACAACCTAT
TAATTTCCCCTCGTCAAAAATAAGGTTATCAAGTGAGAAATCACCATGAGTGACGACTGAATCCGGTGAGAATGGCAAAA
GTTTATGCATTTCTTTCCAGACTTGTTCAACAGGCCAGCCATTACGCTCGTCATCAAAATCACTCGCATCAACCAAACCG
TTATTCATTCGTGATTGCGCCTGAGCCAGACGAAATACGCGATCGCTGTTAAAAGGACAATTACAAACAGGAATCGAATG
CAACCGGCGCAGGAACACTGCCAGCGCATCAACAATATTTTCACCTGAATCAGGATATTCTTCTAATACCTGGAATGCTG
TTTTTCCGGGGATCGCAGTGGTGAGTAACCATGCATCATCAGGAGTACGGATAAAATGCTTGATGGTCGGAAGAGGCATA
AATTCCGTCAGCCAGTTTAGTCTGACCATCTCATCTGTAACATCATTGGCAACGCTACCTTTGCCATGTTTCAGAAACAA
CTCTGGCGCATCGGGCTTCCCATACAAGCGATAGATTGTCGCACCTGATTGCCCGACATTATCGCGAGCCCATTTATACC
CATATAAATCAGCATCCATGTTGGAATTTAATCGCGGCCTCGACGTTTCCCGTTGAATATGGCTCATAACACCCCTTGTA
TTACTGTTTATGTAAGCAGACAGTTTTATTGTTCATGATGATATATTTTTATCTTGTGCAATGTAACATCAGAGATTTTG
AGACAC

In certain embodiments, the Secondary Promoter Construct comprises nucleotides 1 to 3837 of SEQ ID NO: 49. In certain embodiments, the Secondary Promoter Construct comprises nucleotides 4546 to 7520 of SEQ ID NO: 49. In certain embodiments, the Secondary Promoter Construct comprises nucleotides 1 to 3837 and nucleotides 4546 to 7520 of SEQ ID NO: 49. SEQ ID NO: 49 is provided below.

[SEQ ID NO: 49]
GGTACCACATTAAAAACACAAAATCCTACGGAAATACTGAAGAATGAGTCTCAGCACTAAGGAAAAGCCTCCAGCAGCTC
CTGCTTTCTGAGGGTGAAGGATAGACGCTGTGGCTCTGCATGACTCACTAGCACTCTATCACGGCCATATTCTGGCAGGG
TCAGTGGCTCCAACTAACATTTGTTTGGTACTTTACAGTTTATTAAATAGATGTTTATATGGAGAAGCTCTCATTTCTTT
CTCAGAAGAGCCTGGCTAGGAAGGTGGATGAGGCACCATATTCATTTTGCAGGTGAAATTCCTGAGATGTAAGGAGCTGC
TGTGACTTGCTCAAGGCCTTATATCGAGTAAACGGTAGTGCTGGGGCTTAGACGCAGGTGTTCTGATTTATAGTTCAAAA
CCTCTATCAATGAGAGAGCAATCTCCTGGTAATGTGATAGATTTCCCAACTTAATGCCAACATACCATAAACCTCCCATT
CTGCTAATGCCCAGCCTAAGTTGGGGAGACCACTCCAGATTCCAAGATGTACAGTTTGCTTTGCTGGGCCTTTTTCCCAT
GCCTGCCTTTACTCTGCCAGAGTTATATTGCTGGGGTTTTGAAGAAGATCCTATTAAATAAAAGAATAAGCAGTATTATT
AAGTAGCCCTGCATTTCAGGTTTCCTTGAGTGGCAGGCCAGGCCTGGCCGTGAACGTTCACTGAAATCATGGCCTCTTGG
CCAAGATTGATAGCTTGTGCCTGTCCCTGAGTCCCAGTCCATCACGAGCAGCTGGTTTCTAAGATGCTATTTCCCGTATA
AAGCATGAGACCGTGACTTGCCAGCCCCACAGAGCCCCGCCCTTGTCCATCACTGGCATCTGGACTCCAGCCTGGGTTGG
GGCAAAGAGGGAAATGAGATCATGTCCTAACCCTGATCCTCTTGTCCCACAGATATCCAGAACCCTGACCCTGCCGTGTA
CCAGCTGAGAGACTCTAAATCCAGTGACAAGTCTGTCTGCCTATTCGAATTCGGCTCCGGAGCCACTAACTTCTCCCTGT
TGAAACAGGCTGGCGATGTTGAAGAAAACCCCGGTCCTATGGCCACCGGCTCTAGAACAAGCCTGCTGCTCGCTTTTGGC
CTGCTCTGCCTCCCATGGCTCCAAGAAGGATCTGCTGATGCTGGAATCACCCAGAGCCCAAGATACAAGATCACAGAGAC
AGGAAGGCAGGTGACCTTGATGTGTCACCAGACTTGGAGCCACAGCTATATGTTCTGGTATCGACAAGACCTGGGACATG
GGCTGAGGCTGATCTATTACTCAGCAGCTGCTGATATTACAGATAAAGGAGAAGTCTCCGATGGCTATGTTGTCTCCAGA
TCCAAGACAGAGAATTTCCCCCTCACTCTGGAGTCAGCTACCCGCTCCCAGACATCTGTGTATTTCTGCGCCAGCAGTGA
GGACAGTTACGAGCAGTACTTCGGGCCGGGCACCAGGCTCACGGTCACAGAGGACCTGAAAAACGTGTTCCCTCCAAAAG
TGGCCGTGTTCGAGCCTTCTGAGGCCGAGATCAGCCACACACAGAAAGCCACACTCGTGTGTCTGGCTACCGGCTTCTAC
CCCGATCACGTGGAACTGTCTTGGTGGGTCAACGGCAAAGAGGTGCACAGCGGCGTCAGCACAGATCCCCAGCCTCTGAA
AGAACAGCCCGCTCTGAACGACAGCCGCTACTGCCTGTCTAGCAGACTGAGAGTGTCCGCCACCTTCTGGCAGAACCCCA
GAAACCACTTCAGATGCCAGGTCCAGTTCTACGGCCTGAGCGAGAACGATGAGTGGACCCAGGACAGAGCCAAGCCTGTG
ACACAGATCGTGTCTGCCGAAGCCTGGGGCAGAGCCGATTGTGGCTTTACCAGCGAGTCATACCAGCAGGGCGTGCTGTC
TGCCACCATCCTGTATGAGATCCTGCTCGGCAAGGCCACACTGTACGCTGTGCTGGTGTCTGCTCTGGTGCTGATGGCTA
TGGTCTCCCGGGAGCGCATCCCCGAGGCCCGGGCCAAGCGGGGCAGCGGCGCCACCAACTTCAGCCTGCTGAAGCAGGCC
GGCGACGTGGAGGAGAACCCCGGCCCTATGGCCACAGGCAGCAGAACATCTCTGCTGCTGGCCTTCGGACTGCTGTGTCT
GCCTTGGCTGCAAGAGGGTTCCGCCGCCCAGTCAGTGACCCAGCCTGACATCCACATCACTGTCTCTGAAGGAGCCTCAC
TGGAGTTGAGATGTAACTATTCCTATGGGGCAACACCTTATCTCTTCTGGTATGTCCAGTCCCCCGGCCAAGGCCTCCAG
CTGCTCCTGAAGTACTTTTCAGGAGACACTCTGGTTCAAGGCATTAAAGGCTTTGAGGCTGAATTTAAGAGGAGTCAATC
TTCCTTCAATCTGAGGAAACCCTCTGTGCATTGGAGTGATGCTGCTGAGTACTTCTGTGCTGTGGGTGAATTGGACACAG
GCTTTCAGAAACTTGTATTTGGAACTGGCACCCGACTTCTGGTCAGTCCAAATATTCAGAACCCCGATCCTGCCGTGTAC
CAGCTGAGAGACAGCAAGAGCAGCGACAAGAGCGTGTGCCTGTTCACCGACTTCGACAGCCAGACCAACGTGTCCCAGAG
CAAGGACAGCGACGTGTACATCACCGACAAGACCGTGCTGGACATGCGGAGCATGGACTTCAAGAGCAACAGCGCCGTGG
CCTGGTCCAACAAGAGCGATTTCGCCTGCGCCAACGCCTTCAACAACAGCATTATCCCCGAGGACACATTCTTCCCAAGT
CCTGAGAGCAGCTGCGACGTGAAGCTGGTGGAAAAGAGCTTCGAGACAGACACCAACCTGAACTTCCAGAACCTGTCCGT
GATCGGCTTCCGCATCCTGCTGCTGAAAGTGGCCGGCTTCAACCTGCTGATGACCCTGAGACTGTGGTCCAGCTGATGTG
CCTTCTAGTTGCCAGCCATCTGTTGTTTGCCCCTCCCCCGTGCCTTCCTTGACCCTGGAAGGTGCCACTCCCACTGTCCT
TTCCTAATAAAATGAGGAAATTGCATCGCATTGTCTGAGTAGGTGTCATTCTATTCTGGGGGGTGGGGTGGGGCAGGACA
GCAAGGGGGAGGATTGGGAAGACAATAGCAGGCATGCTGGGGATGCGGTGGGCTCTATGGCGTCGACGAGCTCACGGGGA
CAGCCCCCCCCCAAAGCCCCCAGGGATGTAATTACGTCCCTCCCCCGCTAGGGGGCAGCAGCGAGCCGCCCGGGGCTCCG
CTCCGGTCCGGCGCTCCCCCCGCATCCCCGAGCCGGCAGCGTGCGGGGACAGCCCGGGCACGGGGAAGGTGGCACGGGAT
CGCTTTCCTCTGAACGCTTCTCGCTGCTCTTTGAGCCTGCAGACACCTGGGGGGATACGGGGAAAAAGCTTCTCGAGGAA
CAGAGAAACAGGAGAATATGGGCCAAACAGGATATCTGTGGTAAGCAGTTCCTGCCCCGGCTCAGGGCCAAGAACAGTTG
GAACAGCAGAATATGGGCCAAACAGGATATCTGTGGTAAGCAGTTCCTGCCCCGGCTCAGGGCCAAGAACAGATGGTCCC
CAGATGCGGTCCCGCCCTCAGCAGTTTCTAGAGAACCATCAGATGTTTCCAGGGTGCCCCAAGGACCTGAAATGACCCTG
TGCCTTATTTGAACTAACCAATCAGTTCGCTTCTCGCTTCTGTTCGCGCGCTTCTGCTCCCCGAGCTCTATATAAGCAGA
GCTCGTTTAGTGAACCGTCAGATCGCCTGGAGACGCCATCCACGCTGTTTTGACTTCCATAGAAGGGATCCGCCACCATG
GTGTCCAAGGGCGAAGAGGACAACATGGCCATCATCAAAGAGTTCATGCGGTTCAAGGTGCACATGGAAGGCAGCGTGAA
CGGCCACGAGTTCGAGATTGAAGGCGAAGGCGAGGGCAGACCTTACGAGGGAACACAGACCGCCAAGCTGAAAGTGACCA
AAGGCGGCCCTCTGCCTTTTGCCTGGGACATTCTGAGCCCTCAGTTTATGTACGGCAGCAAGGCCTACGTGAAGCACCCC
GCCGATATTCCCGACTACCTGAAGCTGAGCTTCCCCGAGGGCTTCAAGTGGGAGAGAGTGATGAACTTCGAGGACGGCGG
CGTGGTCACCGTGACTCAAGATAGCTCTCTGCAGGACGGCGAGTTCATCTACAAAGTGAAGCTGCGGGGCACCAACTTTC
CCTCTGATGGCCCCGTGATGCAGAAAAAGACAATGGGCTGGGAAGCCAGCAGCGAGAGAATGTACCCTGAAGATGGCGCC
CTGAAGGGCGAGATCAAGCAGCGGCTGAAACTGAAGGATGGCGGCCACTACGACGCCGAAGTGAAAACCACCTACAAGGC
CAAGAAACCCGTGCAGCTGCCTGGCGCCTACAACGTGAACATCAAGCTGGACATCACCAGCCACAACGAGGACTACACCA
TCGTGGAACAGTACGAGAGAGCCGAAGGCAGACACAGCACAGGCGGAATGGACGAGCTGTACAAGTGACATATGGATAAT
CAACCTCTGGATTACAAAATTTGTGAAAGATTGACTGGTATTCTTAACTATGTTGCTCCTTTTACGCTATGTGGATACGC
TGCTTTAATGCCTTTGTATCATGCTATTGCTTCCCGTATGGCTTTCATTTTCTCCTCCTTGTATAAATCCTGGTTAGTTC
TTGCCACGGCGGAACTCATCGCCGCCTGCCTTGCCCGCTGCTGGACAGGGGCTCGGCTGTTGGGCACTGACAATTCCGTG
GTGTTTATTTGTGAAATTTGTGATGCTATTGCTTTATTTGTAACCATTCTAGCTTTATTTGTGAAATTTGTGATGCTATT
GCTTTATTTGTAACCATTATAAGCTGCAATAAACAAGTTAACAACAACAATTGCATTCATTTTATGTTTCAGGTTCAGGG
GGAGATGTGGGAGGTTTTTTAAAGCACCGATTTTGATTCTCAAACAAATGTGTCACAAAGTAAGGATTCTGATGTGTATA
TCACAGACAAAACTGTGCTAGACATGAGGTCTATGGACTTCAAGAGCAACAGTGCTGTGGCCTGGAGCAACAAATCTGAC
TTTGCATGTGCAAACGCCTTCAACAACAGCATTATTCCAGAAGACACCTTCTTCCCCAGCCCAGGTAAGGGCAGCTTTGG
TGCCTTCGCAGGCTGTTTCCTTGCTTCAGGAATGGCCAGGTTCTGCCCAGAGCTCTGGTCAATGATGTCTAAAACTCCTC
TGATTGGTGGTCTCGGCCTTATCCATTGCCACCAAAACCCTCTTTTTACTAAGAAACAGTGAGCCTTGTTCTGGCAGTCC
AGAGAATGACACGGGAAAAAAGCAGATGAAGAGAAGGTGGCAGGAGAGGGCACGTGGCCCAGCCTCAGTCTCTCCAACTG
AGTTCCTGCCTGCCTGCCTTTGCTCAGACTGTTTGCCCCTTACTGCTCTTCTAGGCCTCATTCTAAGCCCCTTCTCCAAG
TTGCCTCTCCTTATTTCTCCCTGTCTGCCAAAAAATCTTTCCCAGCTCACTAAGTCAGTCTCACGCAGTCACTCATTAAC
CCACCAATCACTGATTGTGCCGGCACATGAATGCACCAGGTGTTGAAGTGGAGGAATTAAAAAGTCAGATGAGGGGTGTG
CCCAGAGGAAGCACCATTCTAGTTGGGGGAGCCCATCTGTCAGCTGGGAAAAGTCCAAATAACTTCAGATTGGAATGTGT
TTTAACTCAGGGTTGAGAAAACAGCTACCTTCAGGACAAAAGTCAGGGAAGGGCTCTCTGAAGAAATGCTACTTGAAGAT
ACCAGCCCTACCAAGGGCAGGGAGAGGACCCTATAGAGGCCTGGGACAGGAGCTCAATGAGAAAGGAGAAGAGCAGCAGG
CATGAGTTGAATGAAGGAGGCAGGGCCGGGTCACAGGGCCTTCTAGGCCATGAGAGGGTAGACAGGCTAGCCGCGTTGCT
GGCGTTTTTCCATAGGCTCCGCCCCCCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACGCTGTAGGTATCTCAGTTCGGTGTAGGTCGT
TCGCTCCAAGCTGGGCTGTGTGCACGAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGATTAGCAGAGCGAGGTATGTAGGCGGT
GCTACAGAGTTCTTGAAGTGGTGGCCTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATCTTTAGAAAAACTCATCGAGCATCAAATGAA
AGGCAGTTCCATAGGATGGCAAGATCCTGGTATCGGTCTGCGATTCCGACTCGTCCAACATCAATACAACCTATTAATTT
CCCCTCGTCAAAAATAAGGTTATCAAGTGAGAAATCACCATGAGTGACGACTGAATCCGGTGAGAATGGCAAAAGTTTAT
GCATTTCTTTCCAGACTTGTTCAACAGGCCAGCCATTACGCTCGTCATCAAAATCACTCGCATCAACCAAACCGTTATTC
ATTCGTGATTGCGCCTGAGCCAGACGAAATACGCGATCGCTGTTAAAAGGACAATTACAAACAGGAATCGAATGCAACCG
GCGCAGGAACACTGCCAGCGCATCAACAATATTTTCACCTGAATCAGGATATTCTTCTAATACCTGGAATGCTGTTTTTC
CGGGGATCGCAGTGGTGAGTAACCATGCATCATCAGGAGTACGGATAAAATGCTTGATGGTCGGAAGAGGCATAAATTCC
GTCAGCCAGTTTAGTCTGACCATCTCATCTGTAACATCATTGGCAACGCTACCTTTGCCATGTTTCAGAAACAACTCTGG
CGCATCGGGCTTCCCATACAAGCGATAGATTGTCGCACCTGATTGCCCGACATTATCGCGAGCCCATTTATACCCATATA
AATCAGCATCCATGTTGGAATTTAATCGCGGCCTCGACGTTTCCCGTTGAATATGGCTCATAACACCCCTTGTATTACTG
TTTATGTAAGCAGACAGTTTTATTGTTCATGATGATATATTTTTATCTTGTGCAATGTAACATCAGAGATTTTGAGACAC

In certain embodiments, the Secondary Promoter Construct comprises nucleotides 1 to 3402 of SEQ ID NO: 50. In certain embodiments, the Secondary Promoter Construct comprises nucleotides 4111 to 7051 of SEQ ID NO: 50. In certain embodiments, the Secondary Promoter Construct comprises nucleotides 1 to 3402 and nucleotides 4111 to 7051 of SEQ ID NO: 50. SEQ ID NO: 50 is provided below.

[SEQ ID NO: 50]
GGTACCACATTAAAAACACAAAATCCTACGGAAATACTGAAGAATGAGTCTCAGCACTAAGGAAAAGCCTCCAGCAGCTC
CTGCTTTCTGAGGGTGAAGGATAGACGCTGTGGCTCTGCATGACTCACTAGCACTCTATCACGGCCATATTCTGGCAGGG
TCAGTGGCTCCAACTAACATTTGTTTGGTACTTTACAGTTTATTAAATAGATGTTTATATGGAGAAGCTCTCATTTCTTT
CTCAGAAGAGCCTGGCTAGGAAGGTGGATGAGGCACCATATTCATTTTGCAGGTGAAATTCCTGAGATGTAAGGAGCTGC
TGTGACTTGCTCAAGGCCTTATATCGAGTAAACGGTAGTGCTGGGGCTTAGACGCAGGTGTTCTGATTTATAGTTCAAAA
CCTCTATCAATGAGAGAGCAATCTCCTGGTAATGTGATAGATTTCCCAACTTAATGCCAACATACCATAAACCTCCCATT
CTGCTAATGCCCAGCCTAAGTTGGGGAGACCACTCCAGATTCCAAGATGTACAGTTTGCTTTGCTGGGCCTTTTTCCCAT
GCCTGCCTTTACTCTGCCAGAGTTATATTGCTGGGGTTTTGAAGAAGATCCTATTAAATAAAAGAATAAGCAGTATTATT
AAGTAGCCCTGCATTTCAGGTTTCCTTGAGTGGCAGGCCAGGCCTGGCCGTGAACGTTCACTGAAATCATGGCCTCTTGG
CCAAGATTGATAGCTTGTGCCTGTCCCTGAGTCCCAGTCCATCACGAGCAGCTGGTTTCTAAGATGCTATTTCCCGTATA
AAGCATGAGACCGTGACTTGCCAGCCCCACAGAGCCCCGCCCTTGTCCATCACTGGCATCTGGACTCCAGCCTGGGTTGG
GGCAAAGAGGGAAATGAGATCATGTCCTAACCCTGATCCTCTTGTCCCACAGATATCCAGAACCCTGACCCTGCCGTGTA
CCAGCTGAGAGACTCTAAATCCAGTGACAAGTCTGTCTGCCTATTCGAATTCGGCTCCGGAGCCACTAACTTCTCCCTGT
TGAAACAGGCTGGCGATGTTGAAGAAAACCCCGGTCCTATGGCCACCGGCTCTAGAACAAGCCTGCTGCTCGCTTTTGGC
CTGCTCTGCCTCCCATGGCTCCAAGAAGGATCTGCTGATGCTGGAATCACCCAGAGCCCAAGATACAAGATCACAGAGAC
AGGAAGGCAGGTGACCTTGATGTGTCACCAGACTTGGAGCCACAGCTATATGTTCTGGTATCGACAAGACCTGGGACATG
GGCTGAGGCTGATCTATTACTCAGCAGCTGCTGATATTACAGATAAAGGAGAAGTCTCCGATGGCTATGTTGTCTCCAGA
TCCAAGACAGAGAATTTCCCCCTCACTCTGGAGTCAGCTACCCGCTCCCAGACATCTGTGTATTTCTGCGCCAGCAGTGA
GGACAGTTACGAGCAGTACTTCGGGCCGGGCACCAGGCTCACGGTCACAGAGGACCTGAAAAACGTGTTCCCTCCAAAAG
TGGCCGTGTTCGAGCCTTCTGAGGCCGAGATCAGCCACACACAGAAAGCCACACTCGTGTGTCTGGCTACCGGCTTCTAC
CCCGATCACGTGGAACTGTCTTGGTGGGTCAACGGCAAAGAGGTGCACAGCGGCGTCAGCACAGATCCCCAGCCTCTGAA
AGAACAGCCCGCTCTGAACGACAGCCGCTACTGCCTGTCTAGCAGACTGAGAGTGTCCGCCACCTTCTGGCAGAACCCCA
GAAACCACTTCAGATGCCAGGTCCAGTTCTACGGCCTGAGCGAGAACGATGAGTGGACCCAGGACAGAGCCAAGCCTGTG
ACACAGATCGTGTCTGCCGAAGCCTGGGGCAGAGCCGATTGTGGCTTTACCAGCGAGTCATACCAGCAGGGCGTGCTGTC
TGCCACCATCCTGTATGAGATCCTGCTCGGCAAGGCCACACTGTACGCTGTGCTGGTGTCTGCTCTGGTGCTGATGGCTA
TGGTCTCCCGGGAGCGCATCCCCGAGGCCCGGGCCAAGCGGGGCAGCGGCGCCACCAACTTCAGCCTGCTGAAGCAGGCC
GGCGACGTGGAGGAGAACCCCGGCCCTATGGCCACAGGCAGCAGAACATCTCTGCTGCTGGCCTTCGGACTGCTGTGTCT
GCCTTGGCTGCAAGAGGGTTCCGCCGCCCAGTCAGTGACCCAGCCTGACATCCACATCACTGTCTCTGAAGGAGCCTCAC
TGGAGTTGAGATGTAACTATTCCTATGGGGCAACACCTTATCTCTTCTGGTATGTCCAGTCCCCCGGCCAAGGCCTCCAG
CTGCTCCTGAAGTACTTTTCAGGAGACACTCTGGTTCAAGGCATTAAAGGCTTTGAGGCTGAATTTAAGAGGAGTCAATC
TTCCTTCAATCTGAGGAAACCCTCTGTGCATTGGAGTGATGCTGCTGAGTACTTCTGTGCTGTGGGTGAATTGGACACAG
GCTTTCAGAAACTTGTATTTGGAACTGGCACCCGACTTCTGGTCAGTCCAAATATTCAGAACCCCGATCCTGCCGTGTAC
CAGCTGAGAGACAGCAAGAGCAGCGACAAGAGCGTGTGCCTGTTCACCGACTTCGACAGCCAGACCAACGTGTCCCAGAG
CAAGGACAGCGACGTGTACATCACCGACAAGACCGTGCTGGACATGCGGAGCATGGACTTCAAGAGCAACAGCGCCGTGG
CCTGGTCCAACAAGAGCGATTTCGCCTGCGCCAACGCCTTCAACAACAGCATTATCCCCGAGGACACATTCTTCCCAAGT
CCTGAGAGCAGCTGCGACGTGAAGCTGGTGGAAAAGAGCTTCGAGACAGACACCAACCTGAACTTCCAGAACCTGTCCGT
GATCGGCTTCCGCATCCTGCTGCTGAAAGTGGCCGGCTTCAACCTGCTGATGACCCTGAGACTGTGGTCCAGCTGAGTCG
ACGCTTTAAAAAACCTCCCACATCTCCCCCTGAACCTGAAACATAAAATGAATGCAATTGTTGTTGTTAACTTGTTTATT
GCAGCTTATAATGGTTACAAATAAAGCAATAGCATCACAAATTTCACAAATAAAGCTAGAATGGTTACAAATAAAGCAAT
AGCATCACAAATTTCACAAATAAACACCACGGAATTGTCAGTGCCCAACAGCCGAGCCCCTGTCCAGCAGCGGGCAAGGC
AGGCGGCGATGAGTTCCGCCGTGGCAAGAACTAACCAGGATTTATACAAGGAGGAGAAAATGAAAGCCATACGGGAAGCA
ATAGCATGATACAAAGGCATTAAAGCAGCGTATCCACATAGCGTAAAAGGAGCAACATAGTTAAGAATACCAGTCAATCT
TTCACAAATTTTGTAATCCAGAGGTTGATTATCCATATGTCACTTGTACAGCTCGTCCATTCCGCCTGTGCTGTGTCTGC
CTTCGGCTCTCTCGTACTGTTCCACGATGGTGTAGTCCTCGTTGTGGCTGGTGATGTCCAGCTTGATGTTCACGTTGTAG
GCGCCAGGCAGCTGCACGGGTTTCTTGGCCTTGTAGGTGGTTTTCACTTCGGCGTCGTAGTGGCCGCCATCCTTCAGTTT
CAGCCGCTGCTTGATCTCGCCCTTCAGGGCGCCATCTTCAGGGTACATTCTCTCGCTGCTGGCTTCCCAGCCCATTGTCT
TTTTCTGCATCACGGGGCCATCAGAGGGAAAGTTGGTGCCCCGCAGCTTCACTTTGTAGATGAACTCGCCGTCCTGCAGA
GAGCTATCTTGAGTCACGGTGACCACGCCGCCGTCCTCGAAGTTCATCACTCTCTCCCACTTGAAGCCCTCGGGGAAGCT
CAGCTTCAGGTAGTCGGGAATATCGGCGGGGTGCTTCACGTAGGCCTTGCTGCCGTACATAAACTGAGGGCTCAGAATGT
CCCAGGCAAAAGGCAGAGGGCCGCCTTTGGTCACTTTCAGCTTGGCGGTCTGTGTTCCCTCGTAAGGTCTGCCCTCGCCT
TCGCCTTCAATCTCGAACTCGTGGCCGTTCACGCTGCCTTCCATGTGCACCTTGAACCGCATGAACTCTTTGATGATGGC
CATGTTGTCCTCTTCGCCCTTGGACACCATGGTGGCGGATCCCTTCTATGGAAGTCAAAACAGCGTGGATGGCGTCTCCA
GGCGATCTGACGGTTCACTAAACGAGCTCTGCTTATATAGAGCTCGGGGAGCAGAAGCGCGCGAACAGAAGCGAGAAGCG
AACTGATTGGTTAGTTCAAATAAGGCACAGGGTCATTTCAGGTCCTTGGGGCACCCTGGAAACATCTGATGGTTCTCTAG
AAACTGCTGAGGGCGGGACCGCATCTGGGGACCATCTGTTCTTGGCCCTGAGCCGGGGCAGGAACTGCTTACCACAGATA
TCCTGTTTGGCCCATATTCTGCTGTTCCAACTGTTCTTGGCCCTGAGCCGGGGCAGGAACTGCTTACCACAGATATCCTG
TTTGGCCCATATTCTCCTGTTTCTCTGTTCCTCGAGACCGATTTTGATTCTCAAACAAATGTGTCACAAAGTAAGGATTC
TGATGTGTATATCACAGACAAAACTGTGCTAGACATGAGGTCTATGGACTTCAAGAGCAACAGTGCTGTGGCCTGGAGCA
ACAAATCTGACTTTGCATGTGCAAACGCCTTCAACAACAGCATTATTCCAGAAGACACCTTCTTCCCCAGCCCAGGTAAG
GGCAGCTTTGGTGCCTTCGCAGGCTGTTTCCTTGCTTCAGGAATGGCCAGGTTCTGCCCAGAGCTCTGGTCAATGATGTC
TAAAACTCCTCTGATTGGTGGTCTCGGCCTTATCCATTGCCACCAAAACCCTCTTTTTACTAAGAAACAGTGAGCCTTGT
TCTGGCAGTCCAGAGAATGACACGGGAAAAAAGCAGATGAAGAGAAGGTGGCAGGAGAGGGCACGTGGCCCAGCCTCAGT
CTCTCCAACTGAGTTCCTGCCTGCCTGCCTTTGCTCAGACTGTTTGCCCCTTACTGCTCTTCTAGGCCTCATTCTAAGCC
CCTTCTCCAAGTTGCCTCTCCTTATTTCTCCCTGTCTGCCAAAAAATCTTTCCCAGCTCACTAAGTCAGTCTCACGCAGT
CACTCATTAACCCACCAATCACTGATTGTGCCGGCACATGAATGCACCAGGTGTTGAAGTGGAGGAATTAAAAAGTCAGA
TGAGGGGTGTGCCCAGAGGAAGCACCATTCTAGTTGGGGGAGCCCATCTGTCAGCTGGGAAAAGTCCAAATAACTTCAGA
TTGGAATGTGTTTTAACTCAGGGTTGAGAAAACAGCTACCTTCAGGACAAAAGTCAGGGAAGGGCTCTCTGAAGAAATGC
TACTTGAAGATACCAGCCCTACCAAGGGCAGGGAGAGGACCCTATAGAGGCCTGGGACAGGAGCTCAATGAGAAAGGAGA
AGAGCAGCAGGCATGAGTTGAATGAAGGAGGCAGGGCCGGGTCACAGGGCCTTCTAGGCCATGAGAGGGTAGACAGGCTA
GCCGCGTTGCTGGCGTTTTTCCATAGGCTCCGCCCCCCTGACGAGCATCACAAAAATCGACGCTCAAGTCAGAGGTGGCG
AAACCCGACAGGACTATAAAGATACCAGGCGTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCCCGACCGCTGCGCCTTATCCGGTAACTA
TCGTCTTGAGTCCAACCCGGTAAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGAACAGTATTTGGTATCTGCGCT
CTGCTGAAGCCAGTTACCTTCGGAAAAAGAGTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAGATCCTTTGATCTTTAGAAAAACTCATCGAG
CATCAAATGAAACTGCAATTTATTCATATCAGGATTATCAATACCATATTTTTGAAAAAGCCGTTTCTGTAATGAAGGAG
AAAACTCACCGAGGCAGTTCCATAGGATGGCAAGATCCTGGTATCGGTCTGCGATTCCGACTCGTCCAACATCAATACAA
CCTATTAATTTCCCCTCGTCAAAAATAAGGTTATCAAGTGAGAAATCACCATGAGTGACGACTGAATCCGGTGAGAATGG
CAAAAGTTTATGCATTTCTTTCCAGACTTGTTCAACAGGCCAGCCATTACGCTCGTCATCAAAATCACTCGCATCAACCA
AACCGTTATTCATTCGTGATTGCGCCTGAGCCAGACGAAATACGCGATCGCTGTTAAAAGGACAATTACAAACAGGAATC
GAATGCAACCGGCGCAGGAACACTGCCAGCGCATCAACAATATTTTCACCTGAATCAGGATATTCTTCTAATACCTGGAA
TGCTGTTTTTCCGGGGATCGCAGTGGTGAGTAACCATGCATCATCAGGAGTACGGATAAAATGCTTGATGGTCGGAAGAG
GCATAAATTCCGTCAGCCAGTTTAGTCTGACCATCTCATCTGTAACATCATTGGCAACGCTACCTTTGCCATGTTTCAGA
AACAACTCTGGCGCATCGGGCTTCCCATACAAGCGATAGATTGTCGCACCTGATTGCCCGACATTATCGCGAGCCCATTT
ATACCCATATAAATCAGCATCCATGTTGGAATTTAATCGCGGCCTCGACGTTTCCCGTTGAATATGGCTCATAACACCCC
TTGTATTACTGTTTATGTAAGCAGACAGTTTTATTGTTCATGATGATATATTTTTATCTTGTGCAATGTAACATCAGAGA
TTTTGAGACAC

In certain embodiments, the Secondary Promoter Construct comprises nucleotides 1 to 3931 of SEQ ID NO: 51. In certain embodiments, the Secondary Promoter Construct comprises nucleotides 4640 to 7258 of SEQ ID NO: 51. In certain embodiments, the Secondary Promoter Construct comprises nucleotides 1 to 4086 and nucleotides 4795 to 7413 of SEQ ID NO: 51. SEQ ID NO: 51 is provided below.

[SEQ ID NO: 51]
GGTACCACATTAAAAACACAAAATCCTACGGAAATACTGAAGAATGAGTCTCAGCACTAAGGAAAAGCCTCCAGCAGCTC
CTGCTTTCTGAGGGTGAAGGATAGACGCTGTGGCTCTGCATGACTCACTAGCACTCTATCACGGCCATATTCTGGCAGGG
TCAGTGGCTCCAACTAACATTTGTTTGGTACTTTACAGTTTATTAAATAGATGTTTATATGGAGAAGCTCTCATTTCTTT
CTCAGAAGAGCCTGGCTAGGAAGGTGGATGAGGCACCATATTCATTTTGCAGGTGAAATTCCTGAGATGTAAGGAGCTGC
TGTGACTTGCTCAAGGCCTTATATCGAGTAAACGGTAGTGCTGGGGCTTAGACGCAGGTGTTCTGATTTATAGTTCAAAA
CCTCTATCAATGAGAGAGCAATCTCCTGGTAATGTGATAGATTTCCCAACTTAATGCCAACATACCATAAACCTCCCATT
CTGCTAATGCCCAGCCTAAGTTGGGGAGACCACTCCAGATTCCAAGATGTACAGTTTGCTTTGCTGGGCCTTTTTCCCAT
GCCTGCCTTTACTCTGCCAGAGTTATATTGCTGGGGTTTTGAAGAAGATCCTATTAAATAAAAGAATAAGCAGTATTATT
AAGTAGCCCTGCATTTCAGGTTTCCTTGAGTGGCAGGCCAGGCCTGGCCGTGAACGTTCACTGAAATCATGGCCTCTTGG
CCAAGATTGATAGCTTGTGCCTGTCCCTGAGTCCCAGTCCATCACGAGCAGCTGGTTTCTAAGATGCTATTTCCCGTATA
AAGCATGAGACCGTGACTTGCCAGCCCCACAGAGCCCCGCCCTTGTCCATCACTGGCATCTGGACTCCAGCCTGGGTTGG
GGCAAAGAGGGAAATGAGATCATGTCCTAACCCTGATCCTCTTGTCCCACAGATATCCAGAACCCTGACCCTGCCGTGTA
CCAGCTGAGAGACTCTAAATCCAGTGACAAGTCTGTCTGCCTATTCGAATTCGGGCTGGGGCGGTCCCCTCCCATTTCCA
TGACGTCATGGTTACCAAGAGGGGCAAGTAGGGCACCCTTTGAAGCTCTCCCGCAGAAGCCACATCCTCTGGAAAGAAGA
GTTTATAATACTGAGTTAGAGATAGCATCGCCCCAGGCCACGTGCCGAGGGGAGCAGGCTGGGCCGTTACACCACCCCCC
AACCGCAGGTGCAGCAAGGCCAACATGCCAGGCTGGGAGGGGCTGCCGGCCCCTCGTTGAGGGCTCCGGAGCCACTAACT
TCTCCCTGTTGAAACAGGCTGGCGATGTTGAAGAAAACCCCGGTCCTATGGCCACCGGCTCTAGAACAAGCCTGCTGCTC
GCTTTTGGCCTGCTCTGCCTCCCATGGCTCCAAGAAGGATCTGCTGATGCTGGAATCACCCAGAGCCCAAGATACAAGAT
CACAGAGACAGGAAGGCAGGTGACCTTGATGTGTCACCAGACTTGGAGCCACAGCTATATGTTCTGGTATCGACAAGACC
TGGGACATGGGCTGAGGCTGATCTATTACTCAGCAGCTGCTGATATTACAGATAAAGGAGAAGTCTCCGATGGCTATGTT
GTCTCCAGATCCAAGACAGAGAATTTCCCCCTCACTCTGGAGTCAGCTACCCGCTCCCAGACATCTGTGTATTTCTGCGC
CAGCAGTGAGGACAGTTACGAGCAGTACTTCGGGCCGGGCACCAGGCTCACGGTCACAGAGGACCTGAAAAACGTGTTCC
CTCCAAAAGTGGCCGTGTTCGAGCCTTCTGAGGCCGAGATCAGCCACACACAGAAAGCCACACTCGTGTGTCTGGCTACC
GGCTTCTACCCCGATCACGTGGAACTGTCTTGGTGGGTCAACGGCAAAGAGGTGCACAGCGGCGTCAGCACAGATCCCCA
GCCTCTGAAAGAACAGCCCGCTCTGAACGACAGCCGCTACTGCCTGTCTAGCAGACTGAGAGTGTCCGCCACCTTCTGGC
AGAACCCCAGAAACCACTTCAGATGCCAGGTCCAGTTCTACGGCCTGAGCGAGAACGATGAGTGGACCCAGGACAGAGCC
AAGCCTGTGACACAGATCGTGTCTGCCGAAGCCTGGGGCAGAGCCGATTGTGGCTTTACCAGCGAGTCATACCAGCAGGG
CGTGCTGTCTGCCACCATCCTGTATGAGATCCTGCTCGGCAAGGCCACACTGTACGCTGTGCTGGTGTCTGCTCTGGTGC
TGATGGCTATGGTCTCCCGGGAGCGCATCCCCGAGGCCCGGGCCAAGCGGGGCAGCGGCGCCACCAACTTCAGCCTGCTG
AAGCAGGCCGGCGACGTGGAGGAGAACCCCGGCCCTATGGCCACAGGCAGCAGAACATCTCTGCTGCTGGCCTTCGGACT
GCTGTGTCTGCCTTGGCTGCAAGAGGGTTCCGCCGCCCAGTCAGTGACCCAGCCTGACATCCACATCACTGTCTCTGAAG
GAGCCTCACTGGAGTTGAGATGTAACTATTCCTATGGGGCAACACCTTATCTCTTCTGGTATGTCCAGTCCCCCGGCCAA
GGCCTCCAGCTGCTCCTGAAGTACTTTTCAGGAGACACTCTGGTTCAAGGCATTAAAGGCTTTGAGGCTGAATTTAAGAG
GAGTCAATCTTCCTTCAATCTGAGGAAACCCTCTGTGCATTGGAGTGATGCTGCTGAGTACTTCTGTGCTGTGGGTGAAT
TGGACACAGGCTTTCAGAAACTTGTATTTGGAACTGGCACCCGACTTCTGGTCAGTCCAAATATTCAGAACCCCGATCCT
GCCGTGTACCAGCTGAGAGACAGCAAGAGCAGCGACAAGAGCGTGTGCCTGTTCACCGACTTCGACAGCCAGACCAACGT
GTCCCAGAGCAAGGACAGCGACGTGTACATCACCGACAAGACCGTGCTGGACATGCGGAGCATGGACTTCAAGAGCAACA
GCGCCGTGGCCTGGTCCAACAAGAGCGATTTCGCCTGCGCCAACGCCTTCAACAACAGCATTATCCCCGAGGACACATTC
TTCCCAAGTCCTGAGAGCAGCTGCGACGTGAAGCTGGTGGAAAAGAGCTTCGAGACAGACACCAACCTGAACTTCCAGAA
CCTGTCCGTGATCGGCTTCCGCATCCTGCTGCTGAAAGTGGCCGGCTTCAACCTGCTGATGACCCTGAGACTGTGGTCCA
GCTGATGTGCCTTCTAGTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATTCTGGGGGGTGGGGTGG
GGCAGGACAGCAAGGGGGAGGATTGGGAAGACAATAGCAGGCATGCTGGGGATGCGGTGGGCTCTATGGCGTCGACGAGC
TCACGGGGACAGCCCCCCCCCAAAGCCCCCAGGGATGTAATTACGTCCCTCCCCCGCTAGGGGGCAGCAGCGAGCCGCCC
GGGGCTCCGCTCCGGTCCGGCGCTCCCCCCGCATCCCCGAGCCGGCAGCGTGCGGGGACAGCCCGGGCACGGGGAAGGTG
GCACGGGATCGCTTTCCTCTGAACGCTTCTCGCTGCTCTTTGAGCCTGCAGACACCTGGGGGGATACGGGGAAAAAGCTT
CTCGACGAACAGAGAAACAGGAGAATATGGGCCAAACAGGATATCTGTGGTAAGCAGTTCCTGCCCCGGCTCAGGGCCAA
GAACAGTTGGAACAGCAGAATATGGGCCAAACAGGATATCTGTGGTAAGCAGTTCCTGCCCCGGCTCAGGGCCAAGAACA
GATGGTCCCCAGATGCGGTCCCGCCCTCAGCAGTTTCTAGAGAACCATCAGATGTTTCCAGGGTGCCCCAAGGACCTGAA
ATGACCCTGTGCCTTATTTGAACTAACCAATCAGTTCGCTTCTCGCTTCTGTTCGCGCGCTTCTGCTCCCCGAGCTCTAT
ATAAGCAGAGCTCGTTTAGTGAACCGTCAGATCGCCTGGAGACGCCATCCACGCTGTTTTGACTTCCATAGAAGGGATCC
GCCACCATGGTGTCCAAGGGCGAAGAGGACAACATGGCCATCATCAAAGAGTTCATGCGGTTCAAGGTGCACATGGAAGG
CAGCGTGAACGGCCACGAGTTCGAGATTGAAGGCGAAGGCGAGGGCAGACCTTACGAGGGAACACAGACCGCCAAGCTGA
AAGTGACCAAAGGCGGCCCTCTGCCTTTTGCCTGGGACATTCTGAGCCCTCAGTTTATGTACGGCAGCAAGGCCTACGTG
AAGCACCCCGCCGATATTCCCGACTACCTGAAGCTGAGCTTCCCCGAGGGCTTCAAGTGGGAGAGAGTGATGAACTTCGA
GGACGGCGGCGTGGTCACCGTGACTCAAGATAGCTCTCTGCAGGACGGCGAGTTCATCTACAAAGTGAAGCTGCGGGGCA
CCAACTTTCCCTCTGATGGCCCCGTGATGCAGAAAAAGACAATGGGCTGGGAAGCCAGCAGCGAGAGAATGTACCCTGAA
GATGGCGCCCTGAAGGGCGAGATCAAGCAGCGGCTGAAACTGAAGGATGGCGGCCACTACGACGCCGAAGTGAAAACCAC
CTACAAGGCCAAGAAACCCGTGCAGCTGCCTGGCGCCTACAACGTGAACATCAAGCTGGACATCACCAGCCACAACGAGG
ACTACACCATCGTGGAACAGTACGAGAGAGCCGAAGGCAGACACAGCACAGGCGGAATGGACGAGCTGTACAAGAGAGCC
AAACGGGGGTCCGGCGCTACCAATTTCAGTTTGCTCAAACAGGCCGGAGACGTCGAGGAAAATCCTGGCCCCCATATGAC
CGATTTTGATTCTCAAACAAATGTGTCACAAAGTAAGGATTCTGATGTGTATATCACAGACAAAACTGTGCTAGACATGA
GGTCTATGGACTTCAAGAGCAACAGTGCTGTGGCCTGGAGCAACAAATCTGACTTTGCATGTGCAAACGCCTTCAACAAC
AGCATTATTCCAGAAGACACCTTCTTCCCCAGCCCAGGTAAGGGCAGCTTTGGTGCCTTCGCAGGCTGTTTCCTTGCTTC
AGGAATGGCCAGGTTCTGCCCAGAGCTCTGGTCAATGATGTCTAAAACTCCTCTGATTGGTGGTCTCGGCCTTATCCATT
GCCACCAAAACCCTCTTTTTACTAAGAAACAGTGAGCCTTGTTCTGGCAGTCCAGAGAATGACACGGGAAAAAAGCAGAT
GAAGAGAAGGTGGCAGGAGAGGGCACGTGGCCCAGCCTCAGTCTCTCCAACTGAGTTCCTGCCTGCCTGCCTTTGCTCAG
ACTGTTTGCCCCTTACTGCTCTTCTAGGCCTCATTCTAAGCCCCTTCTCCAAGTTGCCTCTCCTTATTTCTCCCTGTCTG
CCAAAAAATCTTTCCCAGCTCACTAAGTCAGTCTCACGCAGTCACTCATTAACCCACCAATCACTGATTGTGCCGGCACA
TGAATGCACCAGGTGTTGAAGTGGAGGAATTAAAAAGTCAGATGAGGGGTGTGCCCAGAGGAAGCACCATTCTAGTTGGG
GGAGCCCATCTGTCAGCTGGGAAAAGTCCAAATAACTTCAGATTGGAATGTGTTTTAACTCAGGGTTGAGAAAACAGCTA
CCTTCAGGACAAAAGTCAGGGAAGGGCTCTCTGAAGAAATGCTACTTGAAGATACCAGCCCTACCAAGGGCAGGGAGAGG
ACCCTATAGAGGCCTGGGACAGGAGCTCAATGAGAAAGGAGAAGAGCAGCAGGCATGAGTTGAATGAAGGAGGCAGGGCC
GGGTCACAGGGCCTTCTAGGCCATGAGAGGGTAGACAGGCTAGCCGCGTTGCTGGCGTTTTTCCATAGGCTCCGCCCCCC
TGACGAGCATCACAAAAATCGACGCTCAAGTCAGAGGTGGCGAAACCCGACAGGACTATAAAGATACCAGGCGTTTCCCC
CTGGAAGCTCCCTCGTGCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTCCAACCCGGTAAGACACGACTTATCGC
CACTGGCAGCAGCCACTGGTAACAGGATTAGCAGAGCGAGGTATGTAGGCGGTGCTACAGAGTTCTTGAAGTGGTGGCCT
AACTACGGCTACACTAGAAGAACAGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCAGCAGATTACGCGCAGAAAAAAAG
GATCTCAAGAAGATCCTTTGATCTTTAGAAAAACTCATCGAGCATCAAATGAAACTGCAATTTATTCATATCAGGATTAT
CAATACCATATTTTTGAAAAAGCCGTTTCTGTAATGAAGGAGAAAACTCACCGAGGCAGTTCCATAGGATGGCAAGATCC
TGGTATCGGTCTGCGATTCCGACTCGTCCAACATCAATACAACCTATTAATTTCCCCTCGTCAAAAATAAGGTTATCAAG
TGAGAAATCACCATGAGTGACGACTGAATCCGGTGAGAATGGCAAAAGTTTATGCATTTCTTTCCAGACTTGTTCAACAG
GCCAGCCATTACGCTCGTCATCAAAATCACTCGCATCAACCAAACCGTTATTCATTCGTGATTGCGCCTGAGCCAGACGA
AATACGCGATCGCTGTTAAAAGGACAATTACAAACAGGAATCGAATGCAACCGGCGCAGGAACACTGCCAGCGCATCAAC
AATATTTTCACCTGAATCAGGATATTCTTCTAATACCTGGAATGCTGTTTTTCCGGGGATCGCAGTGGTGAGTAACCATG
CATCATCAGGAGTACGGATAAAATGCTTGATGGTCGGAAGAGGCATAAATTCCGTCAGCCAGTTTAGTCTGACCATCTCA
TCTGTAACATCATTGGCAACGCTACCTTTGCCATGTTTCAGAAACAACTCTGGCGCATCGGGCTTCCCATACAAGCGATA
GATTGTCGCACCTGATTGCCCGACATTATCGCGAGCCCATTTATACCCATATAAATCAGCATCCATGTTGGAATTTAATC
GCGGCCTCGACGTTTCCCGTTGAATATGGCTCATAACACCCCTTGTATTACTGTTTATGTAAGCAGACAGTTTTATTGTT
CATGATGATATATTTTTTATCTTGTGCAATGTAACATCAGAGATTTTGAGACAC

In certain embodiments, the Secondary Promoter Construct comprises nucleotides 1 to 4140 of SEQ ID NO: 52. In certain embodiments, the Secondary Promoter Construct comprises nucleotides 4849 to 7467 of SEQ ID NO: 52. In certain embodiments, the Secondary Promoter Construct comprises nucleotides 1 to 4140 and nucleotides 4849 to 7467 of SEQ ID NO: 52. SEQ ID NO: 52 is provided below.

[SEQ ID NO: 52]
GGTACCACATTAAAAACACAAAATCCTACGGAAATACTGAAGAATGAGTCTCAGCACTAAGGAAAAGCCTCCAGCAGCTC
CTGCTTTCTGAGGGTGAAGGATAGACGCTGTGGCTCTGCATGACTCACTAGCACTCTATCACGGCCATATTCTGGCAGGG
TCAGTGGCTCCAACTAACATTTGTTTGGTACTTTACAGTTTATTAAATAGATGTTTATATGGAGAAGCTCTCATTTCTTT
CTCAGAAGAGCCTGGCTAGGAAGGTGGATGAGGCACCATATTCATTTTGCAGGTGAAATTCCTGAGATGTAAGGAGCTGC
TGTGACTTGCTCAAGGCCTTATATCGAGTAAACGGTAGTGCTGGGGCTTAGACGCAGGTGTTCTGATTTATAGTTCAAAA
CCTCTATCAATGAGAGAGCAATCTCCTGGTAATGTGATAGATTTCCCAACTTAATGCCAACATACCATAAACCTCCCATT
CTGCTAATGCCCAGCCTAAGTTGGGGAGACCACTCCAGATTCCAAGATGTACAGTTTGCTTTGCTGGGCCTTTTTCCCAT
GCCTGCCTTTACTCTGCCAGAGTTATATTGCTGGGGTTTTGAAGAAGATCCTATTAAATAAAAGAATAAGCAGTATTATT
AAGTAGCCCTGCATTTCAGGTTTCCTTGAGTGGCAGGCCAGGCCTGGCCGTGAACGTTCACTGAAATCATGGCCTCTTGG
CCAAGATTGATAGCTTGTGCCTGTCCCTGAGTCCCAGTCCATCACGAGCAGCTGGTTTCTAAGATGCTATTTCCCGTATA
AAGCATGAGACCGTGACTTGCCAGCCCCACAGAGCCCCGCCCTTGTCCATCACTGGCATCTGGACTCCAGCCTGGGTTGG
GGCAAAGAGGGAAATGAGATCATGTCCTAACCCTGATCCTCTTGTCCCACAGATATCCAGAACCCTGACCCTGCCGTGTA
CCAGCTGAGAGACTCTAAATCCAGTGACAAGTCTGTCTGCCTATTCGAATTCCGTTACATAACTTACGTTAAATGGCCCG
CCTGGCTGACCGCCCAACGACCCCCGCCCATTGACGTCAATAATGATGTATGTTCCCATAGTAACGCCAATAGGGACTTT
CCATTGACGTCAATGGGTGGAGTATTTACGGTAAACTGCCCACTTGGCAGTACATCAAGTGTATCATATCCAAGTACGCC
CCCTATTGACGTCAATGACGGTAAATGGCCCGCCTGGCATTATGCCCAGTACATGACCTTATGGGACTTTCCTACTTGGC
AGTACATCTACGTATTAGTCATCGCTATTACCATGGGCTCCGGAGCCACTAACTTCTCCCTGTTGAAACAGGCTGGCGAT
GTTGAAGAAAACCCCGGTCCTATGGCCACCGGCTCTAGAACAAGCCTGCTGCTCGCTTTTGGCCTGCTCTGCCTCCCATG
GCTCCAAGAAGGATCTGCTGATGCTGGAATCACCCAGAGCCCAAGATACAAGATCACAGAGACAGGAAGGCAGGTGACCT
TGATGTGTCACCAGACTTGGAGCCACAGCTATATGTTCTGGTATCGACAAGACCTGGGACATGGGCTGAGGCTGATCTAT
TACTCAGCAGCTGCTGATATTACAGATAAAGGAGAAGTCTCCGATGGCTATGTTGTCTCCAGATCCAAGACAGAGAATTT
CCCCCTCACTCTGGAGTCAGCTACCCGCTCCCAGACATCTGTGTATTTCTGCGCCAGCAGTGAGGACAGTTACGAGCAGT
ACTTCGGGCCGGGCACCAGGCTCACGGTCACAGAGGACCTGAAAAACGTGTTCCCTCCAAAAGTGGCCGTGTTCGAGCCT
TCTGAGGCCGAGATCAGCCACACACAGAAAGCCACACTCGTGTGTCTGGCTACCGGCTTCTACCCCGATCACGTGGAACT
GTCTTGGTGGGTCAACGGCAAAGAGGTGCACAGCGGCGTCAGCACAGATCCCCAGCCTCTGAAAGAACAGCCCGCTCTGA
ACGACAGCCGCTACTGCCTGTCTAGCAGACTGAGAGTGTCCGCCACCTTCTGGCAGAACCCCAGAAACCACTTCAGATGC
CAGGTCCAGTTCTACGGCCTGAGCGAGAACGATGAGTGGACCCAGGACAGAGCCAAGCCTGTGACACAGATCGTGTCTGC
CGAAGCCTGGGGCAGAGCCGATTGTGGCTTTACCAGCGAGTCATACCAGCAGGGCGTGCTGTCTGCCACCATCCTGTATG
AGATCCTGCTCGGCAAGGCCACACTGTACGCTGTGCTGGTGTCTGCTCTGGTGCTGATGGCTATGGTCTCCCGGGAGCGC
ATCCCCGAGGCCCGGGCCAAGCGGGGCAGCGGCGCCACCAACTTCAGCCTGCTGAAGCAGGCCGGCGACGTGGAGGAGAA
CCCCGGCCCTATGGCCACAGGCAGCAGAACATCTCTGCTGCTGGCCTTCGGACTGCTGTGTCTGCCTTGGCTGCAAGAGG
GTTCCGCCGCCCAGTCAGTGACCCAGCCTGACATCCACATCACTGTCTCTGAAGGAGCCTCACTGGAGTTGAGATGTAAC
TATTCCTATGGGGCAACACCTTATCTCTTCTGGTATGTCCAGTCCCCCGGCCAAGGCCTCCAGCTGCTCCTGAAGTACTT
TTCAGGAGACACTCTGGTTCAAGGCATTAAAGGCTTTGAGGCTGAATTTAAGAGGAGTCAATCTTCCTTCAATCTGAGGA
AACCCTCTGTGCATTGGAGTGATGCTGCTGAGTACTTCTGTGCTGTGGGTGAATTGGACACAGGCTTTCAGAAACTTGTA
TTTGGAACTGGCACCCGACTTCTGGTCAGTCCAAATATTCAGAACCCCGATCCTGCCGTGTACCAGCTGAGAGACAGCAA
GAGCAGCGACAAGAGCGTGTGCCTGTTCACCGACTTCGACAGCCAGACCAACGTGTCCCAGAGCAAGGACAGCGACGTGT
ACATCACCGACAAGACCGTGCTGGACATGCGGAGCATGGACTTCAAGAGCAACAGCGCCGTGGCCTGGTCCAACAAGAGC
GATTTCGCCTGCGCCAACGCCTTCAACAACAGCATTATCCCCGAGGACACATTCTTCCCAAGTCCTGAGAGCAGCTGCGA
CGTGAAGCTGGTGGAAAAGAGCTTCGAGACAGACACCAACCTGAACTTCCAGAACCTGTCCGTGATCGGCTTCCGCATCC
TGCTGCTGAAAGTGGCCGGCTTCAACCTGCTGATGACCCTGAGACTGTGGTCCAGCTGATGTGCCTTCTAGTTGCCAGCC
ATCTGTTGTTTGCCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAGGGGGAGGATTGG
GAAGACAATAGCAGGCATGCTGGGGATGCGGTGGGCTCTATGGCGTCGACGAGCTCACGGGGACAGCCCCCCCCCAAAGC
CCCCAGGGATGTAATTACGTCCCTCCCCCGCTAGGGGGCAGCAGCGAGCCGCCCGGGGCTCCGCTCCGGTCCGGCGCTCC
CCCCGCATCCCCGAGCCGGCAGCGTGCGGGGACAGCCCGGGCACGGGGAAGGTGGCACGGGATCGCTTTCCTCTGAACGC
TTCTCGCTGCTCTTTGAGCCTGCAGACACCTGGGGGGATACGGGGAAAAAGCTTCTCGAGGAACAGAGAAACAGGAGAAT
ATGGGCCAAACAGGATATCTGTGGTAAGCAGTTCCTGCCCCGGCTCAGGGCCAAGAACAGTTGGAACAGCAGAATATGGG
CCAAACAGGATATCTGTGGTAAGCAGTTCCTGCCCCGGCTCAGGGCCAAGAACAGATGGTCCCCAGATGCGGTCCCGCCC
TCAGCAGTTTCTAGAGAACCATCAGATGTTTCCAGGGTGCCCCAAGGACCTGAAATGACCCTGTGCCTTATTTGAACTAA
CCAATCAGTTCGCTTCTCGCTTCTGTTCGCGCGCTTCTGCTCCCCGAGCTCTATATAAGCAGAGCTCGTTTAGTGAACCG
TCAGATCGCCTGGAGACGCCATCCACGCTGTTTTGACTTCCATAGAAGGGATCCGCCACCATGGTGTCCAAGGGCGAAGA
GGACAACATGGCCATCATCAAAGAGTTCATGCGGTTCAAGGTGCACATGGAAGGCAGCGTGAACGGCCACGAGTTCGAGA
TTGAAGGCGAAGGCGAGGGCAGACCTTACGAGGGAACACAGACCGCCAAGCTGAAAGTGACCAAAGGCGGCCCTCTGCCT
TTTGCCTGGGACATTCTGAGCCCTCAGTTTATGTACGGCAGCAAGGCCTACGTGAAGCACCCCGCCGATATTCCCGACTA
CCTGAAGCTGAGCTTCCCCGAGGGCTTCAAGTGGGAGAGAGTGATGAACTTCGAGGACGGCGGCGTGGTCACCGTGACTC
AAGATAGCTCTCTGCAGGACGGCGAGTTCATCTACAAAGTGAAGCTGCGGGGCACCAACTTTCCCTCTGATGGCCCCGTG
ATGCAGAAAAAGACAATGGGCTGGGAAGCCAGCAGCGAGAGAATGTACCCTGAAGATGGCGCCCTGAAGGGCGAGATCAA
GCAGCGGCTGAAACTGAAGGATGGCGGCCACTACGACGCCGAAGTGAAAACCACCTACAAGGCCAAGAAACCCGTGCAGC
TGCCTGGCGCCTACAACGTGAACATCAAGCTGGACATCACCAGCCACAACGAGGACTACACCATCGTGGAACAGTACGAG
AGAGCCGAAGGCAGACACAGCACAGGCGGAATGGACGAGCTGTACAAGAGAGCCAAACGGGGGTCCGGCGCTACCAATTT
CAGTTTGCTCAAACAGGCCGGAGACGTCGAGGAAAATCCTGGCCCCCATATGACCGATTTTGATTCTCAAACAAATGTGT
CACAAAGTAAGGATTCTGATGTGTATATCACAGACAAAACTGTGCTAGACATGAGGTCTATGGACTTCAAGAGCAACAGT
GCTGTGGCCTGGAGCAACAAATCTGACTTTGCATGTGCAAACGCCTTCAACAACAGCATTATTCCAGAAGACACCTTCTT
CCCCAGCCCAGGTAAGGGCAGCTTTGGTGCCTTCGCAGGCTGTTTCCTTGCTTCAGGAATGGCCAGGTTCTGCCCAGAGC
TCTGGTCAATGATGTCTAAAACTCCTCTGATTGGTGGTCTCGGCCTTATCCATTGCCACCAAAACCCTCTTTTTACTAAG
AAACAGTGAGCCTTGTTCTGGCAGTCCAGAGAATGACACGGGAAAAAAGCAGATGAAGAGAAGGTGGCAGGAGAGGGCAC
GTGGCCCAGCCTCAGTCTCTCCAACTGAGTTCCTGCCTGCCTGCCTTTGCTCAGACTGTTTGCCCCTTACTGCTCTTCTA
GGCCTCATTCTAAGCCCCTTCTCCAAGTTGCCTCTCCTTATTTCTCCCTGTCTGCCAAAAAATCTTTCCCAGCTCACTAA
GTCAGTCTCACGCAGTCACTCATTAACCCACCAATCACTGATTGTGCCGGCACATGAATGCACCAGGTGTTGAAGTGGAG
GAATTAAAAAGTCAGATGAGGGGTGTGCCCAGAGGAAGCACCATTCTAGTTGGGGGAGCCCATCTGTCAGCTGGGAAAAG
TCCAAATAACTTCAGATTGGAATGTGTTTTAACTCAGGGTTGAGAAAACAGCTACCTTCAGGACAAAAGTCAGGGAAGGG
CTCTCTGAAGAAATGCTACTTGAAGATACCAGCCCTACCAAGGGCAGGGAGAGGACCCTATAGAGGCCTGGGACAGGAGC
TCAATGAGAAAGGAGAAGAGCAGCAGGCATGAGTTGAATGAAGGAGGCAGGGCCGGGTCACAGGGCCTTCTAGGCCATGA
GAGGGTAGACAGGCTAGCCGCGTTGCTGGCGTTTTTCCATAGGCTCCGCCCCCCTGACGAGCATCACAAAAATCGACGCT
CAAGTCAGAGGTGGCGAAACCCGACAGGACTATAAAGATACCAGGCGTTTCCCCCTGGAAGCTCCCTCGTGCGCTCTCCT
GTTCCGACCCTGCCGCTTACCGGATACCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGAACCCCCCGTTCAGCCCGACCGCTGCG
CCTTATCCGGTAACTATCGTCTTGAGTCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCTAACTACGGCTACACTAGAAGAACAGT
ATTTGGTATCTGCGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGATCCTTTGATCTTT
AGAAAAACTCATCGAGCATCAAATGAAACTGCAATTTATTCATATCAGGATTATCAATACCATATTTTTGAAAAAGCCGT
TTCTGTAATGAAGGAGAAAACTCACCGAGGCAGTTCCATAGGATGGCAAGATCCTGGTATCGGTCTGCGATTCCGACTCG
TCCAACATCAATACAACCTATTAATTTCCCCTCGTCAAAAATAAGGTTATCAAGTGAGAAATCACCATGAGTGACGACTG
AATCCGGTGAGAATGGCAAAAGTTTATGCATTTCTTTCCAGACTTGTTCAACAGGCCAGCCATTACGCTCGTCATCAAAA
TCACTCGCATCAACCAAACCGTTATTCATTCGTGATTGCGCCTGAGCCAGACGAAATACGCGATCGCTGTTAAAAGGACA
ATTACAAACAGGAATCGAATGCAACCGGCGCAGGAACACTGCCAGCGCATCAACAATATTTTCACCTGAATCAGGATATT
CTTCTAATACCTGGAATGCTGTTTTTCCGGGGATCGCAGTGGTGAGTAACCATGCATCATCAGGAGTACGGATAAAATGC
TTGATGGTCGGAAGAGGCATAAATTCCGTCAGCCAGTTTAGTCTGACCATCTCATCTGTAACATCATTGGCAACGCTACC
TTTGCCATGTTTCAGAAACAACTCTGGCGCATCGGGCTTCCCATACAAGCGATAGATTGTCGCACCTGATTGCCCGACAT
TATCGCGAGCCCATTTATACCCATATAAATCAGCATCCATGTTGGAATTTAATCGCGGCCTCGACGTTTCCCGTTGAATA
TGGCTCATAACACCCCTTGTATTACTGTTTATGTAAGCAGACAGTTTTATTGTTCATGATGATATATTTTTATCTTGTGC
AATGTAACATCAGAGATTTTGAGACAC

In certain embodiments, the Secondary Promoter Construct comprises nucleotides 1 to 4004 of SEQ ID NO: 53. In certain embodiments, the Secondary Promoter Construct comprises nucleotides 4713 to 7331 of SEQ ID NO: 53. In certain embodiments, the Secondary Promoter Construct comprises nucleotides 1 to 4004 and nucleotides 4713 to 7331 of SEQ ID NO: 53 SEQ ID NO: 53 is provided below.

[SEQ ID NO: 53]
GGTACCACATTAAAAACACAAAATCCTACGGAAATACTGAAGAATGAGTCTCAGCACTAAGGAAAAGCCTCCAGCAGCTC
CTGCTTTCTGAGGGTGAAGGATAGACGCTGTGGCTCTGCATGACTCACTAGCACTCTATCACGGCCATATTCTGGCAGGG
TCAGTGGCTCCAACTAACATTTGTTTGGTACTTTACAGTTTATTAAATAGATGTTTATATGGAGAAGCTCTCATTTCTTT
CTCAGAAGAGCCTGGCTAGGAAGGTGGATGAGGCACCATATTCATTTTGCAGGTGAAATTCCTGAGATGTAAGGAGCTGC
TGTGACTTGCTCAAGGCCTTATATCGAGTAAACGGTAGTGCTGGGGCTTAGACGCAGGTGTTCTGATTTATAGTTCAAAA
CCTCTATCAATGAGAGAGCAATCTCCTGGTAATGTGATAGATTTCCCAACTTAATGCCAACATACCATAAACCTCCCATT
CTGCTAATGCCCAGCCTAAGTTGGGGAGACCACTCCAGATTCCAAGATGTACAGTTTGCTTTGCTGGGCCTTTTTCCCAT
GCCTGCCTTTACTCTGCCAGAGTTATATTGCTGGGGTTTTGAAGAAGATCCTATTAAATAAAAGAATAAGCAGTATTATT
AAGTAGCCCTGCATTTCAGGTTTCCTTGAGTGGCAGGCCAGGCCTGGCCGTGAACGTTCACTGAAATCATGGCCTCTTGG
CCAAGATTGATAGCTTGTGCCTGTCCCTGAGTCCCAGTCCATCACGAGCAGCTGGTTTCTAAGATGCTATTTCCCGTATA
AAGCATGAGACCGTGACTTGCCAGCCCCACAGAGCCCCGCCCTTGTCCATCACTGGCATCTGGACTCCAGCCTGGGTTGG
GGCAAAGAGGGAAATGAGATCATGTCCTAACCCTGATCCTCTTGTCCCACAGATATCCAGAACCCTGACCCTGCCGTGTA
CCAGCTGAGAGACTCTAAATCCAGTGACAAGTCTGTCTGCCTATTCGAATTCCGTTACATAACTTACGTTAAATGGCCCG
CCTGGCTGACCGCCCAACGACCCCCGCCCATTGACGTCAATAATGATGTATGTTCCCATAGTAACGCCAATAGGGACTTT
CCATTGACGTCAATGGGTGGAGTATTTACGGTAAACTGCCCACTTGGCAGTACATCAAGTGTATCATATCCAAGTACGCC
CCCTATTGACGTCAATGACGGTAAATGGCCCGCCTGGCATTATGCCCAGTACATGACCTTATGGGACTTTCCTACTTGGC
AGTACATCTACGTATTAGTCATCGCTATTACCATGGGCTCCGGAGCCACTAACTTCTCCCTGTTGAAACAGGCTGGCGAT
GTTGAAGAAAACCCCGGTCCTATGGCCACCGGCTCTAGAACAAGCCTGCTGCTCGCTTTTGGCCTGCTCTGCCTCCCATG
GCTCCAAGAAGGATCTGCTGATGCTGGAATCACCCAGAGCCCAAGATACAAGATCACAGAGACAGGAAGGCAGGTGACCT
TGATGTGTCACCAGACTTGGAGCCACAGCTATATGTTCTGGTATCGACAAGACCTGGGACATGGGCTGAGGCTGATCTAT
TACTCAGCAGCTGCTGATATTACAGATAAAGGAGAAGTCTCCGATGGCTATGTTGTCTCCAGATCCAAGACAGAGAATTT
CCCCCTCACTCTGGAGTCAGCTACCCGCTCCCAGACATCTGTGTATTTCTGCGCCAGCAGTGAGGACAGTTACGAGCAGT
ACTTCGGGCCGGGCACCAGGCTCACGGTCACAGAGGACCTGAAAAACGTGTTCCCTCCAAAAGTGGCCGTGTTCGAGCCT
TCTGAGGCCGAGATCAGCCACACACAGAAAGCCACACTCGTGTGTCTGGCTACCGGCTTCTACCCCGATCACGTGGAACT
GTCTTGGTGGGTCAACGGCAAAGAGGTGCACAGCGGCGTCAGCACAGATCCCCAGCCTCTGAAAGAACAGCCCGCTCTGA
ACGACAGCCGCTACTGCCTGTCTAGCAGACTGAGAGTGTCCGCCACCTTCTGGCAGAACCCCAGAAACCACTTCAGATGC
CAGGTCCAGTTCTACGGCCTGAGCGAGAACGATGAGTGGACCCAGGACAGAGCCAAGCCTGTGACACAGATCGTGTCTGC
CGAAGCCTGGGGCAGAGCCGATTGTGGCTTTACCAGCGAGTCATACCAGCAGGGCGTGCTGTCTGCCACCATCCTGTATG
AGATCCTGCTCGGCAAGGCCACACTGTACGCTGTGCTGGTGTCTGCTCTGGTGCTGATGGCTATGGTCTCCCGGGAGCGC
ATCCCCGAGGCCCGGGCCAAGCGGGGCAGCGGCGCCACCAACTTCAGCCTGCTGAAGCAGGCCGGCGACGTGGAGGAGAA
CCCCGGCCCTATGGCCACAGGCAGCAGAACATCTCTGCTGCTGGCCTTCGGACTGCTGTGTCTGCCTTGGCTGCAAGAGG
GTTCCGCCGCCCAGTCAGTGACCCAGCCTGACATCCACATCACTGTCTCTGAAGGAGCCTCACTGGAGTTGAGATGTAAC
TATTCCTATGGGGCAACACCTTATCTCTTCTGGTATGTCCAGTCCCCCGGCCAAGGCCTCCAGCTGCTCCTGAAGTACTT
TTCAGGAGACACTCTGGTTCAAGGCATTAAAGGCTTTGAGGCTGAATTTAAGAGGAGTCAATCTTCCTTCAATCTGAGGA
AACCCTCTGTGCATTGGAGTGATGCTGCTGAGTACTTCTGTGCTGTGGGTGAATTGGACACAGGCTTTCAGAAACTTGTA
TTTGGAACTGGCACCCGACTTCTGGTCAGTCCAAATATTCAGAACCCCGATCCTGCCGTGTACCAGCTGAGAGACAGCAA
GAGCAGCGACAAGAGCGTGTGCCTGTTCACCGACTTCGACAGCCAGACCAACGTGTCCCAGAGCAAGGACAGCGACGTGT
ACATCACCGACAAGACCGTGCTGGACATGCGGAGCATGGACTTCAAGAGCAACAGCGCCGTGGCCTGGTCCAACAAGAGC
GATTTCGCCTGCGCCAACGCCTTCAACAACAGCATTATCCCCGAGGACACATTCTTCCCAAGTCCTGAGAGCAGCTGCGA
CGTGAAGCTGGTGGAAAAGAGCTTCGAGACAGACACCAACCTGAACTTCCAGAACCTGTCCGTGATCGGCTTCCGCATCC
TGCTGCTGAAAGTGGCCGGCTTCAACCTGCTGATGACCCTGAGACTGTGGTCCAGCTGATGTGCCTTCTAGTTGCCAGCC
ATCTGTTGTTTGCCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAGGGGGAGGATTGG
GAAGACAATAGCAGGCATGCTGGGGATGCGGTGGGCTCTATGGCGTCGACGAGCTCACGGGGACAGCCCCCCCCCAAAGC
CCCCAGGGATGTAATTACGTCCCTCCCCCGCTAGGGGGCAGCAGCGAGCCGCCCGGGGCTCCGCTCCGGTCCGGCGCTCC
CCCCGCATCCCCGAGCCGGCAGCGTGCGGGGACAGCCCGGGCACGGGGAAGGTGGCACGGGATCGCTTTCCTCTGAACGC
TTCTCGCTGCTCTTTGAGCCTGCAGACACCTGGGGGGATACGGGGAAAAAGCTTCTCGAGTGAGTCAGTGACTCAGTGAG
TCAGTGACTCAGTGAGTCACTGACTCACTGAGTCAGTGACTCAGGAATTCAGCTTGAATAAAATGAATATTAGAAGCTGT
TAGAATAAGAGAAAATGACAGAGGAAAACTGAAAGGGAGAACTGAAAGTGGGAAATTCCTCTGAGGCAGAAAGGACCATC
CCTTATAAATAGCACAGGCCATGAAGGAAGATCATTCTCACTGCAGCCTTTGACAGCCTTTGCCTCATCTTGGGATCCGC
CACCATGGTGTCCAAGGGCGAAGAGGACAACATGGCCATCATCAAAGAGTTCATGCGGTTCAAGGTGCACATGGAAGGCA
GCGTGAACGGCCACGAGTTCGAGATTGAAGGCGAAGGCGAGGGCAGACCTTACGAGGGAACACAGACCGCCAAGCTGAAA
GTGACCAAAGGCGGCCCTCTGCCTTTTGCCTGGGACATTCTGAGCCCTCAGTTTATGTACGGCAGCAAGGCCTACGTGAA
GCACCCCGCCGATATTCCCGACTACCTGAAGCTGAGCTTCCCCGAGGGCTTCAAGTGGGAGAGAGTGATGAACTTCGAGG
ACGGCGGCGTGGTCACCGTGACTCAAGATAGCTCTCTGCAGGACGGCGAGTTCATCTACAAAGTGAAGCTGCGGGGCACC
AACTTTCCCTCTGATGGCCCCGTGATGCAGAAAAAGACAATGGGCTGGGAAGCCAGCAGCGAGAGAATGTACCCTGAAGA
TGGCGCCCTGAAGGGCGAGATCAAGCAGCGGCTGAAACTGAAGGATGGCGGCCACTACGACGCCGAAGTGAAAACCACCT
ACAAGGCCAAGAAACCCGTGCAGCTGCCTGGCGCCTACAACGTGAACATCAAGCTGGACATCACCAGCCACAACGAGGAC
TACACCATCGTGGAACAGTACGAGAGAGCCGAAGGCAGACACAGCACAGGCGGAATGGACGAGCTGTACAAGAGAGCCAA
ACGGGGGTCCGGCGCTACCAATTTCAGTTTGCTCAAACAGGCCGGAGACGTCGAGGAAAATCCTGGCCCCCATATGACCG
ATTTTGATTCTCAAACAAATGTGTCACAAAGTAAGGATTCTGATGTGTATATCACAGACAAAACTGTGCTAGACATGAGG
TCTATGGACTTCAAGAGCAACAGTGCTGTGGCCTGGAGCAACAAATCTGACTTTGCATGTGCAAACGCCTTCAACAACAG
CATTATTCCAGAAGACACCTTCTTCCCCAGCCCAGGTAAGGGCAGCTTTGGTGCCTTCGCAGGCTGTTTCCTTGCTTCAG
GAATGGCCAGGTTCTGCCCAGAGCTCTGGTCAATGATGTCTAAAACTCCTCTGATTGGTGGTCTCGGCCTTATCCATTGC
CACCAAAACCCTCTTTTTACTAAGAAACAGTGAGCCTTGTTCTGGCAGTCCAGAGAATGACACGGGAAAAAAGCAGATGA
AGAGAAGGTGGCAGGAGAGGGCACGTGGCCCAGCCTCAGTCTCTCCAACTGAGTTCCTGCCTGCCTGCCTTTGCTCAGAC
TGTTTGCCCCTTACTGCTCTTCTAGGCCTCATTCTAAGCCCCTTCTCCAAGTTGCCTCTCCTTATTTCTCCCTGTCTGCC
AAAAAATCTTTCCCAGCTCACTAAGTCAGTCTCACGCAGTCACTCATTAACCCACCAATCACTGATTGTGCCGGCACATG
AATGCACCAGGTGTTGAAGTGGAGGAATTAAAAAGTCAGATGAGGGGTGTGCCCAGAGGAAGCACCATTCTAGTTGGGGG
AGCCCATCTGTCAGCTGGGAAAAGTCCAAATAACTTCAGATTGGAATGTGTTTTAACTCAGGGTTGAGAAAACAGCTACC
TTCAGGACAAAAGTCAGGGAAGGGCTCTCTGAAGAAATGCTACTTGAAGATACCAGCCCTACCAAGGGCAGGGAGAGGAC
CCTATAGAGGCCTGGGACAGGAGCTCAATGAGAAAGGAGAAGAGCAGCAGGCATGAGTTGAATGAAGGAGGCAGGGCCGG
GTCACAGGGCCTTCTAGGCCATGAGAGGGTAGACAGGCTAGCCGCGTTGCTGGCGTTTTTCCATAGGCTCCGCCCCCCTG
ACGAGCATCACAAAAATCGACGCTCAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCCGCCTTTCTCCCTTCGGGAAGCGT
GGCGCTTTCTCATAGCTCACGCTGTAGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAACCCGGTAAGACACGACTTATCGCCA
CTGGCAGCAGCCACTGGTAACAGGATTAGCAGAGCGAGGTATGTAGGCGGTGCTACAGAGTTCTTGAAGTGGTGGCCTAA
CTACGGCTACACTAGAAGAACAGTATTTGGTATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGCAAGCAGCAGATTACGCGCAGAAAAAAAGGA
TCTCAAGAAGATCCTTTGATCTTTAGAAAAACTCATCGAGCATCAAATGAAACTGCAATTTATTCATATCAGGATTATCA
ATACCATATTTTTGAAAAAGCCGTTTCTGTAATGAAGGAGAAAACTCACCGAGGCAGTTCCATAGGATGGCAAGATCCTG
GTATCGGTCTGCGATTCCGACTCGTCCAACATCAATACAACCTATTAATTTCCCCTCGTCAAAAATAAGGTTATCAAGTG
AGAAATCACCATGAGTGACGACTGAATCCGGTGAGAATGGCAAAAGTTTATGCATTTCTTTCCAGACTTGTTCAACAGGC
CAGCCATTACGCTCGTCATCAAAATCACTCGCATCAACCAAACCGTTATTCATTCGTGATTGCGCCTGAGCCAGACGAAA
TACGCGATCGCTGTTAAAAGGACAATTACAAACAGGAATCGAATGCAACCGGCGCAGGAACACTGCCAGCGCATCAACAA
TATTTTCACCTGAATCAGGATATTCTTCTAATACCTGGAATGCTGTTTTTCCGGGGATCGCAGTGGTGAGTAACCATGCA
TCATCAGGAGTACGGATAAAATGCTTGATGGTCGGAAGAGGCATAAATTCCGTCAGCCAGTTTAGTCTGACCATCTCATC
TGTAACATCATTGGCAACGCTACCTTTGCCATGTTTCAGAAACAACTCTGGCGCATCGGGCTTCCCATACAAGCGATAGA
TTGTCGCACCTGATTGCCCGACATTATCGCGAGCCCATTTATACCCATATAAATCAGCATCCATGTTGGAATTTAATCGC
GGCCTCGACGTTTCCCGTTGAATATGGCTCATAACACCCCTTGTATTACTGTTTATGTAAGCAGACAGTTTTATTGTTCA
TGATGATATATTTTTATCTTGTGCAATGTAACATCAGAGATTTTGAGACAC

In certain embodiments, the Secondary Promoter Construct comprises nucleotides 1 to 4938 of SEQ ID NO: 54. In certain embodiments, the Secondary Promoter Construct comprises nucleotides 5647 to 8265 of SEQ ID NO: 54. In certain embodiments, the Secondary Promoter Construct comprises nucleotides 1 to 4938 and nucleotides 5647 to 8265 of SEQ ID NO: 54 SEQ ID NO: 54 is provided below.

[SEQ ID NO: 54]
GGTACCACATTAAAAACACAAAATCCTACGGAAATACTGAAGAATGAGTCTCAGCACTAAGGAAAAGCCTCCAGCAGCTC
CTGCTTTCTGAGGGTGAAGGATAGACGCTGTGGCTCTGCATGACTCACTAGCACTCTATCACGGCCATATTCTGGCAGGG
TCAGTGGCTCCAACTAACATTTGTTTGGTACTTTACAGTTTATTAAATAGATGTTTATATGGAGAAGCTCTCATTTCTTT
CTCAGAAGAGCCTGGCTAGGAAGGTGGATGAGGCACCATATTCATTTTGCAGGTGAAATTCCTGAGATGTAAGGAGCTGC
TGTGACTTGCTCAAGGCCTTATATCGAGTAAACGGTAGTGCTGGGGCTTAGACGCAGGTGTTCTGATTTATAGTTCAAAA
CCTCTATCAATGAGAGAGCAATCTCCTGGTAATGTGATAGATTTCCCAACTTAATGCCAACATACCATAAACCTCCCATT
CTGCTAATGCCCAGCCTAAGTTGGGGAGACCACTCCAGATTCCAAGATGTACAGTTTGCTTTGCTGGGCCTTTTTCCCAT
GCCTGCCTTTACTCTGCCAGAGTTATATTGCTGGGGTTTTGAAGAAGATCCTATTAAATAAAAGAATAAGCAGTATTATT
AAGTAGCCCTGCATTTCAGGTTTCCTTGAGTGGCAGGCCAGGCCTGGCCGTGAACGTTCACTGAAATCATGGCCTCTTGG
CCAAGATTGATAGCTTGTGCCTGTCCCTGAGTCCCAGTCCATCACGAGCAGCTGGTTTCTAAGATGCTATTTCCCGTATA
AAGCATGAGACCGTGACTTGCCAGCCCCACAGAGCCCCGCCCTTGTCCATCACTGGCATCTGGACTCCAGCCTGGGTTGG
GGCAAAGAGGGAAATGAGATCATGTCCTAACCCTGATCCTCTTGTCCCACAGATATCCAGAACCCTGACCCTGCCGTGTA
CCAGCTGAGAGACTCTAAATCCAGTGACAAGTCTGTCTGCCTATTCGAATTCCGTTACATAACTTACGTTAAATGGCCCG
CCTGGCTGACCGCCCAACGACCCCCGCCCATTGACGTCAATAATGATGTATGTTCCCATAGTAACGCCAATAGGGACTTT
CCATTGACGTCAATGGGTGGAGTATTTACGGTAAACTGCCCACTTGGCAGTACATCAAGTGTATCATATCCAAGTACGCC
CCCTATTGACGTCAATGACGGTAAATGGCCCGCCTGGCATTATGCCCAGTACATGACCTTATGGGACTTTCCTACTTGGC
AGTACATCTACGTATTAGTCATCGCTATTACCATGGGCTCCGGAGCCACTAACTTCTCCCTGTTGAAACAGGCTGGCGAT
GTTGAAGAAAACCCCGGTCCTATGGCCACCGGCTCTAGAACAAGCCTGCTGCTCGCTTTTGGCCTGCTCTGCCTCCCATG
GCTCCAAGAAGGATCTGCTGATGCTGGAATCACCCAGAGCCCAAGATACAAGATCACAGAGACAGGAAGGCAGGTGACCT
TGATGTGTCACCAGACTTGGAGCCACAGCTATATGTTCTGGTATCGACAAGACCTGGGACATGGGCTGAGGCTGATCTAT
TACTCAGCAGCTGCTGATATTACAGATAAAGGAGAAGTCTCCGATGGCTATGTTGTCTCCAGATCCAAGACAGAGAATTT
CCCCCTCACTCTGGAGTCAGCTACCCGCTCCCAGACATCTGTGTATTTCTGCGCCAGCAGTGAGGACAGTTACGAGCAGT
ACTTCGGGCCGGGCACCAGGCTCACGGTCACAGAGGACCTGAAAAACGTGTTCCCTCCAAAAGTGGCCGTGTTCGAGCCT
TCTGAGGCCGAGATCAGCCACACACAGAAAGCCACACTCGTGTGTCTGGCTACCGGCTTCTACCCCGATCACGTGGAACT
GTCTTGGTGGGTCAACGGCAAAGAGGTGCACAGCGGCGTCAGCACAGATCCCCAGCCTCTGAAAGAACAGCCCGCTCTGA
ACGACAGCCGCTACTGCCTGTCTAGCAGACTGAGAGTGTCCGCCACCTTCTGGCAGAACCCCAGAAACCACTTCAGATGC
CAGGTCCAGTTCTACGGCCTGAGCGAGAACGATGAGTGGACCCAGGACAGAGCCAAGCCTGTGACACAGATCGTGTCTGC
CGAAGCCTGGGGCAGAGCCGATTGTGGCTTTACCAGCGAGTCATACCAGCAGGGCGTGCTGTCTGCCACCATCCTGTATG
AGATCCTGCTCGGCAAGGCCACACTGTACGCTGTGCTGGTGTCTGCTCTGGTGCTGATGGCTATGGTCTCCCGGGAGCGC
ATCCCCGAGGCCCGGGCCAAGCGGGGCAGCGGCGCCACCAACTTCAGCCTGCTGAAGCAGGCCGGCGACGTGGAGGAGAA
CCCCGGCCCTATGGCCACAGGCAGCAGAACATCTCTGCTGCTGGCCTTCGGACTGCTGTGTCTGCCTTGGCTGCAAGAGG
GTTCCGCCGCCCAGTCAGTGACCCAGCCTGACATCCACATCACTGTCTCTGAAGGAGCCTCACTGGAGTTGAGATGTAAC
TATTCCTATGGGGCAACACCTTATCTCTTCTGGTATGTCCAGTCCCCCGGCCAAGGCCTCCAGCTGCTCCTGAAGTACTT
TTCAGGAGACACTCTGGTTCAAGGCATTAAAGGCTTTGAGGCTGAATTTAAGAGGAGTCAATCTTCCTTCAATCTGAGGA
AACCCTCTGTGCATTGGAGTGATGCTGCTGAGTACTTCTGTGCTGTGGGTGAATTGGACACAGGCTTTCAGAAACTTGTA
TTTGGAACTGGCACCCGACTTCTGGTCAGTCCAAATATTCAGAACCCCGATCCTGCCGTGTACCAGCTGAGAGACAGCAA
GAGCAGCGACAAGAGCGTGTGCCTGTTCACCGACTTCGACAGCCAGACCAACGTGTCCCAGAGCAAGGACAGCGACGTGT
ACATCACCGACAAGACCGTGCTGGACATGCGGAGCATGGACTTCAAGAGCAACAGCGCCGTGGCCTGGTCCAACAAGAGC
GATTTCGCCTGCGCCAACGCCTTCAACAACAGCATTATCCCCGAGGACACATTCTTCCCAAGTCCTGAGAGCAGCTGCGA
CGTGAAGCTGGTGGAAAAGAGCTTCGAGACAGACACCAACCTGAACTTCCAGAACCTGTCCGTGATCGGCTTCCGCATCC
TGCTGCTGAAAGTGGCCGGCTTCAACCTGCTGATGACCCTGAGACTGTGGTCCAGCTGATGTGCCTTCTAGTTGCCAGCC
ATCTGTTGTTTGCCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAGGGGGAGGATTGG
GAAGACAATAGCAGGCATGCTGGGGATGCGGTGGGCTCTATGGCGTCGACGAGCTCACGGGGACAGCCCCCCCCCAAAGC
CCCCAGGGATGTAATTACGTCCCTCCCCCGCTAGGGGGCAGCAGCGAGCCGCCCGGGGCTCCGCTCCGGTCCGGCGCTCC
CCCCGCATCCCCGAGCCGGCAGCGTGCGGGGACAGCCCGGGCACGGGGAAGGTGGCACGGGATCGCTTTCCTCTGAACGC
TTCTCGCTGCTCTTTGAGCCTGCAGACACCTGGGGGGATACGGGGAAAAAGCTTGCGGCCGCCGTGAGGCTCCGGTGCCC
GTCAGTGGGCAGAGCGCACATCGCCCACAGTCCCCGAGAAGTTGGGGGGAGGGGTCGGCAATTGAACCGGTGCCTAGAGA
AGGTGGCGCGGGGTAAACTGGGAAAGTGATGTCGTGTACTGGCTCCGCCTTTTTCCCGAGGGTGGGGGAGAACCGTATAT
AAGTGCAGTAGTCGCCGTGAACGTTCTTTTTCGCAACGGGTTTGCCGCCAGAACACAGGTAAGTGCCGTGTGTGGTTCCC
GCGGGCCTGGCCTCTTTACGGGTTATGGCCCTTGCGTGCCTTGAATTACTTCCACCTGGCTGCAGTACGTGATTCTTGAT
CCCGAGCTTCGGGTTGGAAGTGGGTGGGAGAGTTCGAGGCCTTGCGCTTAAGGAGCCCCTTCGCCTCGTGCTTGAGTTGA
GGCCTGGCCTGGGCGCTGGGGCCGCCGCGTGCGAATCTGGTGGCACCTTCGCGCCTGTCTCGCTGCTTTCGATAAGTCTC
TAGCCATTTAAAATTTTTGATGACCTGCTGCGACGCTTTTTTTCTGGCAAGATAGTCTTGTAAATGCGGGCCAAGATCTG
CACACTGGTATTTCGGTTTTTGGGGCCGCGGGCGGCGACGGGGCCCGTGCGTCCCAGCGCACATGTTCGGCGAGGCGGGG
CCTGCGAGCGCGGCCACCGAGAATCGGACGGGGGTAGTCTCAAGCTGGCCGGCCTGCTCTGGTGCCTGGCCTCGCGCCGC
CGTGTATCGCCCCGCCCTGGGCGGCAAGGCTGGCCCGGTCGGCACCAGTTGCGTGAGCGGAAAGATGGCCGCTTCCCGGC
CCTGCTGCAGGGAGCTCAAAATGGAGGACGCGGCGCTCGGGAGAGCGGGCGGGTGAGTCACCCACACAAAGGAAAAGGGC
CTTTCCGTCCTCAGCCGTCGCTTCATGTGACTCCACGGAGTACCGGGCGCCGTCCAGGCACCTCGATTAGTTCTCGAGCT
TTTGGAGTACGTCGTCTTTAGGTTGGGGGGAGGGGTTTTATGCGATGGAGTTTCCCCACACTGAGTGGGTGGAGACTGAA
GTTAGGCCAGCTTGGCACTTGATGTAATTCTCCTTGGAATTTGCCCTTTTTGAGTTTGGATCTTGGTTCATTCTCAAGCC
TCAGACAGTGGTTCAAAGTTTTTTTCTTCCATTTCAGGTGTCGTGAGGATCCGCCACCATGGTGTCCAAGGGCGAAGAGG
ACAACATGGCCATCATCAAAGAGTTCATGCGGTTCAAGGTGCACATGGAAGGCAGCGTGAACGGCCACGAGTTCGAGATT
GAAGGCGAAGGCGAGGGCAGACCTTACGAGGGAACACAGACCGCCAAGCTGAAAGTGACCAAAGGCGGCCCTCTGCCTTT
TGCCTGGGACATTCTGAGCCCTCAGTTTATGTACGGCAGCAAGGCCTACGTGAAGCACCCCGCCGATATTCCCGACTACC
TGAAGCTGAGCTTCCCCGAGGGCTTCAAGTGGGAGAGAGTGATGAACTTCGAGGACGGCGGCGTGGTCACCGTGACTCAA
GATAGCTCTCTGCAGGACGGCGAGTTCATCTACAAAGTGAAGCTGCGGGGCACCAACTTTCCCTCTGATGGCCCCGTGAT
GCAGAAAAAGACAATGGGCTGGGAAGCCAGCAGCGAGAGAATGTACCCTGAAGATGGCGCCCTGAAGGGCGAGATCAAGC
AGCGGCTGAAACTGAAGGATGGCGGCCACTACGACGCCGAAGTGAAAACCACCTACAAGGCCAAGAAACCCGTGCAGCTG
CCTGGCGCCTACAACGTGAACATCAAGCTGGACATCACCAGCCACAACGAGGACTACACCATCGTGGAACAGTACGAGAG
AGCCGAAGGCAGACACAGCACAGGCGGAATGGACGAGCTGTACAAGAGAGCCAAACGGGGGTCCGGCGCTACCAATTTCA
GTTTGCTCAAACAGGCCGGAGACGTCGAGGAAAATCCTGGCCCCCATATGACCGATTTTGATTCTCAAACAAATGTGTCA
CAAAGTAAGGATTCTGATGTGTATATCACAGACAAAACTGTGCTAGACATGAGGTCTATGGACTTCAAGAGCAACAGTGC
TGTGGCCTGGAGCAACAAATCTGACTTTGCATGTGCAAACGCCTTCAACAACAGCATTATTCCAGAAGACACCTTCTTCC
CCAGCCCAGGTAAGGGCAGCTTTGGTGCCTTCGCAGGCTGTTTCCTTGCTTCAGGAATGGCCAGGTTCTGCCCAGAGCTC
TGGTCAATGATGTCTAAAACTCCTCTGATTGGTGGTCTCGGCCTTATCCATTGCCACCAAAACCCTCTTTTTACTAAGAA
ACAGTGAGCCTTGTTCTGGCAGTCCAGAGAATGACACGGGAAAAAAGCAGATGAAGAGAAGGTGGCAGGAGAGGGCACGT
GGCCCAGCCTCAGTCTCTCCAACTGAGTTCCTGCCTGCCTGCCTTTGCTCAGACTGTTTGCCCCTTACTGCTCTTCTAGG
CCTCATTCTAAGCCCCTTCTCCAAGTTGCCTCTCCTTATTTCTCCCTGTCTGCCAAAAAATCTTTCCCAGCTCACTAAGT
CAGTCTCACGCAGTCACTCATTAACCCACCAATCACTGATTGTGCCGGCACATGAATGCACCAGGTGTTGAAGTGGAGGA
ATTAAAAAGTCAGATGAGGGGTGTGCCCAGAGGAAGCACCATTCTAGTTGGGGGAGCCCATCTGTCAGCTGGGAAAAGTC
CAAATAACTTCAGATTGGAATGTGTTTTAACTCAGGGTTGAGAAAACAGCTACCTTCAGGACAAAAGTCAGGGAAGGGCT
CTCTGAAGAAATGCTACTTGAAGATACCAGCCCTACCAAGGGCAGGGAGAGGACCCTATAGAGGCCTGGGACAGGAGCTC
AATGAGAAAGGAGAAGAGCAGCAGGCATGAGTTGAATGAAGGAGGCAGGGCCGGGTCACAGGGCCTTCTAGGCCATGAGA
GGGTAGACAGGCTAGCCGCGTTGCTGGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGCGCTTTCTCATAGCTCACGCTGTA
GGTATCTCAGTTCGGTGTAGGTCGTTCGCTCCAAGCTGGGCTGTGTGCACGAACCCCCCGTTCAGCCCGACCGCTGCGCC
TTATCCGGTAACTATCGTCTTGAGTCCAACCCGGTAAGACACGACTTATCGCCACTGGCAGCAGCCACTGGTAACAGGAT
TAGCAGAGCGAGGTATGTAGGCGGTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCTCAAGAAGATCCTTTGATCTTTAG
AAAAACTCATCGAGCATCAAATGAAACTGCAATTTATTCATATCAGGATTATCAATACCATATTTTTGAAAAAGCCGTTT
CTGTAATGAAGGAGAAAACTCACCGAGGCAGTTCCATAGGATGGCAAGATCCTGGTATCGGTCTGCGATTCCGACTCGTC
CAACATCAATACAACCTATTAATTTCCCCTCGTCAAAAATAAGGTTATCAAGTGAGAAATCACCATGAGTGACGACTGAA
TCCGGTGAGAATGGCAAAAGTTTATGCATTTCTTTCCAGACTTGTTCAACAGGCCAGCCATTACGCTCGTCATCAAAATC
ACTCGCATCAACCAAACCGTTATTCATTCGTGATTGCGCCTGAGCCAGACGAAATACGCGATCGCTGTTAAAAGGACAAT
TACAAACAGGAATCGAATGCAACCGGCGCAGGAACACTGCCAGCGCATCAACAATATTTTCACCTGAATCAGGATATTCT
TCTAATACCTGGAATGCTGTTTTTCCGGGGATCGCAGTGGTGAGTAACCATGCATCATCAGGAGTACGGATAAAATGCTT
GATGGTCGGAAGAGGCATAAATTCCGTCAGCCAGTTTAGTCTGACCATCTCATCTGTAACATCATTGGCAACGCTACCTT
TGCCATGTTTCAGAAACAACTCTGGCGCATCGGGCTTCCCATACAAGCGATAGATTGTCGCACCTGATTGCCCGACATTA
TCGCGAGCCCATTTATACCCATATAAATCAGCATCCATGTTGGAATTTAATCGCGGCCTCGACGTTTCCCGTTGAATATG
GCTCATAACACCCCTTGTATTACTGTTTATGTAAGCAGACAGTTTTATTGTTCATGATGATATATTTTTATCTTGTGCAA
TGTAACATCAGAGATTTTGAGACAC

In certain embodiments, the Secondary Promoter Construct comprises nucleotides 1 to 4302 of SEQ ID NO: 55. In certain embodiments, the Secondary Promoter Construct comprises nucleotides 5011 to 7629 of SEQ ID NO: 55. In certain embodiments, the Secondary Promoter Construct comprises nucleotides 1 to 4302 and nucleotides 5011 to 7629 of SEQ ID NO: 55 SEQ ID NO: 55 is provided below.

[SEQ ID NO: 55]
GGTACCACATTAAAAACACAAAATCCTACGGAAATACTGA
AGAATGAGTCTCAGCACTAAGGAAAAGCCTCCAGCAGCTC
CTGCTTTCTGAGGGTGAAGGATAGACGCTGTGGCTCTGCA
TGACTCACTAGCACTCTATCACGGCCATATTCTGGCAGGG
TCAGTGGCTCCAACTAACATTTGTTTGGTACTTTACAGTT
TATTAAATAGATGTTTATATGGAGAAGCTCTCATTTCTTT
CTCAGAAGAGCCTGGCTAGGAAGGTGGATGAGGCACCATA
TTCATTTTGCAGGTGAAATTCCTGAGATGTAAGGAGCTGC
TGTGACTTGCTCAAGGCCTTATATCGAGTAAACGGTAGTG
CTGGGGCTTAGACGCAGGTGTTCTGATTTATAGTTCAAAA
CCTCTATCAATGAGAGAGCAATCTCCTGGTAATGTGATAG
ATTTCCCAACTTAATGCCAACATACCATAAACCTCCCATT
CTGCTAATGCCCAGCCTAAGTTGGGGAGACCACTCCAGAT
TCCAAGATGTACAGTTTGCTTTGCTGGGCCTTTTTCCCAT
GCCTGCCTTTACTCTGCCAGAGTTATATTGCTGGGGTTTT
GAAGAAGATCCTATTAAATAAAAGAATAAGCAGTATTATT
AAGTAGCCCTGCATTTCAGGTTTCCTTGAGTGGCAGGCCA
GGCCTGGCCGTGAACGTTCACTGAAATCATGGCCTCTTGG
CCAAGATTGATAGCTTGTGCCTGTCCCTGAGTCCCAGTCC
ATCACGAGCAGCTGGTTTCTAAGATGCTATTTCCCGTATA
AAGCATGAGACCGTGACTTGCCAGCCCCACAGAGCCCCGC
CCTTGTCCATCACTGGCATCTGGACTCCAGCCTGGGTTGG
GGCAAAGAGGGAAATGAGATCATGTCCTAACCCTGATCCT
CTTGTCCCACAGATATCCAGAACCCTGACCCTGCCGTGTA
CCAGCTGAGAGACTCTAAATCCAGTGACAAGTCTGTCTGC
CTATTCGAATTCCGTTACATAACTTACGTTAAATGGCCCG
CCTGGCTGACCGCCCAACGACCCCCGCCCATTGACGTCAA
TAATGATGTATGTTCCCATAGTAACGCCAATAGGGACTTT
CCATTGACGTCAATGGGTGGAGTATTTACGGTAAACTGCC
CACTTGGCAGTACATCAAGTGTATCATATCCAAGTACGCC
CCCTATTGACGTCAATGACGGTAAATGGCCCGCCTGGCAT
TATGCCCAGTACATGACCTTATGGGACTTTCCTACTTGGC
AGTACATCTACGTATTAGTCATCGCTATTACCATGGGCTC
CGGAGCCACTAACTTCTCCCTGTTGAAACAGGCTGGCGAT
GTTGAAGAAAACCCCGGTCCTATGGCCACCGGCTCTAGAA
CAAGCCTGCTGCTCGCTTTTGGCCTGCTCTGCCTCCCATG
GCTCCAAGAAGGATCTGCTGATGCTGGAATCACCCAGAGC
CCAAGATACAAGATCACAGAGACAGGAAGGCAGGTGACCT
TGATGTGTCACCAGACTTGGAGCCACAGCTATATGTTCTG
GTATCGACAAGACCTGGGACATGGGCTGAGGCTGATCTAT
TACTCAGCAGCTGCTGATATTACAGATAAAGGAGAAGTCT
CCGATGGCTATGTTGTCTCCAGATCCAAGACAGAGAATTT
CCCCCTCACTCTGGAGTCAGCTACCCGCTCCCAGACATCT
GTGTATTTCTGCGCCAGCAGTGAGGACAGTTACGAGCAGT
ACTTCGGGCCGGGCACCAGGCTCACGGTCACAGAGGACCT
GAAAAACGTGTTCCCTCCAAAAGTGGCCGTGTTCGAGCCT
TCTGAGGCCGAGATCAGCCACACACAGAAAGCCACACTCG
TGTGTCTGGCTACCGGCTTCTACCCCGATCACGTGGAACT
GTCTTGGTGGGTCAACGGCAAAGAGGTGCACAGCGGCGTC
AGCACAGATCCCCAGCCTCTGAAAGAACAGCCCGCTCTGA
ACGACAGCCGCTACTGCCTGTCTAGCAGACTGAGAGTGTC
CGCCACCTTCTGGCAGAACCCCAGAAACCACTTCAGATGC
CAGGTCCAGTTCTACGGCCTGAGCGAGAACGATGAGTGGA
CCCAGGACAGAGCCAAGCCTGTGACACAGATCGTGTCTGC
CGAAGCCTGGGGCAGAGCCGATTGTGGCTTTACCAGCGAG
TCATACCAGCAGGGCGTGCTGTCTGCCACCATCCTGTATG
AGATCCTGCTCGGCAAGGCCACACTGTACGCTGTGCTGGT
GTCTGCTCTGGTGCTGATGGCTATGGTCTCCCGGGAGCGC
ATCCCCGAGGCCCGGGCCAAGCGGGGCAGCGGCGCCACCA
ACTTCAGCCTGCTGAAGCAGGCCGGCGACGTGGAGGAGAA
CCCCGGCCCTATGGCCACAGGCAGCAGAACATCTCTGCTG
CTGGCCTTCGGACTGCTGTGTCTGCCTTGGCTGCAAGAGG
GTTCCGCCGCCCAGTCAGTGACCCAGCCTGACATCCACAT
CACTGTCTCTGAAGGAGCCTCACTGGAGTTGAGATGTAAC
TATTCCTATGGGGCAACACCTTATCTCTTCTGGTATGTCC
AGTCCCCCGGCCAAGGCCTCCAGCTGCTCCTGAAGTACTT
TTCAGGAGACACTCTGGTTCAAGGCATTAAAGGCTTTGAG
GCTGAATTTAAGAGGAGTCAATCTTCCTTCAATCTGAGGA
AACCCTCTGTGCATTGGAGTGATGCTGCTGAGTACTTCTG
TGCTGTGGGTGAATTGGACACAGGCTTTCAGAAACTTGTA
TTTGGAACTGGCACCCGACTTCTGGTCAGTCCAAATATTC
AGAACCCCGATCCTGCCGTGTACCAGCTGAGAGACAGCAA
GAGCAGCGACAAGAGCGTGTGCCTGTTCACCGACTTCGAC
AGCCAGACCAACGTGTCCCAGAGCAAGGACAGCGACGTGT
ACATCACCGACAAGACCGTGCTGGACATGCGGAGCATGGA
CTTCAAGAGCAACAGCGCCGTGGCCTGGTCCAACAAGAGC
GATTTCGCCTGCGCCAACGCCTTCAACAACAGCATTATCC
CCGAGGACACATTCTTCCCAAGTCCTGAGAGCAGCTGCGA
CGTGAAGCTGGTGGAAAAGAGCTTCGAGACAGACACCAAC
CTGAACTTCCAGAACCTGTCCGTGATCGGCTTCCGCATCC
TGCTGCTGAAAGTGGCCGGCTTCAACCTGCTGATGACCCT
GAGACTGTGGTCCAGCTGATGTGCCTTCTAGTTGCCAGCC
ATCTGTTGTTTGCCCCTCCCCCGTGCCTTCCTTGACCCTG
GAAGGTGCCACTCCCACTGTCCTTTCCTAATAAAATGAGG
AAATTGCATCGCATTGTCTGAGTAGGTGTCATTCTATTCT
GGGGGGTGGGGTGGGGCAGGACAGCAAGGGGGAGGATTGG
GAAGACAATAGCAGGCATGCTGGGGATGCGGTGGGCTCTA
TGGCGTCGACGAGCTCACGGGGACAGCCCCCCCCCAAAGC
CCCCAGGGATGTAATTACGTCCCTCCCCCGCTAGGGGGCA
GCAGCGAGCCGCCCGGGGCTCCGCTCCGGTCCGGCGCTCC
CCCCGCATCCCCGAGCCGGCAGCGTGCGGGGACAGCCCGG
GCACGGGGAAGGTGGCACGGGATCGCTTTCCTCTGAACGC
TTCTCGCTGCTCTTTGAGCCTGCAGACACCTGGGGGGATA
CGGGGAAAAAGCTTTTTAAAACTGCCTGGCCACTCCATGC
CCTCCAAGAGCTCCTTCTGCAGGAGCGTACAGAACCCAGG
GCCCTGGCACCCGTGCAGACCCTGGCCCACCCCACCTGGG
CGCTCAGTGCCCAAGAGATGTCCACACCTAGGATGTCCCG
CGGTGGGTGGGGGGCCCGAGAGACGGGCAGGCCGGGGGCA
GGCCTGGCCATGCGGGGCCGAACCGGGCACTGCCCAGCGT
GGGGCGCGGGGGCCACGGCGCGCGCCCCCAGCCCCCGGGC
CCAGCACCCCAAGGCGGCCAACGCCAAAACTCTCCCTCCT
CCTCTTCCTCAATCTCGCTCTCGCTCTTTTTTTTTTTCGC
AAAAGGAGGGGAGAGGGGGTAAAAAAATGCTGCACTGTGC
GGCGAAGCCGGTGAGTGAGCGGCGCGGGGCCAATCAGCGT
GCGCCGTTCCGAAAGTTGCCTTTTATGGCTCGAGCGGCCG
CGGCGGCGCCCTATAAAACCCAGCGGCGCGACGCGCCACC
ACCGCCGAGACCGCGTCCGCCCCGCGAGCACAGAGCCTCG
CCTTTGCCGAGGATCCGCCACCATGGTGTCCAAGGGCGAA
GAGGACAACATGGCCATCATCAAAGAGTTCATGCGGTTCA
AGGTGCACATGGAAGGCAGCGTGAACGGCCACGAGTTCGA
GATTGAAGGCGAAGGCGAGGGCAGACCTTACGAGGGAACA
CAGACCGCCAAGCTGAAAGTGACCAAAGGCGGCCCTCTGC
CTTTTGCCTGGGACATTCTGAGCCCTCAGTTTATGTACGG
CAGCAAGGCCTACGTGAAGCACCCCGCCGATATTCCCGAC
TACCTGAAGCTGAGCTTCCCCGAGGGCTTCAAGTGGGAGA
GAGTGATGAACTTCGAGGACGGCGGCGTGGTCACCGTGAC
TCAAGATAGCTCTCTGCAGGACGGCGAGTTCATCTACAAA
GTGAAGCTGCGGGGCACCAACTTTCCCTCTGATGGCCCCG
TGATGCAGAAAAAGACAATGGGCTGGGAAGCCAGCAGCGA
GAGAATGTACCCTGAAGATGGCGCCCTGAAGGGCGAGATC
AAGCAGCGGCTGAAACTGAAGGATGGCGGCCACTACGACG
CCGAAGTGAAAACCACCTACAAGGCCAAGAAACCCGTGCA
GCTGCCTGGCGCCTACAACGTGAACATCAAGCTGGACATC
ACCAGCCACAACGAGGACTACACCATCGTGGAACAGTACG
AGAGAGCCGAAGGCAGACACAGCACAGGCGGAATGGACGA
GCTGTACAAGAGAGCCAAACGGGGGTCCGGCGCTACCAAT
TTCAGTTTGCTCAAACAGGCCGGAGACGTCGAGGAAAATC
CTGGCCCCCATATGACCGATTTTGATTCTCAAACAAATGT
GTCACAAAGTAAGGATTCTGATGTGTATATCACAGACAAA
ACTGTGCTAGACATGAGGTCTATGGACTTCAAGAGCAACA
GTGCTGTGGCCTGGAGCAACAAATCTGACTTTGCATGTGC
AAACGCCTTCAACAACAGCATTATTCCAGAAGACACCTTC
TTCCCCAGCCCAGGTAAGGGCAGCTTTGGTGCCTTCGCAG
GCTGTTTCCTTGCTTCAGGAATGGCCAGGTTCTGCCCAGA
GCTCTGGTCAATGATGTCTAAAACTCCTCTGATTGGTGGT
CTCGGCCTTATCCATTGCCACCAAAACCCTCTTTTTACTA
AGAAACAGTGAGCCTTGTTCTGGCAGTCCAGAGAATGACA
CGGGAAAAAAGCAGATGAAGAGAAGGTGGCAGGAGAGGGC
ACGTGGCCCAGCCTCAGTCTCTCCAACTGAGTTCCTGCCT
GCCTGCCTTTGCTCAGACTGTTTGCCCCTTACTGCTCTTC
TAGGCCTCATTCTAAGCCCCTTCTCCAAGTTGCCTCTCCT
TATTTCTCCCTGTCTGCCAAAAAATCTTTCCCAGCTCACT
AAGTCAGTCTCACGCAGTCACTCATTAACCCACCAATCAC
TGATTGTGCCGGCACATGAATGCACCAGGTGTTGAAGTGG
AGGAATTAAAAAGTCAGATGAGGGGTGTGCCCAGAGGAAG
CACCATTCTAGTTGGGGGAGCCCATCTGTCAGCTGGGAAA
AGTCCAAATAACTTCAGATTGGAATGTGTTTTAACTCAGG
GTTGAGAAAACAGCTACCTTCAGGACAAAAGTCAGGGAAG
GGCTCTCTGAAGAAATGCTACTTGAAGATACCAGCCCTAC
CAAGGGCAGGGAGAGGACCCTATAGAGGCCTGGGACAGGA
GCTCAATGAGAAAGGAGAAGAGCAGCAGGCATGAGTTGAA
TGAAGGAGGCAGGGCCGGGTCACAGGGCCTTCTAGGCCAT
GAGAGGGTAGACAGGCTAGCCGCGTTGCTGGCGTTTTTCC
ATAGGCTCCGCCCCCCTGACGAGCATCACAAAAATCGACG
CTCAAGTCAGAGGTGGCGAAACCCGACAGGACTATAAAGA
TACCAGGCGTTTCCCCCTGGAAGCTCCCTCGTGCGCTCTC
CTGTTCCGACCCTGCCGCTTACCGGATACCTGTCCGCCTT
TCTCCCTTCGGGAAGCGTGGCGCTTTCTCATAGCTCACGC
TGTAGGTATCTCAGTTCGGTGTAGGTCGTTCGCTCCAAGC
TGGGCTGTGTGCACGAACCCCCCGTTCAGCCCGACCGCTG
CGCCTTATCCGGTAACTATCGTCTTGAGTCCAACCCGGTA
AGACACGACTTATCGCCACTGGCAGCAGCCACTGGTAACA
GGATTAGCAGAGCGAGGTATGTAGGCGGTGCTACAGAGTT
CTTGAAGTGGTGGCCTAACTACGGCTACACTAGAAGAACA
GTATTTGGTATCTGCGCTCTGCTGAAGCCAGTTACCTTCG
GAAAAAGAGTTGGTAGCTCTTGATCCGGCAAACAAACCAC
CGCTGGTAGCGGTGGTTTTTTTGTTTGCAAGCAGCAGATT
ACGCGCAGAAAAAAAGGATCTCAAGAAGATCCTTTGATCT
TTAGAAAAACTCATCGAGCATCAAATGAAACTGCAATTTA
TTCATATCAGGATTATCAATACCATATTTTTGAAAAAGCC
GTTTCTGTAATGAAGGAGAAAACTCACCGAGGCAGTTCCA
TAGGATGGCAAGATCCTGGTATCGGTCTGCGATTCCGACT
CGTCCAACATCAATACAACCTATTAATTTCCCCTCGTCAA
AAATAAGGTTATCAAGTGAGAAATCACCATGAGTGACGAC
TGAATCCGGTGAGAATGGCAAAAGTTTATGCATTTCTTTC
CAGACTTGTTCAACAGGCCAGCCATTACGCTCGTCATCAA
AATCACTCGCATCAACCAAACCGTTATTCATTCGTGATTG
CGCCTGAGCCAGACGAAATACGCGATCGCTGTTAAAAGGA
CAATTACAAACAGGAATCGAATGCAACCGGCGCAGGAACA
CTGCCAGCGCATCAACAATATTTTCACCTGAATCAGGATA
TTCTTCTAATACCTGGAATGCTGTTTTTCCGGGGATCGCA
GTGGTGAGTAACCATGCATCATCAGGAGTACGGATAAAAT
GCTTGATGGTCGGAAGAGGCATAAATTCCGTCAGCCAGTT
TAGTCTGACCATCTCATCTGTAACATCATTGGCAACGCTA
CCTTTGCCATGTTTCAGAAACAACTCTGGCGCATCGGGCT
TCCCATACAAGCGATAGATTGTCGCACCTGATTGCCCGAC
ATTATCGCGAGCCCATTTATACCCATATAAATCAGCATCC
ATGTTGGAATTTAATCGCGGCCTCGACGTTTCCCGTTGAA
TATGGCTCATAACACCCCTTGTATTACTGTTTATGTAAGC
AGACAGTTTTATTGTTCATGATGATATATTTTTATCTTGT
GCAATGTAACATCAGAGATTTTGAGACAC

In certain embodiments, the Secondary Promoter Construct comprises nucleotides 1 to 4274 of SEQ ID NO: 56. In certain embodiments, the Secondary Promoter Construct comprises nucleotides 4983 to 7601 of SEQ ID NO: 56. In certain embodiments, the Secondary Promoter Construct comprises nucleotides 1 to 4274 and nucleotides 4983 to 7601 of SEQ ID NO: 56 SEQ ID NO: 56 is provided below.

[SEQ ID NO: 56]
GGTACCACATTAAAAACACAAAATCCTACGGAAATACTGA
AGAATGAGTCTCAGCACTAAGGAAAAGCCTCCAGCAGCTC
CTGCTTTCTGAGGGTGAAGGATAGACGCTGTGGCTCTGCA
TGACTCACTAGCACTCTATCACGGCCATATTCTGGCAGGG
TCAGTGGCTCCAACTAACATTTGTTTGGTACTTTACAGTT
TATTAAATAGATGTTTATATGGAGAAGCTCTCATTTCTTT
CTCAGAAGAGCCTGGCTAGGAAGGTGGATGAGGCACCATA
TTCATTTTGCAGGTGAAATTCCTGAGATGTAAGGAGCTGC
TGTGACTTGCTCAAGGCCTTATATCGAGTAAACGGTAGTG
CTGGGGCTTAGACGCAGGTGTTCTGATTTATAGTTCAAAA
CCTCTATCAATGAGAGAGCAATCTCCTGGTAATGTGATAG
ATTTCCCAACTTAATGCCAACATACCATAAACCTCCCATT
CTGCTAATGCCCAGCCTAAGTTGGGGAGACCACTCCAGAT
TCCAAGATGTACAGTTTGCTTTGCTGGGCCTTTTTCCCAT
GCCTGCCTTTACTCTGCCAGAGTTATATTGCTGGGGTTTT
GAAGAAGATCCTATTAAATAAAAGAATAAGCAGTATTATT
AAGTAGCCCTGCATTTCAGGTTTCCTTGAGTGGCAGGCCA
GGCCTGGCCGTGAACGTTCACTGAAATCATGGCCTCTTGG
CCAAGATTGATAGCTTGTGCCTGTCCCTGAGTCCCAGTCC
ATCACGAGCAGCTGGTTTCTAAGATGCTATTTCCCGTATA
AAGCATGAGACCGTGACTTGCCAGCCCCACAGAGCCCCGC
CCTTGTCCATCACTGGCATCTGGACTCCAGCCTGGGTTGG
GGCAAAGAGGGAAATGAGATCATGTCCTAACCCTGATCCT
CTTGTCCCACAGATATCCAGAACCCTGACCCTGCCGTGTA
CCAGCTGAGAGACTCTAAATCCAGTGACAAGTCTGTCTGC
CTATTCGAATTCCGTTACATAACTTACGTTAAATGGCCCG
CCTGGCTGACCGCCCAACGACCCCCGCCCATTGACGTCAA
TAATGATGTATGTTCCCATAGTAACGCCAATAGGGACTTT
CCATTGACGTCAATGGGTGGAGTATTTACGGTAAACTGCC
CACTTGGCAGTACATCAAGTGTATCATATCCAAGTACGCC
CCCTATTGACGTCAATGACGGTAAATGGCCCGCCTGGCAT
TATGCCCAGTACATGACCTTATGGGACTTTCCTACTTGGC
AGTACATCTACGTATTAGTCATCGCTATTACCATGGGCTC
CGGAGCCACTAACTTCTCCCTGTTGAAACAGGCTGGCGAT
GTTGAAGAAAACCCCGGTCCTATGGCCACCGGCTCTAGAA
CAAGCCTGCTGCTCGCTTTTGGCCTGCTCTGCCTCCCATG
GCTCCAAGAAGGATCTGCTGATGCTGGAATCACCCAGAGC
CCAAGATACAAGATCACAGAGACAGGAAGGCAGGTGACCT
TGATGTGTCACCAGACTTGGAGCCACAGCTATATGTTCTG
GTATCGACAAGACCTGGGACATGGGCTGAGGCTGATCTAT
TACTCAGCAGCTGCTGATATTACAGATAAAGGAGAAGTCT
CCGATGGCTATGTTGTCTCCAGATCCAAGACAGAGAATTT
CCCCCTCACTCTGGAGTCAGCTACCCGCTCCCAGACATCT
GTGTATTTCTGCGCCAGCAGTGAGGACAGTTACGAGCAGT
ACTTCGGGCCGGGCACCAGGCTCACGGTCACAGAGGACCT
GAAAAACGTGTTCCCTCCAAAAGTGGCCGTGTTCGAGCCT
TCTGAGGCCGAGATCAGCCACACACAGAAAGCCACACTCG
TGTGTCTGGCTACCGGCTTCTACCCCGATCACGTGGAACT
GTCTTGGTGGGTCAACGGCAAAGAGGTGCACAGCGGCGTC
AGCACAGATCCCCAGCCTCTGAAAGAACAGCCCGCTCTGA
ACGACAGCCGCTACTGCCTGTCTAGCAGACTGAGAGTGTC
CGCCACCTTCTGGCAGAACCCCAGAAACCACTTCAGATGC
CAGGTCCAGTTCTACGGCCTGAGCGAGAACGATGAGTGGA
CCCAGGACAGAGCCAAGCCTGTGACACAGATCGTGTCTGC
CGAAGCCTGGGGCAGAGCCGATTGTGGCTTTACCAGCGAG
TCATACCAGCAGGGCGTGCTGTCTGCCACCATCCTGTATG
AGATCCTGCTCGGCAAGGCCACACTGTACGCTGTGCTGGT
GTCTGCTCTGGTGCTGATGGCTATGGTCTCCCGGGAGCGC
ATCCCCGAGGCCCGGGCCAAGCGGGGCAGCGGCGCCACCA
ACTTCAGCCTGCTGAAGCAGGCCGGCGACGTGGAGGAGAA
CCCCGGCCCTATGGCCACAGGCAGCAGAACATCTCTGCTG
CTGGCCTTCGGACTGCTGTGTCTGCCTTGGCTGCAAGAGG
GTTCCGCCGCCCAGTCAGTGACCCAGCCTGACATCCACAT
CACTGTCTCTGAAGGAGCCTCACTGGAGTTGAGATGTAAC
TATTCCTATGGGGCAACACCTTATCTCTTCTGGTATGTCC
AGTCCCCCGGCCAAGGCCTCCAGCTGCTCCTGAAGTACTT
TTCAGGAGACACTCTGGTTCAAGGCATTAAAGGCTTTGAG
GCTGAATTTAAGAGGAGTCAATCTTCCTTCAATCTGAGGA
AACCCTCTGTGCATTGGAGTGATGCTGCTGAGTACTTCTG
TGCTGTGGGTGAATTGGACACAGGCTTTCAGAAACTTGTA
TTTGGAACTGGCACCCGACTTCTGGTCAGTCCAAATATTC
AGAACCCCGATCCTGCCGTGTACCAGCTGAGAGACAGCAA
GAGCAGCGACAAGAGCGTGTGCCTGTTCACCGACTTCGAC
AGCCAGACCAACGTGTCCCAGAGCAAGGACAGCGACGTGT
ACATCACCGACAAGACCGTGCTGGACATGCGGAGCATGGA
CTTCAAGAGCAACAGCGCCGTGGCCTGGTCCAACAAGAGC
GATTTCGCCTGCGCCAACGCCTTCAACAACAGCATTATCC
CCGAGGACACATTCTTCCCAAGTCCTGAGAGCAGCTGCGA
CGTGAAGCTGGTGGAAAAGAGCTTCGAGACAGACACCAAC
CTGAACTTCCAGAACCTGTCCGTGATCGGCTTCCGCATCC
TGCTGCTGAAAGTGGCCGGCTTCAACCTGCTGATGACCCT
GAGACTGTGGTCCAGCTGATGTGCCTTCTAGTTGCCAGCC
ATCTGTTGTTTGCCCCTCCCCCGTGCCTTCCTTGACCCTG
GAAGGTGCCACTCCCACTGTCCTTTCCTAATAAAATGAGG
AAATTGCATCGCATTGTCTGAGTAGGTGTCATTCTATTCT
GGGGGGTGGGGTGGGGCAGGACAGCAAGGGGGAGGATTGG
GAAGACAATAGCAGGCATGCTGGGGATGCGGTGGGCTCTA
TGGCGTCGACGAGCTCACGGGGACAGCCCCCCCCCAAAGC
CCCCAGGGATGTAATTACGTCCCTCCCCCGCTAGGGGGCA
GCAGCGAGCCGCCCGGGGCTCCGCTCCGGTCCGGCGCTCC
CCCCGCATCCCCGAGCCGGCAGCGTGCGGGGACAGCCCGG
GCACGGGGAAGGTGGCACGGGATCGCTTTCCTCTGAACGC
TTCTCGCTGCTCTTTGAGCCTGCAGACACCTGGGGGGATA
CGGGGAAAAAGCTTCTCGAGTCGAATTCCACGGGGTTGGG
GTTGCGCCTTTTCCAAGGCAGCCCTGGGTTTGCGCAGGGA
CGCGGCTGCTCTGGGCGTGGTTCCGGGAAACGCAGCGGCG
CCGACCCTGGGTCTCGCACATTCTTCACGTCCGTTCGCAG
CGTCACCCGGATCTTCGCCGCTACCCTTGTGGGCCCCCCG
GCGACGCTTCCTGCTCCGCCCCTAAGTCGGGAAGGTTCCT
TGCGGTTCGCGGCGTGCCGGACGTGACAAACGGAAGCCGC
ACGTCTCACTAGTACCCTCGCAGACGGACAGCGCCAGGGA
GCAATGGCAGCGCGCCGACCGCGATGGGCTGTGGCCAATA
GCGGCTGCTCAGCAGGGCGCGCCGAGAGCAGCGGCCGGGA
AGGGGCGGTGCGGGAGGCGGGGTGTGGGGCGGTAGTGTGG
GCCCTGTTCCTGCCCGCGCGGTGTTCCGCATTCTGCAAGC
CTCCGGAGCGCACGTCGGCAGTCGGCTCCCTCGTTGACCG
AATCACCGACCTCTCTCCCCAGGGATCCGCCACCATGGTG
TCCAAGGGCGAAGAGGACAACATGGCCATCATCAAAGAGT
TCATGCGGTTCAAGGTGCACATGGAAGGCAGCGTGAACGG
CCACGAGTTCGAGATTGAAGGCGAAGGCGAGGGCAGACCT
TACGAGGGAACACAGACCGCCAAGCTGAAAGTGACCAAAG
GCGGCCCTCTGCCTTTTGCCTGGGACATTCTGAGCCCTCA
GTTTATGTACGGCAGCAAGGCCTACGTGAAGCACCCCGCC
GATATTCCCGACTACCTGAAGCTGAGCTTCCCCGAGGGCT
TCAAGTGGGAGAGAGTGATGAACTTCGAGGACGGCGGCGT
GGTCACCGTGACTCAAGATAGCTCTCTGCAGGACGGCGAG
TTCATCTACAAAGTGAAGCTGCGGGGCACCAACTTTCCCT
CTGATGGCCCCGTGATGCAGAAAAAGACAATGGGCTGGGA
AGCCAGCAGCGAGAGAATGTACCCTGAAGATGGCGCCCTG
AAGGGCGAGATCAAGCAGCGGCTGAAACTGAAGGATGGCG
GCCACTACGACGCCGAAGTGAAAACCACCTACAAGGCCAA
GAAACCCGTGCAGCTGCCTGGCGCCTACAACGTGAACATC
AAGCTGGACATCACCAGCCACAACGAGGACTACACCATCG
TGGAACAGTACGAGAGAGCCGAAGGCAGACACAGCACAGG
CGGAATGGACGAGCTGTACAAGAGAGCCAAACGGGGGTCC
GGCGCTACCAATTTCAGTTTGCTCAAACAGGCCGGAGACG
TCGAGGAAAATCCTGGCCCCCATATGACCGATTTTGATTC
TCAAACAAATGTGTCACAAAGTAAGGATTCTGATGTGTAT
ATCACAGACAAAACTGTGCTAGACATGAGGTCTATGGACT
TCAAGAGCAACAGTGCTGTGGCCTGGAGCAACAAATCTGA
CTTTGCATGTGCAAACGCCTTCAACAACAGCATTATTCCA
GAAGACACCTTCTTCCCCAGCCCAGGTAAGGGCAGCTTTG
GTGCCTTCGCAGGCTGTTTCCTTGCTTCAGGAATGGCCAG
GTTCTGCCCAGAGCTCTGGTCAATGATGTCTAAAACTCCT
CTGATTGGTGGTCTCGGCCTTATCCATTGCCACCAAAACC
CTCTTTTTACTAAGAAACAGTGAGCCTTGTTCTGGCAGTC
CAGAGAATGACACGGGAAAAAAGCAGATGAAGAGAAGGTG
GCAGGAGAGGGCACGTGGCCCAGCCTCAGTCTCTCCAACT
GAGTTCCTGCCTGCCTGCCTTTGCTCAGACTGTTTGCCCC
TTACTGCTCTTCTAGGCCTCATTCTAAGCCCCTTCTCCAA
GTTGCCTCTCCTTATTTCTCCCTGTCTGCCAAAAAATCTT
TCCCAGCTCACTAAGTCAGTCTCACGCAGTCACTCATTAA
CCCACCAATCACTGATTGTGCCGGCACATGAATGCACCAG
GTGTTGAAGTGGAGGAATTAAAAAGTCAGATGAGGGGTGT
GCCCAGAGGAAGCACCATTCTAGTTGGGGGAGCCCATCTG
TCAGCTGGGAAAAGTCCAAATAACTTCAGATTGGAATGTG
TTTTAACTCAGGGTTGAGAAAACAGCTACCTTCAGGACAA
AAGTCAGGGAAGGGCTCTCTGAAGAAATGCTACTTGAAGA
TACCAGCCCTACCAAGGGCAGGGAGAGGACCCTATAGAGG
CCTGGGACAGGAGCTCAATGAGAAAGGAGAAGAGCAGCAG
GCATGAGTTGAATGAAGGAGGCAGGGCCGGGTCACAGGGC
CTTCTAGGCCATGAGAGGGTAGACAGGCTAGCCGCGTTGC
TGGCGTTTTTCCATAGGCTCCGCCCCCCTGACGAGCATCA
CAAAAATCGACGCTCAAGTCAGAGGTGGCGAAACCCGACA
GGACTATAAAGATACCAGGCGTTTCCCCCTGGAAGCTCCC
TCGTGCGCTCTCCTGTTCCGACCCTGCCGCTTACCGGATA
CCTGTCCGCCTTTCTCCCTTCGGGAAGCGTGGCGCTTTCT
CATAGCTCACGCTGTAGGTATCTCAGTTCGGTGTAGGTCG
TTCGCTCCAAGCTGGGCTGTGTGCACGAACCCCCCGTTCA
GCCCGACCGCTGCGCCTTATCCGGTAACTATCGTCTTGAG
TCCAACCCGGTAAGACACGACTTATCGCCACTGGCAGCAG
CCACTGGTAACAGGATTAGCAGAGCGAGGTATGTAGGCGG
TGCTACAGAGTTCTTGAAGTGGTGGCCTAACTACGGCTAC
ACTAGAAGAACAGTATTTGGTATCTGCGCTCTGCTGAAGC
CAGTTACCTTCGGAAAAAGAGTTGGTAGCTCTTGATCCGG
CAAACAAACCACCGCTGGTAGCGGTGGTTTTTTTGTTTGC
AAGCAGCAGATTACGCGCAGAAAAAAAGGATCTCAAGAAG
ATCCTTTGATCTTTAGAAAAACTCATCGAGCATCAAATGA
AACTGCAATTTATTCATATCAGGATTATCAATACCATATT
TTTGAAAAAGCCGTTTCTGTAATGAAGGAGAAAACTCACC
GAGGCAGTTCCATAGGATGGCAAGATCCTGGTATCGGTCT
GCGATTCCGACTCGTCCAACATCAATACAACCTATTAATT
TCCCCTCGTCAAAAATAAGGTTATCAAGTGAGAAATCACC
ATGAGTGACGACTGAATCCGGTGAGAATGGCAAAAGTTTA
TGCATTTCTTTCCAGACTTGTTCAACAGGCCAGCCATTAC
GCTCGTCATCAAAATCACTCGCATCAACCAAACCGTTATT
CATTCGTGATTGCGCCTGAGCCAGACGAAATACGCGATCG
CTGTTAAAAGGACAATTACAAACAGGAATCGAATGCAACC
GGCGCAGGAACACTGCCAGCGCATCAACAATATTTTCACC
TGAATCAGGATATTCTTCTAATACCTGGAATGCTGTTTTT
CCGGGGATCGCAGTGGTGAGTAACCATGCATCATCAGGAG
TACGGATAAAATGCTTGATGGTCGGAAGAGGCATAAATTC
CGTCAGCCAGTTTAGTCTGACCATCTCATCTGTAACATCA
TTGGCAACGCTACCTTTGCCATGTTTCAGAAACAACTCTG
GCGCATCGGGCTTCCCATACAAGCGATAGATTGTCGCACC
TGATTGCCCGACATTATCGCGAGCCCATTTATACCCATAT
AAATCAGCATCCATGTTGGAATTTAATCGCGGCCTCGACG
TTTCCCGTTGAATATGGCTCATAACACCCCTTGTATTACT
GTTTATGTAAGCAGACAGTTTTATTGTTCATGATGATATA
TTTTTATCTTGTGCAATGTAACATCAGAGATTTTGAGACA
C

In certain embodiments, the Secondary Promoter Construct comprises nucleotides 1 to 4105 of SEQ ID NO: 57. In certain embodiments, the Secondary Promoter Construct comprises nucleotides 4814 to 7432 of SEQ ID NO: 57. In certain embodiments, the Secondary Promoter Construct comprises nucleotides 1 to 4105 and nucleotides 4814 to 7432 of SEQ ID NO: 57 SEQ ID NO: 57 is provided below.

[SEQ ID NO: 57]
GGTACCACATTAAAAACACAAAATCCTACGGAAATACTGA
AGAATGAGTCTCAGCACTAAGGAAAAGCCTCCAGCAGCTC
CTGCTTTCTGAGGGTGAAGGATAGACGCTGTGGCTCTGCA
TGACTCACTAGCACTCTATCACGGCCATATTCTGGCAGGG
TCAGTGGCTCCAACTAACATTTGTTTGGTACTTTACAGTT
TATTAAATAGATGTTTATATGGAGAAGCTCTCATTTCTTT
CTCAGAAGAGCCTGGCTAGGAAGGTGGATGAGGCACCATA
TTCATTTTGCAGGTGAAATTCCTGAGATGTAAGGAGCTGC
TGTGACTTGCTCAAGGCCTTATATCGAGTAAACGGTAGTG
CTGGGGCTTAGACGCAGGTGTTCTGATTTATAGTTCAAAA
CCTCTATCAATGAGAGAGCAATCTCCTGGTAATGTGATAG
ATTTCCCAACTTAATGCCAACATACCATAAACCTCCCATT
CTGCTAATGCCCAGCCTAAGTTGGGGAGACCACTCCAGAT
TCCAAGATGTACAGTTTGCTTTGCTGGGCCTTTTTCCCAT
GCCTGCCTTTACTCTGCCAGAGTTATATTGCTGGGGTTTT
GAAGAAGATCCTATTAAATAAAAGAATAAGCAGTATTATT
AAGTAGCCCTGCATTTCAGGTTTCCTTGAGTGGCAGGCCA
GGCCTGGCCGTGAACGTTCACTGAAATCATGGCCTCTTGG
CCAAGATTGATAGCTTGTGCCTGTCCCTGAGTCCCAGTCC
ATCACGAGCAGCTGGTTTCTAAGATGCTATTTCCCGTATA
AAGCATGAGACCGTGACTTGCCAGCCCCACAGAGCCCCGC
CCTTGTCCATCACTGGCATCTGGACTCCAGCCTGGGTTGG
GGCAAAGAGGGAAATGAGATCATGTCCTAACCCTGATCCT
CTTGTCCCACAGATATCCAGAACCCTGACCCTGCCGTGTA
CCAGCTGAGAGACTCTAAATCCAGTGACAAGTCTGTCTGC
CTATTCGAATTCCGTTACATAACTTACGTTAAATGGCCCG
CCTGGCTGACCGCCCAACGACCCCCGCCCATTGACGTCAA
TAATGATGTATGTTCCCATAGTAACGCCAATAGGGACTTT
CCATTGACGTCAATGGGTGGAGTATTTACGGTAAACTGCC
CACTTGGCAGTACATCAAGTGTATCATATCCAAGTACGCC
CCCTATTGACGTCAATGACGGTAAATGGCCCGCCTGGCAT
TATGCCCAGTACATGACCTTATGGGACTTTCCTACTTGGC
AGTACATCTACGTATTAGTCATCGCTATTACCATGGGCTC
CGGAGCCACTAACTTCTCCCTGTTGAAACAGGCTGGCGAT
GTTGAAGAAAACCCCGGTCCTATGGCCACCGGCTCTAGAA
CAAGCCTGCTGCTCGCTTTTGGCCTGCTCTGCCTCCCATG
GCTCCAAGAAGGATCTGCTGATGCTGGAATCACCCAGAGC
CCAAGATACAAGATCACAGAGACAGGAAGGCAGGTGACCT
TGATGTGTCACCAGACTTGGAGCCACAGCTATATGTTCTG
GTATCGACAAGACCTGGGACATGGGCTGAGGCTGATCTAT
TACTCAGCAGCTGCTGATATTACAGATAAAGGAGAAGTCT
CCGATGGCTATGTTGTCTCCAGATCCAAGACAGAGAATTT
CCCCCTCACTCTGGAGTCAGCTACCCGCTCCCAGACATCT
GTGTATTTCTGCGCCAGCAGTGAGGACAGTTACGAGCAGT
ACTTCGGGCCGGGCACCAGGCTCACGGTCACAGAGGACCT
GAAAAACGTGTTCCCTCCAAAAGTGGCCGTGTTCGAGCCT
TCTGAGGCCGAGATCAGCCACACACAGAAAGCCACACTCG
TGTGTCTGGCTACCGGCTTCTACCCCGATCACGTGGAACT
GTCTTGGTGGGTCAACGGCAAAGAGGTGCACAGCGGCGTC
AGCACAGATCCCCAGCCTCTGAAAGAACAGCCCGCTCTGA
ACGACAGCCGCTACTGCCTGTCTAGCAGACTGAGAGTGTC
CGCCACCTTCTGGCAGAACCCCAGAAACCACTTCAGATGC
CAGGTCCAGTTCTACGGCCTGAGCGAGAACGATGAGTGGA
CCCAGGACAGAGCCAAGCCTGTGACACAGATCGTGTCTGC
CGAAGCCTGGGGCAGAGCCGATTGTGGCTTTACCAGCGAG
TCATACCAGCAGGGCGTGCTGTCTGCCACCATCCTGTATG
AGATCCTGCTCGGCAAGGCCACACTGTACGCTGTGCTGGT
GTCTGCTCTGGTGCTGATGGCTATGGTCTCCCGGGAGCGC
ATCCCCGAGGCCCGGGCCAAGCGGGGCAGCGGCGCCACCA
ACTTCAGCCTGCTGAAGCAGGCCGGCGACGTGGAGGAGAA
CCCCGGCCCTATGGCCACAGGCAGCAGAACATCTCTGCTG
CTGGCCTTCGGACTGCTGTGTCTGCCTTGGCTGCAAGAGG
GTTCCGCCGCCCAGTCAGTGACCCAGCCTGACATCCACAT
CACTGTCTCTGAAGGAGCCTCACTGGAGTTGAGATGTAAC
TATTCCTATGGGGCAACACCTTATCTCTTCTGGTATGTCC
AGTCCCCCGGCCAAGGCCTCCAGCTGCTCCTGAAGTACTT
TTCAGGAGACACTCTGGTTCAAGGCATTAAAGGCTTTGAG
GCTGAATTTAAGAGGAGTCAATCTTCCTTCAATCTGAGGA
AACCCTCTGTGCATTGGAGTGATGCTGCTGAGTACTTCTG
TGCTGTGGGTGAATTGGACACAGGCTTTCAGAAACTTGTA
TTTGGAACTGGCACCCGACTTCTGGTCAGTCCAAATATTC
AGAACCCCGATCCTGCCGTGTACCAGCTGAGAGACAGCAA
GAGCAGCGACAAGAGCGTGTGCCTGTTCACCGACTTCGAC
AGCCAGACCAACGTGTCCCAGAGCAAGGACAGCGACGTGT
ACATCACCGACAAGACCGTGCTGGACATGCGGAGCATGGA
CTTCAAGAGCAACAGCGCCGTGGCCTGGTCCAACAAGAGC
GATTTCGCCTGCGCCAACGCCTTCAACAACAGCATTATCC
CCGAGGACACATTCTTCCCAAGTCCTGAGAGCAGCTGCGA
CGTGAAGCTGGTGGAAAAGAGCTTCGAGACAGACACCAAC
CTGAACTTCCAGAACCTGTCCGTGATCGGCTTCCGCATCC
TGCTGCTGAAAGTGGCCGGCTTCAACCTGCTGATGACCCT
GAGACTGTGGTCCAGCTGATGTGCCTTCTAGTTGCCAGCC
ATCTGTTGTTTGCCCCTCCCCCGTGCCTTCCTTGACCCTG
GAAGGTGCCACTCCCACTGTCCTTTCCTAATAAAATGAGG
AAATTGCATCGCATTGTCTGAGTAGGTGTCATTCTATTCT
GGGGGGTGGGGTGGGGCAGGACAGCAAGGGGGAGGATTGG
GAAGACAATAGCAGGCATGCTGGGGATGCGGTGGGCTCTA
TGGCGTCGACGAGCTCACGGGGACAGCCCCCCCCCAAAGC
CCCCAGGGATGTAATTACGTCCCTCCCCCGCTAGGGGGCA
GCAGCGAGCCGCCCGGGGCTCCGCTCCGGTCCGGCGCTCC
CCCCGCATCCCCGAGCCGGCAGCGTGCGGGGACAGCCCGG
GCACGGGGAAGGTGGCACGGGATCGCTTTCCTCTGAACGC
TTCTCGCTGCTCTTTGAGCCTGCAGACACCTGGGGGGATA
CGGGGAAAAAGCTTCTCGAGAGGAGGAAAAACTGTTTCAT
ACAGAAGGCGTCAATTAGGAGGAAAAACTGTTTCATACAG
AAGGCGTCAATTAGGAGGAAAAACTGTTTCATACAGAAGG
CGTCAATTGGTCCCATCGAATTAGGAGGAAAAACTGTTTC
ATACAGAAGGCGTCAATTAGGAGGAAAAACTGTTTCATAC
AGAAGGCGTCAATTAGGAGGAAAAACTGTTTCATACAGAA
GGCGTCAATTGGTCCCGGGACATTTTGACACCCCCATAAT
ATTTTTCCAGAATTAACAGTATAAATTGCATCTCTTGTTC
AAGAGTTCCCTATCACTCTCTTTAATCACTACTCACAGTA
ACCTCAACTCCTGGGATCCGCCACCATGGTGTCCAAGGGC
GAAGAGGACAACATGGCCATCATCAAAGAGTTCATGCGGT
TCAAGGTGCACATGGAAGGCAGCGTGAACGGCCACGAGTT
CGAGATTGAAGGCGAAGGCGAGGGCAGACCTTACGAGGGA
ACACAGACCGCCAAGCTGAAAGTGACCAAAGGCGGCCCTC
TGCCTTTTGCCTGGGACATTCTGAGCCCTCAGTTTATGTA
CGGCAGCAAGGCCTACGTGAAGCACCCCGCCGATATTCCC
GACTACCTGAAGCTGAGCTTCCCCGAGGGCTTCAAGTGGG
AGAGAGTGATGAACTTCGAGGACGGCGGCGTGGTCACCGT
GACTCAAGATAGCTCTCTGCAGGACGGCGAGTTCATCTAC
AAAGTGAAGCTGCGGGGCACCAACTTTCCCTCTGATGGCC
CCGTGATGCAGAAAAAGACAATGGGCTGGGAAGCCAGCAG
CGAGAGAATGTACCCTGAAGATGGCGCCCTGAAGGGCGAG
ATCAAGCAGCGGCTGAAACTGAAGGATGGCGGCCACTACG
ACGCCGAAGTGAAAACCACCTACAAGGCCAAGAAACCCGT
GCAGCTGCCTGGCGCCTACAACGTGAACATCAAGCTGGAC
ATCACCAGCCACAACGAGGACTACACCATCGTGGAACAGT
ACGAGAGAGCCGAAGGCAGACACAGCACAGGCGGAATGGA
CGAGCTGTACAAGAGAGCCAAACGGGGGTCCGGCGCTACC
AATTTCAGTTTGCTCAAACAGGCCGGAGACGTCGAGGAAA
ATCCTGGCCCCCATATGACCGATTTTGATTCTCAAACAAA
TGTGTCACAAAGTAAGGATTCTGATGTGTATATCACAGAC
AAAACTGTGCTAGACATGAGGTCTATGGACTTCAAGAGCA
ACAGTGCTGTGGCCTGGAGCAACAAATCTGACTTTGCATG
TGCAAACGCCTTCAACAACAGCATTATTCCAGAAGACACC
TTCTTCCCCAGCCCAGGTAAGGGCAGCTTTGGTGCCTTCG
CAGGCTGTTTCCTTGCTTCAGGAATGGCCAGGTTCTGCCC
AGAGCTCTGGTCAATGATGTCTAAAACTCCTCTGATTGGT
GGTCTCGGCCTTATCCATTGCCACCAAAACCCTCTTTTTA
CTAAGAAACAGTGAGCCTTGTTCTGGCAGTCCAGAGAATG
ACACGGGAAAAAAGCAGATGAAGAGAAGGTGGCAGGAGAG
GGCACGTGGCCCAGCCTCAGTCTCTCCAACTGAGTTCCTG
CCTGCCTGCCTTTGCTCAGACTGTTTGCCCCTTACTGCTC
TTCTAGGCCTCATTCTAAGCCCCTTCTCCAAGTTGCCTCT
CCTTATTTCTCCCTGTCTGCCAAAAAATCTTTCCCAGCTC
ACTAAGTCAGTCTCACGCAGTCACTCATTAACCCACCAAT
CACTGATTGTGCCGGCACATGAATGCACCAGGTGTTGAAG
TGGAGGAATTAAAAAGTCAGATGAGGGGTGTGCCCAGAGG
AAGCACCATTCTAGTTGGGGGAGCCCATCTGTCAGCTGGG
AAAAGTCCAAATAACTTCAGATTGGAATGTGTTTTAACTC
AGGGTTGAGAAAACAGCTACCTTCAGGACAAAAGTCAGGG
AAGGGCTCTCTGAAGAAATGCTACTTGAAGATACCAGCCC
TACCAAGGGCAGGGAGAGGACCCTATAGAGGCCTGGGACA
GGAGCTCAATGAGAAAGGAGAAGAGCAGCAGGCATGAGTT
GAATGAAGGAGGCAGGGCCGGGTCACAGGGCCTTCTAGGC
CATGAGAGGGTAGACAGGCTAGCCGCGTTGCTGGCGTTTT
TCCATAGGCTCCGCCCCCCTGACGAGCATCACAAAAATCG
ACGCTCAAGTCAGAGGTGGCGAAACCCGACAGGACTATAA
AGATACCAGGCGTTTCCCCCTGGAAGCTCCCTCGTGCGCT
CTCCTGTTCCGACCCTGCCGCTTACCGGATACCTGTCCGC
CTTTCTCCCTTCGGGAAGCGTGGCGCTTTCTCATAGCTCA
CGCTGTAGGTATCTCAGTTCGGTGTAGGTCGTTCGCTCCA
AGCTGGGCTGTGTGCACGAACCCCCCGTTCAGCCCGACCG
CTGCGCCTTATCCGGTAACTATCGTCTTGAGTCCAACCCG
GTAAGACACGACTTATCGCCACTGGCAGCAGCCACTGGTA
ACAGGATTAGCAGAGCGAGGTATGTAGGCGGTGCTACAGA
GTTCTTGAAGTGGTGGCCTAACTACGGCTACACTAGAAGA
ACAGTATTTGGTATCTGCGCTCTGCTGAAGCCAGTTACCT
TCGGAAAAAGAGTTGGTAGCTCTTGATCCGGCAAACAAAC
CACCGCTGGTAGCGGTGGTTTTTTTGTTTGCAAGCAGCAG
ATTACGCGCAGAAAAAAAGGATCTCAAGAAGATCCTTTGA
TCTTTAGAAAAACTCATCGAGCATCAAATGAAACTGCAAT
CCATAGGATGGCAAGATCCTGGTATCGGTCTGCGATTCCG
ACTCGTCCAACATCAATACAACCTATTAATTTCCCCTCGT
CAAAAATAAGGTTATCAAGTGAGAAATCACCATGAGTGAC
GACTGAATCCGGTGAGAATGGCAAAAGTTTATGCATTTCT
TTCCAGACTTGTTCAACAGGCCAGCCATTACGCTCGTCAT
CAAAATCACTCGCATCAACCAAACCGTTATTCATTCGTGA
TTGCGCCTGAGCCAGACGAAATACGCGATCGCTGTTAAAA
GGACAATTACAAACAGGAATCGAATGCAACCGGCGCAGGA
ACACTGCCAGCGCATCAACAATATTTTCACCTGAATCAGG
ATATTCTTCTAATACCTGGAATGCTGTTTTTCCGGGGATC
GCAGTGGTGAGTAACCATGCATCATCAGGAGTACGGATAA
AATGCTTGATGGTCGGAAGAGGCATAAATTCCGTCAGCCA
GTTTAGTCTGACCATCTCATCTGTAACATCATTGGCAACG
CTACCTTTGCCATGTTTCAGAAACAACTCTGGCGCATCGG
GCTTCCCATACAAGCGATAGATTGTCGCACCTGATTGCCC
GACATTATCGCGAGCCCATTTATACCCATATAAATCAGCA
TCCATGTTGGAATTTAATCGCGGCCTCGACGTTTCCCGTT
GAATATGGCTCATAACACCCCTTGTATTACTGTTTATGTA
AGCAGACAGTTTTATTGTTCATGATGATATATTTTTATCT
TGTGCAATGTAACATCAGAGATTTTGAGACAC

In certain embodiments, the Secondary Promoter Construct comprises nucleotides 1 to 4007 of SEQ ID NO: 58. In certain embodiments, the Secondary Promoter Construct comprises nucleotides 4716 to 7334 of SEQ ID NO: 58. In certain embodiments, the Secondary Promoter Construct comprises nucleotides 1 to 4007 and nucleotides 4716 to 7334 of SEQ ID NO: 58 SEQ ID NO: 58 is provided below.

[SEQ ID NO: 58]
GGTACCACATTAAAAACACAAAATCCTACGGAAATACTGA
AGAATGAGTCTCAGCACTAAGGAAAAGCCTCCAGCAGCTC
CTGCTTTCTGAGGGTGAAGGATAGACGCTGTGGCTCTGCA
TGACTCACTAGCACTCTATCACGGCCATATTCTGGCAGGG
TCAGTGGCTCCAACTAACATTTGTTTGGTACTTTACAGTT
TATTAAATAGATGITTATATGGAGAAGCTCTCATTTCTTT
CTCAGAAGAGCCTGGCTAGGAAGGTGGATGAGGCACCATA
TTCATTTTGCAGGTGAAATTCCTGAGATGTAAGGAGCTGC
TGTGACTTGCTCAAGGCCTTATATCGAGTAAACGGTAGTG
CTGGGGCTTAGACGCAGGTGTTCTGATTTATAGTTCAAAA
CCTCTATCAATGAGAGAGCAATCTCCTGGTAATGTGATAG
ATTTCCCAACTTAATGCCAACATACCATAAACCTCCCATT
CTGCTAATGCCCAGCCTAAGTTGGGGAGACCACTCCAGAT
TCCAAGATGTACAGTTTGCTTTGCTGGGCCTTTTTCCCAT
GCCTGCCTTTACTCTGCCAGAGTTATATTGCTGGGGTTTT
GAAGAAGATCCTATTAAATAAAAGAATAAGCAGTATTATT
AAGTAGCCCTGCATTTCAGGTTTCCTTGAGTGGCAGGCCA
GGCCTGGCCGTGAACGTTCACTGAAATCATGGCCTCTTGG
CCAAGATTGATAGCTTGTGCCTGTCCCTGAGTCCCAGTCC
ATCACGAGCAGCTGGTTTCTAAGATGCTATTTCCCGTATA
AAGCATGAGACCGTGACTTGCCAGCCCCACAGAGCCCCGC
CCTTGTCCATCACTGGCATCTGGACTCCAGCCTGGGTTGG
GGCAAAGAGGGAAATGAGATCATGTCCTAACCCTGATCCT
CTTGTCCCACAGATATCCAGAACCCTGACCCTGCCGTGTA
CCAGCTGAGAGACTCTAAATCCAGTGACAAGTCTGTCTGC
CTATTCGAATTCCGTTACATAACTTACGTTAAATGGCCCG
CCTGGCTGACCGCCCAACGACCCCCGCCCATTGACGTCAA
TAATGATGTATGTTCCCATAGTAACGCCAATAGGGACTTT
CCATTGACGTCAATGGGTGGAGTATTTACGGTAAACTGCC
CACTTGGCAGTACATCAAGTGTATCATATCCAAGTACGCC
CCCTATTGACGTCAATGACGGTAAATGGCCCGCCTGGCAT
TATGCCCAGTACATGACCTTATGGGACTTTCCTACTTGGC
AGTACATCTACGTATTAGTCATCGCTATTACCATGGGCTC
CGGAGCCACTAACTTCTCCCTGTTGAAACAGGCTGGCGAT
GTTGAAGAAAACCCCGGTCCTATGGCCACCGGCTCTAGAA
CAAGCCTGCTGCTCGCTTTTGGCCTGCTCTGCCTCCCATG
GCTCCAAGAAGGATCTGCTGATGCTGGAATCACCCAGAGC
CCAAGATACAAGATCACAGAGACAGGAAGGCAGGTGACCT
TGATGTGTCACCAGACTTGGAGCCACAGCTATATGTTCTG
GTATCGACAAGACCTGGGACATGGGCTGAGGCTGATCTAT
TACTCAGCAGCTGCTGATATTACAGATAAAGGAGAAGTCT
CCGATGGCTATGTTGTCTCCAGATCCAAGACAGAGAATTT
CCCCCTCACTCTGGAGTCAGCTACCCGCTCCCAGACATCT
GTGTATTTCTGCGCCAGCAGTGAGGACAGTTACGAGCAGT
ACTTCGGGCCGGGCACCAGGCTCACGGTCACAGAGGACCT
GAAAAACGTGTTCCCTCCAAAAGTGGCCGTGTTCGAGCCT
TCTGAGGCCGAGATCAGCCACACACAGAAAGCCACACTCG
TGTGTCTGGCTACCGGCTTCTACCCCGATCACGTGGAACT
GTCTTGGTGGGTCAACGGCAAAGAGGTGCACAGCGGCGTC
AGCACAGATCCCCAGCCTCTGAAAGAACAGCCCGCTCTGA
ACGACAGCCGCTACTGCCTGTCTAGCAGACTGAGAGTGTC
CGCCACCTTCTGGCAGAACCCCAGAAACCACTTCAGATGC
CAGGTCCAGTTCTACGGCCTGAGCGAGAACGATGAGTGGA
CCCAGGACAGAGCCAAGCCTGTGACACAGATCGTGTCTGC
CGAAGCCTGGGGCAGAGCCGATTGTGGCTTTACCAGCGAG
TCATACCAGCAGGGCGTGCTGTCTGCCACCATCCTGTATG
AGATCCTGCTCGGCAAGGCCACACTGTACGCTGTGCTGGT
GTCTGCTCTGGTGCTGATGGCTATGGTCTCCCGGGAGCGC
ATCCCCGAGGCCCGGGCCAAGCGGGGCAGCGGCGCCACCA
ACTTCAGCCTGCTGAAGCAGGCCGGCGACGTGGAGGAGAA
CCCCGGCCCTATGGCCACAGGCAGCAGAACATCTCTGCTG
CTGGCCTTCGGACTGCTGTGTCTGCCTTGGCTGCAAGAGG
GTTCCGCCGCCCAGTCAGTGACCCAGCCTGACATCCACAT
CACTGTCTCTGAAGGAGCCTCACTGGAGTTGAGATGTAAC
TATTCCTATGGGGCAACACCTTATCTCTTCTGGTATGTCC
AGTCCCCCGGCCAAGGCCTCCAGCTGCTCCTGAAGTACTT
TTCAGGAGACACTCTGGTTCAAGGCATTAAAGGCTTTGAG
GCTGAATTTAAGAGGAGTCAATCTTCCTTCAATCTGAGGA
AACCCTCTGTGCATTGGAGTGATGCTGCTGAGTACTTCTG
TGCTGTGGGTGAATTGGACACAGGCTTTCAGAAACTTGTA
TTTGGAACTGGCACCCGACTTCTGGTCAGTCCAAATATTC
AGAACCCCGATCCTGCCGTGTACCAGCTGAGAGACAGCAA
GAGCAGCGACAAGAGCGTGTGCCTGTTCACCGACTTCGAC
AGCCAGACCAACGTGTCCCAGAGCAAGGACAGCGACGTGT
ACATCACCGACAAGACCGTGCTGGACATGCGGAGCATGGA
CTTCAAGAGCAACAGCGCCGTGGCCTGGTCCAACAAGAGC
GATTTCGCCTGCGCCAACGCCTTCAACAACAGCATTATCC
CCGAGGACACATTCTTCCCAAGTCCTGAGAGCAGCTGCGA
CGTGAAGCTGGTGGAAAAGAGCTTCGAGACAGACACCAAC
CTGAACTTCCAGAACCTGTCCGTGATCGGCTTCCGCATCC
TGCTGCTGAAAGTGGCCGGCTTCAACCTGCTGATGACCCT
GAGACTGTGGTCCAGCTGATGTGCCTTCTAGTTGCCAGCC
ATCTGTTGTTTGCCCCTCCCCCGTGCCTTCCTTGACCCTG
GAAGGTGCCACTCCCACTGTCCTTTCCTAATAAAATGAGG
AAATTGCATCGCATTGTCTGAGTAGGTGTCATTCTATTCT
GGGGGGTGGGGTGGGGCAGGACAGCAAGGGGGAGGATTGG
GAAGACAATAGCAGGCATGCTGGGGATGCGGTGGGCTCTA
TGGCGTCGACGAGCTCACGGGGACAGCCCCCCCCCAAAGC
CCCCAGGGATGTAATTACGTCCCTCCCCCGCTAGGGGGCA
GCAGCGAGCCGCCCGGGGCTCCGCTCCGGTCCGGCGCTCC
CCCCGCATCCCCGAGCCGGCAGCGTGCGGGGACAGCCCGG
GCACGGGGAAGGTGGCACGGGATCGCTTTCCTCTGAACGC
TTCTCGCTGCTCTTTGAGCCTGCAGACACCTGGGGGGATA
CGGGGAAAAAGCTTCTCGAGGGGACTTTCCGCTTGGGGAC
TTTCCGCTGGGGACTTTCCGCTGGGGACTTTCCGCTGGGG
ACTTTCCGAATTCAGCTTGAATAAAATGAATATTAGAAGC
TGTTAGAATAAGAGAAAATGACAGAGGAAAACTGAAAGGG
AGAACTGAAAGTGGGAAATTCCTCTGAGGCAGAAAGGACC
ATCCCTTATAAATAGCACAGGCCATGAAGGAAGATCATTC
TCACTGCAGCCTTTGACAGCCTTTGCCTCATCTTGGGATC
CGCCACCATGGTGTCCAAGGGCGAAGAGGACAACATGGCC
ATCATCAAAGAGTTCATGCGGTTCAAGGTGCACATGGAAG
GCAGCGTGAACGGCCACGAGTTCGAGATTGAAGGCGAAGG
CGAGGGCAGACCTTACGAGGGAACACAGACCGCCAAGCTG
AAAGTGACCAAAGGCGGCCCTCTGCCTTTTGCCTGGGACA
TTCTGAGCCCTCAGTTTATGTACGGCAGCAAGGCCTACGT
GAAGCACCCCGCCGATATTCCCGACTACCTGAAGCTGAGC
TTCCCCGAGGGCTTCAAGTGGGAGAGAGTGATGAACTTCG
AGGACGGCGGCGTGGTCACCGTGACTCAAGATAGCTCTCT
GCAGGACGGCGAGTTCATCTACAAAGTGAAGCTGCGGGGC
ACCAACTTTCCCTCTGATGGCCCCGTGATGCAGAAAAAGA
CAATGGGCTGGGAAGCCAGCAGCGAGAGAATGTACCCTGA
AGATGGCGCCCTGAAGGGCGAGATCAAGCAGCGGCTGAAA
CTGAAGGATGGCGGCCACTACGACGCCGAAGTGAAAACCA
CCTACAAGGCCAAGAAACCCGTGCAGCTGCCTGGCGCCTA
CAACGTGAACATCAAGCTGGACATCACCAGCCACAACGAG
GACTACACCATCGTGGAACAGTACGAGAGAGCCGAAGGCA
GACACAGCACAGGCGGAATGGACGAGCTGTACAAGAGAGC
CAAACGGGGGTCCGGCGCTACCAATTTCAGTTTGCTCAAA
CAGGCCGGAGACGTCGAGGAAAATCCTGGCCCCCATATGA
CCGATTTTGATTCTCAAACAAATGTGTCACAAAGTAAGGA
TTCTGATGTGTATATCACAGACAAAACTGTGCTAGACATG
AGGTCTATGGACTTCAAGAGCAACAGTGCTGTGGCCTGGA
GCAACAAATCTGACTTTGCATGTGCAAACGCCTTCAACAA
CAGCATTATTCCAGAAGACACCTTCTTCCCCAGCCCAGGT
AAGGGCAGCTTTGGTGCCTTCGCAGGCTGTTTCCTTGCTT
CAGGAATGGCCAGGTTCTGCCCAGAGCTCTGGTCAATGAT
GTCTAAAACTCCTCTGATTGGTGGTCTCGGCCTTATCCAT
TGCCACCAAAACCCTCTTTTTACTAAGAAACAGTGAGCCT
TGTTCTGGCAGTCCAGAGAATGACACGGGAAAAAAGCAGA
TGAAGAGAAGGTGGCAGGAGAGGGCACGTGGCCCAGCCTC
AGTCTCTCCAACTGAGTTCCTGCCTGCCTGCCTTTGCTCA
GACTGTTTGCCCCTTACTGCTCTTCTAGGCCTCATTCTAA
GCCCCTTCTCCAAGTTGCCTCTCCTTATTTCTCCCTGTCT
GCCAAAAAATCTTTCCCAGCTCACTAAGTCAGTCTCACGC
AGTCACTCATTAACCCACCAATCACTGATTGTGCCGGCAC
ATGAATGCACCAGGTGTTGAAGTGGAGGAATTAAAAAGTC
AGATGAGGGGTGTGCCCAGAGGAAGCACCATTCTAGTTGG
GGGAGCCCATCTGTCAGCTGGGAAAAGTCCAAATAACTTC
AGATTGGAATGTGTTTTAACTCAGGGTTGAGAAAACAGCT
ACCTTCAGGACAAAAGTCAGGGAAGGGCTCTCTGAAGAAA
TGCTACTTGAAGATACCAGCCCTACCAAGGGCAGGGAGAG
GACCCTATAGAGGCCTGGGACAGGAGCTCAATGAGAAAGG
AGAAGAGCAGCAGGCATGAGTTGAATGAAGGAGGCAGGGC
CGGGTCACAGGGCCTTCTAGGCCATGAGAGGGTAGACAGG
CTAGCCGCGTTGCTGGCGTTTTTCCATAGGCTCCGCCCCC
CTGACGAGCATCACAAAAATCGACGCTCAAGTCAGAGGTG
GCGAAACCCGACAGGACTATAAAGATACCAGGCGTTTCCC
CCTGGAAGCTCCCTCGTGCGCTCTCCTGTTCCGACCCTGC
CGCTTACCGGATACCTGTCCGCCTTTCTCCCTTCGGGAAG
CGTGGCGCTTTCTCATAGCTCACGCTGTAGGTATCTCAGT
TCGGTGTAGGTCGTTCGCTCCAAGCTGGGCTGTGTGCACG
AACCCCCCGTTCAGCCCGACCGCTGCGCCTTATCCGGTAA
CTATCGTCTTGAGTCCAACCCGGTAAGACACGACTTATCG
CCACTGGCAGCAGCCACTGGTAACAGGATTAGCAGAGCGA
GGTATGTAGGCGGTGCTACAGAGTTCTTGAAGTGGTGGCC
TAACTACGGCTACACTAGAAGAACAGTATTTGGTATCTGC
GCTCTGCTGAAGCCAGTTACCTTCGGAAAAAGAGTTGGTA
GCTCTTGATCCGGCAAACAAACCACCGCTGGTAGCGGTGG
TTTTTTTGTTTGCAAGCAGCAGATTACGCGCAGAAAAAAA
GGATCTCAAGAAGATCCTTTGATCTTTAGAAAAACTCATC
GAGCATCAAATGAAACTGCAATTTATTCATATCAGGATTA
TCAATACCATATTTTTGAAAAAGCCGTTTCTGTAATGAAG
GAGAAAACTCACCGAGGCAGTTCCATAGGATGGCAAGATC
CTGGTATCGGTCTGCGATTCCGACTCGTCCAACATCAATA
CAACCTATTAATTTCCCCTCGTCAAAAATAAGGTTATCAA
GTGAGAAATCACCATGAGTGACGACTGAATCCGGTGAGAA
TGGCAAAAGTTTATGCATTTCTTTCCAGACTTGTTCAACA
GGCCAGCCATTACGCTCGTCATCAAAATCACTCGCATCAA
CCAAACCGTTATTCATTCGTGATTGCGCCTGAGCCAGACG
AAATACGCGATCGCTGTTAAAAGGACAATTACAAACAGGA
ATCGAATGCAACCGGCGCAGGAACACTGCCAGCGCATCAA
CAATATTTTCACCTGAATCAGGATATTCTTCTAATACCTG
GAATGCTGTTTTTCCGGGGATCGCAGTGGTGAGTAACCAT
GCATCATCAGGAGTACGGATAAAATGCTTGATGGTCGGAA
GAGGCATAAATTCCGTCAGCCAGTTTAGTCTGACCATCTC
ATCTGTAACATCATTGGCAACGCTACCTTTGCCATGTTTC
AGAAACAACTCTGGCGCATCGGGCTTCCCATACAAGCGAT
AGATTGTCGCACCTGATTGCCCGACATTATCGCGAGCCCA
TTTATACCCATATAAATCAGCATCCATGTTGGAATTTAAT
CGCGGCCTCGACGTTTCCCGTTGAATATGGCTCATAACAC
CCCTTGTATTACTGTTTATGTAAGCAGACAGTTTTATTGT
TCATGATGATATATTTTTATCTTGTGCAATGTAACATCAG
AGATTTTGAGACAC

In certain embodiments, the Secondary Promoter Construct comprises nucleotides 1 to 2881 of SEQ ID NO: 59. In certain embodiments, the Secondary Promoter Construct comprises nucleotides 3759 to 5679 of SEQ ID NO: 59. In certain embodiments, the Secondary Promoter Construct comprises nucleotides 1 to 2881 and nucleotides 3759 to 5679 of SEQ ID NO: 59. SEQ ID NO: 59 is provided below.

[SEQ ID NO: 59]
GGTACCTCATGGCCTCTTGGCCAAGATTGATAGCTTGTGC
CTGTCCCTGAGTCCCAGTCCATCACGAGCAGCTGGTTTCT
AAGATGCTATTTCCCGTATAAAGCATGAGACCGTGACTTG
CCAGCCCCACAGAGCCCCGCCCTTGTCCATCACTGGCATC
TGGACTCCAGCCTGGGTTGGGGCAAAGAGGGAAATGAGAT
CATGTCCTAACCCTGATCCTCTTGTCCCACAGATATCCAG
AACCCTGACCCTGCCGTGTACCAGCTGAGAGACTCTAAAT
CCAGTGACAAGTCTGTCTGCCTATTCGAATTCGGCTCCGG
AGCCACTAACTTCTCCCTGTTGAAACAGGCTGGCGATGTT
GAAGAAAACCCCGGTCCTATGGCCACCGGCTCTAGAACAA
GCCTGCTGCTCGCTTTTGGCCTGCTCTGCCTCCCATGGCT
CCAAGAAGGATCTGCTGATGCTGGAATCACCCAGAGCCCA
AGATACAAGATCACAGAGACAGGAAGGCAGGTGACCTTGA
TGTGTCACCAGACTTGGAGCCACAGCTATATGTTCTGGTA
TCGACAAGACCTGGGACATGGGCTGAGGCTGATCTATTAC
TCAGCAGCTGCTGATATTACAGATAAAGGAGAAGTCTCCG
ATGGCTATGTTGTCTCCAGATCCAAGACAGAGAATTTCCC
CCTCACTCTGGAGTCAGCTACCCGCTCCCAGACATCTGTG
TATTTCTGCGCCAGCAGTGAGGACAGTTACGAGCAGTACT
TCGGGCCGGGCACCAGGCTCACGGTCACAGAGGACCTGAA
AAACGTGTTCCCTCCAAAAGTGGCCGTGTTCGAGCCTTCT
GAGGCCGAGATCAGCCACACACAGAAAGCCACACTCGTGT
GTCTGGCTACCGGCTTCTACCCCGATCACGTGGAACTGTC
TTGGTGGGTCAACGGCAAAGAGGTGCACAGCGGCGTCAGC
ACAGATCCCCAGCCTCTGAAAGAACAGCCCGCTCTGAACG
ACAGCCGCTACTGCCTGTCTAGCAGACTGAGAGTGTCCGC
CACCTTCTGGCAGAACCCCAGAAACCACTTCAGATGCCAG
GTCCAGTTCTACGGCCTGAGCGAGAACGATGAGTGGACCC
AGGACAGAGCCAAGCCTGTGACACAGATCGTGTCTGCCGA
AGCCTGGGGCAGAGCCGATTGTGGCTTTACCAGCGAGTCA
TACCAGCAGGGCGTGCTGTCTGCCACCATCCTGTATGAGA
TCCTGCTCGGCAAGGCCACACTGTACGCTGTGCTGGTGTC
TGCTCTGGTGCTGATGGCTATGGTCTCCCGGGAGCGCATC
CCCGAGGCCCGGGCCAAGCGGGGCAGCGGCGCCACCAACT
TCAGCCTGCTGAAGCAGGCCGGCGACGTGGAGGAGAACCC
CGGCCCTATGGCCACAGGCAGCAGAACATCTCTGCTGCTG
GCCTTCGGACTGCTGTGTCTGCCTTGGCTGCAAGAGGGTT
CCGCCGCCCAGTCAGTGACCCAGCCTGACATCCACATCAC
TGTCTCTGAAGGAGCCTCACTGGAGTTGAGATGTAACTAT
TCCTATGGGGCAACACCTTATCTCTTCTGGTATGTCCAGT
CCCCCGGCCAAGGCCTCCAGCTGCTCCTGAAGTACTTTTC
AGGAGACACTCTGGTTCAAGGCATTAAAGGCTTTGAGGCT
GAATTTAAGAGGAGTCAATCTTCCTTCAATCTGAGGAAAC
CCTCTGTGCATTGGAGTGATGCTGCTGAGTACTTCTGTGC
TGTGGGTGAATTGGACACAGGCTTTCAGAAACTTGTATTT
GGAACTGGCACCCGACTTCTGGTCAGTCCAAATATTCAGA
ACCCCGATCCTGCCGTGTACCAGCTGAGAGACAGCAAGAG
CAGCGACAAGAGCGTGTGCCTGTTCACCGACTTCGACAGC
CAGACCAACGTGTCCCAGAGCAAGGACAGCGACGTGTACA
TCACCGACAAGACCGTGCTGGACATGCGGAGCATGGACTT
CAAGAGCAACAGCGCCGTGGCCTGGTCCAACAAGAGCGAT
TTCGCCTGCGCCAACGCCTTCAACAACAGCATTATCCCCG
AGGACACATTCTTCCCAAGTCCTGAGAGCAGCTGCGACGT
GAAGCTGGTGGAAAAGAGCTTCGAGACAGACACCAACCTG
AACTTCCAGAACCTGTCCGTGATCGGCTTCCGCATCCTGC
TGCTGAAAGTGGCCGGCTTCAACCTGCTGATGACCCTGAG
ACTGTGGTCCAGCTGATGTGCCTTCTAGTTGCCAGCCATC
TGTTGTTTGCCCCTCCCCCGTGCCTTCCTTGACCCTGGAA
GGTGCCACTCCCACTGTCCTTTCCTAATAAAATGAGGAAA
TTGCATCGCATTGTCTGAGTAGGTGTCATTCTATTCTGGG
GGGTGGGGTGGGGCAGGACAGCAAGGGGGAGGATTGGGAA
GACAATAGCAGGCATGCTGGGGATGCGGTGGGCTCTATGG
CAAGCTTGAACAGAGAAACAGGAGAATATGGGCCAAACAG
GATATCTGTGGTAAGCAGTTCCTGCCCCGGCTCAGGGCCA
AGAACAGTTGGAACAGCAGAATATGGGCCAAACAGGATAT
CTGTGGTAAGCAGTTCCTGCCCCGGCTCAGGGCCAAGAAC
AGATGGTCCCCAGATGCGGTCCCGCCCTCAGCAGTTTCTA
GAGAACCATCAGATGTTTCCAGGGTGCCCCAAGGACCTGA
AATGACCCTGTGCCTTATTTGAACTAACCAATCAGTTCGC
TTCTCGCTTCTGTTCGCGCGCTTCTGCTCCCCGAGCTCTA
TATAAGCAGAGCTCGTTTAGTGAACCGTCAGATCGCCTGG
AGACGCCATCCACGCTGTTTTGACTTCCATAGAAGGGATC
CTCGACTTCTTAACCCAACAGAAGGCTCGAGAAGGTATAT
TGCTGTTGACAGTGAGCGAAAGATGTCAAGATTGAGCCTT
TAGTGAAGCCACAGATGTAAAGGCTCAATCTTGACATCTT
GTGCCTACTGCCTCGGACTTCAAGGGGCTACTTTAGGAGT
CGACGCCACCATGGTGTCCAAGGGCGAAGAGGACAACATG
GCCATCATCAAAGAGTTCATGCGGTTCAAGGTGCACATGG
AAGGCAGCGTGAACGGCCACGAGTTCGAGATTGAAGGCGA
AGGCGAGGGCAGACCTTACGAGGGAACACAGACCGCCAAG
CTGAAAGTGACCAAAGGCGGCCCTCTGCCTTTTGCCTGGG
ACATTCTGAGCCCTCAGTTTATGTACGGCAGCAAGGCCTA
CGTGAAGCACCCCGCCGATATTCCCGACTACCTGAAGCTG
AGCTTCCCCGAGGGCTTCAAGTGGGAGAGAGTGATGAACT
TCGAGGACGGCGGCGTGGTCACCGTGACTCAAGATAGCTC
TCTGCAGGACGGCGAGTTCATCTACAAAGTGAAGCTGCGG
GGCACCAACTTTCCCTCTGATGGCCCCGTGATGCAGAAAA
AGACAATGGGCTGGGAAGCCAGCAGCGAGAGAATGTACCC
TGAAGATGGCGCCCTGAAGGGCGAGATCAAGCAGCGGCTG
AAACTGAAGGATGGCGGCCACTACGACGCCGAAGTGAAAA
CCACCTACAAGGCCAAGAAACCCGTGCAGCTGCCTGGCGC
CTACAACGTGAACATCAAGCTGGACATCACCAGCCACAAC
GAGGACTACACCATCGTGGAACAGTACGAGAGAGCCGAAG
GCAGACACAGCACAGGCGGAATGGACGAGCTGTACAAGCG
GGCTAAGAGAGGCTCTGGAGCCACAAACTTTAGCCTCCTG
AAACAAGCAGGGGATGTGGAAGAGAATCCCGGACCTGCGG
CCGCACCGATTTTGATTCTCAAACAAATGTGTCACAAAGT
AAGGATTCTGATGTGTATATCACAGACAAAACTGTGCTAG
ACATGAGGTCTATGGACTTCAAGAGCAACAGTGCTGTGGC
CTGGAGCAACAAATCTGACTTTGCATGTGCAAACGCCTTC
AACAACAGCATTATTCCAGAAGACACCTTCTTCCCCAGCC
CAGGTAAGGGCAGCTTTGGTGCCTTCGCAGGCTGTTTCCT
TGCTTCAGGAATGGCCAGGTTCTGCCCAGAGCTCTGGTCA
ATGATGTCTAAAACTCCTCTGATTGCTAGCCGCGTTGCTG
GCGTTTTTCCATAGGCTCCGCCCCCCTGACGAGCATCACA
AAAATCGACGCTCAAGTCAGAGGTGGCGAAACCCGACAGG
ACTATAAAGATACCAGGCGTTTCCCCCTGGAAGCTCCCTC
GTGCGCTCTCCTGTTCCGACCCTGCCGCTTACCGGATACC
TGTCCGCCTTTCTCCCTTCGGGAAGCGTGGCGCTTTCTCA
TAGCTCACGCTGTAGGTATCTCAGTTCGGTGTAGGTCGTT
CGCTCCAAGCTGGGCTGTGTGCACGAACCCCCCGTTCAGC
CCGACCGCTGCGCCTTATCCGGTAACTATCGTCTTGAGTC
CAACCCGGTAAGACACGACTTATCGCCACTGGCAGCAGCC
ACTGGTAACAGGATTAGCAGAGCGAGGTATGTAGGCGGTG
CTACAGAGTTCTTGAAGTGGTGGCCTAACTACGGCTACAC
TAGAAGAACAGTATTTGGTATCTGCGCTCTGCTGAAGCCA
GTTACCTTCGGAAAAAGAGTTGGTAGCTCTTGATCCGGCA
AACAAACCACCGCTGGTAGCGGTGGTTTTTTTGTTTGCAA
GCAGCAGATTACGCGCAGAAAAAAAGGATCTCAAGAAGAT
CCTTTGATCTTTAGAAAAACTCATCGAGCATCAAATGAAA
CTGCAATTTATTCATATCAGGATTATCAATACCATATTTT
TGAAAAAGCCGTTTCTGTAATGAAGGAGAAAACTCACCGA
GGCAGTTCCATAGGATGGCAAGATCCTGGTATCGGTCTGC
GATTCCGACTCGTCCAACATCAATACAACCTATTAATTTC
CCCTCGTCAAAAATAAGGTTATCAAGTGAGAAATCACCAT
GAGTGACGACTGAATCCGGTGAGAATGGCAAAAGTTTATG
CATTTCTTTCCAGACTTGTTCAACAGGCCAGCCATTACGC
TCGTCATCAAAATCACTCGCATCAACCAAACCGTTATTCA
TTCGTGATTGCGCCTGAGCCAGACGAAATACGCGATCGCT
GTTAAAAGGACAATTACAAACAGGAATCGAATGCAACCGG
CGCAGGAACACTGCCAGCGCATCAACAATATTTTCACCTG
AATCAGGATATTCTTCTAATACCTGGAATGCTGTTTTTCC
GGGGATCGCAGTGGTGAGTAACCATGCATCATCAGGAGTA
CGGATAAAATGCTTGATGGTCGGAAGAGGCATAAATTCCG
TCAGCCAGTTTAGTCTGACCATCTCATCTGTAACATCATT
GGCAACGCTACCTTTGCCATGTTTCAGAAACAACTCTGGC
GCATCGGGCTTCCCATACAAGCGATAGATTGTCGCACCTG
ATTGCCCGACATTATCGCGAGCCCATTTATACCCATATAA
ATCAGCATCCATGTTGGAATTTAATCGCGGCCTCGACGTT
TCCCGTTGAATATGGCTCATAACACCCCTTGTATTACTGT
TTATGTAAGCAGACAGTTTTATTGTTCATGATGATATATT
TTTATCTTGTGCAATGTAACATCAGAGATTTTGAGACAC

In certain embodiments, the Secondary Promoter Construct comprises nucleotides 1 to 2887 of SEQ ID NO: 60. In certain embodiments, the Secondary Promoter Construct comprises nucleotides 3837 to 5691 of SEQ ID NO: 60. In certain embodiments, the Secondary Promoter Construct comprises nucleotides 1 to 2887 and nucleotides 3837 to 5691 of SEQ ID NO: 60. SEQ ID NO: 60 is provided below.

[SEQ ID NO: 60]
GGTACCTCATGGCCTCTTGGCCAAGATTGATAGCTTGTGC
CTGTCCCTGAGTCCCAGTCCATCACGAGCAGCTGGTTTCT
AAGATGCTATTTCCCGTATAAAGCATGAGACCGTGACTTG
CCAGCCCCACAGAGCCCCGCCCTTGTCCATCACTGGCATC
TGGACTCCAGCCTGGGTTGGGGCAAAGAGGGAAATGAGAT
CATGTCCTAACCCTGATCCTCTTGTCCCACAGATATCCAG
AACCCTGACCCTGCCGTGTACCAGCTGAGAGACTCTAAAT
CCAGTGACAAGTCTGTCTGCCTATTCGAATTCGGCTCCGG
AGCCACTAACTTCTCCCTGTTGAAACAGGCTGGCGATGTT
GAAGAAAACCCCGGTCCTATGGCCACCGGCTCTAGAACAA
GCCTGCTGCTCGCTTTTGGCCTGCTCTGCCTCCCATGGCT
CCAAGAAGGATCTGCTGATGCTGGAATCACCCAGAGCCCA
AGATACAAGATCACAGAGACAGGAAGGCAGGTGACCTTGA
TGTGTCACCAGACTTGGAGCCACAGCTATATGTTCTGGTA
TCGACAAGACCTGGGACATGGGCTGAGGCTGATCTATTAC
TCAGCAGCTGCTGATATTACAGATAAAGGAGAAGTCTCCG
ATGGCTATGTTGTCTCCAGATCCAAGACAGAGAATTTCCC
CCTCACTCTGGAGTCAGCTACCCGCTCCCAGACATCTGTG
TATTTCTGCGCCAGCAGTGAGGACAGTTACGAGCAGTACT
TCGGGCCGGGCACCAGGCTCACGGTCACAGAGGACCTGAA
AAACGTGTTCCCTCCAAAAGTGGCCGTGTTCGAGCCTTCT
GAGGCCGAGATCAGCCACACACAGAAAGCCACACTCGTGT
GTCTGGCTACCGGCTTCTACCCCGATCACGTGGAACTGTC
TTGGTGGGTCAACGGCAAAGAGGTGCACAGCGGCGTCAGC
ACAGATCCCCAGCCTCTGAAAGAACAGCCCGCTCTGAACG
ACAGCCGCTACTGCCTGTCTAGCAGACTGAGAGTGTCCGC
CACCTTCTGGCAGAACCCCAGAAACCACTTCAGATGCCAG
GTCCAGTTCTACGGCCTGAGCGAGAACGATGAGTGGACCC
AGGACAGAGCCAAGCCTGTGACACAGATCGTGTCTGCCGA
AGCCTGGGGCAGAGCCGATTGTGGCTTTACCAGCGAGTCA
TACCAGCAGGGCGTGCTGTCTGCCACCATCCTGTATGAGA
TCCTGCTCGGCAAGGCCACACTGTACGCTGTGCTGGTGTC
TGCTCTGGTGCTGATGGCTATGGTCTCCCGGGAGCGCATC
CCCGAGGCCCGGGCCAAGCGGGGCAGCGGCGCCACCAACT
TCAGCCTGCTGAAGCAGGCCGGCGACGTGGAGGAGAACCC
CGGCCCTATGGCCACAGGCAGCAGAACATCTCTGCTGCTG
GCCTTCGGACTGCTGTGTCTGCCTTGGCTGCAAGAGGGTT
CCGCCGCCCAGTCAGTGACCCAGCCTGACATCCACATCAC
TGTCTCTGAAGGAGCCTCACTGGAGTTGAGATGTAACTAT
TCCTATGGGGCAACACCTTATCTCTTCTGGTATGTCCAGT
CCCCCGGCCAAGGCCTCCAGCTGCTCCTGAAGTACTTTTC
AGGAGACACTCTGGTTCAAGGCATTAAAGGCTTTGAGGCT
GAATTTAAGAGGAGTCAATCTTCCTTCAATCTGAGGAAAC
CCTCTGTGCATTGGAGTGATGCTGCTGAGTACTTCTGTGC
TGTGGGTGAATTGGACACAGGCTTTCAGAAACTTGTATTT
GGAACTGGCACCCGACTTCTGGTCAGTCCAAATATTCAGA
ACCCCGATCCTGCCGTGTACCAGCTGAGAGACAGCAAGAG
CAGCGACAAGAGCGTGTGCCTGTTCACCGACTTCGACAGC
CAGACCAACGTGTCCCAGAGCAAGGACAGCGACGTGTACA
TCACCGACAAGACCGTGCTGGACATGCGGAGCATGGACTT
CAAGAGCAACAGCGCCGTGGCCTGGTCCAACAAGAGCGAT
TTCGCCTGCGCCAACGCCTTCAACAACAGCATTATCCCCG
AGGACACATTCTTCCCAAGTCCTGAGAGCAGCTGCGACGT
GAAGCTGGTGGAAAAGAGCTTCGAGACAGACACCAACCTG
AACTTCCAGAACCTGTCCGTGATCGGCTTCCGCATCCTGC
TGCTGAAAGTGGCCGGCTTCAACCTGCTGATGACCCTGAG
ACTGTGGTCCAGCTGATGTGCCTTCTAGTTGCCAGCCATC
TGTTGTTTGCCCCTCCCCCGTGCCTTCCTTGACCCTGGAA
GGTGCCACTCCCACTGTCCTTTCCTAATAAAATGAGGAAA
TTGCATCGCATTGTCTGAGTAGGTGTCATTCTATTCTGGG
GGGTGGGGTGGGGCAGGACAGCAAGGGGGAGGATTGGGAA
GACAATAGCAGGCATGCTGGGGATGCGGTGGGCTCTATGG
CAAGCTTGAACAGAGAAACAGGAGAATATGGGCCAAACAG
GATATCTGTGGTAAGCAGTTCCTGCCCCGGCTCAGGGCCA
AGAACAGTTGGAACAGCAGAATATGGGCCAAACAGGATAT
CTGTGGTAAGCAGTTCCTGCCCCGGCTCAGGGCCAAGAAC
AGATGGTCCCCAGATGCGGTCCCGCCCTCAGCAGTTTCTA
GAGAACCATCAGATGTTTCCAGGGTGCCCCAAGGACCTGA
AATGACCCTGTGCCTTATTTGAACTAACCAATCAGTTCGC
TTCTCGCTTCTGTTCGCGCGCTTCTGCTCCCCGAGCTCTA
TATAAGCAGAGCTCGTTTAGTGAACCGTCAGATCGCCTGG
AGACGCCATCCACGCTGTTTTGACTTCCATAGAAGGGATC
CGCCACCATGGTGTCCAAGGGCGAAGAGGACAACATGGCC
ATCATCAAAGAGTTCATGCGGTTCAAGGTGCACATGGAAG
GCAGCGTGAACGGCCACGAGTTCGAGATTGAAGGCGAAGG
CGAGGGCAGACCTTACGAGGGAACACAGACCGCCAAGCTG
AAAGTGACCAAAGGCGGCCCTCTGCCTTTTGCCTGGGACA
TTCTGAGCCCTCAGTTTATGTACGGCAGCAAGGCCTACGT
GAAGCACCCCGCCGATATTCCCGACTACCTGAAGCTGAGC
TTCCCCGAGGGCTTCAAGTGGGAGAGAGTGATGAACTTCG
AGGACGGCGGCGTGGTCACCGTGACTCAAGATAGCTCTCT
GCAGGACGGCGAGTTCATCTACAAAGTGAAGCTGCGGGGC
ACCAACTTTCCCTCTGATGGCCCCGTGATGCAGAAAAAGA
CAATGGGCTGGGAAGCCAGCAGCGAGAGAATGTACCCTGA
AGATGGCGCCCTGAAGGGCGAGATCAAGCAGCGGCTGAAA
CTGAAGGATGGCGGCCACTACGACGCCGAAGTGAAAACCA
CCTACAAGGCCAAGAAACCCGTGCAGCTGCCTGGCGCCTA
CAACGTGAACATCAAGCTGGACATCACCAGCCACAACGAG
GACTACACCATCGTGGAACAGTACGAGAGAGCCGAAGGCA
GACACAGCACAGGCGGAATGGACGAGCTGTACAAGCGGGC
TAAGAGAGGCTCTGGAGCCACAAACTTTAGCCTCCTGAAA
CAAGCAGGGGATGTGGAAGAGAATCCCGGACCTACTAGTT
CGACTTCTTAACCCAACAGAAGGCTCGAGAAGGTATATTG
CTGTTGACAGTGAGCGAAAGATGTCAAGATTGAGCCTTTA
GTGAAGCCACAGATGTAAAGGCTCAATCTTGACATCTTGT
GCCTACTGCCTCGGACTTCAAGGGGCTACTTTAGGAGTCG
ACTGCGATGCGGCCGCACCGATTTTGATTCTCAAACAAAT
GTGTCACAAAGTAAGGATTCTGATGTGTATATCACAGACA
AAACTGTGCTAGACATGAGGTCTATGGACTTCAAGAGCAA
CAGTGCTGTGGCCTGGAGCAACAAATCTGACTTTGCATGT
GCAAACGCCTTCAACAACAGCATTATTCCAGAAGACACCT
TCTTCCCCAGCCCAGGTAAGGGCAGCTTTGGTGCCTTCGC
AGGCTGTTTCCTTGCTTCAGGAATGGCCAGGTTCTGCCCA
GAGCTCTGGTCAATGATGTCTAAAACTCCTCTGATTGCTA
GCCGCGTTGCTGGCGTTTTTCCATAGGCTCCGCCCCCCTG
ACGAGCATCACAAAAATCGACGCTCAAGTCAGAGGTGGCG
AAACCCGACAGGACTATAAAGATACCAGGCGTTTCCCCCT
GGAAGCTCCCTCGTGCGCTCTCCTGTTCCGACCCTGCCGC
TTACCGGATACCTGTCCGCCTTTCTCCCTTCGGGAAGCGT
GGCGCTTTCTCATAGCTCACGCTGTAGGTATCTCAGTTCG
GTGTAGGTCGTTCGCTCCAAGCTGGGCTGTGTGCACGAAC
CCCCCGTTCAGCCCGACCGCTGCGCCTTATCCGGTAACTA
TCGTCTTGAGTCCAACCCGGTAAGACACGACTTATCGCCA
CTGGCAGCAGCCACTGGTAACAGGATTAGCAGAGCGAGGT
ATGTAGGCGGTGCTACAGAGTTCTTGAAGTGGTGGCCTAA
CTACGGCTACACTAGAAGAACAGTATTTGGTATCTGCGCT
CTGCTGAAGCCAGTTACCTTCGGAAAAAGAGTTGGTAGCT
CTTGATCCGGCAAACAAACCACCGCTGGTAGCGGTGGTTT
TTTTGTTTGCAAGCAGCAGATTACGCGCAGAAAAAAAGGA
TCTCAAGAAGATCCTTTGATCTTTAGAAAAACTCATCGAG
CATCAAATGAAACTGCAATTTATTCATATCAGGATTATCA
ATACCATATTTTTGAAAAAGCCGTTTCTGTAATGAAGGAG
AAAACTCACCGAGGCAGTTCCATAGGATGGCAAGATCCTG
GTATCGGTCTGCGATTCCGACTCGTCCAACATCAATACAA
CCTATTAATTTCCCCTCGTCAAAAATAAGGTTATCAAGTG
AGAAATCACCATGAGTGACGACTGAATCCGGTGAGAATGG
CAAAAGTTTATGCATTTCTTTCCAGACTTGTTCAACAGGC
CAGCCATTACGCTCGTCATCAAAATCACTCGCATCAACCA
AACCGTTATTCATTCGTGATTGCGCCTGAGCCAGACGAAA
TACGCGATCGCTGTTAAAAGGACAATTACAAACAGGAATC
GAATGCAACCGGCGCAGGAACACTGCCAGCGCATCAACAA
TATTTTCACCTGAATCAGGATATTCTTCTAATACCTGGAA
TGCTGTTTTTCCGGGGATCGCAGTGGTGAGTAACCATGCA
TCATCAGGAGTACGGATAAAATGCTTGATGGTCGGAAGAG
GCATAAATTCCGTCAGCCAGTTTAGTCTGACCATCTCATC
TGTAACATCATTGGCAACGCTACCTTTGCCATGTTTCAGA
AACAACTCTGGCGCATCGGGCTTCCCATACAAGCGATAGA
TTGTCGCACCTGATTGCCCGACATTATCGCGAGCCCATTT
ATACCCATATAAATCAGCATCCATGTTGGAATTTAATCGC
GGCCTCGACGTTTCCCGTTGAATATGGCTCATAACACCCC
TTGTATTACTGTTTATGTAAGCAGACAGTTTTATTGTTCA
TGATGATATATTTTTATCTTGTGCAATGTAACATCAGAGA
TTTTGAGACAC

In certain embodiments, the Secondary Promoter Construct comprises nucleotides 1 to 3007 of SEQ ID NO: 61. In certain embodiments, the Secondary Promoter Construct comprises nucleotides 3165 to 5134 of SEQ ID NO: 61. In certain embodiments, the Secondary Promoter Construct comprises nucleotides 1 to 3007 and nucleotides 3165 to 5134 of SEQ ID NO: 61. SEQ ID NO: 61 is provided below.

[SEQ ID NO: 61]
GGTACCTCATGGCCTCTTGGCCAAGATTGATAGCTTGTGC
CTGTCCCTGAGTCCCAGTCCATCACGAGCAGCTGGTTTCT
AAGATGCTATTTCCCGTATAAAGCATGAGACCGTGACTTG
CCAGCCCCACAGAGCCCCGCCCTTGTCCATCACTGGCATC
TGGACTCCAGCCTGGGTTGGGGCAAAGAGGGAAATGAGAT
CATGTCCTAACCCTGATCCTCTTGTCCCACAGATATCCAG
AACCCTGACCCTGCCGTGTACCAGCTGAGAGACTCTAAAT
CCAGTGACAAGTCTGTCTGCCTATTCGAATTCGGCTCCGG
AGCCACTAACTTCTCCCTGTTGAAACAGGCTGGCGATGTT
GAAGAAAACCCCGGTCCTATGGCCACCGGCTCTAGAACAA
GCCTGCTGCTCGCTTTTGGCCTGCTCTGCCTCCCATGGCT
CCAAGAAGGATCTGCTGATGCTGGAATCACCCAGAGCCCA
AGATACAAGATCACAGAGACAGGAAGGCAGGTGACCTTGA
TGTGTCACCAGACTTGGAGCCACAGCTATATGTTCTGGTA
TCGACAAGACCTGGGACATGGGCTGAGGCTGATCTATTAC
TCAGCAGCTGCTGATATTACAGATAAAGGAGAAGTCTCCG
ATGGCTATGTTGTCTCCAGATCCAAGACAGAGAATTTCCC
CCTCACTCTGGAGTCAGCTACCCGCTCCCAGACATCTGTG
TATTTCTGCGCCAGCAGTGAGGACAGTTACGAGCAGTACT
TCGGGCCGGGCACCAGGCTCACGGTCACAGAGGACCTGAA
AAACGTGTTCCCTCCAAAAGTGGCCGTGTTCGAGCCTTCT
GAGGCCGAGATCAGCCACACACAGAAAGCCACACTCGTGT
GTCTGGCTACCGGCTTCTACCCCGATCACGTGGAACTGTC
TTGGTGGGTCAACGGCAAAGAGGTGCACAGCGGCGTCAGC
ACAGATCCCCAGCCTCTGAAAGAACAGCCCGCTCTGAACG
ACAGCCGCTACTGCCTGTCTAGCAGACTGAGAGTGTCCGC
CACCTTCTGGCAGAACCCCAGAAACCACTTCAGATGCCAG
GTCCAGTTCTACGGCCTGAGCGAGAACGATGAGTGGACCC
AGGACAGAGCCAAGCCTGTGACACAGATCGTGTCTGCCGA
AGCCTGGGGCAGAGCCGATTGTGGCTTTACCAGCGAGTCA
TACCAGCAGGGCGTGCTGTCTGCCACCATCCTGTATGAGA
TCCTGCTCGGCAAGGCCACACTGTACGCTGTGCTGGTGTC
TGCTCTGGTGCTGATGGCTATGGTCTCCCGGGAGCGCATC
CCCGAGGCCCGGGCCAAGCGGGGCAGCGGCGCCACCAACT
TCAGCCTGCTGAAGCAGGCCGGCGACGTGGAGGAGAACCC
CGGCCCTATGGCCACAGGCAGCAGAACATCTCTGCTGCTG
GCCTTCGGACTGCTGTGTCTGCCTTGGCTGCAAGAGGGTT
CCGCCGCCCAGTCAGTGACCCAGCCTGACATCCACATCAC
TGTCTCTGAAGGAGCCTCACTGGAGTTGAGATGTAACTAT
TCCTATGGGGCAACACCTTATCTCTTCTGGTATGTCCAGT
CCCCCGGCCAAGGCCTCCAGCTGCTCCTGAAGTACTTTTC
AGGAGACACTCTGGTTCAAGGCATTAAAGGCTTTGAGGCT
GAATTTAAGAGGAGTCAATCTTCCTTCAATCTGAGGAAAC
CCTCTGTGCATTGGAGTGATGCTGCTGAGTACTTCTGTGC
TGTGGGTGAATTGGACACAGGCTTTCAGAAACTTGTATTT
GGAACTGGCACCCGACTTCTGGTCAGTCCAAATATTCAGA
ACCCCGATCCTGCCGTGTACCAGCTGAGAGACAGCAAGAG
CAGCGACAAGAGCGTGTGCCTGTTCACCGACTTCGACAGC
CAGACCAACGTGTCCCAGAGCAAGGACAGCGACGTGTACA
TCACCGACAAGACCGTGCTGGACATGCGGAGCATGGACTT
CAAGAGCAACAGCGCCGTGGCCTGGTCCAACAAGAGCGAT
TTCGCCTGCGCCAACGCCTTCAACAACAGCATTATCCCCG
AGGACACATTCTTCCCAAGTCCTGAGAGCAGCTGCGACGT
GAAGCTGGTGGAAAAGAGCTTCGAGACAGACACCAACCTG
AACTTCCAGAACCTGTCCGTGATCGGCTTCCGCATCCTGC
TGCTGAAAGTGGCCGGCTTCAACCTGCTGATGACCCTGAG
ACTGTGGTCCAGCTGATGTGCCTTCTAGTTGCCAGCCATC
TGTTGTTTGCCCCTCCCCCGTGCCTTCCTTGACCCTGGAA
GGTGCCACTCCCACTGTCCTTTCCTAATAAAATGAGGAAA
TTGCATCGCATTGTCTGAGTAGGTGTCATTCTATTCTGGG
GGGTGGGGTGGGGCAGGACAGCAAGGGGGAGGATTGGGAA
GACAATAGCAGGCATGCTGGGGATGCGGTGGGCTCTATGG
CAAGCTTGAACAGAGAAACAGGAGAATATGGGCCAAACAG
GATATCTGTGGTAAGCAGTTCCTGCCCCGGCTCAGGGCCA
AGAACAGTTGGAACAGCAGAATATGGGCCAAACAGGATAT
CTGTGGTAAGCAGTTCCTGCCCCGGCTCAGGGCCAAGAAC
AGATGGTCCCCAGATGCGGTCCCGCCCTCAGCAGTTTCTA
GAGAACCATCAGATGTTTCCAGGGTGCCCCAAGGACCTGA
AATGACCCTGTGCCTTATTTGAACTAACCAATCAGTTCGC
TTCTCGCTTCTGTTCGCGCGCTTCTGCTCCCCGAGCTCTA
TATAAGCAGAGCTCGTTTAGTGAACCGTCAGATCGCCTGG
AGACGCCATCCACGCTGTTTTGACTTCCATAGAAGGGATC
CCTGACTACATCACAGCAGCATACGTGGAGATGAGGCGAG
AGGCTTGGGCTAGTAAGGATGCCACCTATACTTCTGCCCG
GACCCTGCTGGCTATCCTGCGCCTTTCCACTGCTCTGGTA
AGTGCCCTCGACTTCTTAACCCAACAGAAGGCTCGAGAAG
GTATATTGCTGTTGACAGTGAGCGAAAGATGTCAAGATTG
AGCCTTTAGTGAAGCCACAGATGTAAAGGCTCAATCTTGA
CATCTTGTGCCTACTGCCTCGGACTTCAAGGGGCTACTTT
AGGATTCCCCTTAGGCACGTCTGAGAATGGTGGATGTGGT
GGAGAAAGAAGATGTGAATGAAGCCATCAGGCTAATGGAG
ATGTCAAAGGACTCTCTTCTAGGAGACAAGGGGCAGACAG
TCGACTGCGATGCGGCCGCACCGATTTTGATTCTCAAACA
AATGTGTCACAAAGTAAGGATTCTGATGTGTATATCACAG
ACAAAACTGTGCTAGACATGAGGTCTATGGACTTCAAGAG
CAACAGTGCTGTGGCCTGGAGCAACAAATCTGACTTTGCA
TGTGCAAACGCCTTCAACAACAGCATTATTCCAGAAGACA
CCTTCTTCCCCAGCCCAGGTAAGGGCAGCTTTGGTGCCTT
CGCAGGCTGTTTCCTTGCTTCAGGAATGGCCAGGTTCTGC
CCAGAGCTCTGGTCAATGATGTCTAAAACTCCTCTGATTG
CTAGCCGCGTTGCTGGCGTTTTTCCATAGGCTCCGCCCCC
CTGACGAGCATCACAAAAATCGACGCTCAAGTCAGAGGTG
GCGAAACCCGACAGGACTATAAAGATACCAGGCGTTTCCC
CCTGGAAGCTCCCTCGTGCGCTCTCCTGTTCCGACCCTGC
CGCTTACCGGATACCTGTCCGCCTTTCTCCCTTCGGGAAG
CGTGGCGCTTTCTCATAGCTCACGCTGTAGGTATCTCAGT
TCGGTGTAGGTCGTTCGCTCCAAGCTGGGCTGTGTGCACG
AACCCCCCGTTCAGCCCGACCGCTGCGCCTTATCCGGTAA
CTATCGTCTTGAGTCCAACCCGGTAAGACACGACTTATCG
CCACTGGCAGCAGCCACTGGTAACAGGATTAGCAGAGCGA
GGTATGTAGGCGGTGCTACAGAGTTCTTGAAGTGGTGGCC
TAACTACGGCTACACTAGAAGAACAGTATTTGGTATCTGC
GCTCTGCTGAAGCCAGTTACCTTCGGAAAAAGAGTTGGTA
GCTCTTGATCCGGCAAACAAACCACCGCTGGTAGCGGTGG
TTTTTTTGTTTGCAAGCAGCAGATTACGCGCAGAAAAAAA
GGATCTCAAGAAGATCCTTTGATCTTTAGAAAAACTCATC
GAGCATCAAATGAAACTGCAATTTATTCATATCAGGATTA
TCAATACCATATTTTTGAAAAAGCCGTTTCTGTAATGAAG
GAGAAAACTCACCGAGGCAGTTCCATAGGATGGCAAGATC
CTGGTATCGGTCTGCGATTCCGACTCGTCCAACATCAATA
CAACCTATTAATTTCCCCTCGTCAAAAATAAGGTTATCAA
GTGAGAAATCACCATGAGTGACGACTGAATCCGGTGAGAA
TGGCAAAAGTTTATGCATTTCTTTCCAGACTTGTTCAACA
GGCCAGCCATTACGCTCGTCATCAAAATCACTCGCATCAA
CCAAACCGTTATTCATTCGTGATTGCGCCTGAGCCAGACG
AAATACGCGATCGCTGTTAAAAGGACAATTACAAACAGGA
ATCGAATGCAACCGGCGCAGGAACACTGCCAGCGCATCAA
CAATATTTTCACCTGAATCAGGATATTCTTCTAATACCTG
GAATGCTGTTTTTCCGGGGATCGCAGTGGTGAGTAACCAT
GCATCATCAGGAGTACGGATAAAATGCTTGATGGTCGGAA
GAGGCATAAATTCCGTCAGCCAGTTTAGTCTGACCATCTC
ATCTGTAACATCATTGGCAACGCTACCTTTGCCATGTTTC
AGAAACAACTCTGGCGCATCGGGCTTCCCATACAAGCGAT
AGATTGTCGCACCTGATTGCCCGACATTATCGCGAGCCCA
TTTATACCCATATAAATCAGCATCCATGTTGGAATTTAAT
CGCGGCCTCGACGTTTCCCGTTGAATATGGCTCATAACAC
CCCTTGTATTACTGTTTATGTAAGCAGACAGTTTTATTGT
TCATGATGATATATTTTTATCTTGTGCAATGTAACATCAG
AGATTTTGAGACAC

In certain embodiments, the Secondary Promoter Construct comprises nucleotides 1 to 312 of SEQ TD NO: 62. In certain embodiments, the Secondary Promoter Construct comprises nucleotides 470 to 4001 of SEQ TD NO: 62. In certain embodiments, the Secondary Promoter Construct comprises nucleotides 1 to 312 and nucleotides 470 to 4001 of SEQ ID NO: 62. SEQ ID NO: 62 is provided below.

[SEQ ID NO: 62]
GGTACCAGAATAAGCAGTATTATTAAGTAGCCCTGCATTT
CAGGTTTCCTTGAGTGGCAGGCCAGGCCTGGCCGTGAACG
TTCACTGAAATCATGGCCTCTTGGCCAAGATTGATAGCTT
GTGCCTGTCCCTGAGTCCCAGTCCATCACGAGCAGCTGGT
TTCTAAGATGCTATTTCCCGTATAAAGCATGAGACCGTGA
CTTGCCAGCCCCACAGAGCCCCGCCCTTGTCCATCACTGG
CATCTGGACTCCAGCCTGGGTTGGGGCAAAGAGGGAAATG
AGATCATGTCCTAACCCTGATCCTCTGAATTCTCGACTTC
TTAACCCAACAGAAGGCTCGAGAAGGTATATTGCTGTTGA
CAGTGAGCGAAAGATGTCAAGATTGAGCCTTTAGTGAAGC
CACAGATGTAAAGGCTCAATCTTGACATCTTGTGCCTACT
GCCTCGGACTTCAAGGGGCTACTTTAGGAGTCGACTGTCC
CACAGATATCCAGAACCCCGACCCCGCCGTGTACCAGCTG
CGGGACAGCAAGAGCAGCGACAAGAGCGTGTGCCTGTTGG
CTCCGGAGCCACTAACTTCTCCCTGTTGAAACAGGCTGGC
GATGTTGAAGAAAACCCCGGTCCTATGGCCACCGGCTCTA
GAACAAGCCTGCTGCTCGCTTTTGGCCTGCTCTGCCTCCC
ATGGCTCCAAGAAGGATCTGCTGATGCTGGAATCACCCAG
AGCCCAAGATACAAGATCACAGAGACAGGAAGGCAGGTGA
CCTTGATGTGTCACCAGACTTGGAGCCACAGCTATATGTT
CTGGTATCGACAAGACCTGGGACATGGGCTGAGGCTGATC
TATTACTCAGCAGCTGCTGATATTACAGATAAAGGAGAAG
TCTCCGATGGCTATGTTGTCTCCAGATCCAAGACAGAGAA
TTTCCCCCTCACTCTGGAGTCAGCTACCCGCTCCCAGACA
TCTGTGTATTTCTGCGCCAGCAGTGAGGACAGTTACGAGC
AGTACTTCGGGCCGGGCACCAGGCTCACGGTCACAGAGGA
CCTGAAAAACGTGTTCCCTCCAAAAGTGGCCGTGTTCGAG
CCTTCTGAGGCCGAGATCAGCCACACACAGAAAGCCACAC
TCGTGTGTCTGGCTACCGGCTTCTACCCCGATCACGTGGA
ACTGTCTTGGTGGGTCAACGGCAAAGAGGTGCACAGCGGC
GTCAGCACAGATCCCCAGCCTCTGAAAGAACAGCCCGCTC
TGAACGACAGCCGCTACTGCCTGTCTAGCAGACTGAGAGT
GTCCGCCACCTTCTGGCAGAACCCCAGAAACCACTTCAGA
TGCCAGGTCCAGTTCTACGGCCTGAGCGAGAACGATGAGT
GGACCCAGGACAGAGCCAAGCCTGTGACACAGATCGTGTC
TGCCGAAGCCTGGGGCAGAGCCGATTGTGGCTTTACCAGC
GAGTCATACCAGCAGGGCGTGCTGTCTGCCACCATCCTGT
ATGAGATCCTGCTCGGCAAGGCCACACTGTACGCTGTGCT
GGTGTCTGCTCTGGTGCTGATGGCTATGGTCTCCCGGGAG
CGCATCCCCGAGGCCCGGGCCAAGCGGGGCAGCGGCGCCA
CCAACTTCAGCCTGCTGAAGCAGGCCGGCGACGTGGAGGA
GAACCCCGGCCCTATGGCCACAGGCAGCAGAACATCTCTG
CTGCTGGCCTTCGGACTGCTGTGTCTGCCTTGGCTGCAAG
AGGGTTCCGCCGCCCAGTCAGTGACCCAGCCTGACATCCA
CATCACTGTCTCTGAAGGAGCCTCACTGGAGTTGAGATGT
AACTATTCCTATGGGGCAACACCTTATCTCTTCTGGTATG
TCCAGTCCCCCGGCCAAGGCCTCCAGCTGCTCCTGAAGTA
CTTTTCAGGAGACACTCTGGTTCAAGGCATTAAAGGCTTT
GAGGCTGAATTTAAGAGGAGTCAATCTTCCTTCAATCTGA
GGAAACCCTCTGTGCATTGGAGTGATGCTGCTGAGTACTT
CTGTGCTGTGGGTGAATTGGACACAGGCTTTCAGAAACTT
GTATTTGGAACTGGCACCCGACTTCTGGTCAGTCCAAATA
TTCAGAACCCCGATCCTGCTGTGTATCAGCTGCGCGACAG
CAAGAGCAGCGACAAGAGCGTGTGTTTGTTCACCGATTTT
GATTCTCAAACAAATGTGTCACAAAGTAAGGATTCTGATG
TGTATATCACAGACAAAACTGTGCTAGACATGAGGTCTAT
GGACTTCAAGAGCAACAGTGCTGTGGCCTGGAGCAACAAA
TCTGACTTTGCATGTGCAAACGCCTTCAACAACAGCATTA
TTCCAGAAGACACCTTCTTCCCCAGCCCAGGTAAGGGCAG
CTTTGGTGCCTTCGCAGGCTGTTTCCTTGCTTCAGGAATG
GCCAGGTTCTGCCCAGAGCTCTGGTCAATGATGTCTAAAA
CTCCTCTGATTGGCGGCCGCTGCGATGCTAGCCGCGTTGC
TGGCGTTTTTCCATAGGCTCCGCCCCCCTGACGAGCATCA
CAAAAATCGACGCTCAAGTCAGAGGTGGCGAAACCCGACA
GGACTATAAAGATACCAGGCGTTTCCCCCTGGAAGCTCCC
TCGTGCGCTCTCCTGTTCCGACCCTGCCGCTTACCGGATA
CCTGTCCGCCTTTCTCCCTTCGGGAAGCGTGGCGCTTTCT
CATAGCTCACGCTGTAGGTATCTCAGTTCGGTGTAGGTCG
TTCGCTCCAAGCTGGGCTGTGTGCACGAACCCCCCGTTCA
GCCCGACCGCTGCGCCTTATCCGGTAACTATCGTCTTGAG
TCCAACCCGGTAAGACACGACTTATCGCCACTGGCAGCAG
CCACTGGTAACAGGATTAGCAGAGCGAGGTATGTAGGCGG
TGCTACAGAGTTCTTGAAGTGGTGGCCTAACTACGGCTAC
ACTAGAAGAACAGTATTTGGTATCTGCGCTCTGCTGAAGC
CAGTTACCTTCGGAAAAAGAGTTGGTAGCTCTTGATCCGG
CAAACAAACCACCGCTGGTAGCGGTGGTTTTTTTGTTTGC
AAGCAGCAGATTACGCGCAGAAAAAAAGGATCTCAAGAAG
ATCCTTTGATCTTTAGAAAAACTCATCGAGCATCAAATGA
AACTGCAATTTATTCATATCAGGATTATCAATACCATATT
TTTGAAAAAGCCGTTTCTGTAATGAAGGAGAAAACTCACC
GAGGCAGTTCCATAGGATGGCAAGATCCTGGTATCGGTCT
GCGATTCCGACTCGTCCAACATCAATACAACCTATTAATT
TCCCCTCGTCAAAAATAAGGTTATCAAGTGAGAAATCACC
ATGAGTGACGACTGAATCCGGTGAGAATGGCAAAAGTTTA
TGCATTTCTTTCCAGACTTGTTCAACAGGCCAGCCATTAC
GCTCGTCATCAAAATCACTCGCATCAACCAAACCGTTATT
CATTCGTGATTGCGCCTGAGCCAGACGAAATACGCGATCG
CTGTTAAAAGGACAATTACAAACAGGAATCGAATGCAACC
GGCGCAGGAACACTGCCAGCGCATCAACAATATTTTCACC
TGAATCAGGATATTCTTCTAATACCTGGAATGCTGTTTTT
CCGGGGATCGCAGTGGTGAGTAACCATGCATCATCAGGAG
TACGGATAAAATGCTTGATGGTCGGAAGAGGCATAAATTC
CGTCAGCCAGTTTAGTCTGACCATCTCATCTGTAACATCA
TTGGCAACGCTACCTTTGCCATGTTTCAGAAACAACTCTG
GCGCATCGGGCTTCCCATACAAGCGATAGATTGTCGCACC
TGATTGCCCGACATTATCGCGAGCCCATTTATACCCATAT
AAATCAGCATCCATGTTGGAATTTAATCGCGGCCTCGACG
TTTCCCGTTGAATATGGCTCATAACACCCCTTGTATTACT
GTTTATGTAAGCAGACAGTTTTATTGTTCATGATGATATA
TTTTTATCTTGTGCAATGTAACATCAGAGATTTTGAGACA
C

In certain embodiments, the Secondary Promoter Construct comprises nucleotides 1 to 2881 of SEQ ID NO: 63. In certain embodiments, the Secondary Promoter Construct comprises nucleotides 3039 to 4893 of SEQ ID NO: 63. In certain embodiments, the Secondary Promoter Construct comprises nucleotides 1 to 2881 and nucleotides 3039 to 4893 of SEQ ID NO: 63. SEQ ID NO: 63 is provided below.

[SEQ ID NO: 63]
GGTACCTCATGGCCTCTTGGCCAAGATTGATAGCTTGTGC
CTGTCCCTGAGTCCCAGTCCATCACGAGCAGCTGGTTTCT
AAGATGCTATTTCCCGTATAAAGCATGAGACCGTGACTTG
CCAGCCCCACAGAGCCCCGCCCTTGTCCATCACTGGCATC
TGGACTCCAGCCTGGGTTGGGGCAAAGAGGGAAATGAGAT
CATGTCCTAACCCTGATCCTCTTGTCCCACAGATATCCAG
AACCCTGACCCTGCCGTGTACCAGCTGAGAGACTCTAAAT
CCAGTGACAAGTCTGTCTGCCTATTCGAATTCGGCTCCGG
AGCCACTAACTTCTCCCTGTTGAAACAGGCTGGCGATGTT
GAAGAAAACCCCGGTCCTATGGCCACCGGCTCTAGAACAA
GCCTGCTGCTCGCTTTTGGCCTGCTCTGCCTCCCATGGCT
CCAAGAAGGATCTGCTGATGCTGGAATCACCCAGAGCCCA
AGATACAAGATCACAGAGACAGGAAGGCAGGTGACCTTGA
TGTGTCACCAGACTTGGAGCCACAGCTATATGTTCTGGTA
TCGACAAGACCTGGGACATGGGCTGAGGCTGATCTATTAC
TCAGCAGCTGCTGATATTACAGATAAAGGAGAAGTCTCCG
ATGGCTATGTTGTCTCCAGATCCAAGACAGAGAATTTCCC
CCTCACTCTGGAGTCAGCTACCCGCTCCCAGACATCTGTG
TATTTCTGCGCCAGCAGTGAGGACAGTTACGAGCAGTACT
TCGGGCCGGGCACCAGGCTCACGGTCACAGAGGACCTGAA
AAACGTGTTCCCTCCAAAAGTGGCCGTGTTCGAGCCTTCT
GAGGCCGAGATCAGCCACACACAGAAAGCCACACTCGTGT
GTCTGGCTACCGGCTTCTACCCCGATCACGTGGAACTGTC
TTGGTGGGTCAACGGCAAAGAGGTGCACAGCGGCGTCAGC
ACAGATCCCCAGCCTCTGAAAGAACAGCCCGCTCTGAACG
ACAGCCGCTACTGCCTGTCTAGCAGACTGAGAGTGTCCGC
CACCTTCTGGCAGAACCCCAGAAACCACTTCAGATGCCAG
GTCCAGTTCTACGGCCTGAGCGAGAACGATGAGTGGACCC
AGGACAGAGCCAAGCCTGTGACACAGATCGTGTCTGCCGA
AGCCTGGGGCAGAGCCGATTGTGGCTTTACCAGCGAGTCA
TACCAGCAGGGCGTGCTGTCTGCCACCATCCTGTATGAGA
TCCTGCTCGGCAAGGCCACACTGTACGCTGTGCTGGTGTC
TGCTCTGGTGCTGATGGCTATGGTCTCCCGGGAGCGCATC
CCCGAGGCCCGGGCCAAGCGGGGCAGCGGCGCCACCAACT
TCAGCCTGCTGAAGCAGGCCGGCGACGTGGAGGAGAACCC
CGGCCCTATGGCCACAGGCAGCAGAACATCTCTGCTGCTG
GCCTTCGGACTGCTGTGTCTGCCTTGGCTGCAAGAGGGTT
CCGCCGCCCAGTCAGTGACCCAGCCTGACATCCACATCAC
TGTCTCTGAAGGAGCCTCACTGGAGTTGAGATGTAACTAT
TCCTATGGGGCAACACCTTATCTCTTCTGGTATGTCCAGT
CCCCCGGCCAAGGCCTCCAGCTGCTCCTGAAGTACTTTTC
AGGAGACACTCTGGTTCAAGGCATTAAAGGCTTTGAGGCT
GAATTTAAGAGGAGTCAATCTTCCTTCAATCTGAGGAAAC
CCTCTGTGCATTGGAGTGATGCTGCTGAGTACTTCTGTGC
TGTGGGTGAATTGGACACAGGCTTTCAGAAACTTGTATTT
GGAACTGGCACCCGACTTCTGGTCAGTCCAAATATTCAGA
ACCCCGATCCTGCCGTGTACCAGCTGAGAGACAGCAAGAG
CAGCGACAAGAGCGTGTGCCTGTTCACCGACTTCGACAGC
CAGACCAACGTGTCCCAGAGCAAGGACAGCGACGTGTACA
TCACCGACAAGACCGTGCTGGACATGCGGAGCATGGACTT
CAAGAGCAACAGCGCCGTGGCCTGGTCCAACAAGAGCGAT
TTCGCCTGCGCCAACGCCTTCAACAACAGCATTATCCCCG
AGGACACATTCTTCCCAAGTCCTGAGAGCAGCTGCGACGT
GAAGCTGGTGGAAAAGAGCTTCGAGACAGACACCAACCTG
AACTTCCAGAACCTGTCCGTGATCGGCTTCCGCATCCTGC
TGCTGAAAGTGGCCGGCTTCAACCTGCTGATGACCCTGAG
ACTGTGGTCCAGCTGATGTGCCTTCTAGTTGCCAGCCATC
TGTTGTTTGCCCCTCCCCCGTGCCTTCCTTGACCCTGGAA
GGTGCCACTCCCACTGTCCTTTCCTAATAAAATGAGGAAA
TTGCATCGCATTGTCTGAGTAGGTGTCATTCTATTCTGGG
GGGTGGGGTGGGGCAGGACAGCAAGGGGGAGGATTGGGAA
GACAATAGCAGGCATGCTGGGGATGCGGTGGGCTCTATGG
CAAGCTTGAACAGAGAAACAGGAGAATATGGGCCAAACAG
GATATCTGTGGTAAGCAGTTCCTGCCCCGGCTCAGGGCCA
AGAACAGTTGGAACAGCAGAATATGGGCCAAACAGGATAT
CTGTGGTAAGCAGTTCCTGCCCCGGCTCAGGGCCAAGAAC
AGATGGTCCCCAGATGCGGTCCCGCCCTCAGCAGTTTCTA
GAGAACCATCAGATGTTTCCAGGGTGCCCCAAGGACCTGA
AATGACCCTGTGCCTTATTTGAACTAACCAATCAGTTCGC
TTCTCGCTTCTGTTCGCGCGCTTCTGCTCCCCGAGCTCTA
TATAAGCAGAGCTCGTTTAGTGAACCGTCAGATCGCCTGG
AGACGCCATCCACGCTGTTTTGACTTCCATAGAAGGGATC
CTCGACTTCTTAACCCAACAGAAGGCTCGAGAAGGTATAT
TGCTGTTGACAGTGAGCGAAAGATGTCAAGATTGAGCCTT
TAGTGAAGCCACAGATGTAAAGGCTCAATCTTGACATCTT
GTGCCTACTGCCTCGGACTTCAAGGGGCTACTTTAGGAGT
CGACTGCGATGCGGCCGCACCGATTTTGATTCTCAAACAA
ATGTGTCACAAAGTAAGGATTCTGATGTGTATATCACAGA
CAAAACTGTGCTAGACATGAGGTCTATGGACTTCAAGAGC
AACAGTGCTGTGGCCTGGAGCAACAAATCTGACTTTGCAT
GTGCAAACGCCTTCAACAACAGCATTATTCCAGAAGACAC
CTTCTTCCCCAGCCCAGGTAAGGGCAGCTTTGGTGCCTTC
GCAGGCTGTTTCCTTGCTTCAGGAATGGCCAGGTTCTGCC
CAGAGCTCTGGTCAATGATGTCTAAAACTCCTCTGATTGC
TAGCCGCGTTGCTGGCGTTTTTCCATAGGCTCCGCCCCCC
TGACGAGCATCACAAAAATCGACGCTCAAGTCAGAGGTGG
CGAAACCCGACAGGACTATAAAGATACCAGGCGTTTCCCC
CTGGAAGCTCCCTCGTGCGCTCTCCTGTTCCGACCCTGCC
GCTTACCGGATACCTGTCCGCCTTTCTCCCTTCGGGAAGC
GTGGCGCTTTCTCATAGCTCACGCTGTAGGTATCTCAGTT
CGGTGTAGGTCGTTCGCTCCAAGCTGGGCTGTGTGCACGA
ACCCCCCGTTCAGCCCGACCGCTGCGCCTTATCCGGTAAC
TATCGTCTTGAGTCCAACCCGGTAAGACACGACTTATCGC
CACTGGCAGCAGCCACTGGTAACAGGATTAGCAGAGCGAG
GTATGTAGGCGGTGCTACAGAGTTCTTGAAGTGGTGGCCT
AACTACGGCTACACTAGAAGAACAGTATTTGGTATCTGCG
CTCTGCTGAAGCCAGTTACCTTCGGAAAAAGAGTTGGTAG
CTCTTGATCCGGCAAACAAACCACCGCTGGTAGCGGTGGT
TTTTTTGTTTGCAAGCAGCAGATTACGCGCAGAAAAAAAG
GATCTCAAGAAGATCCTTTGATCTTTAGAAAAACTCATCG
AGCATCAAATGAAACTGCAATTTATTCATATCAGGATTAT
CAATACCATATTTTTGAAAAAGCCGTTTCTGTAATGAAGG
AGAAAACTCACCGAGGCAGTTCCATAGGATGGCAAGATCC
TGGTATCGGTCTGCGATTCCGACTCGTCCAACATCAATAC
AACCTATTAATTTCCCCTCGTCAAAAATAAGGTTATCAAG
TGAGAAATCACCATGAGTGACGACTGAATCCGGTGAGAAT
GGCAAAAGTTTATGCATTTCTTTCCAGACTTGTTCAACAG
GCCAGCCATTACGCTCGTCATCAAAATCACTCGCATCAAC
CAAACCGTTATTCATTCGTGATTGCGCCTGAGCCAGACGA
AATACGCGATCGCTGTTAAAAGGACAATTACAAACAGGAA
TCGAATGCAACCGGCGCAGGAACACTGCCAGCGCATCAAC
AATATTTTCACCTGAATCAGGATATTCTTCTAATACCTGG
AATGCTGTTTTTCCGGGGATCGCAGTGGTGAGTAACCATG
CATCATCAGGAGTACGGATAAAATGCTTGATGGTCGGAAG
AGGCATAAATTCCGTCAGCCAGTTTAGTCTGACCATCTCA
TCTGTAACATCATTGGCAACGCTACCTTTGCCATGTTTCA
GAAACAACTCTGGCGCATCGGGCTTCCCATACAAGCGATA
GATTGTCGCACCTGATTGCCCGACATTATCGCGAGCCCAT
TTATACCCATATAAATCAGCATCCATGTTGGAATTTAATC
GCGGCCTCGACGTTTCCCGTTGAATATGGCTCATAACACC
CCTTGTATTACTGTTTATGTAAGCAGACAGTTTTATTGTT
CATGATGATATATTTTTATCTTGTGCAATGTAACATCAGA
GATTTTGAGACAC

In certain embodiments, the Secondary Promoter Construct comprises nucleotides 1 to 2736 of SEQ ID NO: 64. In certain embodiments, the Secondary Promoter Construct comprises nucleotides 2788 to 4648 of SEQ ID NO: 64. In certain embodiments, the Secondary Promoter Construct comprises nucleotides 1 to 2736 and nucleotides 2788 to 4648 of SEQ ID NO: 64. SEQ ID NO: 64 is provided below.

[SEQ ID NO: 64]
GGTACCTCATGGCCTCTTGGCCAAGATTGATAGCTTGTGC
CTGTCCCTGAGTCCCAGTCCATCACGAGCAGCTGGTTTCT
AAGATGCTATTTCCCGTATAAAGCATGAGACCGTGACTTG
CCAGCCCCACAGAGCCCCGCCCTTGTCCATCACTGGCATC
TGGACTCCAGCCTGGGTTGGGGCAAAGAGGGAAATGAGAT
CATGTCCTAACCCTGATCCTCTTGTCCCACAGATATCCAG
AACCCTGACCCTGCCGTGTACCAGCTGAGAGACTCTAAAT
CCAGTGACAAGTCTGTCTGCCTATTCGAATTCGGCTCCGG
AGCCACTAACTTCTCCCTGTTGAAACAGGCTGGCGATGTT
GAAGAAAACCCCGGTCCTATGGCCACCGGCTCTAGAACAA
GCCTGCTGCTCGCTTTTGGCCTGCTCTGCCTCCCATGGCT
CCAAGAAGGATCTGCTGATGCTGGAATCACCCAGAGCCCA
AGATACAAGATCACAGAGACAGGAAGGCAGGTGACCTTGA
TGTGTCACCAGACTTGGAGCCACAGCTATATGTTCTGGTA
TCGACAAGACCTGGGACATGGGCTGAGGCTGATCTATTAC
TCAGCAGCTGCTGATATTACAGATAAAGGAGAAGTCTCCG
ATGGCTATGTTGTCTCCAGATCCAAGACAGAGAATTTCCC
CCTCACTCTGGAGTCAGCTACCCGCTCCCAGACATCTGTG
TATTTCTGCGCCAGCAGTGAGGACAGTTACGAGCAGTACT
TCGGGCCGGGCACCAGGCTCACGGTCACAGAGGACCTGAA
AAACGTGTTCCCTCCAAAAGTGGCCGTGTTCGAGCCTTCT
GAGGCCGAGATCAGCCACACACAGAAAGCCACACTCGTGT
GTCTGGCTACCGGCTTCTACCCCGATCACGTGGAACTGTC
TTGGTGGGTCAACGGCAAAGAGGTGCACAGCGGCGTCAGC
ACAGATCCCCAGCCTCTGAAAGAACAGCCCGCTCTGAACG
ACAGCCGCTACTGCCTGTCTAGCAGACTGAGAGTGTCCGC
CACCTTCTGGCAGAACCCCAGAAACCACTTCAGATGCCAG
GTCCAGTTCTACGGCCTGAGCGAGAACGATGAGTGGACCC
AGGACAGAGCCAAGCCTGTGACACAGATCGTGTCTGCCGA
AGCCTGGGGCAGAGCCGATTGTGGCTTTACCAGCGAGTCA
TACCAGCAGGGCGTGCTGTCTGCCACCATCCTGTATGAGA
TCCTGCTCGGCAAGGCCACACTGTACGCTGTGCTGGTGTC
TGCTCTGGTGCTGATGGCTATGGTCTCCCGGGAGCGCATC
CCCGAGGCCCGGGCCAAGCGGGGCAGCGGCGCCACCAACT
TCAGCCTGCTGAAGCAGGCCGGCGACGTGGAGGAGAACCC
CGGCCCTATGGCCACAGGCAGCAGAACATCTCTGCTGCTG
GCCTTCGGACTGCTGTGTCTGCCTTGGCTGCAAGAGGGTT
CCGCCGCCCAGTCAGTGACCCAGCCTGACATCCACATCAC
TGTCTCTGAAGGAGCCTCACTGGAGTTGAGATGTAACTAT
TCCTATGGGGCAACACCTTATCTCTTCTGGTATGTCCAGT
CCCCCGGCCAAGGCCTCCAGCTGCTCCTGAAGTACTTTTC
AGGAGACACTCTGGTTCAAGGCATTAAAGGCTTTGAGGCT
GAATTTAAGAGGAGTCAATCTTCCTTCAATCTGAGGAAAC
CCTCTGTGCATTGGAGTGATGCTGCTGAGTACTTCTGTGC
TGTGGGTGAATTGGACACAGGCTTTCAGAAACTTGTATTT
GGAACTGGCACCCGACTTCTGGTCAGTCCAAATATTCAGA
ACCCCGATCCTGCCGTGTACCAGCTGAGAGACAGCAAGAG
CAGCGACAAGAGCGTGTGCCTGTTCACCGACTTCGACAGC
CAGACCAACGTGTCCCAGAGCAAGGACAGCGACGTGTACA
TCACCGACAAGACCGTGCTGGACATGCGGAGCATGGACTT
CAAGAGCAACAGCGCCGTGGCCTGGTCCAACAAGAGCGAT
TTCGCCTGCGCCAACGCCTTCAACAACAGCATTATCCCCG
AGGACACATTCTTCCCAAGTCCTGAGAGCAGCTGCGACGT
GAAGCTGGTGGAAAAGAGCTTCGAGACAGACACCAACCTG
AACTTCCAGAACCTGTCCGTGATCGGCTTCCGCATCCTGC
TGCTGAAAGTGGCCGGCTTCAACCTGCTGATGACCCTGAG
ACTGTGGTCCAGCTGATGTGCCTTCTAGTTGCCAGCCATC
TGTTGTTTGCCCCTCCCCCGTGCCTTCCTTGACCCTGGAA
GGTGCCACTCCCACTGTCCTTTCCTAATAAAATGAGGAAA
TTGCATCGCATTGTCTGAGTAGGTGTCATTCTATTCTGGG
GGGTGGGGTGGGGCAGGACAGCAAGGGGGAGGATTGGGAA
GACAATAGCAGGCATGCTGGGGATGCGGTGGGCTCTATGG
CAAGCTTGAGGGCCTATTTCCCATGATTCCTTCATATTTG
CATATACGATACAAGGCTGTTAGAGAGATAATTGGAATTA
ATTTGACTGTAAACACAAAGATATTAGTACAAAATACGTG
ACGTAGAAAGTAATAATTTCTTGGGTAGTTTGCAGTTTTA
AAATTATGTTTTAAAATGGACTATCATATGCTTACCGTAA
CTTGAAAGTATTTCGATTTCTTGGCTTTATATATCTTGTG
GAAAGGACGAAACACCGATGTCAAGATTGAGCCTTGCCCT
GACCCAGCAAGGCTCAATCTTGACATCTTTTTTGTCGACT
GCGATGCGGCCGCACCGATTTTGATTCTCAAACAAATGTG
TCACAAAGTAAGGATTCTGATGTGTATATCACAGACAAAA
CTGTGCTAGACATGAGGTCTATGGACTTCAAGAGCAACAG
TGCTGTGGCCTGGAGCAACAAATCTGACTTTGCATGTGCA
AACGCCTTCAACAACAGCATTATTCCAGAAGACACCTTCT
TCCCCAGCCCAGGTAAGGGCAGCTTTGGTGCCTTCGCAGG
CTGTTTCCTTGCTTCAGGAATGGCCAGGTTCTGCCCAGAG
CTCTGGTCAATGATGTCTAAAACTCCTCTGATTGCTAGCC
GCGTTGCTGGCGTTTTTCCATAGGCTCCGCCCCCCTGACG
AGCATCACAAAAATCGACGCTCAAGTCAGAGGTGGCGAAA
CCCGACAGGACTATAAAGATACCAGGCGTTTCCCCCTGGA
AGCTCCCTCGTGCGCTCTCCTGTTCCGACCCTGCCGCTTA
CCGGATACCTGTCCGCCTTTCTCCCTTCGGGAAGCGTGGC
GCTTTCTCATAGCTCACGCTGTAGGTATCTCAGTTCGGTG
TAGGTCGTTCGCTCCAAGCTGGGCTGTGTGCACGAACCCC
CCGTTCAGCCCGACCGCTGCGCCTTATCCGGTAACTATCG
TCTTGAGTCCAACCCGGTAAGACACGACTTATCGCCACTG
GCAGCAGCCACTGGTAACAGGATTAGCAGAGCGAGGTATG
TAGGCGGTGCTACAGAGTTCTTGAAGTGGTGGCCTAACTA
CGGCTACACTAGAAGAACAGTATTTGGTATCTGCGCTCTG
CTGAAGCCAGTTACCTTCGGAAAAAGAGTTGGTAGCTCTT
GATCCGGCAAACAAACCACCGCTGGTAGCGGTGGTTTTTT
TGTTTGCAAGCAGCAGATTACGCGCAGAAAAAAAGGATCT
CAAGAAGATCCTTTGATCTTTAGAAAAACTCATCGAGCAT
CAAATGAAACTGCAATTTATTCATATCAGGATTATCAATA
CCATATTTTTGAAAAAGCCGTTTCTGTAATGAAGGAGAAA
ACTCACCGAGGCAGTTCCATAGGATGGCAAGATCCTGGTA
TCGGTCTGCGATTCCGACTCGTCCAACATCAATACAACCT
ATTAATTTCCCCTCGTCAAAAATAAGGTTATCAAGTGAGA
AATCACCATGAGTGACGACTGAATCCGGTGAGAATGGCAA
AAGTTTATGCATTTCTTTCCAGACTTGTTCAACAGGCCAG
CCATTACGCTCGTCATCAAAATCACTCGCATCAACCAAAC
CGTTATTCATTCGTGATTGCGCCTGAGCCAGACGAAATAC
GCGATCGCTGTTAAAAGGACAATTACAAACAGGAATCGAA
TGCAACCGGCGCAGGAACACTGCCAGCGCATCAACAATAT
TTTCACCTGAATCAGGATATTCTTCTAATACCTGGAATGC
TGTTTTTCCGGGGATCGCAGTGGTGAGTAACCATGCATCA
TCAGGAGTACGGATAAAATGCTTGATGGTCGGAAGAGGCA
TAAATTCCGTCAGCCAGTTTAGTCTGACCATCTCATCTGT
AACATCATTGGCAACGCTACCTTTGCCATGTTTCAGAAAC
AACTCTGGCGCATCGGGCTTCCCATACAAGCGATAGATTG
TCGCACCTGATTGCCCGACATTATCGCGAGCCCATTTATA
CCCATATAAATCAGCATCCATGTTGGAATTTAATCGCGGC
CTCGACGTTTCCCGTTGAATATGGCTCATAACACCCCTTG
TATTACTGTTTATGTAAGCAGACAGTTTTATTGTTCATGA
TGATATATTTTTATCTTGTGCAATGTAACATCAGAGATTT
TGAGACAC

In certain embodiments, the Secondary Promoter Construct comprises a nucleotide sequence that is at least about 80%, about 85%, about 90%, about 95%, about 96%, about 97%, about 98%, about 99% identical to the nucleotide sequence set forth in SEQ ID NOs: 65-98. In certain embodiments, the Secondary Promoter Construct comprises the nucleotide sequence set forth in SEQ ID NOs: 65-98. In certain embodiments, the Secondary Promoter Construct comprises a nucleotide sequence selected from the group consisting of SEQ ID NOs: 65, 67, 69, 71, 73, 75, 77, 79, 81, 83, 85, 87, 89, 91, 93, 95, and 97. In certain embodiments, the Secondary Promoter Construct comprises a nucleotide sequence selected from the group consisting of SEQ ID NOs: 66, 68, 70, 72, 74, 76, 78, 80, 82, 84, 86, 88, 90, 92, 94, 96, and 98. In certain embodiments, the Secondary Promoter Construct comprises a first nucleotide sequence selected from the group consisting of SEQ ID NOs: 65, 67, 69, 71, 73, 75, 77, 79, 81, 83, 85, 87, 89, 91, 93, 95, and 97, and a second nucleotide sequence selected from the group consisting of SEQ ID NOs: 66, 68, 70, 72, 74, 76, 78, 80, 82, 84, 86, 88, 90, 92, 94, 96, and 98.

3.2. Methods of Producing Secondary Promoter Products with a Young Phenotype

In certain embodiments, the present disclosure relates, in part, to the production of engineered “young” T cells comprising the Secondary Promoter Constructs disclosed herein. In certain embodiments, the present disclosure comprises methods for producing antigen-specific cells, e.g., T cells, ex vivo, comprising activating, engineering, and expanding antigen-specific cells originally obtained from a subject or isolated from such sample.

In certain embodiments, the methods for activating cells comprise the steps of activating the TCR/CD3 complex. For example, without limitation, the T cells can be incubated and/or cultured with CD3 agonists, CD28 agonists, or a combination thereof.

In certain embodiments, engineered activated antigen-specific cells, e.g., engineered activated T cells, can be expanded by culturing the engineered activated antigen-specific cells, e.g., T cells, with cytokines, chemokine, soluble peptides, or combination thereof. In certain embodiments, the engineered activated antigen-specific cells, e.g., engineered activated T cells, can be cultured with one or more cytokines. In certain embodiments, the cytokines can be IL2, IL7, IL15, or combinations thereof. For example, engineered activated antigen-specific cells, e.g., engineered activated T cells, can be cultured with IL7 and IL15. In certain embodiments, the cytokine used in connection with the engineered activated antigen-specific cell, e.g., engineered activated T cell, culture can be present at a concentration from about 1 pg/ml to about 1 g/ml, from about 1 ng/ml to about 1 g/ml, from about 1 g/ml to about 1 g/ml, or from about 1 mg/ml to about 1 g/ml, and any values in between.

3.3. Gene-Editing Methods

In certain embodiments, the present disclosure involves, in part, methods of engineering human cells, e.g., engineered T cells or engineered human stem cells, comprising the Secondary Promoter Constructs disclosed herein. In certain embodiments, the present disclosure involves, in part, methods of engineering human cells, e.g., NK cells, NKT cells, macrophages, hematopoietic stem cells (HSCs), cells derived from HSCs, or dendritic/antigen-presenting cells. In certain embodiments, such engineering involves genome editing. For example, but not by way of limitation, such genome editing can be accomplished with nucleases targeting one or more endogenous loci, e.g., TCR alpha (TCRα) locus and TCR beta (TCRβ) locus. In certain embodiments, the nucleases can generate single-stranded DNA nicks or double-stranded DNA breaks in an endogenous target sequence. In certain embodiments, the nuclease can target coding or non-coding portions of the genome, e.g., exons, introns. In certain embodiments, the nucleases contemplated herein comprise homing endonuclease, meganuclease, megaTAL nuclease, transcription activator-like effector nuclease (TALEN), zinc-finger nuclease (ZFN), and clustered regularly interspaced short palindromic repeats (CRISPR)/Cas nuclease. In certain embodiments, the nucleases can themselves be engineered, e.g., via the introduction of amino acid substitutions and/or deletions, to increase the efficiency of the cutting activity.

In certain embodiments, a CRISPR/Cas nuclease system is used to engineer human cells. In certain embodiments, the CRISPR/Cas nuclease system comprises a Cas nuclease and one or more RNAs that recruit the Cas nuclease to the endogenous target sequence, e.g., single guide RNA. In certain embodiments, the Cas nuclease and the RNA are introduced in the cell separately, e.g. using different vectors or compositions, or together, e.g., in a polycistronic construct or a single protein-RNA complex. In certain embodiments, the Cas nuclease is Cas9 or Cas12a. In certain embodiments, the Cas9 polypeptide is obtained from a bacterial species including, without limitation, Streptococcus pyogenes or Neisseria menengitidis. Additional examples of CRISPR/Cas systems are known in the art. See Adli, Mazhar. “The CRISPR tool kit for genome editing and beyond.” Nature communications vol. 9, 1 1911 (2018), herein incorporated by reference for all that it teaches.

In certain embodiments, genome editing occurs at one or more genome loci that regulate immunological responses. In certain embodiments, the loci include, without limitation, TCR alpha (TCRα) locus, TCR beta (TCRβ) locus, TCR gamma (TCRγ), and TCR delta (TCRδ). In certain embodiments, the locus for inserting a Secondary Promoter Construct is anywhere in the genome. In certain embodiments, the locus for inserting a Secondary Promoter Construct is the TRAC locus. In certain embodiments, the locus for inserting a Secondary Promoter Construct is one of the two TRBC loci. In certain embodiments, the locus for inserting a Secondary Promoter Construct is a locus other than the TRAC locus or TRAB loci. In certain embodiments, the Secondary Promoter Construct is inserted into a gene locus wherein such gene is knocked out.

In certain embodiments, genome editing is performed by using non-viral delivery systems. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for the delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

In certain embodiments, genome editing is performed by using viral delivery systems. In certain embodiments, the viral methods include targeted integration (including but not limited to AAV) and random integration (including but not limited to lentiviral approaches). In certain embodiments, the viral delivery would be accomplished without integration of the nuclease. In such embodiments, the viral delivery system can be Lentiflash or another similar delivery system.

3.4. Compositions and Vectors

The presently disclosed subject matter provides compositions comprising cells (e.g., Secondary Promoter Cells) disclosed herein.

In certain embodiments, the presently disclosed subject matter provides nucleic acid compositions comprising a polynucleotide encoding the NeoTCR disclosed herein. In certain embodiments, the nucleic acid compositions disclosed herein comprise a polynucleotide encoding a Secondary Promoter Construct disclosed herein. Also provided are cells comprising such nucleic acid compositions.

In certain embodiments, the nucleic acid composition further comprises a promoter that is operably linked to the NeoTCR disclosed herein. In certain embodiments, the nucleic acid composition further comprises a promoter that is operably linked to the Secondary Promoter Construct disclosed herein.

In certain embodiments, the promoter is endogenous or exogenous. In certain embodiments, the exogenous promoter is selected from the group consisting of an elongation factor (EF)-1 promoter, a CMV promoter, an SV40 promoter, a PGK promoter, a long terminal repeat (LTR) promoter, and a metallothionein promoter. In certain embodiments, the promoter is an inducible promoter. In certain embodiments, the inducible promoter is selected from the group consisting of an NFAT transcriptional response element (TRE) promoter, a CD69 promoter, a CD25 promoter, an IL-2 promoter, an IL-12 promoter, a p40 promoter, and a Bcl-xL promoter.

The compositions and nucleic acid compositions can be administered to subjects or and/delivered into cells by art-known methods or as described herein. Genetic modification of a cell (e.g., a T cell) can be accomplished by transducing a substantially homogeneous cell composition with a recombinant DNA construct. In certain embodiments, a retroviral vector (either a gamma-retroviral vector or a lentiviral vector) is employed for the introduction of the DNA construct into the cell. Non-viral vectors may be used as well.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for the delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

Polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

3.5. Cells

The presently disclosed subject matter provides cells comprising a presently disclosed Secondary Promoter Constructs. In certain embodiments, the cell is an immune cell (e.g., a lymphocyte). For example, but without any limitation, the cells can be T cells, Natural Killer (NK) cells, B cells, dendritic cells, hematopoietic stem cells, or pluripotent stem cells.

In certain embodiments, the cell is a T cell. Non-limiting examples of T cells encompassed by the present disclosure include helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the T cell is a CD4+ T cell. In certain embodiments, the T cell is a CD8+ T cell.

In certain embodiments, the T cell is a peripheral T cell. Peripheral T cells are differentiated T cells that have undergone the maturation process. Peripheral T cells can be found in peripheral blood. In certain embodiments, the T cell is not a naïve T cell. Naïve T cells are precursors for effector and memory T cell subsets. Phenotypically, naïve T cells are small cells with little cytoplasm; they express surface markers, such as CD45RA, CCR7, CD62L, CD127, and CD132. Naïve T cells lack expression of markers of previous activation, such as CD25, CD44, CD69, CD45RO, or HLA-DR.

In certain embodiments, the cell is a NK cell. Natural killer (NK) cells can be lymphocytes that are part of cell-mediated immunity and act during the innate immune response. NK cells do not require prior activation to perform their cytotoxic effect on target cells.

In certain embodiments, the cells are autologous. In certain embodiments, the cells can be transduced with the presently disclosed Secondary Promoter Constructs to express an exogenous TCR (e.g., NeoTCR) and a Payload.

3.6. Pharmaceutical Formulations.

In certain embodiments, the present disclosure provides a pharmaceutical formulation comprising the Secondary Promoter Cells disclosed herein. In certain embodiments, pharmaceutical formulations of the Secondary Promoter Products are prepared by combining the Secondary Promoter Cells in a solution that can preserve the ‘young’ phenotype of the cells in a cryopreserved state.

In certain embodiments, pharmaceutically acceptable carriers, buffers, stabilizers, and/or preservatives can be added to the cryopreservation solution. Any cryopreservation agent and/or media can be used to cryopreserve the Secondary Promoter Product, including but not limited to CryoStor, CryoStor CS5, CELLBANKER, and custom cryopreservation media that optionally include DMSO.

### 4. Methods of Treatment

The presently disclosed subject matter provides methods for inducing and/or increasing an immune response in a subject in need thereof. The Secondary Promoter Products can be used for treating and/or preventing a cancer in a subject. The Secondary Promoter Products can be used for prolonging the survival of a subject suffering from a cancer. The Secondary Promoter Products can also be used for treating and/or preventing a cancer in a subject. The Secondary Promoter Products can also be used for reducing tumor burden in a subject. Such methods comprise administering the Secondary Promoter Products in an amount effective or a composition (e.g., a pharmaceutical composition or formulation disclosed herein) comprising thereof to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the Secondary Promoter Products can be used for treating viral or bacterial diseases. In certain embodiments, the Secondary Promoter Products can be used for treating autoimmune diseases.

In certain embodiments, an effective amount of the Secondary Promoter Products are delivered through intravenous (IV) administration. In certain embodiments, the Secondary Promoter Products are delivered through intravenous administration in a single administration. In certain embodiments, the Secondary Promoter Products are delivered through intravenous administration in multiple administrations. In certain embodiments, the Secondary Promoter Products are delivered through intravenous administration in two or more administrations. In certain embodiments, the Secondary Promoter Products are delivered through intravenous administration in two administrations. In certain embodiments, the Secondary Promoter Products are delivered through intravenous administration in three administrations.

The presently disclosed subject matter provides methods for treating and/or preventing cancer in a subject. In certain embodiments, the method comprises administering an effective amount of the Secondary Promoter Products to a subject having cancer.

Non-limiting examples of cancer include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed Secondary Promoter Products comprising young T cells can be used for treating and/or preventing blood cancers (e.g., leukemias, lymphomas, and myelomas) or ovarian cancer, which are not amenable to conventional therapeutic interventions.

In certain embodiments, the neoplasia is a solid cancer or a solid tumor. In certain embodiments, the solid tumor or solid cancer is selected from the group consisting of glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, lung adenocarcinoma, stomach adenocarcinoma, cervical and endocervical cancer, kidney clear cell carcinoma, testicular germ cell tumors, and aggressive B-cell lymphomas.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor can be detected based on tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, to palliate their condition. Ideally, reduction in tumor mass occurs as a result, but any clinical improvement constitutes a benefit. Clinical improvement includes decreased risk or rate of progression or reduction in pathological consequences of the tumor.

### 5. Articles of Manufacture

The Secondary Promoter Products can be used in combination with articles of manufacture. Such articles of manufacture can be useful for the prevention or treatment of proliferative disorders (e.g., cancer). Examples of articles of manufacture include but are not limited to containers (e.g., infusion bags, bottles, storage containers, flasks, vials, syringes, tubes, and IV solution bags) and a label or package insert on or associated with the container. The containers may be made of any material that is acceptable for the storage and preservation of the Secondary Promoter Cells within the Secondary Promoter Products. In certain embodiments, the container may be an intravenous solution bag or a vial having a stopper pierceable by a hypodermic injection needle. For example, the container may be a CryoMACS freezing bag. The label or package insert indicates that the Secondary Promoter Products are used for treating the condition of choice and the patient of origin. The patient is identified on the container of the Secondary Promoter Product because the Secondary Promoter Products is made from autologous cells and engineered as a patient-specific and individualized treatment.

In certain embodiments, the article of manufacture can comprise: 1) a first container with a Secondary Promoter Product contained therein.

In certain embodiments, the article of manufacture can comprise: 1) a first container with a Secondary Promoter Product contained therein; and 2) a second container with the same Secondary Promoter Product as the first container contained therein. Optionally, additional containers with the same Secondary Promoter Product as the first and second containers can be prepared and made. Optionally, additional containers containing a composition comprising a different cytotoxic or otherwise therapeutic agent may also be combined with the containers described above.

In certain embodiments, the article of manufacture can comprise: 1) a first container with a Secondary Promoter Product contained therein; and 2) a second container with a composition contained therein, wherein the composition comprises a further cytotoxic or otherwise therapeutic agent.

In certain embodiments, the article of manufacture can comprise: 1) a first container with two Secondary Promoter Products contained therein; and 2) a second container with a composition contained therein, wherein the composition comprises a further cytotoxic or otherwise therapeutic agent.

In certain embodiments, the article of manufacture can comprise: 1) a first container with a Secondary Promoter Product contained therein; 2) a second container with a second Secondary Promoter Product contained therein; and 3) optionally a third container with a composition contained therein, wherein the composition comprises a further cytotoxic or otherwise therapeutic agent. In certain embodiments, the first and second Secondary Promoter Products are different Secondary Promoter Products. In certain embodiments, the first and second Secondary Promoter Products are the same Secondary Promoter Products.

In certain embodiments, the article of manufacture can comprise: 1) a first container with three Secondary Promoter Products contained therein; and 2) optionally a second container with a composition contained therein, wherein the composition comprises a further cytotoxic or otherwise therapeutic agent.

In certain embodiments, the article of manufacture can comprise: 1) a first container with a Secondary Promoter Product contained therein; 2) a second container with a second Secondary Promoter Product contained therein; 3) a third container with a third Secondary Promoter Product contained therein; and 4) optionally a fourth container with a composition contained therein, wherein the composition comprises a further cytotoxic or otherwise therapeutic agent. In certain embodiments, the first, second, and third Secondary Promoter Products are different Secondary Promoter Products. In certain embodiments, the first, second, and third Secondary Promoter Products are the same Secondary Promoter Products. In certain embodiments, two of the first, second, and third Secondary Promoter Products are the same Secondary Promoter Products.

In certain embodiments, the article of manufacture can comprise: 1) a first container with four Secondary Promoter Products contained therein; and 2) optionally a second container with a composition contained therein, wherein the composition comprises a further cytotoxic or otherwise therapeutic agent.

In certain embodiments, the article of manufacture can comprise: 1) a first container with a Secondary Promoter Product contained therein; 2) a second container with a second Secondary Promoter Product contained therein; 3) a third container with a third Secondary Promoter Product contained therein; 4) a fourth container with a fourth Secondary Promoter Product contained therein; and 5) optionally a fifth container with a composition contained therein, wherein the composition comprises a further cytotoxic or otherwise therapeutic agent. In certain embodiments, the first, second, third, and fourth Secondary Promoter Products are different Secondary Promoter Products. In certain embodiments, the first, second, third, and fourth Secondary Promoter Products are the same NeoTCR Products. In certain embodiments, two of the first, second, third, and fourth Secondary Promoter Products are the same NeoTCR Products. In certain embodiments, three of the first, second, third, and fourth Secondary Promoter Products are the same Secondary Promoter Products.

In certain embodiments, the article of manufacture can comprise: 1) a first container with five or more Secondary Promoter Products contained therein; and 2) optionally a second container with a composition contained therein, wherein the composition comprises a further cytotoxic or otherwise therapeutic agent.

In certain embodiments, the article of manufacture can comprise: 1) a first container with a Secondary Promoter Product contained therein; 2) a second container with a second Secondary Promoter Product contained therein; 3) a third container with a third Secondary Promoter Product contained therein; 4) a fourth container with a fourth Secondary Promoter Product contained therein; 5) a fifth container with a fifth Secondary Promoter Product contained therein; 6) optionally a sixth or more additional container with a sixth or more Secondary Promoter Product contained therein; and 7) optionally an additional container with a composition contained therein, wherein the composition comprises a further cytotoxic or otherwise therapeutic agent. In certain embodiments, all of the containers of Secondary Promoter Products are different Secondary Promoter Products. In certain embodiments, all of the containers of Secondary Promoter Products are the same Secondary Promoter Products. In certain embodiments, there can be any combination of same or different Secondary Promoter Products in the five or more containers based on the availability of detectable Secondary Promoters in a patient's tumor sample(s), the need and/or desire to have multiple Secondary Promoter Products for the patient, and the availability of any Secondary Promoter Product that may require or benefit from one or more container.

In certain embodiments, the article of manufacture can comprise: 1) a first container with a Secondary Promoter Product contained therein; 2) a second container with a second Secondary Promoter Product contained therein; 3) a third container with a third Secondary Promoter Product contained therein.

In certain embodiments, the article of manufacture can comprise: 1) a first container with a Secondary Promoter Product contained therein; 2) a second container with a second Secondary Promoter Product contained therein; 3) a third container with a third Secondary Promoter Product contained therein; 4) optionally a fourth container with a fourth Secondary Promoter Product contained therein.

In certain embodiments, the article of manufacture can comprise: 1) a first container with a Secondary Promoter Product contained therein; 2) a second container with a second Secondary Promoter Product contained therein; 3) a third container with a third Secondary Promoter Product contained therein; 4) a fourth container with a fourth Secondary Promoter Product contained therein; 5) optionally a fifth container with a fourth Secondary Promoter Product contained therein.

In certain embodiments, the article of manufacture can comprise a container with one Secondary Promoter Product contained therein. The article of manufacture may comprise a container with two Secondary Promoter Products contained therein. The article of manufacture can comprise a container with three Secondary Promoter Products contained therein. The article of manufacture can comprise a container with four Secondary Promoter Products contained therein. The article of manufacture can comprise a container with five Secondary Promoter Products contained therein.

In certain embodiments, the article of manufacture can comprise 1) a first container with one Secondary Promoter Product contained therein, and 2) a second container with two Secondary Promoter Products contained therein. In certain embodiments, the article of manufacture can comprise 1) a first container with two Secondary Promoter Products contained therein, and 2) a second container with one Secondary Promoter Product contained therein. In the examples above, a third and/or fourth container comprising one or more additional Secondary Promoter Products may be included in the article of manufacture. Additionally, a fifth container comprising one or more additional Secondary Promoter Products may be included in the article of manufacture.

Furthermore, any container of Secondary Promoter Product described herein can be split into two, three, or four separate containers for multiple time points of administration and/or based on the appropriate dose for the patient.

In certain embodiments, the Secondary Promoter Products are provided in a kit. The kit can, by means of non-limiting examples, contain package insert(s), labels, instructions for using the Secondary Promoter Product(s), syringes, disposal instructions, administration instructions, tubing, needles, and anything else a clinician would need in order to properly administer the Secondary Promoter Product(s).

### 6. Therapeutic Compositions and Methods of Manufacturing

As described herein, plasmid DNA-mediated precision genome engineering process for Good Manufacturing Practice (GMP) manufacturing of Secondary Promoter Products was developed. Targeted integration of the patient-specific NeoTCR was accomplished by electroporating CRISPR endonuclease ribonucleoproteins (RNPs) together with the personalized NeoTCR gene cassette, encoded by the plasmid DNA. In addition to the NeoTCR, the Secondary Promoter Constructs were inserted by incorporating them into the NeoTCR vector and then electroporating with CRISPR endonuclease ribonucleoproteins (RNPs) as described above.

In certain embodiments, the Secondary Promoter Products can be formulated into a drug product using the clinical manufacturing process. Under this process, the Secondary Promoter Products are cryopreserved in CryoMACS Freezing Bags. One or more bags may be shipped to the site for each patient depending on patient needs. In certain embodiments, the product is composed of apheresis-derived, patient-autologous, and CD4/CD8 T cells that have been precision genome engineered to express one or more autologous NeoTCRs targeting a neoepitope (e.g., against a private neoantigen) complexed to one of the endogenous HLA receptors presented exclusively on the surface of that patient's tumor cells and a Payload. In certain embodiments, the cell includes a Secondary Promoter Construct disclosed herein.

In certain embodiments, the final product contains about 5% dimethyl sulfoxide (DMSO), human serum albumin, and Plasma-Lyte. In certain embodiments, the final cell product contains the list of components provided in Table 1.

TABLE 1
Composition of the Secondary Promoter Product

| Component | Specification/Grade |
|---|---|
| Total nucleated NeoTCR Cells | cGMP manufactured |
| Plasma-Lyte A | USP |
| Human Serum Albumin in 0.02-0.08M | USP |
| sodium caprylate and sodium | |
| tryptophanate | |
| CryoStor CS10 | cGMP manufactured with USP |
| | grade materials |

### 7. Kits

The presently disclosed subject matter provides kits for inducing and/or enhancing immune response and/or treating and/or preventing cancer or a pathogen infection in a subject. In certain embodiments, the kit comprises an effective amount of presently disclosed cells (e.g. Secondary Promoter Cells) or a pharmaceutical composition comprising thereof (e.g., Secondary Promoter Products). In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding a presently disclosed HR template.

If desired, the cells and/or nucleic acid molecules are provided together with instructions for administering the cells or nucleic acid molecules to a subject having or at risk of developing cancer or pathogen, or immune disorder. The instructions generally include information about the use of the composition for the treatment and/or prevention of cancer or a pathogen infection. In certain embodiments, the instructions include at least one of the following: description of the therapeutic agent; dosage schedule and administration for treatment or prevention of a neoplasia, pathogen infection, or immune disorder or symptoms thereof, precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container. The resulting cells can be grown under conditions similar to those for unmodified cells, whereby the modified cells can be expanded and used for a variety of purposes.

### 8. Exemplary Embodiments

In certain embodiments, the present disclosure provides a cell comprising an exogenous polynucleotide comprising an exogenous enhancer, an insulator, a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising an exogenous enhancer, a pause element, a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising a Woodchuck Hepatitis Virus Posttranscriptional Regulatory Element (WPRE), a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising an insulator, a Woodchuck Hepatitis Virus Posttranscriptional Regulatory Element (WPRE), a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising an insulator, a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising a sequence encoding an exogenous TCR and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter.

In certain embodiments, the present disclosure provides a cell comprising an exogenous polynucleotide comprising a sequence encoding an exogenous TCR, a first sequence encoding a Payload, and a second sequence encoding a Payload, wherein the exogenous polynucleotide is integrated at an endogenous locus within the genome of the cell, wherein the sequence encoding an exogenous TCR and the first sequence encoding a Payload are under control of an endogenous promoter and the second sequence encoding a Payload is under control of an exogenous promoter.

In certain embodiments, the present disclosure provides a cell comprising an exogenous polynucleotide comprising a sequence encoding an exogenous TCR and a sequence encoding at least one Payload, wherein the exogenous polynucleotide is integrated at an endogenous locus within the genome of the cell and is under control of an endogenous promoter.

In certain embodiments of the cells disclosed herein, the insulator is an HS4 insulator or an IS2 insulator. In certain embodiments of the cells disclosed herein, the insulator comprises a nucleotide sequence that is at least about 80% identical to the nucleotide sequence set forth in SEQ ID NO: 18 or SEQ ID NO: 19. In certain embodiments of the cells disclosed herein, the insulator comprises the nucleotide sequence set forth in SEQ ID NO: 18 or SEQ ID NO: 19.

In certain embodiments of the cells disclosed herein, the WPRE comprises a nucleotide sequence that is at least about 80% identical to the nucleotide sequence set forth in SEQ ID NO: 20. In certain embodiments of the cells disclosed herein, the WPRE comprises the nucleotide sequence set forth in SEQ ID NO: 20.

In certain embodiments of the cells disclosed herein, the exogenous enhancer is a CMV enhancer, a TCRα enhancer, or a TCRβ enhancer. In certain embodiments of the cells disclosed herein, the exogenous enhancer is a TCRα enhancer. In certain embodiments of the cells disclosed herein, the TCRα enhancer comprises a nucleotide sequence that is at least about 80% identical to the nucleotide sequence set forth in SEQ ID NO: 32. In certain embodiments of the cells disclosed herein, the TCRα enhancer comprises the nucleotide sequence set forth in SEQ ID NO: 32. In certain embodiments of the cells disclosed herein, the exogenous enhancer is a CMV enhancer. In certain embodiments of the cells disclosed herein, the CMV enhancer comprises a nucleotide sequence that is at least about 80% identical to the nucleotide sequence set forth in SEQ ID NO: 33. In certain embodiments of the cells disclosed herein, the CMV enhancer comprises the nucleotide sequence set forth in SEQ ID NO: 33.

In certain embodiments of the cells disclosed herein, the exogenous promoter is a constitutive promoter. In certain embodiments of the cells disclosed herein, the constitutive promoter is an MDN promoter, an EF1α promoter, an ACTB promoter, a PGK promoter, or a U6 promoter. In certain embodiments of the cells disclosed herein, the constitutive promoter is an MDN promoter. In certain embodiments of the cells disclosed herein, the constitutive promoter is an EF1α promoter. In certain embodiments of the cells disclosed herein, the exogenous promoter is an inducible promoter. In certain embodiments of the cells disclosed herein, the inducible promoter is an AP1 promoter, an NFAT promoter, an NF-κB promoter, or an NR4A-responsive promoter.

In certain embodiments of the cells disclosed herein, the sequence encoding an exogenous TCR comprises a TCRα gene sequence or a TCRβ gene sequence. In certain embodiments of the cells disclosed herein, the sequence encoding an exogenous TCR comprises a TCRα gene sequence and a TCRβ gene sequence. In certain embodiments of the cells disclosed herein, the sequence encoding an exogenous TCR further comprises a sequence encoding a P2A peptide, a sequence encoding a signal sequence, a sequence encoding a protease cleavage peptide, or a combination thereof. In certain embodiments of the cells disclosed herein, the sequence encoding an exogenous TCR comprises a TCRα gene sequence, a TCRβ gene sequence, a sequence encoding a P2A peptide, a sequence encoding a signal sequence, a sequence encoding a protease cleavage peptide, or a combination thereof.

In certain embodiments of the cells disclosed herein, the sequence encoding an exogenous TCR comprises, from 5′ end to 3′ end, a first sequence encoding a P2A peptide, a first sequence encoding a signal sequence, a TCRβ gene sequence, a sequence encoding a protease cleavage peptide, a second sequence encoding a P2A peptide, a second sequence encoding a signal sequence, and a TCRα gene sequence. In certain embodiments of the cells disclosed herein, the sequence encoding an exogenous TCR comprises, from 5′ end to 3′ end, a first sequence encoding a P2A peptide, a first sequence encoding a signal sequence, a TCRβ gene sequence, a sequence encoding a protease cleavage peptide, a second sequence encoding a P2A peptide, a second sequence encoding a signal sequence, a TCRα gene sequence, and a poly-adenylation sequence. In certain embodiments of the cells disclosed herein, the sequence encoding an exogenous TCR comprises, from 5′ end to 3′ end, a first sequence encoding a P2A peptide, a first sequence encoding a signal sequence, a TCRα gene sequence, a sequence encoding a protease cleavage peptide, a second sequence encoding a P2A peptide, a second sequence encoding a signal sequence, a TCRβ gene sequence, and a poly-adenylation sequence.

In certain embodiments of the cells disclosed herein, the at least one Payload is selected from the group consisting of a cytokine receptors trap, a ligand trap, an angiogenesis factor, an apoptotic factor, an inhibitory protein, an extracellular matrix modulator, a soluble TCR, a homing signal, an enzyme, a modulator of reactive oxygen species, a competitive ligand inhibitor, a protein that binds to receptors and sterically hinders receptor function, and an inhibitory RNA molecule.

In certain embodiments of the cells disclosed herein, the 3′ of the at least one Payload comprises a STOP codon. In certain embodiments of the cells disclosed herein, the 3′ of the at least one Payload comprises a sequence encoding a 2A peptide and a sequence encoding a protease cleavage peptide. In certain embodiments of the cells disclosed herein, the 3′ of the at least one Payload comprises a poly-adenylation sequence.

In certain embodiments of the cells disclosed herein, the at least one Payload is an inhibitory RNA molecule. In certain embodiments of the cells disclosed herein, the inhibitory RNA molecule is a shRNA, a miRNA, or a miRNA cluster. In certain embodiments of the cells disclosed herein, the inhibitory RNA molecule is a miRNA. In certain embodiments of the cells disclosed herein, the miRNA comprises a first flanking sequence and a second flanking sequence. In certain embodiments of the cells disclosed herein, the first flanking sequence and the second flanking sequence are derived from miR-155, miR-30, miR-17/92, miR-122, or miR-21. In certain embodiments of the cells disclosed herein, the inhibitory RNA molecule is flanked by a splice donor site or a splice acceptor site. In certain embodiments of the cells disclosed herein, the inhibitory RNA molecule is flanked by a splice donor site and a splice acceptor site.

In certain embodiments of the cells disclosed herein, the sequences encoding a P2A peptide are codon diverged. In certain embodiments of the cells disclosed herein, the sequences encoding a protease cleavage peptide are codon diverged. In certain embodiments of the cells disclosed herein, the sequences encoding a signal sequence are codon diverged.

In certain embodiments of the cells disclosed herein, the exogenous TCR recognizes a cancer neoantigen. In certain embodiments of the cells disclosed herein, the neoantigen is a private neoantigen. In certain embodiments of the cells disclosed herein, the sequence encoding an exogenous TCR is obtained from a subject.

In certain embodiments of the cells disclosed herein, the cell is a primary cell. In certain embodiments of the cells disclosed herein, the cell is a patient-derived cell. In certain embodiments of the cells disclosed herein, the cell is a lymphocyte. In certain embodiments of the cells disclosed herein, the cell is a T cell. In certain embodiments of the cells disclosed herein, the cell is a young T cell. In certain embodiments of the cells disclosed herein, the cell is CD45RA+, CD62L+, CD28+, CD95−, CCR7+, and CD27+. In certain embodiments of the cells disclosed herein, the cell is CD45RA+, CD62L+, CD28+, CD95+, CD27+, CCR7+. In certain embodiments of the cells disclosed herein, the cell is CD45RO+, CD62L+, CD28+, CD95+, CCR7+, CD27+, CD127+.

In certain embodiments of the cells disclosed herein, the endogenous locus within the genome of the cell is a TCR locus. In certain embodiments of the cells disclosed herein, the TCR locus is a TRAC locus or a TRBC locus. In certain embodiments of the cells disclosed herein, the TCR locus is a TRAC locus and a TRBC locus. In certain embodiments of the cells disclosed herein, the endogenous promoter is a TRAC promoter. In certain embodiments of the cells disclosed herein, the endogenous promoter is a TRBC promoter.

In certain embodiments of the cells disclosed herein, the cell, the sequence encoding an exogenous TCR, and the sequence of the neoantigen are obtained from the same subject.

In certain embodiments of the polynucleotides disclosed herein, the present disclosure provides a polynucleotide comprising an exogenous enhancer, an insulator, a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising an exogenous enhancer, a pause element, a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising a Woodchuck Hepatitis Virus Posttranscriptional Regulatory Element (WPRE), a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising an insulator, a Woodchuck Hepatitis Virus Posttranscriptional Regulatory Element (WPRE), a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising an insulator, a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter.

In certain embodiments, the present disclosure provides a polynucleotide comprising a first homology arm, a second homology arm, a sequence encoding an exogenous TCR, a first sequence encoding a Payload, and a second sequence encoding a Payload, wherein the first and second homology arms are homologous to a TRAC or TRBC locus and wherein the second sequence encoding a Payload is under control of an exogenous promoter.

In certain embodiments, the present disclosure provides a polynucleotide comprising a sequence encoding a first homology arm, a second homology arm, an exogenous TCR, and a sequence encoding at least one Payload, wherein the first and second homology arms are homologous to a TRAC or TRBC locus.

In certain embodiments of the polynucleotides disclosed herein, the insulator is an HS4 insulator or an IS2 insulator. In certain embodiments of the polynucleotides disclosed herein, the insulator comprises a nucleotide sequence that is at least about 80% identical to the nucleotide sequence set forth in SEQ ID NO: 18 or SEQ ID NO: 19. In certain embodiments of the polynucleotides disclosed herein, the insulator comprises the nucleotide sequence set forth in SEQ ID NO: 18 or SEQ ID NO: 18. In certain embodiments of the polynucleotides disclosed herein, the WPRE comprises a nucleotide sequence that is at least about 80% identical to the nucleotide sequence set forth in SEQ ID NO: 20. In certain embodiments of the polynucleotides disclosed herein, the WPRE comprises the nucleotide sequence set forth in SEQ ID NO: 20.

In certain embodiments of the polynucleotides disclosed herein, the exogenous enhancer is a CMV enhancer, a TCRα enhancer, or a TCRβ enhancer. In certain embodiments of the polynucleotides disclosed herein, the exogenous enhancer is a TCRα enhancer. In certain embodiments of the polynucleotides disclosed herein, the TCRα enhancer comprises a nucleotide sequence that is at least about 80% identical to the nucleotide sequence set forth in SEQ ID NO: 32. In certain embodiments of the polynucleotides disclosed herein, the TCRα enhancer comprises the nucleotide sequence set forth in SEQ ID NO: 32. In certain embodiments of the polynucleotides disclosed herein, the exogenous enhancer is a CMV enhancer. In certain embodiments of the polynucleotides disclosed herein, the CMV enhancer comprises a nucleotide sequence that is at least about 80% identical to the nucleotide sequence set forth in SEQ ID NO: 33. In certain embodiments of the polynucleotides disclosed herein, the CMV enhancer comprises the nucleotide sequence set forth in SEQ ID NO: 33.

In certain embodiments of the polynucleotides disclosed herein, the exogenous promoter is a constitutive promoter. In certain embodiments of the polynucleotides disclosed herein, the constitutive promoter is an MDN promoter, an EF1α promoter, an ACTB promoter, a PGK promoter, or a U6 promoter. In certain embodiments of the polynucleotides disclosed herein, the constitutive promoter is an MDN promoter. In certain embodiments of the polynucleotides disclosed herein, the constitutive promoter is an EF1α promoter. In certain embodiments of the polynucleotides disclosed herein, the exogenous promoter is an inducible promoter. In certain embodiments of the polynucleotides disclosed herein, the inducible promoter is an AP1 promoter, an NFAT promoter, an NF-κB promoter, or an NR4A-responsive promoter.

In certain embodiments of the polynucleotides disclosed herein, the sequence encoding an exogenous TCR comprises a TCRα gene sequence or a TCRβ gene sequence. In certain embodiments of the polynucleotides disclosed herein, the sequence encoding an exogenous TCR comprises a TCRα gene sequence and a TCRβ gene sequence. In certain embodiments of the polynucleotides disclosed herein, the sequence encoding an exogenous TCR further comprises a sequence encoding a P2A peptide, a sequence encoding a signal sequence, a sequence encoding a protease cleavage peptide, or a combination thereof. In certain embodiments of the polynucleotides disclosed herein, the sequence encoding an exogenous TCR comprises a TCRα gene sequence, a TCRβ gene sequence, a sequence encoding a P2A peptide, a sequence encoding a signal sequence, a sequence encoding a protease cleavage peptide, or a combination thereof.

In certain embodiments of the polynucleotides disclosed herein, the sequence encoding an exogenous TCR comprises, from 5′ end to 3′ end, a first sequence encoding a P2A peptide, a first sequence encoding a signal sequence, a TCRβ gene sequence, a sequence encoding a protease cleavage peptide, a second sequence encoding a P2A peptide, a second sequence encoding a signal sequence, and a TCRα gene sequence. In certain embodiments of the polynucleotides disclosed herein, the sequence encoding an exogenous TCR comprises, from 5′ end to 3′ end, a first sequence encoding a P2A peptide, a first sequence encoding a signal sequence, a TCRβ gene sequence, a sequence encoding a protease cleavage peptide, a second sequence encoding a P2A peptide, a second sequence encoding a signal sequence, a TCRα gene sequence, and a poly-adenylation sequence. In certain embodiments of the polynucleotides disclosed herein, the sequence encoding an exogenous TCR comprises, from 5′ end to 3′ end, a first sequence encoding a P2A peptide, a first sequence encoding a signal sequence, a TCRα gene sequence, a sequence encoding a protease cleavage peptide, a second sequence encoding a P2A peptide, a second sequence encoding a signal sequence, a TCRβ gene sequence, and a poly-adenylation sequence. In certain embodiments of the polynucleotides disclosed herein, the at least one Payload is selected from the group consisting of a cytokine receptors trap, a ligand trap, an angiogenesis factor, an apoptotic factor, an inhibitory protein, an extracellular matrix modulator, a soluble TCR, a homing signal, an enzyme, a modulator of reactive oxygen species, a competitive ligand inhibitor, a protein that binds to receptors and sterically hinders receptor function, and an inhibitory RNA molecule.

In certain embodiments of the polynucleotides disclosed herein, the 3′ of the at least one Payload comprises a STOP codon. In certain embodiments of the polynucleotides disclosed herein, the 3′ of the at least one Payload comprises a sequence encoding a 2A peptide and a sequence encoding a protease cleavage peptide. In certain embodiments of the polynucleotides disclosed herein, the 3′ of the at least one Payload comprises a poly-adenylation sequence.

In certain embodiments of the polynucleotides disclosed herein, the at least one Payload is an inhibitory RNA molecule. In certain embodiments of the polynucleotides disclosed herein, the inhibitory RNA molecule is a shRNA, a miRNA, or a miRNA cluster. In certain embodiments of the polynucleotides disclosed herein, the inhibitory RNA molecule is a miRNA. In certain embodiments of the polynucleotides disclosed herein, the miRNA comprises a first flanking sequence and a second flanking sequence. In certain embodiments of the polynucleotides disclosed herein, the first flanking sequence and the second flanking sequence are derived from miR-155, miR-30, miR-17/92, miR-122, or miR-21. In certain embodiments of the polynucleotides disclosed herein, the inhibitory RNA molecule is flanked by a splice donor site or a splice acceptor site. In certain embodiments of the polynucleotides disclosed herein, the inhibitory RNA molecule is flanked by a splice donor site and a splice acceptor site.

In certain embodiments of the polynucleotides disclosed herein, the sequences encoding a P2A peptide are codon diverged. In certain embodiments of the polynucleotides disclosed herein, the sequences encoding a protease cleavage peptide are codon diverged. In certain embodiments of the polynucleotides disclosed herein, the sequences encoding a signal sequence are codon diverged. In certain embodiments of the polynucleotides disclosed herein, the exogenous TCR recognizes a cancer neoantigen. In certain embodiments of the polynucleotides disclosed herein, the neoantigen is a private neoantigen. In certain embodiments of the polynucleotides disclosed herein, the sequence encoding an exogenous TCR is obtained from a single subject.

In certain embodiments of the polynucleotides disclosed herein, the polynucleotide is a circular polynucleotide. In certain embodiments of the polynucleotides disclosed herein, the circular polynucleotide is a plasmid or a nanoplasmid. In certain embodiments of the polynucleotides disclosed herein, the polynucleotide is a linear polynucleotide.

In certain embodiments, the present disclosure further provides a vector comprising any one of the polynucleotide disclosed herein. In certain embodiments of the vectors disclosed herein, the vector is a non-viral vector. In certain embodiments, the present disclosure provides a cell comprising any one of the polynucleotides or vectors disclosed herein.

In certain embodiments, the present disclosure provides a method of modifying a cell. In certain embodiments of the methods disclosed herein, the method comprises introducing into the cell any one of the polynucleotides or vectors disclosed herein. In certain embodiments of the methods disclosed herein, the method further comprises recombining the polynucleotide or vector into an endogenous locus of the cell.

In certain embodiments of the methods disclosed herein, the introducing occurs via electroporation. In certain embodiments of the methods disclosed herein, the recombining comprises cleavage of the endogenous locus by a nuclease. In certain embodiments of the methods disclosed herein, the recombining further comprises recombination of the polynucleotide into the endogenous locus by homology-directed repair.

In certain embodiments of the methods disclosed herein, the nuclease is a Clustered Regularly Interspaced Short Palindromic Repeats (CRISPR) family nuclease, or a functional fragment thereof. In certain embodiments of the methods disclosed herein, the nuclease further comprises a gRNA.

In certain embodiments of the methods disclosed herein, the method further comprises culturing the cell in the presence of at least one cytokine. In certain embodiments of the methods disclosed herein, the at least one cytokine comprises IL2, IL7, IL15, or a combination thereof. In certain embodiments of the methods disclosed herein, the at least one cytokine comprises IL7 and IL15.

In certain embodiments of the methods disclosed herein, the cell is a primary cell. In certain embodiments of the methods disclosed herein, the cell is a patient-derived cell. In certain embodiments of the methods disclosed herein, the cell is a lymphocyte. In certain embodiments of the methods disclosed herein, the cell is a T cell. In certain embodiments of the methods disclosed herein, the cell is a young T cell. In certain embodiments of the methods disclosed herein, the cell is CD45RA+, CD62L+, CD28+, CD95−, CCR7+, and CD27+. In certain embodiments of the methods disclosed herein, the cell is CD45RA+, CD62L+, CD28+, CD95+, CD27+, CCR7+. In certain embodiments of the methods disclosed herein, the cell is CD45RO+, CD62L+, CD28+, CD95+, CCR7+, CD27+, CD127+.

In certain embodiments, the present disclosure also provides a cell modified by any one of the methods disclosed herein.

In certain embodiments, the present disclosure provides a composition comprising an effective amount of the any one of the cells disclosed herein. In certain embodiments of the compositions disclosed herein, the composition is a pharmaceutical composition that further comprises a pharmaceutically acceptable excipient. In certain embodiments of the compositions disclosed herein, the composition is administered to a patient in need thereof for the treatment of cancer. In certain embodiments of the compositions disclosed herein, the composition comprises a cryopreservation agent. In certain embodiments of the compositions disclosed herein, the composition comprises serum albumin. In certain embodiments of the compositions disclosed herein, the composition comprises Plasma-Lyte A, HSA, and CryoStor CS10.

In certain embodiments, the present disclosure provides methods of treating cancer in a subject in need thereof. In certain embodiments of the methods disclosed herein, the method comprises administering a therapeutically effective amount of any one of the cells disclosed herein. In certain embodiments of the methods disclosed herein, the method comprises administering a therapeutically effective amount of any one of the compositions disclosed herein. In certain embodiments of the methods disclosed herein, prior to administering, a non-myeloablative lymphodepletion regimen is administered to the subject.

In certain embodiments of the methods disclosed herein, the cancer is a solid tumor or a liquid tumor. In certain embodiments of the methods disclosed herein, the solid tumor is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinomas, renal cell cancers, testicular cancer, sarcomas, and colorectal cancer. In certain embodiments of the methods disclosed herein, the liquid tumor is selected from the group consisting of follicular lymphoma, leukemia, and multiple myeloma.

In certain embodiments, the present disclosure provides a kit comprising any one of the cells, the polynucleotides, the vectors, or the compositions disclosed herein. In certain embodiments of the kits disclosed herein, the kit further comprises written instructions for treating cancer.

In certain embodiments, the present disclosure provides a method of modifying a cell. In certain embodiments of the methods disclosed herein, the method comprises introducing into the cell a homologous recombination (HR) template nucleic acid sequence, wherein the HR template comprises a Secondary Promoter Construct, and recombining the HR template nucleic acid into an endogenous locus of the cell. In certain embodiments of the methods disclosed herein, the Secondary Promoter Construct comprises Format 1, 2, or 3. In certain embodiments of the methods disclosed herein, the Secondary Promoter Construct comprises Format 4.

In certain embodiments, the present disclosure provides a cell modified by any of the methods disclosed herein. In certain embodiments, the present disclosure provides a composition comprising a Secondary Cell Product made according to any of the methods disclosed herein. In certain embodiments of the compositions disclosed herein, the composition is a pharmaceutical composition that further comprises a pharmaceutically acceptable excipient. In certain embodiments of the compositions disclosed herein, the composition is administered to a patient in need thereof for the treatment of cancer.

In certain embodiments, the present disclosure provides a method of treating cancer in a subject in need thereof, the method comprising administering a therapeutically effective amount of any one of the cells or compositions disclosed herein. In certain embodiments of the methods disclosed herein, prior to administering, a non-myeloablative lymphodepletion regimen is administered to the subject. In certain embodiments of the methods disclosed herein, the cancer is a solid tumor. In certain embodiments of the methods disclosed herein, the cancer is liquid tumor. In certain embodiments of the methods disclosed herein, the solid tumor is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinomas, renal cell cancers, testicular cancer, sarcomas, and colorectal cancer. In certain embodiments of the methods disclosed herein, the liquid tumor is selected from the group consisting of follicular lymphoma, leukemia, and multiple myeloma.

In certain embodiments, the present disclosure provides a composition or a method of any of the embodiments described herein.

### EXAMPLES

The following are examples of methods and compositions of the invention. It is understood that various other embodiments may be practiced, given the general description provided above.

### Example 1. Generation of NeoTCR Products

Neoepitope-specific TCRs identified by the imPACT Isolation Technology described in PCT/US2020/17887 (which is herein incorporated by reference in its entirety) were used to generate homologous recombination (HR) DNA templates. These HR templates were transfected into primary human T cells in tandem with site-specific nucleases (see FIGS. 1A-1C). The single-step non-viral precision genome engineering resulted in the seamless replacement of the endogenous TCR with the patient's neoepitope-specific TCR, expressed by the endogenous promoter. The TCR expressed on the surface is entirely native in sequence.

The precision of NeoTCR-T cell genome engineering was evaluated by Targeted Locus Amplification (TLA) for off-target integration hot spots or translocations, and by next-generation sequencing based off-target cleavage assays and found to lack evidence of unintended outcomes.

As shown in FIGS. 1A-1C, constructs containing genes of interest were inserted into endogenous loci. This was accomplished with the use of homologous repair templates containing the coding sequence of the gene of interest flanked by left and right HR arms. In addition to the HR arms, the gene of interest was sandwiched between 2A peptides, a protease cleavage site that is upstream of the 2A peptide to remove the 2A peptide from the upstream translated gene of interest, and signal sequences (FIG. 1B). Once integrated into the genome, the gene of interested expression gene cassette was transcribed as single messenger RNA. During the translation of this gene of interest in messenger RNA, the flanking regions were unlinked from the gene of interest by the self-cleaving 2A peptide and the protease cleavage site was cleaved for the removal of the 2A peptide upstream from the translated gene of interest (FIG. 1C). In addition to the 2A peptide and protease cleavage site, a Gly-Ser-Gly (GSG) linker was inserted before each 2A peptide to further enhance the separation of the gene of interest from the other elements in the expression cassette.

It was determined that P2A peptides were superior to other 2A peptides for Cell Products because of their efficient cleavage. Accordingly, two (2) P2A peptides and codon divergence were used to express the gene of interest without introducing any exogenous epitopes from the remaining amino acids on either end of the gene of interest from the P2A peptide. The benefit of the gene-edited cell having no exogenous epitopes (i.e., no flanking P2A peptide amino acids on either side of the gene of interest) is that immunogenicity is drastically decreased and there is less likelihood of a patient infused with a Cell Product containing the gene-edited cell to have an immune reaction against the gene-edited cell.

As described in PCT/US/2018/058230, NeoTCRs were integrated into the TCRα locus of T cells. Specifically, a homologous repair template containing a NeoTCR coding sequence flanked by left and right HR Arms was used. In addition, the endogenous TCRβ locus was disrupted leading to the expression of only TCR sequences encoded by the NeoTCR construct. The general strategy was applied using circular HR templates as well as linear templates.

The target TCRα locus (Ca) is shown along with the plasmid HR template, and the resulting edited sequence and downstream mRNA/protein products in FIGS. 1B and 1C. The target TCRα locus (endogenous TRAC) and its CRISPR Cas9 target site (horizontal stripe, cleavage site designated by arrow) are shown (FIGS. 1A-1C). The circular plasmid HR template with the polynucleotide encoding the NeoTCR is located between left and right homology arms (“LHA” and “RHA” respectively). The region of the TRAC introduced by the HR template that was codon-optimized is shown (vertical stripe). The TCRβ constant domain was derived from TRBC2, which is indicated as being functionally equivalent to TRBC1. Other elements in the NeoTCR cassette include 2A=2A ribosome skipping element (by way of non-limiting example, the 2A peptides used in the cassette are both P2A sequences that are used in combination with codon divergence to eliminate any otherwise occurring non-endogenous epitopes in the translated product); P=protease cleavage site upstream of 2A that removes the 2A tag from the upstream TCRβ protein (by way of non-limiting example the protease cleavage site can be a furin protease cleavage site); SS=signal sequences (by way of non-limited example the protease cleavage site can be a human growth hormone signal sequence). The HR template of the NeoTCR expression gene cassette includes two flanking homology arms to direct insertion into the TCRα genomic locus targeted by the CRISPR Cas9 nuclease RNP with the TCRα guide RNA. These homology arms (LHA and RHA) flank the neoE-specific TCR sequences of the NeoTCR expression gene cassette. While the protease cleavage site used in this example was a furin protease cleavage site, any appropriate protease cleavage site known to one of skill in the art could be used. Similarly, while HGH was the signal sequence chosen for this example, any signal sequence known to one of skill in the art could be selected based on the desired trafficking and used.

Once integrated into the genome (FIG. 1C), the NeoTCR expression gene cassette is transcribed as a single messenger RNA from the endogenous TCRα promoter, which still includes a portion of the endogenous TCRα polypeptide from that individual T cell (FIG. 1C). During ribosomal polypeptide translation of this single NeoTCR messenger RNA, the NeoTCR sequences are unlinked from the endogenous, CRISPR-disrupted TCRα polypeptide by self-cleavage at a P2A peptide (FIG. 1C). The encoded NeoTCRα and NeoTCRβ polypeptides are also unlinked from each other through cleavage by the endogenous cellular human furin protease and a second self-cleaving P2A sequence motifs included in the NeoTCR expression gene cassette (FIG. 1C). The NeoTCRα and NeoTCRβ polypeptides are separately targeted by signal leader sequences (derived from the human growth hormone, HGH) to the endoplasmic reticulum for multimer assembly and trafficking of the NeoTCR protein complexes to the T cell surface. The inclusion of the furin protease cleavage site facilitates the removal of the 2A sequence from the upstream TCRβ chain to reduce potential interference with TCRβ function. The inclusion of a Gly-Ser-Gly linker before each 2A (not shown) further enhances the separation of the three polypeptides.

Additionally, three repeated protein sequences are codon diverged within the HR template to promote genomic stability. The two P2A are codon diverged relative to each other, as well as the two HGH signal sequences relative to each other, within the TCR gene cassette to promote stability of the introduced NeoTCR cassette sequences within the genome of the ex vivo engineered T cells. Similarly, the re-introduced 5′ end of TRAC exon 1 (vertical stripe) reduces the likelihood of the entire cassette being lost over time through the removal of an intervening sequence of two direct repeats.

In addition to NeoTCR Products, this method can be used for any Secondary Promoter Product.

In-Out PCR was used to confirm the precise target integration of the NeoE TCR cassette. Agarose gels show the results of a PCR using primers specific to the integration cassette and site generate products of the expected size only for cells treated with both nuclease and DNA template (KOKI and KOKIKO), demonstrating site-specific and precise integration.

Furthermore, Targeted Locus Amplification (TLA) was used to confirm the specificity of targeted integration. Crosslinking, ligation, and use of primers specific to the NeoTCR insert were used to obtain sequences around the site(s) of integration. The reads mapped to the genome are binned in 10 kb intervals. Significant read depths were obtained only around the intended site the integration site on chromosome 14, showing no evidence of common off-target insertion sites.

Antibody staining for endogenous TCR and peptide-HLA staining for NeoTCR revealed that the engineering results in high frequency knock-in of the NeoTCR, with some TCR-cells and few WT T cells remaining. Knock-in is evidenced by NeoTCR expression in the absence of an exogenous promoter. Engineering was carried out multiple times using the same NeoTCR with similar results. Therefore, efficient and consistent expression of the NeoTCR and knockout of the endogenous TCR in engineered T cells was achieved.

### Example 2. Generation of Secondary Promoter Constructs

Format 1. Format 1 of the modular Secondary Promoter Constructs is exemplified in FIGS. 2A, 2B, 3A, and 3B. Format 1 is designed so that the secondary transcript (i.e., the transcript of the Payload) appears in the same orientation as the TCR transcript. Another important feature of Format 1 is that the secondary transcript (i.e., the transcript of the Payload) uses the endogenous poly-A. In this format, the TCR transcript and secondary transcript both start at the 5′ end of the sense strand. It is shown that the promoter region is followed by a Payload of interest and the right homology arm.

The 2A sequences of Format 1 can be two P2As, one P2A, and one T2A, or two T2As. However, the preferred format used to design the construct and make mCherry Payload Secondary Promoters and resulting Secondary Promoter Cells and Products used two P2As.

In Format 1, each of the 2A sequences is preceded by a GSG linker or functionally equivalent linker and the Secondary Promoter region may be followed by a Kozak sequence.

As shown in FIG. 3A, an exemplary Secondary Promoter Construct was designed and made into Secondary Promoter Cells and Products as follows (listed in order of each element): Left homology arm; GSG linker; P2A sequence; HGH signal sequence; full length TCR beta gene; Furin cleavage sequence; GSG linker; P2A sequence; HGH signal sequence; full length TCR alpha gene; poly-A signal sequence; insulator sequence; promoter region; Kozak sequence; Payload of interest; right homology arm—all retained within a backbone.

As shown in FIG. 2A, as a modular construct, one of skill in the art can select the appropriate element for each modular component (i.e., which 2A sequence, which promoter, which signal sequence, which protease cleavage site, etc.) based on the expression needs of the Secondary Promoter Construct which will be dependent on the Payload.

Furthermore, FIGS. 2B and 3B provide an alternate version of Format 1 with modular optionality (FIG. 2B) and by example (FIG. 3B).

Format 2. Format 2 of the modular Secondary Promoter Constructs is exemplified in FIGS. 4A-C, and 5A-5C. Format 2 is designed so that the secondary transcript (i.e., the transcript of the Payload) appears in the same orientation as the TCR transcript much like in Format 1. However, unlike in Format 1, the secondary transcript uses its own poly-A rather than the endogenous poly-A.

The 2A sequences of Format 2 can be two P2As, one P2A, and one T2A, or two T2As. However, the preferred format used to design the construct and make mCherry Payload Secondary Promoters and resulting Secondary Promoter Cells and Products used two P2As.

In Format 2, each of the 2A sequences is preceded by a GSG linker or functionally equivalent linker and the Secondary Promoter region may be followed by a Kozak sequence.

As shown in FIG. 5A, an exemplary Secondary Promoter Construct was designed and made into Secondary Promoter Cells and Products as follows (listed in order of each element): Left homology arm; GSG linker; P2A sequence; HGH signal sequence; full length TCR beta gene; Furin cleavage sequence; GSG linker; P2A sequence; HGH signal sequence; full length TCR alpha gene; poly-A signal sequence; insulator sequence; promoter region; Kozak sequence; Payload of interest; WPRE; poly-A signal sequence; right homology arm—all retained within a backbone.

As shown in FIG. 4A, as a modular construct, one of skill in the art can select the appropriate element for each modular component (i.e., which 2A sequence, which promoter, which signal sequence, which protease cleavage site, etc.) based on the expression needs of the Secondary Promoter Construct which will be dependent on the Payload.

Furthermore, FIGS. 4B, 4C, 5B, and 5C provide an alternate version of Format 1 with modular optionality (FIGS. 4B and 4C) and by example ((FIGS. 5B and 5C).

Format 3. Format 3 of the modular Secondary Promoter Constructs is exemplified in FIGS. 6 and 7. Format 3 is designed so that the secondary transcript (i.e., the transcript of the Payload) appears in the reversed orientation as compared to the TCR transcript. Much like in Format 2, the secondary transcript does not use the endogenous poly-A. Instead, the secondary transcript and TCR transcript share the same poly-A (e.g., a bidirectional poly-A such as SV40).

The 2A sequences of Format 3 can be two P2As, one P2A, and one T2A, or two T2As. However, the preferred format used to design the construct and make mCherry Payload Secondary Promoters and resulting Secondary Promoter Cells and Products used two P2As.

In Format 3, each of the 2A sequences is preceded by a GSG linker or functionally equivalent linker and the Secondary Promoter region may be followed by a Kozak sequence.

As shown in FIG. 7, an exemplary Secondary Promoter Construct was designed and made into Secondary Promoter Cells and Products as follows (listed in order of each element): Left homology arm; GSG linker; P2A sequence; HGH signal sequence; full length TCR beta gene; Furin cleavage sequence; GSG linker; P2A sequence; HGH signal sequence; full length TCR alpha gene; poly-A signal sequence; WPRE; Payload of interest; Kozak sequence; promoter region; right homology arm—all retained within a backbone.

As shown in FIG. 6, as a modular construct, one of skill in the art can select the appropriate element for each modular component (i.e., which 2A sequence, which promoter, which signal sequence, which protease cleavage site, etc.) based on the expression needs of the Secondary Promoter Construct which will be dependent on the Payload.

### Example 3. Generation of Secondary Promoter Products

T cell Isolation and Editing. CD4 and CD8 T cells were isolated from healthy donor PBMCs using the Miltenyi Prodigy or Miltenyi MACS separation columns according to the manufacturers' instructions. Positively-selected CD4 and CD8 T cells were used fresh or cryopreserved in 1% human serum albumin (Gemini), 49% Plasmalyte (Baxter), and 50% CS10 (Sigma). Cryopreserved cells were thawed, washed in media, and seeded at a density of 2×10⁶ cells per mL culture medium. One day after thaw, or immediately if used fresh, the cells were washed and re-seeded at a density of 1.46×106 cells per mL in culture medium+12.5 ng/mL IL7+12.5 ng/mL IL15+a T cell activation reagent by volume. Two days after activation, T cells were electroporated with a plasmid (containing a Secondary Promoter Construct) for the production of Secondary Promoter Cells and a Secondary Promoter Product. T cells were electroporated expanded in culture medium supplemented with 12.5 ng/mL IL7+12.5 ng/mL IL15. Supplemented medium was exchanged every 2-3 days or as needed until the end of the study, 13 days after activation.

comPACT and comPACT-Dextramer preparation. Neoantigen-specific peptide-HLA complex polypeptides (each a “comPACT”) were prepared according to the method as described in PCT/US2019/025415, hereby incorporated by reference in its entirety. A comPACT-dextramer complex was made for the labeling of NeoTCR expressing T cells. Biotinylated comPACT protein was incubated with a streptavidin-conjugated fluorophore for 10 min at room temperature (RT). Biotin-40-dextran (NANOCS) was added to the mixture and incubated at RT for an additional 10 minutes. The comPACT-Dextramer was stored at 4° C.

Confirmation of comPACT binding to NeoTCR edited T cells. T cells were stained for flow cytometry. Cells were first stained with viability dye for 20 minutes at 4° C., then washed and stained with the comPACT-dextramer for 10 minutes at 4° C. Surface antibodies (anti-CD8a, anti-CD80, anti-CD4) were added to the suspension of cells and comPACT-dextramer, and the cells are incubated for an additional 20 minutes at 4° C. Cells were then washed and fixed in intracellular fixation buffer (BD Biosciences). All cells were acquired on an Attune NxT Flow Cytometer (ThermoFisher Scientific) and data were analyzed with either FCS Express or FlowJo.

Cytometric Bead Array (CBA). Streptavidin-coated plates (Eagle Biosciences) were washed 3 times with wash buffer (PBS supplemented with 1% BSA and 0.05% tween20) and then coated with comPACTs at different concentrations ranging from 100-0.01 ng/well. Wells with no comPACT and wells coated with mismatched comPACT were used as controls. The plates were incubated for 2 hr at room temperature, washed three times with wash buffer, and then washed three times with TexMACS supplemented with 3% human AB serum to remove the tween20. T cells were given two washes with TexMACS supplemented with 3% human AB serum and resuspended at 1 million cells/mL in TexMACS supplemented with 3% human AB serum and 1×penicillin-streptomycin solution. T cells were plated onto the comPACT coated plate at 100 μL/well and incubated at 37° C., 5% CO2. After 24 h the supernatant was collected, and the cytokine concentrations were analyzed using the BD Cytometric Bead Array (CBA) Human Th1/Th2 Cytokine Kit II (Catalog No. 551809) following the manufacturer's protocol. Capture beads were mixed with culture supernatant, incubated with the detection reagent for 3 hr at RT protected from light, washed, and resuspended in wash buffer. Samples were assayed on an Attune NxT Flow Cytometer and data analyzed with FlowJo. The EC50 represents the concentration of cognate comPACT that elicits 50% of the maximum response and is calculated utilizing a least-squares fit of IFNγ secretion over a range of comPACT concentrations.

Intracellular Staining. T cells were stained for flow cytometry on the indicated days. T cells are first stained with viability dye for 20 minutes at 4° C., then washed and incubated with surface antibodies (anti-CD8α, anti-CD8β, anti-CD4) for an additional 20 minutes at 4° C. T cells are then washed and permeabilized for intracellular staining. T cells are stained with anti-2A peptide or with anti-IFNγ, anti-TNF, or anti-IL2 in permeabilization buffer for 20 minutes at 4° C. T cells are fixed in intracellular fixation buffer (BD Biosciences). Samples are assayed on an Attune NxT Flow Cytometer (ThermoFisher Scientific) and data analyzed with either FCS Express or FlowJo.

T cell Proliferation Assay. Edited CD4 and CD8 T cells are labeled with the e450 proliferation dye (eBioscience) according to the manufacturer's instructions. Labeled cells were stimulated on comPACT coated plates with a range of concentrations as described above. T cells were harvested over 48-96 hours and analyzed for proliferation as measured by dilution of the e450 dye.

T cell Killing Assay. HLA-matched cell lines were pulsed with the cognate neoantigen peptide or mismatched peptide for 1 h at 37° C., 5% C02. The cells were washed 3 times with media to remove any unbound peptide and then co-cultured with edited CD4 and CD8 T cells that are labeled with the e450 proliferation dye described above. Co-cultures were incubated for 48 h at 37° C. with 5% C02 before harvest. Cells were washed and stained with a fixable viability dye to determine killing efficiency. The e450 proliferation dye was used to distinguish edited T cells from target cells.

### Example 4. Decoupling of NeoTCR Expression from a Secondary Payload

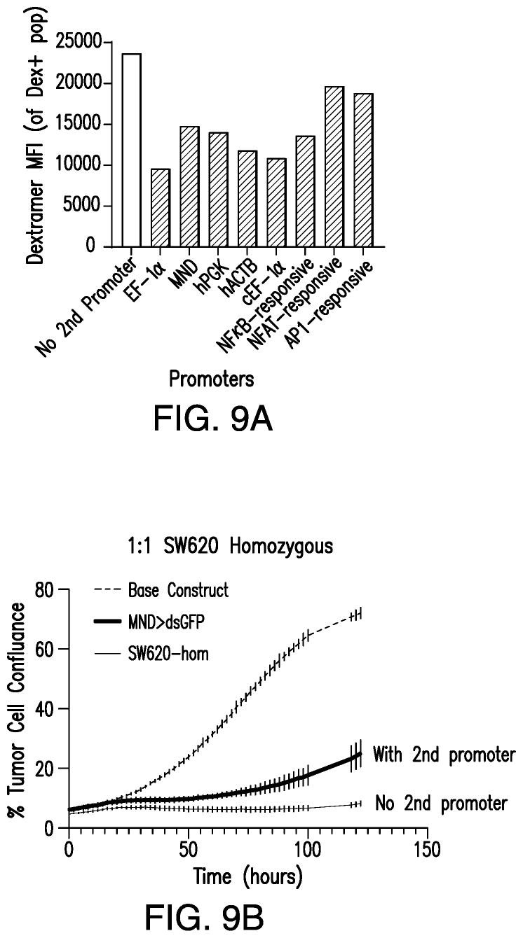

The present example provides several Secondary Promoter Constructs in order to decouple the expression of exogenous TCRs (e.g., NeoTCR) from the expression of a secondary Payload. It was observed that the introduction of a second promoter to express a Payload (i.e., a Secondary Promoter Construct) had a negative impact on the expression of the exogenous TCR (see FIG. 9A) and on the killing activity against tumor cells (see FIG. 9B). Thus, studies were performed to identify genomic elements that could improve the expression of NeoTCR in edited cells.

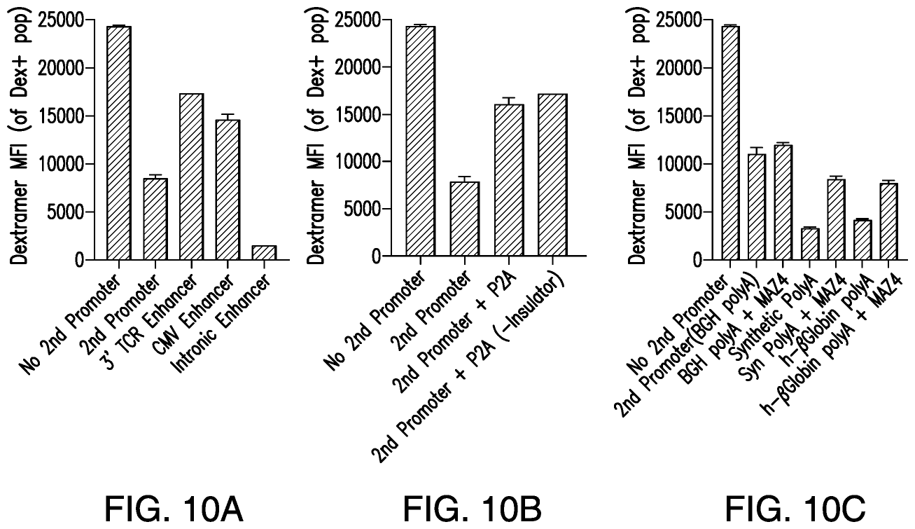

FIG. 10A shows that the use of a secondary promoter significantly reduced the expression of the NeoTCR and that the addition of the TCRα enhancer or a CMV-derived enhancer mitigated the reduction in NeoTCR expression. In addition, it was determined that the replacement of a STOP codon with a sequence encoding Furin and P2A peptides after the Payload improved the expression of NeoTCR (see FIG. 10B). Notably, the removal of an HS4 insulator between the TCR gene sequence and the Payload has little to no effect on the NeoTCR expression. Finally, it was observed that the insertion of a MAZ4 pause element after the poly-A signal sequence increased the NeoTCR expression (see FIG. 10C).

### Example 5. Design of Secondary Promoter Constructs

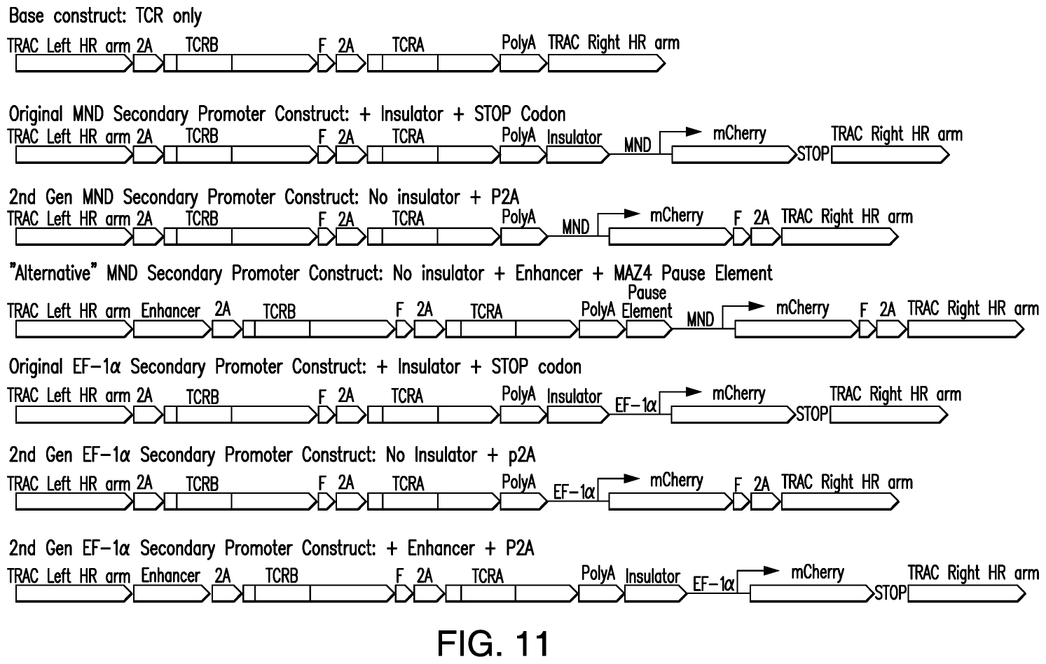

Based on the observations from Example 4, several Secondary Promoter Constructs were designed in order to decouple the expression of the NeoTCR from the Payload. The used constructs are depicted in FIG. 11.

The “base construct” encodes for a T cell receptor and includes a left homology arm, a sequence encoding a GSG linker, a sequence encoding a P2A sequence, a sequence encoding an HGH signal sequence, a full length TCRβ gene sequence, a sequence encoding a Furin cleavage sequence, a sequence encoding a second GSG linker, a sequence encoding a second P2A sequence, a full length TCRα gene sequence, a polyadenylation sequence, and a right homology arm.

The “original MND” Secondary Promoter Construct encodes for a T cell receptor and a Payload and includes a left homology arm, a sequence encoding a GSG linker, a sequence encoding a P2A sequence, a sequence encoding an HGH signal sequence, a full length TCRβ gene sequence, a sequence encoding a Furin cleavage sequence, a sequence encoding a second GSG linker, a sequence encoding a second P2A sequence, a full length TCRα gene sequence, a polyadenylation sequence, an insulator, an MND promoter, a Payload, a STOP codon, and a right homology arm. The original MND Secondary Promoter Construct encoding mCherry as Payload is also identified as “TCR-HS4-MND>mCherry-STOP.”

The “second generation MND” Secondary Promoter Construct encodes for a T cell receptor and a Payload and includes a left homology arm, a sequence encoding a GSG linker, a sequence encoding a P2A sequence, a sequence encoding an HGH signal sequence, a full length TCRβ gene sequence, a sequence encoding a Furin cleavage sequence, a sequence encoding a second GSG linker, a sequence encoding a second P2A sequence, a full length TCRα gene sequence, a polyadenylation sequence, an MND promoter, a Payload, a sequence encoding a second Furin cleavage sequence, a sequence encoding a third GSG linker, a sequence encoding a third P2A sequence, and a right homology arm. The second-generation MND Secondary Promoter Construct encoding mCherry as Payload is also identified as “TCR-MND>mCherry-2A.”

The “alternative MND” Secondary Promoter Construct encodes for a T cell receptor and a Payload and includes a left homology arm, a TCR gene enhancer, a sequence encoding a GSG linker, a sequence encoding a P2A sequence, a sequence encoding an HGH signal sequence, a full length TCRβ gene sequence, a sequence encoding a Furin cleavage sequence, a sequence encoding a second GSG linker, a sequence encoding a second P2A sequence, a full length TCRα gene sequence, a poly-adenylation sequence, a pause element, an MND promoter, a Payload, a sequence encoding a second Furin cleavage sequence, a sequence encoding a third GSG linker, a sequence encoding a third P2A sequence, and a right homology arm. The alternative MND Secondary Promoter Construct encoding mCherry as Payload is also identified as “3E-TCR-HS4-MND>mCherry-2A.”

The “original EF-1α” Secondary Promoter Construct encodes for a T cell receptor and a Payload and includes a left homology arm, a sequence encoding a GSG linker, a sequence encoding a P2A sequence, a sequence encoding an HGH signal sequence, a full length TCRβ gene sequence, a sequence encoding a Furin cleavage sequence, a sequence encoding a second GSG linker, a sequence encoding a second P2A sequence, a full length TCRα gene sequence, a polyadenylation sequence, an insulator, an EF-1α promoter, a Payload, a STOP codon, and a right homology arm. The original EF-1α Secondary Promoter Construct encoding mCherry as Payload is also identified as “TCR-HS4-cEF1α>mCherry-STOP.”

The “second generation EF-1α” Secondary Promoter Construct encodes for a T cell receptor and a Payload and includes a left homology arm, a sequence encoding a GSG linker, a sequence encoding a P2A sequence, a sequence encoding an HGH signal sequence, a full length TCRβ gene sequence, a sequence encoding a Furin cleavage sequence, a sequence encoding a second GSG linker, a sequence encoding a second P2A sequence, a full length TCRα gene sequence, a polyadenylation sequence, an EF-1α promoter, a Payload, a sequence encoding a second Furin cleavage sequence, a sequence encoding a third GSG linker, a sequence encoding a third P2A sequence, and a right homology arm. The second generation EF-1α Secondary Promoter Construct encoding mCherry as Payload is also identified as “TCR-cEF1α>mCherry-2A.”

Alternatively, the second generation EF-1α Secondary Promoter Construct includes a left homology arm, a TCRα gene enhancer, a sequence encoding a GSG linker, a sequence encoding a P2A sequence, a sequence encoding an HGH signal sequence, a full length TCRβ gene sequence, a sequence encoding a Furin cleavage sequence, a sequence encoding a second GSG linker, a sequence encoding a second P2A sequence, a full length TCRα gene sequence, a polyadenylation sequence, an insulator, an EF-1α promoter, a Payload, a STOP codon, and a right homology arm. The second generation EF-1α Secondary Promoter Construct encoding mCherry as Payload and including enhancer and insulator is also identified as “3E-TCR-HS4-cEF1α>mCherry-STOP.”

### Example 6. Effects of Secondary Promoter Constructs on Gene Expression Decoupling

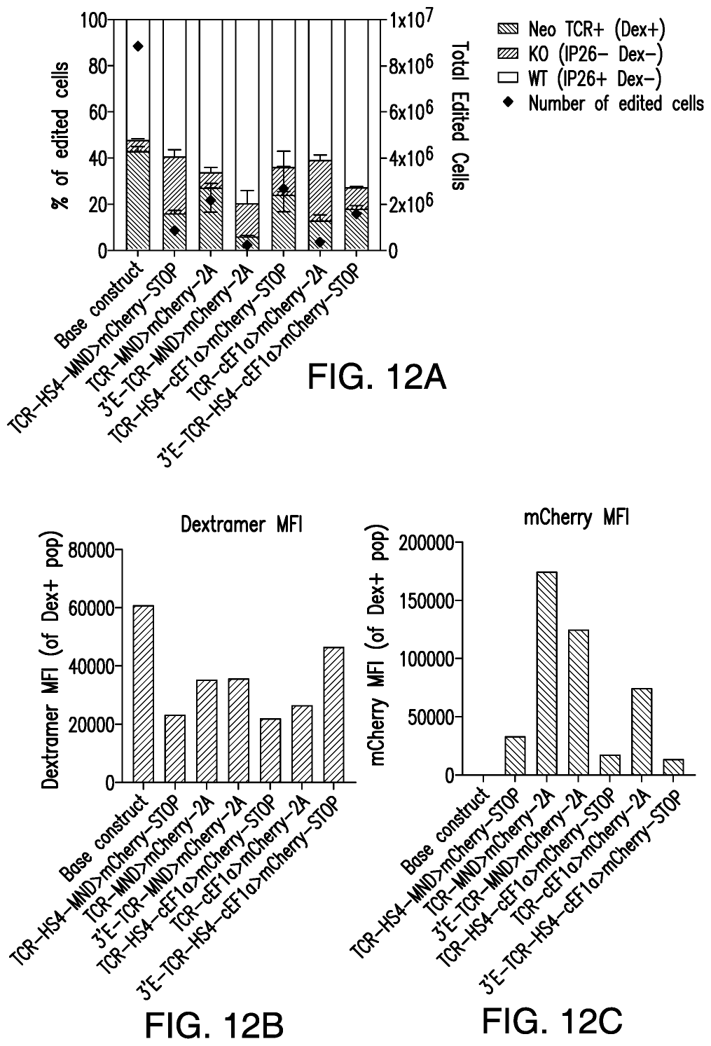

The Secondary Promoter Constructs described in Example 5 were used to determine the decoupling of gene expression of NeoTCR and Payload. As seen in FIG. 12A, cells transduced with the alternative MND Secondary Promoter Construct had a low gene editing efficacy. To confirm whether this effect was related to other features (e.g., the backbone of the plasmid) rather than the construct, the expression levels of the NeoTCR and the mCherry Payload were determined. Surprisingly, these constructs had similar NeoTCR expression levels and reduced mCherry expression. These results are in contrast to the TCR-cEF1α>mCherry-2A construct, which had improved NeoTCR expression and no significant reduction in mCherry. See FIGS. 12B and 12C.

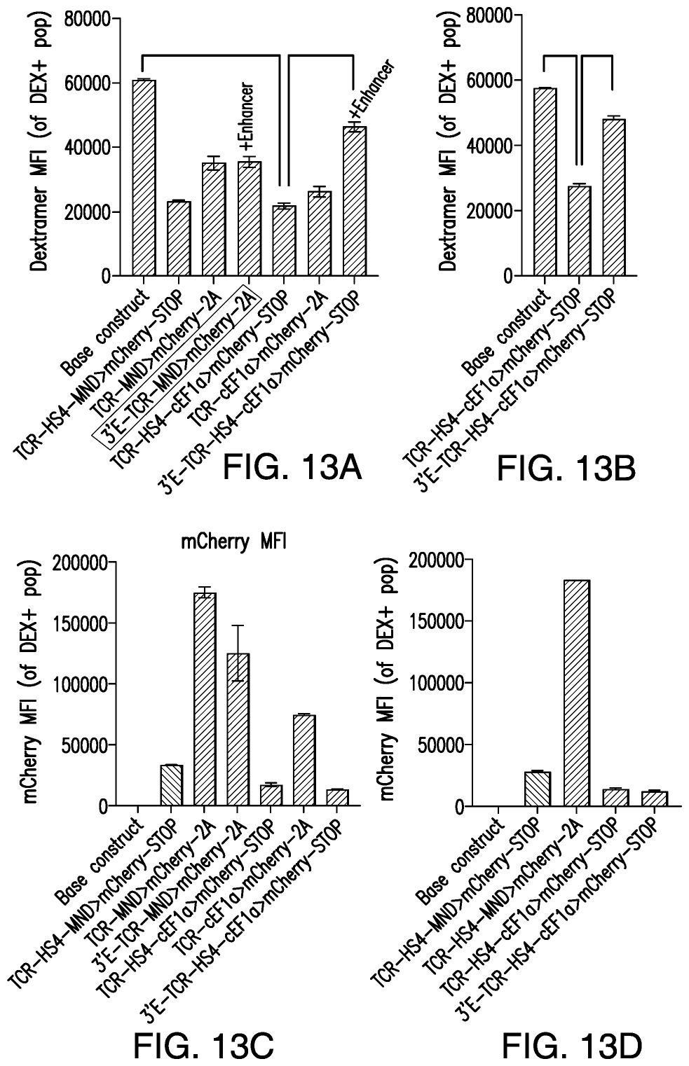

Next, experiments were conducted to confirm whether the TCRα enhancer (e.g., the 3′ portion of the protein coding sequence) could increase the expression of NeoTCR when using an EF-1α promoter. As shown in FIGS. 13A and 13B, the Secondary Promoter Constructs including the TCRα enhancer had increased NeoTCR expression levels in presence of the EF-1α promoter. Notably, the TCRα enhancer in presence of MND promoters did not improve NeoTCR expression beyond the addition of P2A alone. In addition, it was observed that Secondary Promoter Constructs including a sequence encoding a P2A after the Payload significantly increased the expression of the Payload mCherry (see FIGS. 13C and 13D).

These results show that removal of STOP codon improves the expression of TCR genes (e.g., NeoTCR) and Payload in all tested constructs. Further, the addition of enhancers did not improve the NeoTCR expression in presence of an MND promoter.

### Example 7. Effects of Secondary Promoter Constructs Including an Enhancer on Gene Expression Decoupling

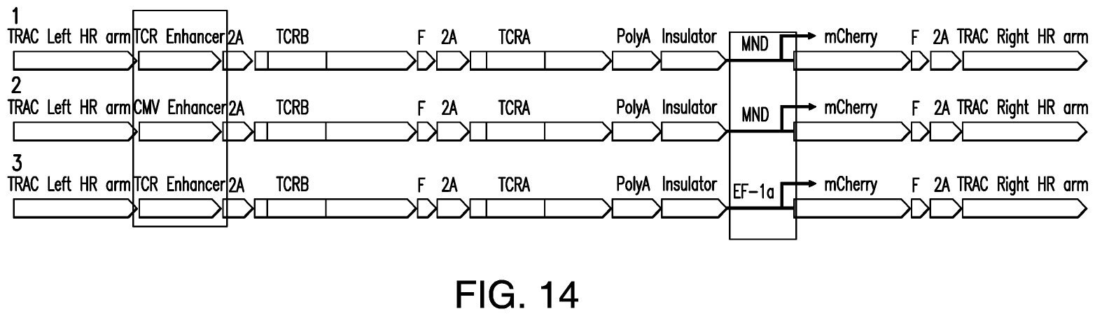

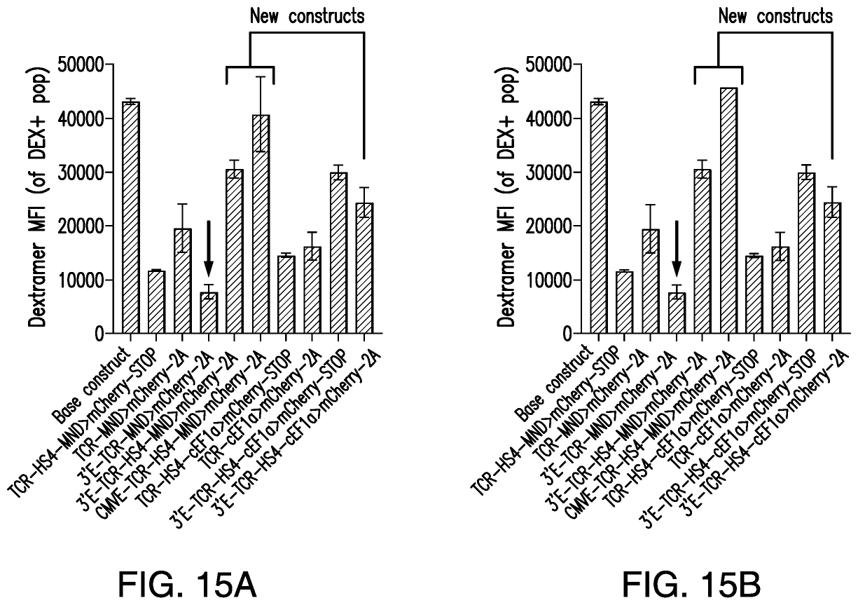

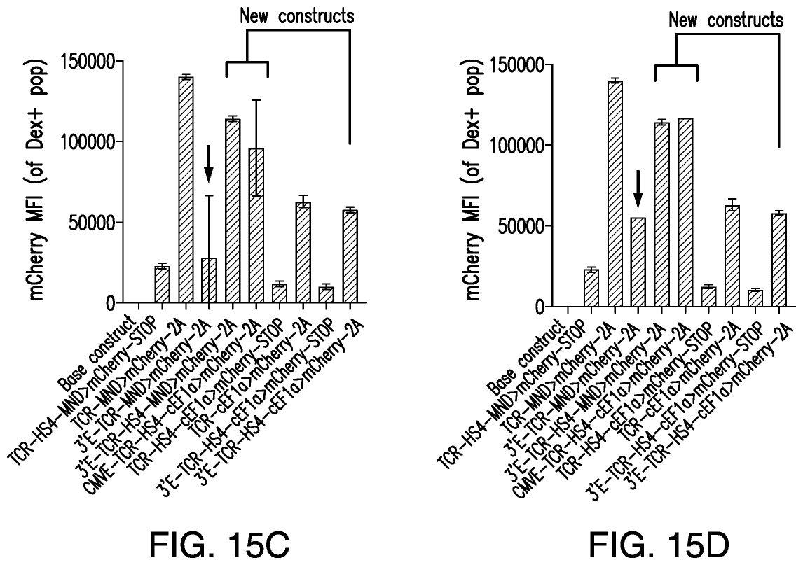

Since Secondary Promoter Constructs including an enhancer had improved NeoTCR expression, new constructs were designed to determine the relationship between enhancers and promoters. As seen in FIG. 14, three new Secondary Promoter Constructs were designed. The “construct 1” or “3E-TCR-HS4-MND>mCherry-2A” included a TCR enhancer and the MND promoter; “construct 2” or “CMVE-TCR-HS4-MND>mCherry-2A” included a CMV enhancer and the MND promoter; and “construct 3” or “3E-TCR-HS4-cEF1α>mCherry-2A” included a TCR enhancer and the EF-1α promoter. All the tested constructs included an insulator after the polyadenylation sequence. As seen in FIGS. 15A-15B, constructs with CMV enhancers and insulators showed comparable NeoTCR expression to the base constructs. In particular, the data in FIGS. 15A and 15B show that the inclusion of insulators in Secondary Promoter Constructs with an MND promoter enhanced the NeoTCR expression and that the CMV enhancer induced significantly elevated NeoTCR expression levels (i.e., similar to the base constructs). Finally, the addition of P2A sequence in constructs with EF-1α did not improve the NeoTCR expression as compared to constructs having an enhancer (see FIGS. 15C and 15D).

These data show that Secondary Promoter Constructs including an MND promoter perform best when in presence of a CMV enhancer, an HS4 insulator, and P2A peptides.

### Example 8. Secondary Promoter Construct Including Inhibitory RNA Molecules

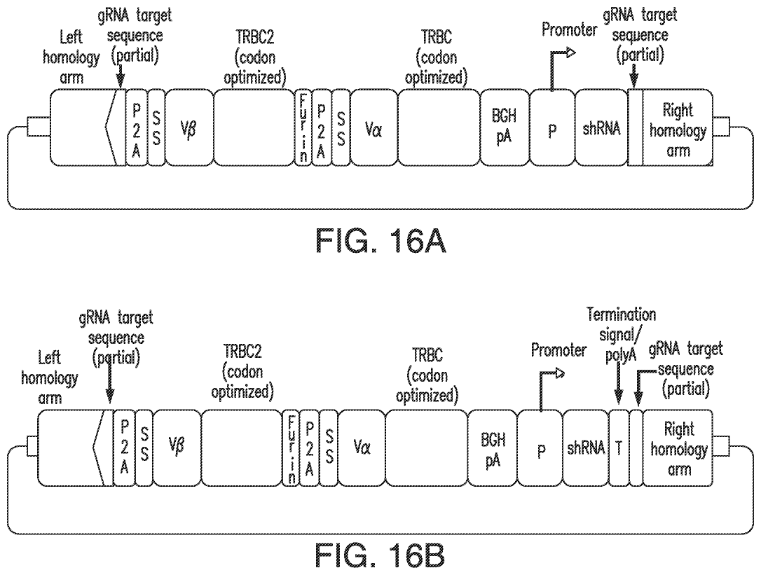

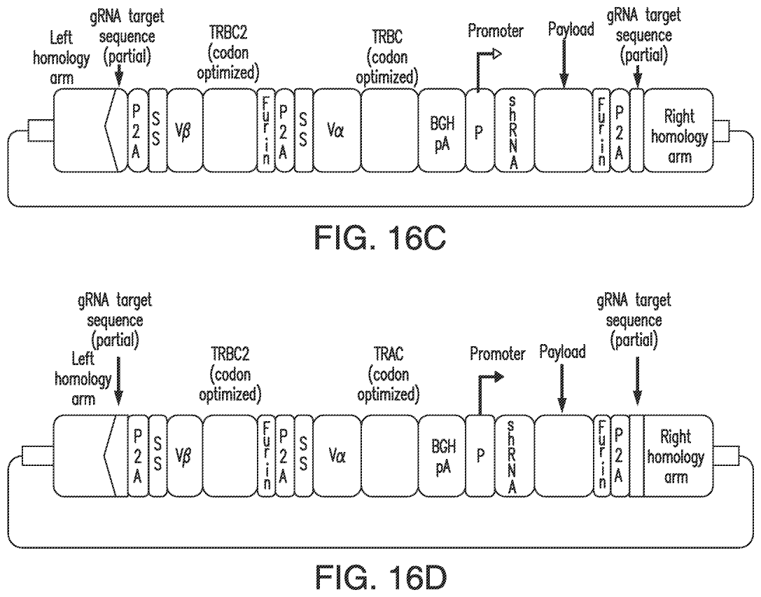

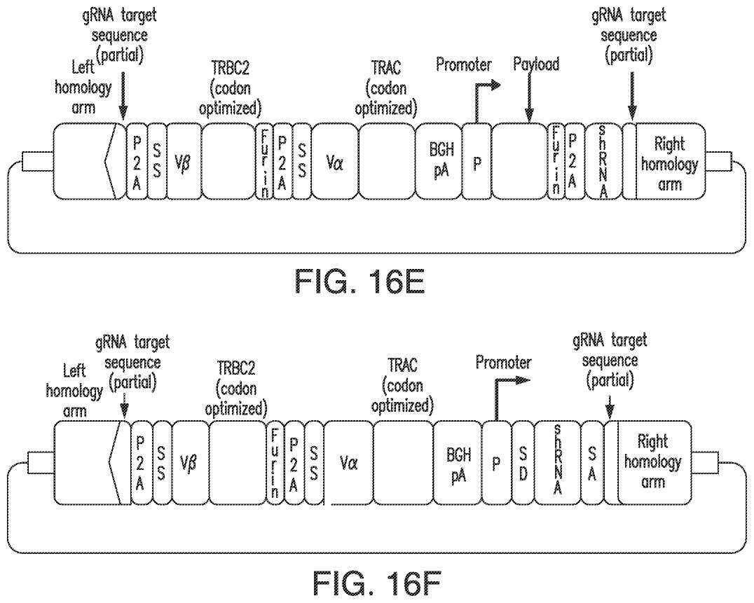

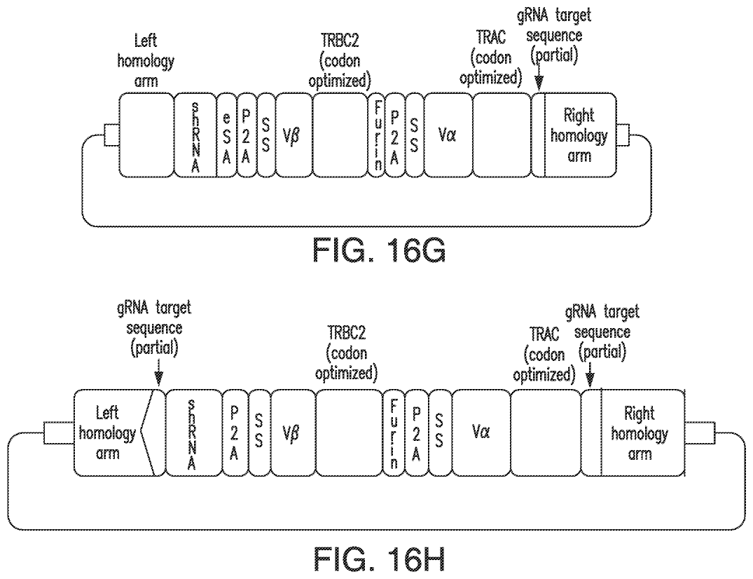

The present example discloses additional Secondary Promoter Constructs including an inhibitory RNA molecule disclosed herein. FIG. 16A shows a Secondary Promoter Construct including a left homology arm for TRAC locus, a first partial gRNA target sequence (e.g., used for integration of the construct in the TRAC locus), a first sequence encoding GSG and P2A peptides, a first sequence encoding a signal sequence, a TCRβ gene sequence, a sequence encoding a furin cleavage site, a second sequence encoding GSG and P2A peptides, a second sequence encoding a signal sequence, a TCRα gene sequence, a BGH polyadenylation signal, a secondary promoter, an inhibitory RNA, a second partial gRNA target sequence (e.g., used for integration of the construct in the TRAC locus), and a right homology arm for TRAC locus.

FIG. 16B shows a Secondary Promoter Construct including a left homology arm for TRAC locus, a first partial gRNA target sequence (e.g., used for integration of the construct in the TRAC locus), a first sequence encoding GSG and P2A peptides, a first sequence encoding a signal sequence, a TCRβ gene sequence, a sequence encoding a furin cleavage site, a second sequence encoding GSG and P2A peptides, a second sequence encoding a signal sequence, a TCRα gene sequence, a BGH polyadenylation signal, a secondary promoter, an inhibitory RNA, a termination signal or a polyadenylation site, a second partial gRNA target sequence (e.g., used for integration of the construct in the TRAC locus), and a right homology arm for TRAC locus.

FIGS. 16C and 16D show a Secondary Promoter Construct including a left homology arm for TRAC locus, a first partial gRNA target sequence (e.g., used for integration of the construct in the TRAC locus), a first sequence encoding GSG and P2A peptides, a first sequence encoding a signal sequence, a TCRβ gene sequence, a sequence encoding a furin cleavage site, a second sequence encoding GSG and P2A peptides, a second sequence encoding a signal sequence, a TCRα gene sequence, a BGH polyadenylation signal, a secondary promoter, an inhibitory RNA, a second Payload, a second sequence encoding a furin cleavage site, a third sequence encoding GSG and P2A peptides, a second partial gRNA target sequence (e.g., used for integration of the construct in the TRAC locus), and a right homology arm for TRAC locus.

FIG. 16E shows a Secondary Promoter Construct including a left homology arm for TRAC locus, a first partial gRNA target sequence (e.g., used for integration of the construct in the TRAC locus), a first sequence encoding GSG and P2A peptides, a first sequence encoding a signal sequence, a TCRβ gene sequence, a sequence encoding a furin cleavage site, a second sequence encoding GSG and P2A peptides, a second sequence encoding a signal sequence, a TCRα gene sequence, a BGH polyadenylation signal, a secondary promoter, a second Payload, a second sequence encoding a furin cleavage site, a third sequence encoding GSG and P2A peptides, an inhibitory RNA, a second partial gRNA target sequence (e.g., used for integration of the construct in the TRAC locus), and a right homology arm for TRAC locus.

FIG. 16F shows a Secondary Promoter Construct including a left homology arm for TRAC locus, a first partial gRNA target sequence (e.g., used for integration of the construct in the TRAC locus), a first sequence encoding GSG and P2A peptides, a first sequence encoding a signal sequence, a TCRβ gene sequence, a sequence encoding a furin cleavage site, a second sequence encoding GSG and P2A peptides, a second sequence encoding a signal sequence, a TCRα gene sequence, a BGH polyadenylation signal, a secondary promoter, a splice donor site, an inhibitory RNA, a splice acceptor site, a second partial gRNA target sequence (e.g., used for integration of the construct in the TRAC locus), and a right homology arm for TRAC locus.

FIG. 16G shows a Secondary Promoter Construct including a left homology arm for TRAC locus, an inhibitory RNA, a splice acceptor site, a first sequence encoding GSG and P2A peptides, a first sequence encoding a signal sequence, a TCRβ gene sequence, a sequence encoding a furin cleavage site, a second sequence encoding GSG and P2A peptides, a second sequence encoding a signal sequence, a TCRα gene sequence, a partial gRNA target sequence (e.g., used for integration of the construct in the TRAC locus), and a right homology arm for TRAC locus.

FIG. 16H shows a Secondary Promoter Construct including a left homology arm for TRAC locus, a first partial gRNA target sequence (e.g., used for integration of the construct in the TRAC locus), an inhibitory RNA, a first sequence encoding GSG and P2A peptides, a first sequence encoding a signal sequence, a TCRβ gene sequence, a sequence encoding a furin cleavage site, a second sequence encoding GSG and P2A peptides, a second sequence encoding a signal sequence, a TCRα gene sequence, a second partial gRNA target sequence (e.g., used for integration of the construct in the TRAC locus), and a right homology arm for TRAC locus.

### Example 9. Effects of Secondary Promoter Constructs Including an Inhibitory RNA on Gene Expression Decoupling

The present example describes several approaches to include an inhibitory RNA in a Secondary Promoter Construct. CBLB was used as target gene for knockdown analysis.

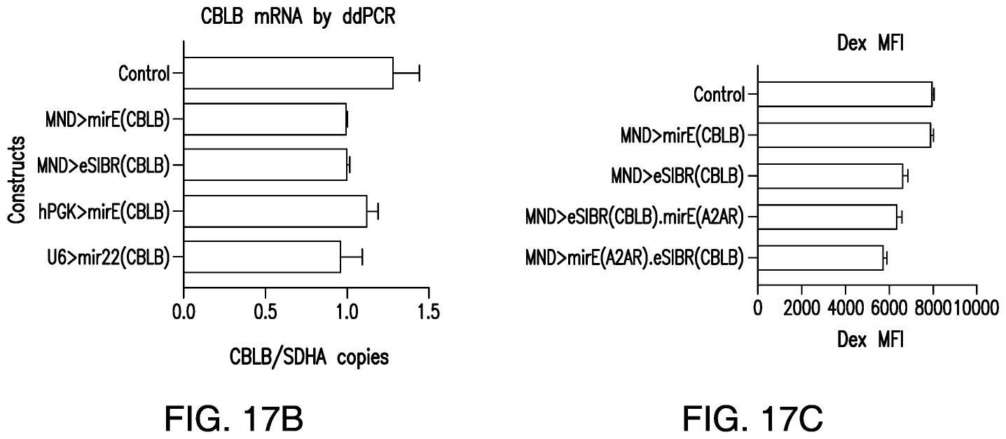

Initially, it was determined whether NeoTCR expression could be improved in Secondary Promoter Constructs including an inhibitory RNA molecule as a Payload. FIG. 17A shows the construct having different inhibitory RNA scaffolds and used in this example. Gene expression profile analysis was performed by droplet digital PCR (ddPCR) and showed that both scaffolds were able to knock down the expression of the gene of interest CBLB. Here it is knocking down the expression of a gene called CBLB (FIG. 17B). Further, it was determined whether these constructs would lead to a reduction in NeoTCR. As seen in FIG. 17C, Secondary Promoter Constructs including an inhibitory RNA having mir-E scaffold did not reduce the expression of NeoTCR. Thus, use of inhibitory RNA molecules including the mir-E scaffold improves the NeoTCR expression.

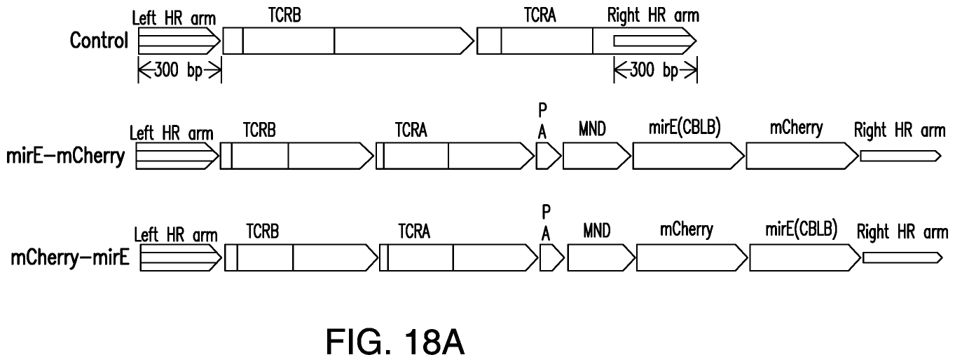

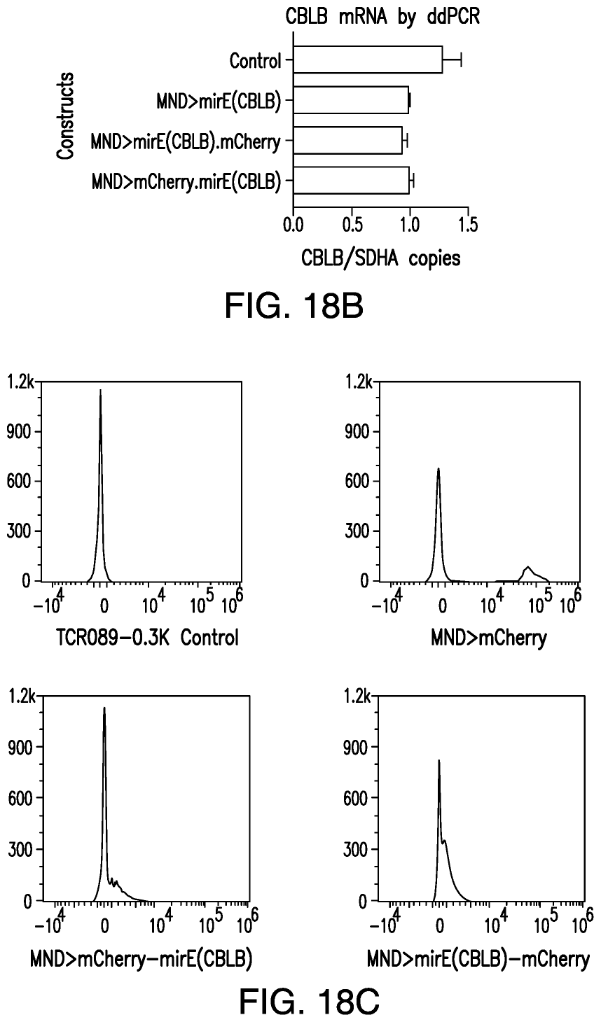

Next, it was verified whether addition of a second Payload could be achieved. FIG. 18A shows the constructs used for this aim. As seen in FIGS. 18B and 18C, Secondary Promoter Constructs including an inhibitory RNA positioned before the Payload (mCherry) had knockdown of the CBLB gene as well as expression of the Payload (mCherry).

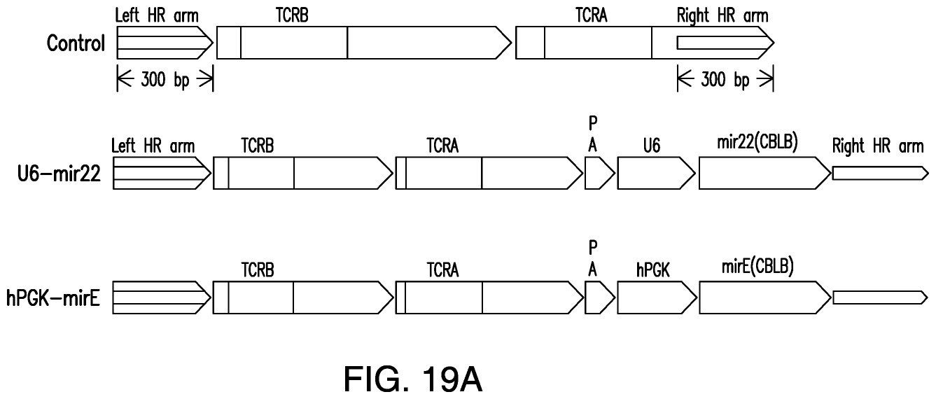

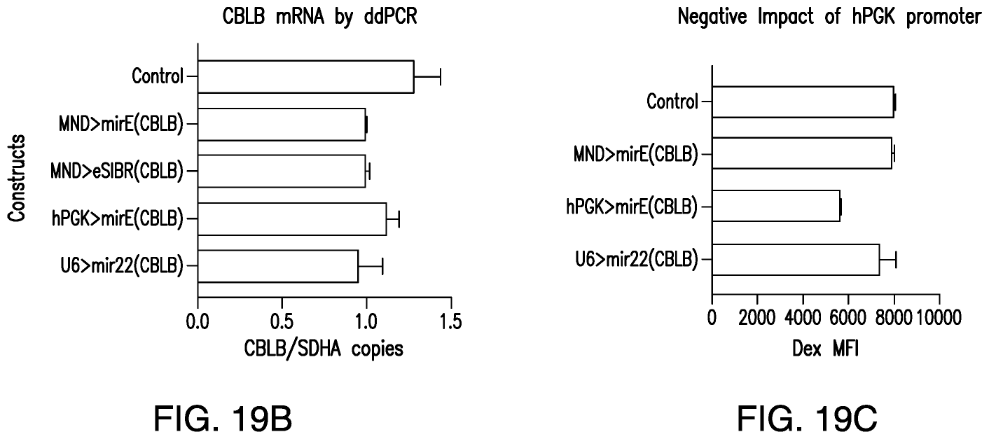

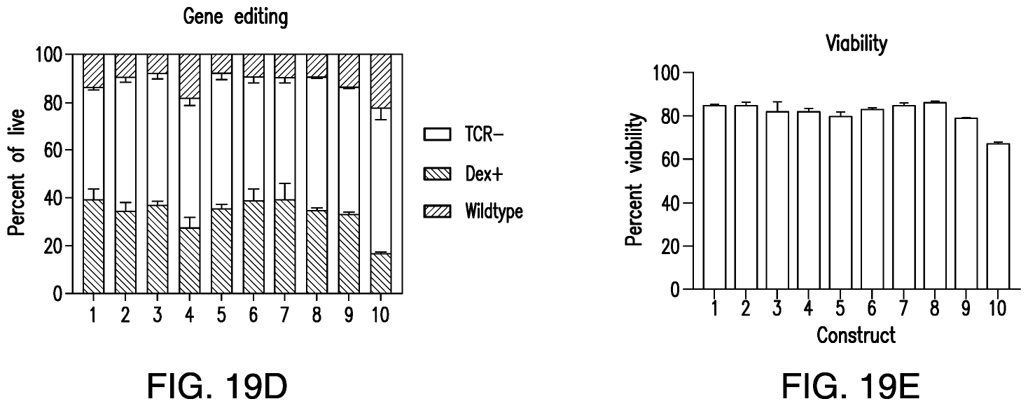

Next, it was tested whether addition of certain secondary promoters upstream of an inhibitory RNA molecules would increase the knockdown of the CBLB target gene. Analysis of gene expression levels confirmed that U6 promoters significantly reduced the expression of the CBLB target gene without negatively impacting the expression of the NeoTCRs (FIGS. 19A-19E).

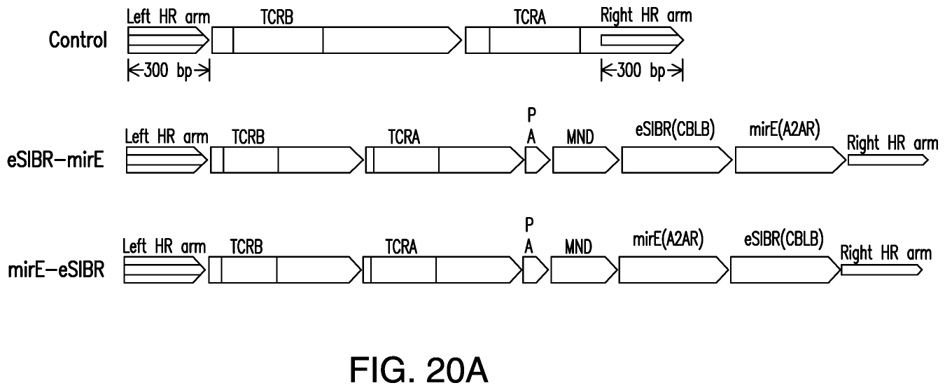

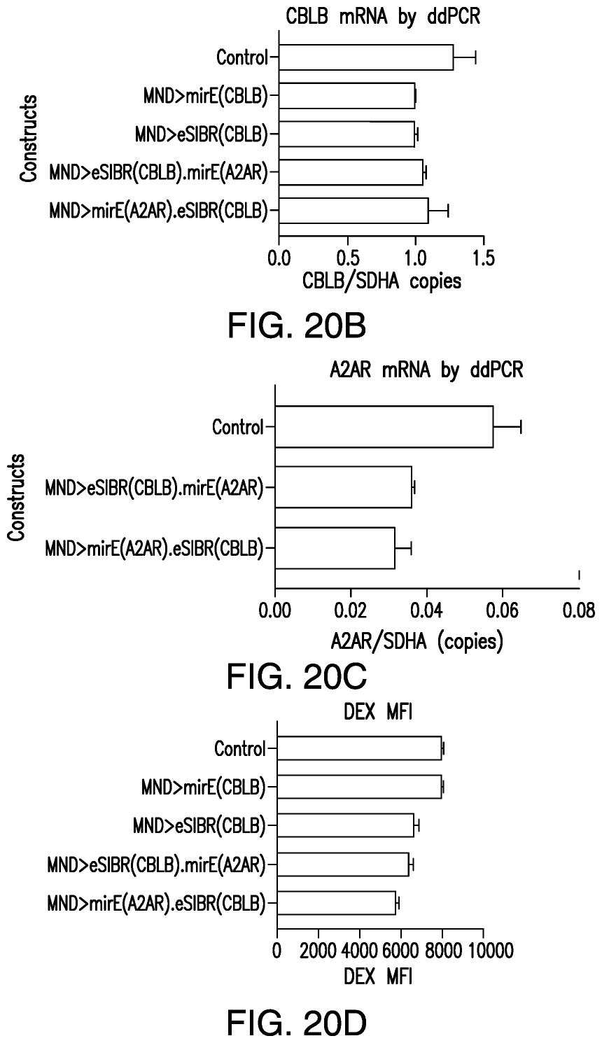

Further, Secondary Promoter Constructs including multiple inhibitory RNA molecules were designed and tested to evaluate the knockdown of the multiple target genes such as CBLB and A2AR (FIG. 20A). Using ddPCR, it was observed that miRNA were effective in knocking down the gene expression of the targets with relatively minimal effect on the NeoTCR expression (FIGS. 20B-20D). These data demonstrated that Secondary Promoter Constructs including inhibitory RNA molecules can reduce gene expression of a target without significantly alter the expression of NeoTCRs.

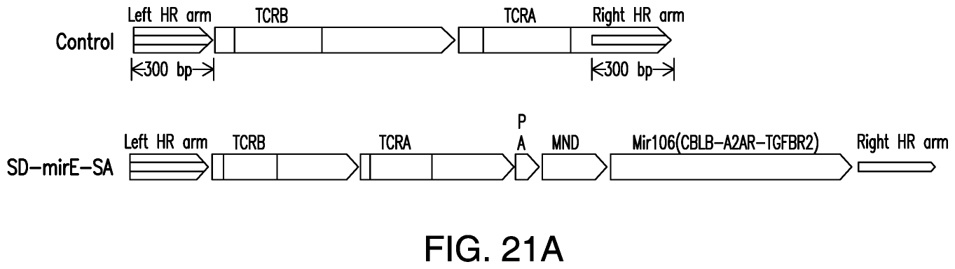

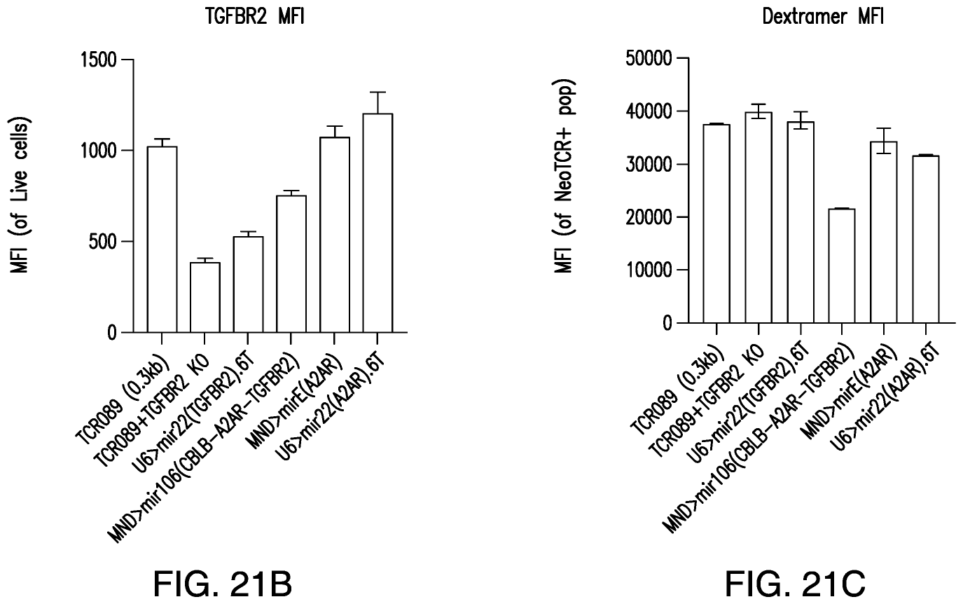

Finally, it was determined whether miRNA clusters could target multiple genes. Cells were transfected with the constructs depicted in FIG. 21A including a miRNA cluster based on mir-106 targeting TGFBR2, A2AR, and CBLB. Analysis of expression levels of TGFB2R and NeoTCR demonstrated that the use of this specific miRNA cluster reduced the expression levels of the NeoTCR.

Collectively, these data demonstrated that the inclusion of promoters upstream of Payloads improved the knockdown of target genes induced by inhibitory RNA molecules without altering the expression of NeoTCRs.

### Example 10. Effects of Secondary Promoter Constructs Including Splice Acceptor Sites and Splice Donor Site

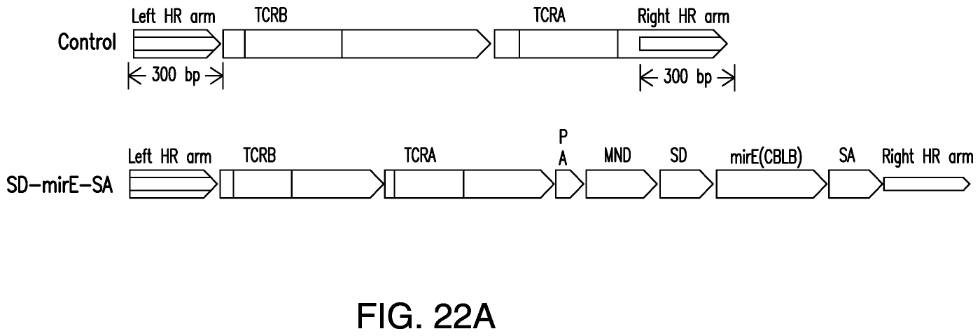

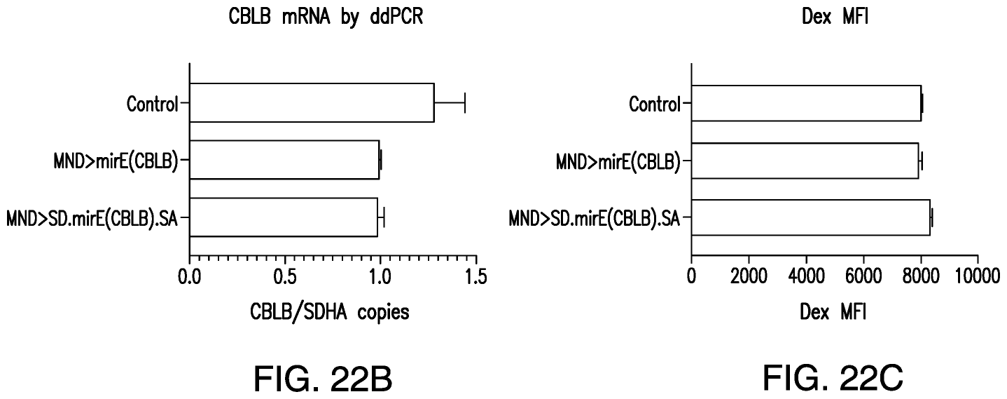

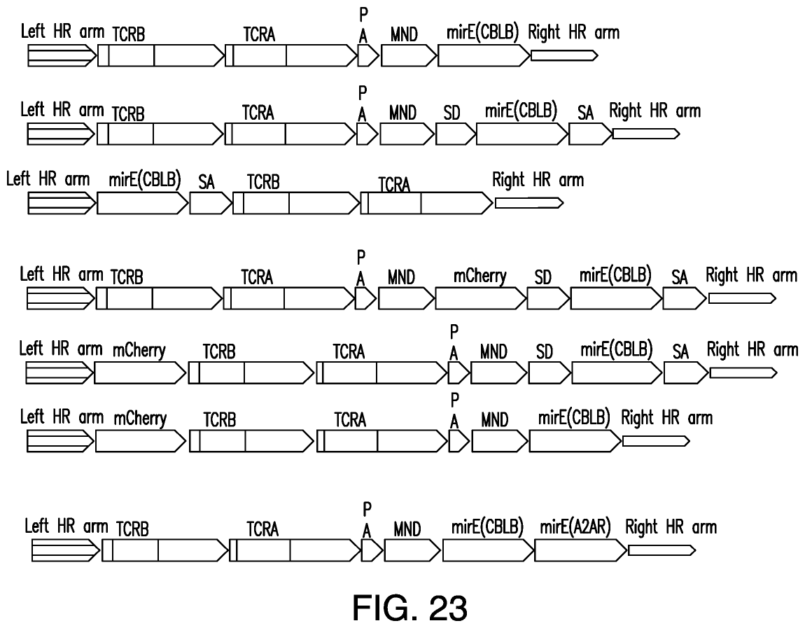

Next, it was hypothesized that flanking inhibitory RNA molecules by splice acceptor sites (SA) and/or splice donor sites (SD) could improve the knockdown of target genes. FIG. 22A shows the Secondary Promoter Constructs used in a first experiment. Notably, addition of SA and SD sites had no impact on the knockdown of the target genes nor on the expression of the NeoTCR (FIGS. 22B and 22C). These data showed that addition of splice donor site and splice acceptor site can be useful for Secondary Promoter Constructs that include additional Payloads without impacting on the expression of NeoTCRs and knockdown of target genes.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A cell comprising an exogenous polynucleotide comprising
a) an exogenous enhancer, an insulator, a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter;
b) an exogenous enhancer, a pause element, a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter;
c) a Woodchuck Hepatitis Virus Posttranscriptional Regulatory Element (WPRE), a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter;
d) an insulator, a Woodchuck Hepatitis Virus Posttranscriptional Regulatory Element (WPRE), a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter;
e) an insulator, a sequence encoding an exogenous TCR, and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter;
f) a sequence encoding an exogenous TCR and a sequence encoding at least one Payload, wherein the exogenous polynucleotide is integrated at an endogenous locus within the genome of the cell, wherein the sequence encoding an exogenous TCR is under control of an endogenous promoter and the sequence encoding at least one Payload is under control of an exogenous promoter;
g) a sequence encoding an exogenous TCR, a first sequence encoding a Payload, and a second sequence encoding a Payload, wherein the exogenous polynucleotide is integrated at an endogenous locus within the genome of the cell, wherein the sequence encoding an exogenous TCR and the first sequence encoding a Payload are under control of an endogenous promoter and the second sequence encoding a Payload is under control of an exogenous promoter; and/or
h) a sequence encoding an exogenous TCR and a sequence encoding at least one Payload, wherein the exogenous polynucleotide is integrated at an endogenous locus within the genome of the cell and is under control of an endogenous promoter.

2. The cell of claim 1, wherein
a) the insulator is an HS4 insulator or an IS2 insulator;
b) the exogenous enhancer is a CMV enhancer, a TCRα enhancer, or a TCRβ enhancer;
c) the exogenous promoter is a constitutive promoter selected from the group consisting of an MDN promoter, an EF1α promoter, an ACTB promoter, a PGK promoter, and a U6 promoter; and/or
d) the exogenous promoter is an inducible promoter selected from the group consisting of an AP1 promoter, an NFAT promoter, an NF-κB promoter, and an NR4A-responsive promoter.

3. The cell of claim 1, wherein
a) the insulator comprises the nucleotide sequence set forth in SEQ ID NO: 18 or SEQ ID NO: 19;
b) the WPRE comprises the nucleotide sequence set forth in SEQ ID NO: 20; and/or
c) the exogenous enhancer comprises the nucleotide sequence set forth in SEQ ID NO: 32 or SEQ ID NO: 33.

4. The cell of claim 1, wherein the at least one Payload is selected from the group consisting of a cytokine receptors trap, a ligand trap, an angiogenesis factor, an apoptotic factor, an inhibitory protein, an extracellular matrix modulator, a soluble TCR, a homing signal, an enzyme, a modulator of reactive oxygen species, a competitive ligand inhibitor, a protein that binds to receptors and sterically hinders receptor function, and an inhibitory RNA molecule.

5. The cell of claim 1, wherein the 3′ of the at least one Payload comprises
a) a STOP codon;
b) a sequence encoding a 2A peptide and a sequence encoding a protease cleavage peptide; and/or
c) a poly-adenylation sequence.

6. The cell of claim 1, wherein the at least one Payload is an inhibitory RNA molecule selected from the group consisting of shRNA, miRNA, and miRNA cluster.

7. The cell of claim 7, wherein the inhibitory RNA molecule is a miRNA comprising a first flanking sequence and a second flanking sequence, optionally wherein the first flanking sequence and the second flanking sequence are derived from miR-155, miR-30, miR-17/92, miR-122, or miR-21.

8. The cell of claim 1, wherein the exogenous TCR recognizes a cancer neoantigen.

9. The cell of claim 1, wherein the endogenous locus within the genome of the cell is a TCR locus.

10. A polynucleotide comprising
a) an exogenous enhancer, an insulator, a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter;
b) an exogenous enhancer, a pause element, a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter;
c) a Woodchuck Hepatitis Virus Posttranscriptional Regulatory Element (WPRE), a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter;
d) an insulator, a Woodchuck Hepatitis Virus Posttranscriptional Regulatory Element (WPRE), a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter;
e) an insulator, a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter;
f) a first homology arm, a second homology arm, a sequence encoding an exogenous TCR, and a sequence encoding at least one Payload, wherein the first and second homology arms are homologous to a TRAC or TRBC locus and wherein the sequence encoding at least one Payload is under control of an exogenous promoter;
g) a first homology arm, a second homology arm, a sequence encoding an exogenous TCR, a first sequence encoding a Payload, and a second sequence encoding a Payload, wherein the first and second homology arms are homologous to a TRAC or TRBC locus and wherein the second sequence encoding a Payload is under control of an exogenous promoter; and/or
h) a sequence encoding a first homology arm, a second homology arm, an exogenous TCR, and a sequence encoding at least one Payload, wherein the first and second homology arms are homologous to a TRAC or TRBC locus.

11. The polynucleotide of claim 10, wherein the sequence encoding an exogenous TCR comprises a TCRα gene sequence and a TCRβ gene sequence.

12. The polynucleotide of claim 10, wherein the sequence encoding an exogenous TCR further comprises a sequence encoding a P2A peptide, a sequence encoding a signal sequence, a sequence encoding a protease cleavage peptide, or a combination thereof.

13. The polynucleotide of claim 10, wherein the at least one Payload is selected from the group consisting of a cytokine receptors trap, a ligand trap, an angiogenesis factor, an apoptotic factor, an inhibitory protein, an extracellular matrix modulator, a soluble TCR, a homing signal, an enzyme, a modulator of reactive oxygen species, a competitive ligand inhibitor, a protein that binds to receptors and sterically hinders receptor function, and an inhibitory RNA molecule.

14. A vector comprising the polynucleotide of claim 10.

15. A method of modifying a cell, the method comprising:
a) introducing by electroporation into the cell the polynucleotide of claim 13; and
b) recombining the polynucleotide or vector into an endogenous locus of the cell.

16. The method of claim 15, wherein the method further comprises culturing the cell in the presence of at least one cytokine selected from the group consisting of IL2, IL7, IL15, and a combination thereof.

17. A composition comprising an effective amount of the cell of claim 1, wherein the composition is a pharmaceutical composition that further comprises a pharmaceutically acceptable excipient.

18. A method of treating cancer in a subject in need thereof, the method comprising administering a therapeutically effective amount of the cell of claim 1.

19. The method of claim 18, wherein the cancer is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinomas, renal cell cancers, testicular cancer, sarcomas, colorectal cancer, follicular lymphoma, leukemia, and multiple myeloma.

20. A kit comprising the cell of claim 1.


SEQUENCE LISTING (98 sequences)

SEQ ID NO: 1 - protein, 199 aa, synthetic construct
  REGION 1..199: note = Description of sequence: cytokine receptor trap
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 2 - DNA, 597 bp, synthetic construct
  misc_feature 1..597: note = Description of sequence: cytokine receptor trap
  source 1..597: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttca

SEQ ID NO: 3 - protein, 255 aa, synthetic construct
  REGION 1..255: note = Description of sequence: anti-PDl svFv
MYRMQLLSCIALSLALVTNSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 4 - DNA, 765 bp, synthetic construct
  misc_feature 1..765: note = Description of sequence: anti-PDl svFv
  source 1..765: mol_type = other DNA
atgtacaggatgcaactcctgtcttgcattgcactaagtcttgcacttgtcacaaacagtgagatcgtgctgactcagtcccctgctaccctgtccctgtcccccggtgagcgtgctaccctgtcctgccgcgcttcccagagcgtgtcctcctacctggcctggtatcaacaaaagcccggccaagctccccgcctgctgatctacgacgcctccaaccgcgctactggcatccctgctcgcttctccggttccggttccggcactgacttcactctgaccatctcctccctggagcccgaggatttcgctgtgtactactgccagcagtcctccaactggccccgtaccttcggtcagggcaccaaggtggagatcaagggtggaggcggttcaggcggaggtggctctggcggtggcggaagccaggtccagctggtggagagcggtggtggtgtggtgcagcccggtcgttccctgcgtctggattgcaaagcctccggcatcaccttctccaacagcggcatgcactgggtgcgtcaggctcctggtaagggcctggagtgggtggctgtgatctggtacgatggctccaagcgctactacgccgactccgtgaagggtcgtttcaccatctcccgcgacaacagcaagaacaccctgttcctgcagatgaactccctgcgtgccgaggacaccgccgtctactactgcgccaccaacgacgactactggggtcagggcactctggtgaccgtctcctcc

SEQ ID NO: 5 - DNA, 157 bp, synthetic construct
  misc_feature 1..157: note = Description of sequence: inhibitory molecule
  source 1..157: mol_type = other DNA
tcgacttcttaacccaacagaaggctcgagaaggtatattgctgttgacagtgagcgaaagatgtcaagattgagcctttagtgaagccacagatgtaaaggctcaatcttgacatcttgtgcctactgcctcggacttcaaggggctactttagga

SEQ ID NO: 6 - DNA, 159 bp, synthetic construct
  misc_feature 1..159: note = Description of sequence: inhibitory molecule
  source 1..159: mol_type = other DNA
gtcgactggaggcttgctttgggctgtatgctgaaggctcaatcttgacatcttgttttggcctctgactgacaagattcagattgagccttggacacaaggccctttatcagcactcacatggaacaaatggccaccgtgggaggatgacaactcgag

SEQ ID NO: 7 - DNA, 51 bp, synthetic construct
  misc_feature 1..51: note = Description of sequence: inhibitory molecule
  source 1..51: mol_type = other DNA
gatgtcaagattgagccttgccctgacccagcaaggctcaatcttgacatc

SEQ ID NO: 8 - DNA, 310 bp, synthetic construct
  misc_feature 1..310: note = Description of sequence: inhibitory molecule
  source 1..310: mol_type = other DNA
tggaggcttgctttgggctgtatgctgaaggctcaatcttgacatcttgttttggcctctgactgacaagattcagattgagccttggacacaaggccctttatcagcactcacatggaacaaatggccaccgtgggaggatgacaagtcgactcgacttcttaacccaacagaaggctcgagaaggtatattgctgttgacagtgagcgccagaccttccgcaagatcatttagtgaagccacagatgtaaatgatcttgcggaaggtctggtgcctactgcctcggacttcaaggggctactttagga

SEQ ID NO: 9 - DNA, 310 bp, synthetic construct
  misc_feature 1..310: note = Description of sequence: inhibitory molecule
  source 1..310: mol_type = other DNA
tcgacttcttaacccaacagaaggctcgagaaggtatattgctgttgacagtgagcgccagaccttccgcaagatcatttagtgaagccacagatgtaaatgatcttgcggaaggtctggtgcctactgcctcggacttcaaggggctactttaggagtcgactggaggcttgctttgggctgtatgctgaaggctcaatcttgacatcttgttttggcctctgactgacaagattcagattgagccttggacacaaggccctttatcagcactcacatggaacaaatggccaccgtgggaggatgacaa

SEQ ID NO: 10 - DNA, 979 bp, synthetic construct
  misc_feature 1..979: note = Description of sequence: inhibitory molecule
  source 1..979: mol_type = other DNA
ctgactacatcacagcagcatacgtggagatgaggcgagaggcttgggctagtaaggatgccacctatacttctgcccggaccctgctggctatcctgcgcctttccactgctctggtaagtgcccaaattgctggagggccatctgttttgacccttaaaggggtagctccttaccgtgctctcattgccgcctccccacctcccgctcgagccctgccggggcgatgtcaagattgagccttgctttgtgtaggcaaggctcaatcttgacatcgctccagcagggcacgcaatgcttcgtggagggaaaggccttttccccacttcttaaccttcactgagagggtggttggggtctgtttcactccatgtgtcctagatcctgtgctacagaccttcctttctgtcctcccgtcttggaattcagtcctgggggctggaatgatcttgcggaaggtctgtgtgattacccaaccagacctccgcaagtatcatatatagcccccgggttccgttctctctgccaattgtcttcttggctgagctccccaagctccatctgtcatgctggggagcccagtggcgttcaaaagggtctggtctccctcacaggacagctgaactctagaagtcctgggggctcgatgaagaaagtctcaccaggctgtgattacccaacgcctggtagactttacttcagcgcgagcccccgggacacaccgcggatgctggggggaggggggattccactcctgttttgtgagtaggcgacccatgggctgcccagccttaaagccagaacaagggtgtcccctgacctcgttccactgccctcctcccgttcccatctttcccccctaccttccccttaggcacgtctgagaatggtggatgtggtggagaaagaagatgtgaatgaagccatcaggctaatggagatgtcaaaggactctcttctaggagacaaggggcagaca

SEQ ID NO: 11 - DNA, 115 bp, synthetic construct
  misc_feature 1..115: note = Description of sequence: splice acceptor site
  source 1..115: mol_type = other DNA
ttccccttaggcacgtctgagaatggtggatgtggtggagaaagaagatgtgaatgaagccatcaggctaatggagatgtcaaaggactctcttctaggagacaaggggcagaca

SEQ ID NO: 12 - DNA, 83 bp, synthetic construct
  misc_feature 1..83: note = Description of sequence: splice acceptor site
  source 1..83: mol_type = other DNA
tgtcccacagatatccagaaccccgaccccgccgtgtaccagctgcgggacagcaagagcagcgacaagagcgtgtgcctgtt

SEQ ID NO: 13 - DNA, 126 bp, synthetic construct
  misc_feature 1..126: note = Description of sequence: Payload construct
  source 1..126: mol_type = other DNA
ctgactacatcacagcagcatacgtggagatgaggcgagaggcttgggctagtaaggatgccacctatacttctgcccggaccctgctggctatcctgcgcctttccactgctctggtaagtgccc

SEQ ID NO: 14 - DNA, 225 bp, synthetic construct
  misc_feature 1..225: note = Description of sequence: poly-A signal sequence
  source 1..225: mol_type = other DNA
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 15 - DNA, 134 bp, synthetic construct
  misc_feature 1..134: note = Description of sequence: poly-A signal sequence
  source 1..134: mol_type = other DNA
gctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 16 - DNA, 44 bp, synthetic construct
  misc_feature 1..44: note = Description of sequence: poly-A signal sequence
  source 1..44: mol_type = other DNA
tttatttgtgaaatttgtgatgctattgctttatttgtaaccat

SEQ ID NO: 17 - skipped

SEQ ID NO: 18 - DNA, 244 bp, synthetic construct
  misc_feature 1..244: note = Description of sequence: an insulator
  source 1..244: mol_type = other DNA
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctt

SEQ ID NO: 19 - DNA, 1010 bp, synthetic construct
  misc_feature 1..1010: note = Description of sequence: an insulator
  source 1..1010: mol_type = other DNA
cggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagaatatttaaataaacttataaattgtgagagaaattaatgaatgtctaagttaatgcagaaacggaggctcctcatttatttttgaacttaaagacttaatattgtgaaggtatactttctttaataataagcctgcgcccaatatgttcaccccaaaaaagctgtttgttaacttgtcaacctcatttaaaatatataagaaacagcccaaagacaataacaaaagaataataaaaaagaatgaaatatgtaattctttcagagtaaaaatcacacccatgacctggccactgagggcttgatcaattcactttgaatttggcattaaataccattaaggtatattaactgattttaaaataagatatattc

SEQ ID NO: 20 - DNA, 249 bp, synthetic construct
  misc_feature 1..249: note = Description of sequence: WPRE 3
  source 1..249: mol_type = other DNA
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtg

SEQ ID NO: 21 - DNA, 143 bp, synthetic construct
  misc_feature 1..143: note = Description of sequence: MAZ4 pause element
  source 1..143: mol_type = other DNA
cctggccttgggggagggggaggccagaatgagagctcctggccttgggggagggggaggccagaatgactcgacctggccttgggggagggggaggccagaatgagagctcctggccttgggggagggggaggccagaatga

SEQ ID NO: 22 - DNA, 1184 bp, synthetic construct
  misc_feature 1..1184: note = Description of sequence: constitutive promoter
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 23 - DNA, 233 bp, synthetic construct
  misc_feature 1..233: note = Description of sequence: constitutive promoter
  source 1..233: mol_type = other DNA
gaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 24 - DNA, 550 bp, synthetic construct
  misc_feature 1..550: note = Description of sequence: constitutive promoter
  source 1..550: mol_type = other DNA
actgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccga

SEQ ID NO: 25 - DNA, 522 bp, synthetic construct
  misc_feature 1..522: note = Description of sequence: constitutive promoter
  source 1..522: mol_type = other DNA
tcgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 26 - DNA, 388 bp, synthetic construct
  misc_feature 1..388: note = Description of sequence: constitutive promoter
  source 1..388: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaag

SEQ ID NO: 27 - DNA, 249 bp, synthetic construct
  misc_feature 1..249: note = Description of sequence: constitutive promoter
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 28 - DNA, 252 bp, synthetic construct
  misc_feature 1..252: note = Description of sequence: inducible promoter
  source 1..252: mol_type = other DNA
tgagtcagtgactcagtgagtcagtgactcagtgagtcagtgactcagtgagtcagtgactcaggaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttg

SEQ ID NO: 29 - DNA, 353 bp, synthetic construct
  misc_feature 1..353: note = Description of sequence: inducible promoter
  source 1..353: mol_type = other DNA
aggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 30 - DNA, 255 bp, synthetic construct
  misc_feature 1..255: note = Description of sequence: inducible promoter
  source 1..255: mol_type = other DNA
gggactttccgcttggggactttccgctggggactttccgctggggactttccgctggggactttccgaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttg

SEQ ID NO: 31 - DNA, 118 bp, synthetic construct
  misc_feature 1..118: note = Description of sequence: inducible promoter
  source 1..118: mol_type = other DNA
ctcgagaaaggtcaccaattaaaggtcaccaattaaaggtcaccaattaaaggtcaccaattaaaggtcaccaattaaaggtcaccaattaaaggtcaccaattaaaggtcaccaatt

SEQ ID NO: 32 - DNA, 249 bp, synthetic construct
  misc_feature 1..249: note = Description of sequence: enhancer
  source 1..249: mol_type = other DNA
gggctggggcggtcccctcccatttccatgacgtcatggttaccaagaggggcaagtagggcaccctttgaagctctcccgcagaagccacatcctctggaaagaagagtttataatactgagttagagatagcatcgccccaggccacgtgccgaggggagcaggctgggccgttacaccaccccccaaccgcaggtgcagcaaggccaacatgccaggctgggaggggctgccggcccctcgttgag

SEQ ID NO: 33 - DNA, 303 bp, synthetic construct
  misc_feature 1..303: note = Description of sequence: enhancer
  source 1..303: mol_type = other DNA
cgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatg

SEQ ID NO: 34 - skipped

SEQ ID NO: 35 - DNA, 1000 bp, synthetic construct
  misc_feature 1..1000: note = Description of sequence: homology arm
  source 1..1000: mol_type = other DNA
acattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattc

SEQ ID NO: 36 - DNA, 1000 bp, synthetic construct
  misc_feature 1..1000: note = Description of sequence: homology arm
  source 1..1000: mol_type = other DNA
accgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacag

SEQ ID NO: 37-38 - skipped (2 entries)

SEQ ID NO: 39 - protein, 19 aa, synthetic construct
  REGION 1..19: note = Description of sequence: P2A peptide
ATNFSLLKQAGDVEENPGP

SEQ ID NO: 40 - DNA, 57 bp, synthetic construct
  misc_feature 1..57: note = Description of sequence: P2A peptide
  source 1..57: mol_type = other DNA
gccaccaacttcagcctgctgaagcaggccggcgacgtggaggagaaccccggccct

SEQ ID NO: 41 - DNA, 57 bp, synthetic construct
  misc_feature 1..57: note = Description of sequence: P2A peptide
  source 1..57: mol_type = other DNA
gccactaacttctccctgttgaaacaggctggcgatgttgaagaaaaccccggtcct

SEQ ID NO: 42 - DNA, 12 bp, synthetic construct
  misc_feature 1..12: note = Description of sequence: Furin cleavage site
  source 1..12: mol_type = other DNA
cgggccaagcgg

SEQ ID NO: 43 - protein, 26 aa, synthetic construct
  REGION 1..26: note = Description of sequence: HGH signal sequence
MATGSRTSLLLAFGLLCLPWLQEGSA

SEQ ID NO: 44 - DNA, 78 bp, synthetic construct
  misc_feature 1..78: note = Description of sequence: HGH signal sequence
  source 1..78: mol_type = other DNA
atggccacaggcagcagaacatctctgctgctggccttcggactgctgtgtctgccttggctgcaagagggttccgcc

SEQ ID NO: 45 - DNA, 78 bp, synthetic construct
  misc_feature 1..78: note = Description of sequence: HGH signal sequence
  source 1..78: mol_type = other DNA
atggccaccggctctagaacaagcctgctgctcgcttttggcctgctctgcctcccatggctccaagaaggatctgct

SEQ ID NO: 46 - DNA, 873 bp, synthetic construct
  misc_feature 1..873: note = Description of sequence: Secondary Promoter Construct
  source 1..873: mol_type = other DNA
gatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcc

SEQ ID NO: 47 - protein, 291 aa, synthetic construct
  REGION 1..291: note = Description of sequence: Secondary Promoter Construct
DAGITQSPRYKITETGRQVTLMCHQTWSHSYMFWYRQDLGHGLRLIYYSAAADITDKGEVSDGYVVSRSKTENFPLTLESATRSQTSVYFCASSEDSYEQYFGPGTRLTVTEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVSRERIPEA

SEQ ID NO: 48 - DNA, 7046 bp, synthetic construct
  misc_feature 1..7046: note = Description of sequence: Secondary Promoter Construct
  source 1..7046: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagtgacatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 49 - DNA, 7520 bp, synthetic construct
  misc_feature 1..7520: note = Description of sequence: Secondary Promoter Construct
  source 1..7520: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagtgacatatggataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 50 - DNA, 7051 bp, synthetic construct
  misc_feature 1..7051: note = Description of sequence: Secondary Promoter Construct
  source 1..7051: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgagtcgacgctttaaaaaacctcccacatctccccctgaacctgaaacataaaatgaatgcaattgttgttgttaacttgtttattgcagcttataatggttacaaataaagcaatagcatcacaaatttcacaaataaagctagaatggttacaaataaagcaatagcatcacaaatttcacaaataaacaccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatccatatgtcacttgtacagctcgtccattccgcctgtgctgtgtctgccttcggctctctcgtactgttccacgatggtgtagtcctcgttgtggctggtgatgtccagcttgatgttcacgttgtaggcgccaggcagctgcacgggtttcttggccttgtaggtggttttcacttcggcgtcgtagtggccgccatccttcagtttcagccgctgcttgatctcgcccttcagggcgccatcttcagggtacattctctcgctgctggcttcccagcccattgtctttttctgcatcacggggccatcagagggaaagttggtgccccgcagcttcactttgtagatgaactcgccgtcctgcagagagctatcttgagtcacggtgaccacgccgccgtcctcgaagttcatcactctctcccacttgaagccctcggggaagctcagcttcaggtagtcgggaatatcggcggggtgcttcacgtaggccttgctgccgtacataaactgagggctcagaatgtcccaggcaaaaggcagagggccgcctttggtcactttcagcttggcggtctgtgttccctcgtaaggtctgccctcgccttcgccttcaatctcgaactcgtggccgttcacgctgccttccatgtgcaccttgaaccgcatgaactctttgatgatggccatgttgtcctcttcgcccttggacaccatggtggcggatcccttctatggaagtcaaaacagcgtggatggcgtctccaggcgatctgacggttcactaaacgagctctgcttatatagagctcggggagcagaagcgcgcgaacagaagcgagaagcgaactgattggttagttcaaataaggcacagggtcatttcaggtccttggggcaccctggaaacatctgatggttctctagaaactgctgagggcgggaccgcatctggggaccatctgttcttggccctgagccggggcaggaactgcttaccacagatatcctgtttggcccatattctgctgttccaactgttcttggccctgagccggggcaggaactgcttaccacagatatcctgtttggcccatattctcctgtttctctgttcctcgagaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 51 - DNA, 7413 bp, synthetic construct
  misc_feature 1..7413: note = Description of sequence: Secondary Promoter Construct
  source 1..7413: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcgggctggggcggtcccctcccatttccatgacgtcatggttaccaagaggggcaagtagggcaccctttgaagctctcccgcagaagccacatcctctggaaagaagagtttataatactgagttagagatagcatcgccccaggccacgtgccgaggggagcaggctgggccgttacaccaccccccaaccgcaggtgcagcaaggccaacatgccaggctgggaggggctgccggcccctcgttgagggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagagagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 52 - DNA, 7467 bp, synthetic construct
  misc_feature 1..7467: note = Description of sequence: Secondary Promoter Construct
  source 1..7467: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagagagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 53 - DNA, 7331 bp, synthetic construct
  misc_feature 1..7331: note = Description of sequence: Secondary Promoter Construct
  source 1..7331: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgagtgagtcagtgactcagtgagtcagtgactcagtgagtcactgactcactgagtcagtgactcaggaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagagagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 54 - DNA, 8265 bp, synthetic construct
  misc_feature 1..8265: note = Description of sequence: Secondary Promoter Construct
  source 1..8265: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttgcggccgccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagagagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 55 - DNA, 7629 bp, synthetic construct
  misc_feature 1..7629: note = Description of sequence: Secondary Promoter Construct
  source 1..7629: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttttaaaactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgaggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagagagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 56 - DNA, 7601 bp, synthetic construct
  misc_feature 1..7601: note = Description of sequence: Secondary Promoter Construct
  source 1..7601: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgagtcgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagagagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 57 - DNA, 7432 bp, synthetic construct
  misc_feature 1..7432: note = Description of sequence: Secondary Promoter Construct
  source 1..7432: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgagaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagagagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 58 - DNA, 7334 bp, synthetic construct
  misc_feature 1..7334: note = Description of sequence: Secondary Promoter Construct
  source 1..7334: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgaggggactttccgcttggggactttccgctggggactttccgctggggactttccgctggggactttccgaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagagagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 59 - DNA, 5679 bp, synthetic construct
  misc_feature 1..5679: note = Description of sequence: Secondary Promoter Construct
  source 1..5679: mol_type = other DNA
ggtacctcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagcttgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatcctcgacttcttaacccaacagaaggctcgagaaggtatattgctgttgacagtgagcgaaagatgtcaagattgagcctttagtgaagccacagatgtaaaggctcaatcttgacatcttgtgcctactgcctcggacttcaaggggctactttaggagtcgacgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagcgggctaagagaggctctggagccacaaactttagcctcctgaaacaagcaggggatgtggaagagaatcccggacctgcggccgcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 60 - DNA, 5691 bp, synthetic construct
  misc_feature 1..5691: note = Description of sequence: Secondary Promoter Construct
  source 1..5691: mol_type = other DNA
ggtacctcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagcttgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccgccaccatggtgtccaagggcgaagaggacaacatggccatcatcaaagagttcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagacaatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagcgggctaagagaggctctggagccacaaactttagcctcctgaaacaagcaggggatgtggaagagaatcccggacctactagttcgacttcttaacccaacagaaggctcgagaaggtatattgctgttgacagtgagcgaaagatgtcaagattgagcctttagtgaagccacagatgtaaaggctcaatcttgacatcttgtgcctactgcctcggacttcaaggggctactttaggagtcgactgcgatgcggccgcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 61 - DNA, 5134 bp, synthetic construct
  misc_feature 1..5134: note = Description of sequence: Secondary Promoter Construct
  source 1..5134: mol_type = other DNA
ggtacctcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagcttgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccctgactacatcacagcagcatacgtggagatgaggcgagaggcttgggctagtaaggatgccacctatacttctgcccggaccctgctggctatcctgcgcctttccactgctctggtaagtgccctcgacttcttaacccaacagaaggctcgagaaggtatattgctgttgacagtgagcgaaagatgtcaagattgagcctttagtgaagccacagatgtaaaggctcaatcttgacatcttgtgcctactgcctcggacttcaaggggctactttaggattccccttaggcacgtctgagaatggtggatgtggtggagaaagaagatgtgaatgaagccatcaggctaatggagatgtcaaaggactctcttctaggagacaaggggcagacagtcgactgcgatgcggccgcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 62 - DNA, 4001 bp, synthetic construct
  misc_feature 1..4001: note = Description of sequence: Secondary Promoter Construct
  source 1..4001: mol_type = other DNA
ggtaccagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctctgaattctcgacttcttaacccaacagaaggctcgagaaggtatattgctgttgacagtgagcgaaagatgtcaagattgagcctttagtgaagccacagatgtaaaggctcaatcttgacatcttgtgcctactgcctcggacttcaaggggctactttaggagtcgactgtcccacagatatccagaaccccgaccccgccgtgtaccagctgcgggacagcaagagcagcgacaagagcgtgtgcctgttggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgctgtgtatcagctgcgcgacagcaagagcagcgacaagagcgtgtgtttgttcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggcggccgctgcgatgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 63 - DNA, 4893 bp, synthetic construct
  misc_feature 1..4893: note = Description of sequence: Secondary Promoter Construct
  source 1..4893: mol_type = other DNA
ggtacctcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagcttgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatcctcgacttcttaacccaacagaaggctcgagaaggtatattgctgttgacagtgagcgaaagatgtcaagattgagcctttagtgaagccacagatgtaaaggctcaatcttgacatcttgtgcctactgcctcggacttcaaggggctactttaggagtcgactgcgatgcggccgcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 64 - DNA, 4648 bp, synthetic construct
  misc_feature 1..4648: note = Description of sequence: Payload construct
  source 1..4648: mol_type = other DNA
ggtacctcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagcttgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatgtcaagattgagccttgccctgacccagcaaggctcaatcttgacatcttttttgtcgactgcgatgcggccgcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 65 - DNA, 3794 bp, synthetic construct
  misc_feature 1..3794: note = Description of sequence: Payload construct
  source 1..3794: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccgccacc

SEQ ID NO: 66 - DNA, 2544 bp, synthetic construct
  misc_feature 1..2544: note = Description of sequence: Payload construct
  source 1..2544: mol_type = other DNA
tgacatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 67 - DNA, 3738 bp, synthetic construct
  misc_feature 1..3738: note = Description of sequence: Payload construct
  source 1..3738: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgct

SEQ ID NO: 68 - DNA, 2975 bp, synthetic construct
  misc_feature 1..2975: note = Description of sequence: Payload construct
  source 1..2975: mol_type = other DNA
tgacatatggataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 69 - DNA, 3402 bp, synthetic construct
  misc_feature 1..3402: note = Description of sequence: Payload construct
  source 1..3402: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgagtcgacgctttaaaaaacctcccacatctccccctgaacctgaaacataaaatgaatgcaattgttgttgttaacttgtttattgcagcttataatggttacaaataaagcaatagcatcacaaatttcacaaataaagctagaatggttacaaataaagcaatagcatcacaaatttcacaaataaacaccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatccatatgtca

SEQ ID NO: 70 - DNA, 2941 bp, synthetic construct
  misc_feature 1..2941: note = Description of sequence: Payload construct
  source 1..2941: mol_type = other DNA
ggtggcggatcccttctatggaagtcaaaacagcgtggatggcgtctccaggcgatctgacggttcactaaacgagctctgcttatatagagctcggggagcagaagcgcgcgaacagaagcgagaagcgaactgattggttagttcaaataaggcacagggtcatttcaggtccttggggcaccctggaaacatctgatggttctctagaaactgctgagggcgggaccgcatctggggaccatctgttcttggccctgagccggggcaggaactgcttaccacagatatcctgtttggcccatattctgctgttccaactgttcttggccctgagccggggcaggaactgcttaccacagatatcctgtttggcccatattctcctgtttctctgttcctcgagaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 71 - DNA, 3931 bp, synthetic construct
  misc_feature 1..3931: note = Description of sequence: Payload construct
  source 1..3931: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcgggctggggcggtcccctcccatttccatgacgtcatggttaccaagaggggcaagtagggcaccctttgaagctctcccgcagaagccacatcctctggaaagaagagtttataatactgagttagagatagcatcgccccaggccacgtgccgaggggagcaggctgggccgttacaccaccccccaaccgcaggtgcagcaaggccaacatgccaggctgggaggggctgccggcccctcgttgagggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtg

SEQ ID NO: 72 - DNA, 2774 bp, synthetic construct
  misc_feature 1..2774: note = Description of sequence: Payload construct
  source 1..2774: mol_type = other DNA
cctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagagagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 73 - DNA, 4140 bp, synthetic construct
  misc_feature 1..4140: note = Description of sequence: Payload construct
  source 1..4140: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccgccacc

SEQ ID NO: 74 - DNA, 2618 bp, synthetic construct
  misc_feature 1..2618: note = Description of sequence: Payload construct
  source 1..2618: mol_type = other DNA
gagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 75 - DNA, 4004 bp, synthetic construct
  misc_feature 1..4004: note = Description of sequence: Payload construct
  source 1..4004: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgagtgagtcagtgactcagtgagtcagtgactcagtgagtcactgactcactgagtcagtgactcaggaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgggatccgccacc

SEQ ID NO: 76 - DNA, 2619 bp, synthetic construct
  misc_feature 1..2619: note = Description of sequence: Payload construct
  source 1..2619: mol_type = other DNA
agagccaaacgggggtccggcgctaccaatttcagtttgctcaaacaggccggagacgtcgaggaaaatcctggcccccatatgaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacaggctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 77 - DNA, 4938 bp, synthetic construct
  misc_feature 1..4938: note = Description of sequence: Payload construct
  source 1..4938: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttgcggccgccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 78 - DNA, 2619 bp, synthetic construct; identical to SEQ ID NO: 76
  misc_feature 1..2619: note = Description of sequence: Payload construct
  source 1..2619: mol_type = other DNA

SEQ ID NO: 79 - DNA, 4302 bp, synthetic construct
  misc_feature 1..4302: note = Description of sequence: Payload construct
  source 1..4302: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttttaaaactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgaggatccgccacc

SEQ ID NO: 80 - DNA, 2619 bp, synthetic construct; identical to SEQ ID NO: 76
  misc_feature 1..2619: note = Description of sequence: Payload construct
  source 1..2619: mol_type = other DNA

SEQ ID NO: 81 - DNA, 4274 bp, synthetic construct
  misc_feature 1..4274: note = Description of sequence: Payload construct
  source 1..4274: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgagtcgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggatccgccacc

SEQ ID NO: 82 - DNA, 2619 bp, synthetic construct; identical to SEQ ID NO: 76
  misc_feature 1..2619: note = Description of sequence: Payload construct
  source 1..2619: mol_type = other DNA

SEQ ID NO: 83 - DNA, 4105 bp, synthetic construct
  misc_feature 1..4105: note = Description of sequence: Payload construct
  source 1..4105: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgagaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgggatccgccacc

SEQ ID NO: 84 - DNA, 2619 bp, synthetic construct; identical to SEQ ID NO: 76
  misc_feature 1..2619: note = Description of sequence: Payload construct
  source 1..2619: mol_type = other DNA

SEQ ID NO: 85 - DNA, 4007 bp, synthetic construct
  misc_feature 1..4007: note = Description of sequence: Payload construct
  source 1..4007: mol_type = other DNA
ggtaccacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattccgttacataacttacgttaaatggcccgcctggctgaccgcccaacgacccccgcccattgacgtcaataatgatgtatgttcccatagtaacgccaatagggactttccattgacgtcaatgggtggagtatttacggtaaactgcccacttggcagtacatcaagtgtatcatatccaagtacgccccctattgacgtcaatgacggtaaatggcccgcctggcattatgcccagtacatgaccttatgggactttcctacttggcagtacatctacgtattagtcatcgctattaccatgggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgtcgacgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcgaggggactttccgcttggggactttccgctggggactttccgctggggactttccgctggggactttccgaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgggatccgccacc

SEQ ID NO: 86 - DNA, 2619 bp, synthetic construct; identical to SEQ ID NO: 76
  misc_feature 1..2619: note = Description of sequence: Payload construct
  source 1..2619: mol_type = other DNA

SEQ ID NO: 87 - DNA, 2881 bp, synthetic construct
  misc_feature 1..2881: note = Description of sequence: Payload construct
  source 1..2881: mol_type = other DNA
ggtacctcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagcttgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatcc

SEQ ID NO: 88 - DNA, 2121 bp, synthetic construct
  misc_feature 1..2121: note = Description of sequence: Payload construct
  source 1..2121: mol_type = other DNA
tgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaagcgggctaagagaggctctggagccacaaactttagcctcctgaaacaagcaggggatgtggaagagaatcccggacctgcggccgcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 89 - DNA, 2887 bp, synthetic construct
  misc_feature 1..2887: note = Description of sequence: Payload construct
  source 1..2887: mol_type = other DNA
ggtacctcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagcttgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccgccacc

SEQ ID NO: 90 - DNA, 1855 bp, synthetic construct
  misc_feature 1..1855: note = Description of sequence: Payload construct
  source 1..1855: mol_type = other DNA
gtcgactgcgatgcggccgcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 91 - DNA, 3007 bp, synthetic construct
  misc_feature 1..3007: note = Description of sequence: Payload construct
  source 1..3007: mol_type = other DNA
ggtacctcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagcttgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagggatccctgactacatcacagcagcatacgtggagatgaggcgagaggcttgggctagtaaggatgccacctatacttctgcccggaccctgctggctatcctgcgcctttccactgctctggtaagtgccc

SEQ ID NO: 92 - DNA, 1970 bp, synthetic construct
  misc_feature 1..1970: note = Description of sequence: Payload construct
  source 1..1970: mol_type = other DNA
ttccccttaggcacgtctgagaatggtggatgtggtggagaaagaagatgtgaatgaagccatcaggctaatggagatgtcaaaggactctcttctaggagacaaggggcagacagtcgactgcgatgcggccgcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 93 - DNA, 312 bp, synthetic construct
  misc_feature 1..312: note = Description of sequence: Payload construct
  source 1..312: mol_type = other DNA
ggtaccagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctctgaattc

SEQ ID NO: 94 - DNA, 3532 bp, synthetic construct
  misc_feature 1..3532: note = Description of sequence: Payload construct
  source 1..3532: mol_type = other DNA
gtcgactgtcccacagatatccagaaccccgaccccgccgtgtaccagctgcgggacagcaagagcagcgacaagagcgtgtgcctgttggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgctgtgtatcagctgcgcgacagcaagagcagcgacaagagcgtgtgtttgttcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggcggccgctgcgatgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac

SEQ ID NO: 95 - DNA, 2881 bp, synthetic construct; identical to SEQ ID NO: 87
  misc_feature 1..2881: note = Description of sequence: Payload construct
  source 1..2881: mol_type = other DNA

SEQ ID NO: 96 - DNA, 1855 bp, synthetic construct; identical to SEQ ID NO: 90
  misc_feature 1..1855: note = Description of sequence: Payload construct
  source 1..1855: mol_type = other DNA

SEQ ID NO: 97 - DNA, 2736 bp, synthetic construct
  misc_feature 1..2736: note = Description of sequence: Payload construct
  source 1..2736: mol_type = other DNA
ggtacctcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcgaattcggctccggagccactaacttctccctgttgaaacaggctggcgatgttgaagaaaaccccggtcctatggccaccggctctagaacaagcctgctgctcgcttttggcctgctctgcctcccatggctccaagaaggatctgctgatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtctccgatggctatgttgtctccagatccaagacagagaatttccccctcactctggagtcagctacccgctcccagacatctgtgtatttctgcgccagcagtgaggacagttacgagcagtacttcgggccgggcaccaggctcacggtcacagaggacctgaaaaacgtgttccctccaaaagtggccgtgttcgagccttctgaggccgagatcagccacacacagaaagccacactcgtgtgtctggctaccggcttctaccccgatcacgtggaactgtcttggtgggtcaacggcaaagaggtgcacagcggcgtcagcacagatccccagcctctgaaagaacagcccgctctgaacgacagccgctactgcctgtctagcagactgagagtgtccgccaccttctggcagaaccccagaaaccacttcagatgccaggtccagttctacggcctgagcgagaacgatgagtggacccaggacagagccaagcctgtgacacagatcgtgtctgccgaagcctggggcagagccgattgtggctttaccagcgagtcataccagcagggcgtgctgtctgccaccatcctgtatgagatcctgctcggcaaggccacactgtacgctgtgctggtgtctgctctggtgctgatggctatggtctcccgggagcgcatccccgaggcccgggccaagcggggcagcggcgccaccaacttcagcctgctgaagcaggccggcgacgtggaggagaaccccggccctatggccacaggcagcagaacatctctgctgctggccttcggactgctgtgtctgccttggctgcaagagggttccgccgcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgaattggacacaggctttcagaaacttgtatttggaactggcacccgacttctggtcagtccaaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagcttgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 98 - DNA, 1861 bp, synthetic construct
  misc_feature 1..1861: note = Description of sequence: Payload construct
  source 1..1861: mol_type = other DNA
ttttttgtcgactgcgatgcggccgcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattgctagccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacac